(19) United States
(12) Patent Application Publication
(10) Pub. No.: US 2023/0241213 A1
(43) Pub. Date: Aug. 3, 2023

(54) COMPOSITIONS AND METHODS FOR THERAPEUTIC DELIVERY

(71) Applicant: CYTONUS THERAPEUTICS, INC., Columbus, OH (US)

(72) Inventors: Remo MOOMIAIE, Columbus, OH (US); Richard KLEMKE, Columbus, OH (US)

(21) Appl. No.: 18/176,259

(22) Filed: Feb. 28, 2023

Related U.S. Application Data
(63) Continuation of application No. PCT/US2022/018007, filed on Feb. 25, 2022.
(60) Provisional application No. 63/154,591, filed on Feb. 26, 2021.
(60) Provisional application No. 63/193,949, filed on May 27, 2021.

(51) Int. Cl. A61K 39/00; A61P 35/00

(52) U.S. Cl. CPC A61K 39/4636; A61K 39/4635; A61K 39/464499; A61K 39/461; A61P 35/00; A61K 39/4633

(57) ABSTRACT

Described herein are compositions for delivering single-domain antibodies or antigen-binding fragments thereof to a subject. The single-domain antibodies may be therapeutic agents for treatment of a disease or a condition in the subject, such as a disease or a condition of affecting the lungs of the subject. The compositions comprise enucleated cells that are extensively engineered to produce the single-domain antibodies or antigen-binding fragment thereof, and optionally, contain additional components, such as a targeting moiety, immune system evading moiety, or additional therapeutic agents or adjuvants. Methods of producing the compositions described herein are provided, which involve methods of enucleating a parent cell to obtain the enucleated cell comprising the single-domain antibody or antigen-binding fragment thereof. Also provided are kits and methods for using the compositions described herein to treat the disease or a condition by administering one or more of the compositions to the subject.

### CROSS-REFERENCE

This application is a continuation application of International Application No. PCT/US2022/018007, filed Feb. 25, 2022, which claims the benefit of U.S. Provisional Application Ser. No. 63/154,591 filed on Feb. 26, 2021, and U.S. Provisional Application Ser. No. 63/193,949, filed on May 27, 2021, each of which is hereby incorporated by reference herein in its entirety.

### SEQUENCE LISTING

The instant application contains a Sequence Listing which has been submitted electronically in XML file format and is hereby incorporated by reference in its entirety. Said XML copy, created on Dec. 21, 2022, is named 53712-710_301_SL.xml and is 1,030,169 bytes in size.

### SUMMARY

Described herein, in some aspects, is an enucleated cell, the enucleated cell comprising: a single-domain antibody or fragment thereof that binds an immune checkpoint molecule; and one or more intracellular organelles configured to translate an exogenous messenger ribonucleic acid (mRNA) molecule encoding the single-domain antibody or fragment thereof. In some embodiments, the single-domain antibody or fragment thereof is contained in the enucleated cell. In some embodiments, the single-domain antibody or fragment thereof is released by the enucleated cell. In some embodiments, the enucleated cell further comprises a cell membrane, wherein the single-domain antibody or fragment thereof is expressed on an exoplasmic side of the cell membrane. In some embodiments, the enucleated cell further comprises a cell membrane, wherein the cell membrane comprises a transmembrane moiety that is coupled to the single-domain antibody or fragment thereof. In some embodiments, the transmembrane moiety comprises a transmembrane polypeptide. In some embodiments, the single-domain antibody or fragment thereof is coupled with a N-terminus or a C-terminus of the transmembrane polypeptide. In some embodiments, the single-domain antibody or fragment is coupled to an anchor molecule coupled to a cell surface of the enucleated cell, wherein the anchor molecule comprises glycosylphosphatidylinositol, farnesyl, palmitate, myristate, or any combination thereof. In some embodiments, the enucleated further comprises a fusion protein configured to transfer the single-domain antibody or fragment thereof from the enucleated cell to another cell. In some embodiments, the single-domain antibody or fragment thereof is coupled to a cytotoxic drug. In some embodiments, the immune checkpoint molecule comprises programmed cell death protein 1 (PD-1 or PDCD-1), programmed death-ligand 1 (PD-L1), cytotoxic T-lymphocyte-associated protein 4 (CTLA-4, also known as cluster of differentiation 152 or CD152), V-domain Ig suppressor of T cell activation (VISTA), Programmed cell death 1 ligand 2 (PDCD1LG2, also known as cluster of differentiation 273 or CD273), B7 homolog 3 (B7-H3, also known as cluster of differentiation 276 or CD276), adenosine A2A receptor (A2AR), cluster of differentiation 27 (CD27), lymphocyte-activation gene 3 (LAG3), T-cell immunoglobulin and mucin-domain containing-3 (TIM-3, also known as Hepatitis A virus cellular receptor 2 or HAVCR2), T cell immunoreceptor with Ig and ITIM domains (TIGIT), cluster of differentiation 73 (CD73), CD94/NK group 2 member A (NKG2A, also known as cluster of differentiation 159 or CD159), Poliovirus receptor related immunoglobulin domain containing (PVRIG), Poliovirus receptor-related 2 (PVRL2), carcinoembryonic antigen-related cell adhesion molecule 1 (CEACAM1), Carcinoembryonic antigen-related cell adhesion molecule 5 (CEACAM5), carcinoembryonic antigen-related cell adhesion molecule 6 (CEACAM6), focal adhesion kinase (FAK), C-C chemokine receptor type 2 (CCR-2), chemokine (C-C motif) ligand 2 (CCL-2), leukemia inhibitory factor (LIF), cluster of differentiation 47 (CD47), signal-regulatory protein alpha (SIRPα), macrophage colony-stimulating factor (M-CSF), colony stimulating factor 1 receptor (CSF-1R), interleukin 3 (IL-3), Interleukin-1 receptor accessory protein (IL-1RAP), interleukin 8 (IL-8), semaphorin-4D (SEMA4D), angiopoietin-2, CLEVER-1, tyrosine-protein kinase receptor UFO (Axl), phosphatidylserine or a fragment thereof. In some embodiments, the immune checkpoint molecule comprises PD-L1. In some embodiments, the immune checkpoint molecule comprises CTLA-4. In some embodiments, the immune checkpoint molecule comprises an amino acid sequence that is greater than or equal to about 80% identical to any one of SEQ ID NOs: 155-164, 203, 204, 315-322, 511, 531-535, 551-554, 571, 594, 611-619, or 711. In some embodiments, the single-domain antibody or fragment thereof is encoded by a deoxyribonucleic acid (DNA) sequence that is greater than or equal to about 50%, 55%, 60%, 65%, 70%, 75%, 80%, 85%, 90%, 95%, or 99% identical to SEQ ID NO: 801. In some embodiments, the single-domain antibody or fragment thereof comprises an amino acid sequence that is greater than or equal to about 50%, 55%, 60%, 65%, 70%, 75%, 80%, 85%, 90%, 95%, or 99% identical to SEQ ID NO: 851. In some embodiments, the single-domain antibody or fragment thereof is encoded from a DNA sequence that is greater than or equal to about 50%, 55%, 60%, 65%, 70%, 75%, 80%, 85%, 90%, 95%, or 99% identical to SEQ ID NO: 901. In some embodiments, the single-domain antibody or fragment thereof comprises an amino acid sequence that is greater than or equal to about 50%, 55%, 60%, 65%, 70%, 75%, 80%, 85%, 90%, 95%, or 99% identical to SEQ ID NO: 951. In some embodiments, the enucleated cell further comprises a targeting moiety. In some embodiments, the targeting moiety comprises a homing receptor specific to a ligand expressed by a cell in lung tissue. In some embodiments, the targeting moiety comprises an antibody or antigen-binding fragment thereof, wherein said antibody or antigen binding fragment thereof is different from the single-domain antibody or fragment thereof. In some embodiments, the targeting moiety comprises a chemokine receptor. In some embodiments, the cell is a cancer cell. In some embodiments, the cancer cell is a cell of non-small cell lung cancer (NSCLC), small cell lung cancer (SCLC), adenocarcinoma, squamous carcinoma, large cell (undifferentiated) carcinoma, large cell neuroendocrine carcinoma, adenosquamous carcinoma, or sarcomatoid carcinoma. In some embodiments, the cancer cell is a cell of benign lung tumor. In some embodiments, the cancer cell is a cell of hamartoma. In some embodiments, the enucleated cell further comprises a therapeutic agent. In some embodiments, the therapeutic agent comprises interleukin 12 (IL-12). In some embodiments, the enucleated cell further comprises an immune evasion moiety comprising cluster of differentiation (CD47), PD-L1, major histocompatibility complex, class I, E (HLA-E), major histocompatibility complex, class I, G (HLA-G), a fragment thereof, or a combination thereof. In some embodiments, the enucleated cell has a diameter comprising between about 1 micrometers (μm) to about 100 μm. In some embodiments, the diameter comprises between about 5 μm to 25 μm. In some embodiments, the diameter comprises between about 8 μm to 12 μm. In some embodiments, the enucleated cell exhibits a diameter that reduced relative to an otherwise identical nucleated cell, wherein the diameter is reduced by great than or equal to about 50%. In some embodiments, the enucleated cell further comprises an exogenous tumor necrosis factor (TNF) superfamily member polypeptide or a catalytically active fragment thereof. In some embodiments, the exogenous TNF superfamily member polypeptide or the catalytically active fragment thereof is soluble in aqueous conditions. In some embodiments, the exogenous TNF superfamily member polypeptide comprises tumor necrosis factor superfamily member 14 (LIGHT), or a catalytically active fragment thereof. In some embodiments, the enucleated cell was obtained from a parent cell, wherein the parent cell comprises a stem cell. In some embodiments, the stem cell comprises an induced pluripotent stem cell (iPSC), an adult stem cell, a mesenchymal stromal cell, an embryonic stem cell, or a fibroblast. In some embodiments, the enucleated cell is purified. In some embodiments, the enucleated cell is lyophilized.

Described herein, in some aspects, is a plurality of cells, comprising: a plurality of enucleated cells comprising: a single-domain antibody or fragment thereof that binds an immune checkpoint molecule; and one or more intracellular organelles configured to translate an exogenous messenger ribonucleic acid (mRNA) molecule encoding the single-domain antibody or fragment thereof.

Described herein, in some aspects, is a pharmaceutical formulation, comprising: an enucleated cell comprising: a single-domain antibody or fragment thereof that binds an immune checkpoint molecule; and one or more intracellular organelles configured to translate an exogenous messenger ribonucleic acid (mRNA) molecule encoding the single-domain antibody or fragment thereof; and a pharmaceutically acceptable excipient, carrier or diluent.

Described herein, in some aspects, is a method of delivering an enucleated cell comprising: a single-domain antibody or fragment thereof that binds an immune checkpoint molecule; and one or more intracellular organelles configured to translate an exogenous messenger ribonucleic acid (mRNA) molecule encoding the single-domain antibody or fragment thereof to a subject, the method comprising: delivering to the subject an enucleated cell described herein. In some embodiments, the enucleated cell is an autologous cell. In some embodiments, the enucleated cell is an allogenic cell. In some embodiments, the administering is performed by systemic administration. In some embodiments, following the administering, the enucleated cell is viable in the subject for fewer than or equal to 5 days.

Described herein, in some aspects, is a method of treating cancer in a subject, the method comprising: administering to the subject a therapeutically effective amount of an enucleated cell the enucleated cell comprising: a single-domain antibody or fragment thereof that binds an immune checkpoint molecule; and one or more intracellular organelles configured to translate an exogenous messenger ribonucleic acid (mRNA) molecule encoding the single-domain antibody or fragment thereof; or pharmaceutical formulation described herein, thereby treating the cancer in the subject. In some embodiments, the enucleated cell is an autologous cell. In some embodiments, the enucleated cell is an allogenic cell. In some embodiments, the administering is performed by systemic administration. In some embodiments, following the administering, the enucleated cell is viable in the subject for fewer than or equal to 5 days.

Described herein, in some aspects, an enucleated cell, the enucleated cell comprising: a single-domain antibody or fragment thereof that binds a connective tissue growth factor (CTGF); and one or more intracellular organelles configured to (i) translate an exogenous mRNA molecule encoding the single-domain antibody or fragment thereof, and (ii) release the single-domain antibody or fragment thereof from the enucleated cell. In some embodiments, the single-domain antibody or fragment thereof comprises a polypeptide sequence that is greater than or equal to about 50%, 55%, 60%, 65%, 70%, 75%, 80%, 85%, 90%, 95%, or 99% identical to SEQ ID NO: 1701. In some embodiments, the single-domain antibody or fragment thereof binds to an amino acid sequence of CTGF, wherein the amino acid sequence of CTGF comprises SEQ ID NO: 1601 or SEQ ID NO: 1602. In some embodiments, the enucleated cell, further comprises a targeting moiety specific to a ligand expressed by a cell in lung tissue. In some embodiments, the targeting moiety comprises a homing receptor specific to the ligand expressed by the cell in lung tissue. In some embodiments, the cell is an alveolar epithelial cell (AEC). In some embodiments, the cell is a bronchial cell. In some embodiments, the enucleated cell further comprises an immune evasion moiety comprising CD47, PD-L1, HLA-E, HLA-G, a fragment thereof, or any combination thereof. In some embodiments, the targeting moiety comprises a chemokine receptor. In some embodiments, the targeting moiety comprises an adhesion molecule. In some embodiments, the target moiety comprises an antibody or antigen-binding fragment thereof, wherein said antibody or antigen binding fragment thereof is different from the single-domain antibody or fragment thereof. In some embodiments, the enucleated cell has a diameter comprising between about 1 micrometers (μm) to about 100 μm. In some embodiments, the diameter is between about 5 μm to 25 μm. In some embodiments, the diameter comprises between about 8 μm to 12 μm. In some embodiments, the enucleated cell exhibits a diameter that reduced relative to an otherwise identical nucleated cell, wherein the diameter is reduced by great than or equal to about 50%. In some embodiments, the enucleated cell further comprises an exogenous tumor necrosis factor (TNF) superfamily member polypeptide or a catalytically active fragment thereof. In some embodiments, the exogenous TNF superfamily member polypeptide or the catalytically active fragment thereof is soluble in aqueous conditions. In some embodiments, the exogenous TNF superfamily member polypeptide comprises LIGHT or catalytically active fragment thereof. In some embodiments, the enucleated cell was obtained from a parent cell, wherein the parent cell comprises a stem cell. In some embodiments, the stem cell comprises an induced pluripotent stem cell (iPSC), an adult stem cell, a mesenchymal stromal cell, an embryonic stem cell, or a fibroblast. In some embodiments, the enucleated cell is purified. In some embodiments, the enucleated cell is lyophilized.

Described herein, in some aspects, is a plurality of cells, comprising: a plurality of the enucleated cells comprising: a single-domain antibody or fragment thereof that binds a connective tissue growth factor (CTGF); and one or more intracellular organelles configured to (i) translate an exogenous mRNA molecule encoding the single-domain antibody or fragment thereof, and (ii) release the single-domain antibody or fragment thereof from the enucleated cell.

Described herein, in some aspects, is a pharmaceutical formulation, comprising: an enucleated cell comprising: a single-domain antibody or fragment thereof that binds a connective tissue growth factor (CTGF); and one or more intracellular organelles configured to (i) translate an exogenous mRNA molecule encoding the single-domain antibody or fragment thereof, and (ii) release the single-domain antibody or fragment thereof from the enucleated cell; and a pharmaceutically acceptable excipient, carrier, or diluent.

Described herein, in some aspects, is a method of delivering an enucleated cell to a subject, the method comprising: delivering to the subject the enucleated cell comprising: a single-domain antibody or fragment thereof that binds a connective tissue growth factor (CTGF); and one or more intracellular organelles configured to (i) translate an exogenous mRNA molecule encoding the single-domain antibody or fragment thereof, and (ii) release the single-domain antibody or fragment thereof from the enucleated cell or the pharmaceutical formulation described herein. In some embodiments, the enucleated cell is an autologous cell. In some embodiments, the enucleated cell is an allogenic cell. In some embodiments, the administering is performed by systemic administration. In some embodiments, following the administering, the enucleated cell is viable in the subject for fewer than or equal to 5 days.

Described herein, in some aspects, is a method of treating idiopathic pulmonary fibrosis (IPF) in a subject in need thereof, the method comprising: administering to the subject a therapeutically effective amount of an enucleated cell comprising: a single-domain antibody or fragment thereof that binds a connective tissue growth factor (CTGF); and one or more intracellular organelles configured to (i) translate an exogenous mRNA molecule encoding the single-domain antibody or fragment thereof, and (ii) release the single-domain antibody or fragment thereof from the enucleated cell or the pharmaceutical formulation described herein. In some embodiments, the enucleated cell is an autologous cell. In some embodiments, the enucleated cell is an allogenic cell. In some embodiments, the administering is performed by systemic administration. In some embodiments, following the administering, the enucleated cell is viable in the subject for fewer than or equal to 5 days.

Described herein, in some aspects, is a method of treating a disease or condition in a subject in need thereof, the method comprising: administering to the subject having the disease or condition associated with a target cell in the subject a therapeutically effective amount of an enucleated cell comprising: a single-domain antibody or fragment thereof that binds a connective tissue growth factor (CTGF); and one or more intracellular organelles configured to (i) translate an exogenous mRNA molecule encoding the single-domain antibody or fragment thereof, and (ii) release the single-domain antibody or fragment thereof from the enucleated cell, wherein the exogenous TNF superfamily member polypeptide or the catalytically active fragment thereof normalizes a vasculature associated with the disease or condition, and wherein normalizing the vasculature increases therapeutic efficacy of treating the disease or condition compared to a therapeutic efficacy of a comparable method without normalizing the vasculature.

Described herein, in some aspects, is a method of treating a disease or condition characterized, at least in part, by an abnormal vasculature in a subject, the method comprising: administering to the subject having the disease or the condition an enucleated cell comprising: a single-domain antibody or fragment thereof that binds a connective tissue growth factor (CTGF); and one or more intracellular organelles configured to (i) translate an exogenous mRNA molecule encoding the single-domain antibody or fragment thereof, and (ii) release the single-domain antibody or fragment thereof from the enucleated cell, wherein the exogenous tumor necrosis factor (TNF) superfamily member polypeptide or the catalytically active fragment thereof synthesized or released by the enucleated cell is therapeutically effective to normalize the abnormal vasculature in the subject.

Provided herein, in some embodiments, are enucleated cells comprising an antibody such as a single-domain antibody. Also provided are methods of delivering the enucleated cells described herein to a subject such as, for example to treat a disease or a condition of the subject. The enucleated cells and pharmaceutical compositions containing such enucleated cells, and methods of their use offer several benefits over previous cell-based therapeutics, including, safety, defined lifespan, no risk of nuclear-encoded gene transfer to host, and effective delivery of therapeutic cargo to target cells or tissues even when administered systemically.

Aspects disclosed herein provide enucleated cells obtained from a parent cell with a nucleus, the enucleated cell comprising: one or more intracellular organelles for synthesis of an exogenous single-domain antibody or fragment thereof in absence of the nucleus. In some embodiments, the exogenous single-domain antibody or fragment thereof is encapsulated in the enucleated cell. In some embodiments, the exogenous single-domain antibody or fragment thereof is expressed on an exoplasmic side of a cell membrane of the enucleated cell by the one or more intracellular organelles. In some embodiments, the exogenous single-domain antibody or fragment thereof is expressed on a cytosolic side of a cell membrane of the enucleated cell by the one or more intracellular organelles. In some embodiments, the exogenous single-domain antibody or fragment thereof is complexed with a transmembrane moiety. In some embodiments, the transmembrane moiety comprises a transmembrane polypeptide. In some embodiments, the exogenous single-domain antibody or fragment thereof is complexed with N-terminus of the transmembrane polypeptide. In some embodiments, the exogenous single-domain antibody or fragment thereof is complexed with C-terminus of the transmembrane polypeptide. In some embodiments, the exogenous single-domain antibody or fragment thereof comprises a modification relative to an otherwise identical reference single-domain antibody or fragment thereof, wherein the modification anchors the exogenous single-domain antibody or fragment thereof to an exoplasmic or a cytosolic side of a cell membrane of the enucleated cell. In some embodiments, the modification comprises complexing the exogenous single-domain antibody or fragment thereof to glycosylphosphatidylinositol, farnesyl, palmitate, myristate, or a combination thereof. In some embodiments, the exogenous single-domain antibody or fragment thereof is released by the enucleated cell by secreting the exogenous single-domain antibody or fragment thereof from the enucleated cell. In some embodiments, the exogenous single-domain antibody or fragment thereof is released upon death of the enucleated cell. In some embodiments, the exogenous single-domain antibody or fragment thereof is released upon rupture of the enucleated cell. In some embodiments, the exogenous single-domain antibody or fragment thereof is transferred from the enucleated cell to another cell by fusing the enucleated cell with the another cell. In some embodiments, the exogenous single-domain antibody or fragment thereof is conjugated to a cytotoxic drug. In some embodiments, the enucleated cell comprises an exogenous nucleotide having a polypeptide sequence that encodes the exogenous single-domain antibody or fragment thereof. In some embodiments, the polypeptide sequence comprises a sequence provided in SEQ ID NOs: 1-36, 101-111, 121-123, 165-192, 195, 205, 206, 211-213, 221-231, 241-245, 325-331, and 401-404. In some embodiments, the exogenous single-domain antibody or fragment thereof is specific to an antigen encoded by at least one nucleic acid in SEQ ID NOs: 131-134, 142-152, 201-202, 301-312, 501, 521-526, 541-545, 561, 584, 591-601, and 701-705. In some embodiments, the exogenous single-domain antibody or fragment thereof is specific to an antigen comprising a peptide sequence encoding PD-L1. In some embodiments, the exogenous single-domain antibody or fragment thereof is specific to an antigen comprising at least one peptide sequence in SEQ ID NOs: 155-164, 203, 204, 315-322, 511, 531-535, 551-554, 571, 594, 611-619, and 711. In some embodiments, the exogenous single-domain antibody or fragment thereof is specific to an antigen associated with at least one pathogen in Table 1. In some embodiments, the exogenous single-domain antibody or fragment thereof is specific to an antigen comprising a peptide sequence encoding Connective tissue growth factor (CTGF) also known as Cellular Communication Network Factor 2 (CCN2). In some embodiments, the exogenous single-domain antibody or fragment thereof is specific to an antigen comprising at least one peptide sequence in SEQ ID NOs: 1601 and 1602.

In some embodiments, the exogenous single-domain antibody or fragment thereof is encoded from a nucleic acid sequence that is greater than or equal to about 50%, 55%, 60%, 65%, 70%, 75%, 80%, 85%, 90%, 95%, or 99% identical to SEQ ID NO: 801. In some embodiments, the exogenous single-domain antibody or fragment thereof comprises a polypeptide sequence that is greater than or equal to about 50%, 55%, 60%, 65%, 70%, 75%, 80%, 85%, 90%, 95%, or 99% identical to SEQ ID NO: 851. In some embodiments, the exogenous single-domain antibody or fragment thereof is encoded from a nucleic acid sequence that is greater than or equal to about 50%, 55%, 60%, 65%, 70%, 75%, 80%, 85%, 90%, 95%, or 99% identical to SEQ ID NO: 901. In some embodiments, the exogenous single-domain antibody or fragment thereof comprises a polypeptide sequence that is greater than or equal to about 50%, 55%, 60%, 65%, 70%, 75%, 80%, 85%, 90%, 95%, or 99% identical to SEQ ID NO: 951. In some embodiments, the exogenous single-domain antibody or fragment thereof comprises a polypeptide sequence that is greater than or equal to about 50%, 55%, 60%, 65%, 70%, 75%, 80%, 85%, 90%, 95%, or 99% identical to SEQ ID NO: 1701. In some embodiments, the exogenous single-domain antibody or fragment thereof is specific to an antigen expressed by a cancer cell in lung tissue. In some embodiments, the antigen expressed by the cancer cell in lung tissue is PD-L1. In some embodiments, the antigen expressed by the cancer cell in lung tissue is CTGF. In some embodiments, the cancer cell is a non-small cell lung cancer (NSCLC) cell. In some embodiments, the cancer cell is a cell of adenocarcinoma, squamous carcinoma, large cell (undifferentiated) carcinoma, large cell neuroendocrine carcinoma, adenosquamous carcinoma, or sarcomatoid carcinoma. In some embodiments, the cancer cell is a cell of small cell lung cancer (SCLC). In some embodiments, the cancer cell is a cell of lung carcinoid tumor. In some embodiments, the cancer cell is a cell of adenoid cystic carcinoma. In some embodiments, the cancer cell is a cell of lymphoma. In some embodiments, the cancer cell is a cell of sarcoma. In some embodiments, the cancer cell is a cell of benign lung tumor. In some embodiments, the cancer cell is a cell of hamartoma. In some embodiments, the exogenous single-domain antibody or fragment thereof is specific to an antigen expressed by a cell associated with idiopathic pulmonary fibrosis. In some embodiments, the exogenous single-domain antibody or fragment thereof is specific to an antigen expressed by the cell associated with idiopathic pulmonary fibrosis is a lung cell. In some embodiments, the exogenous single-domain antibody or fragment thereof is specific to an antigen expressed by the cell associated with idiopathic pulmonary fibrosis is an immune cell. In some embodiments, the exogenous single-domain antibody or fragment thereof is specific to an antigen expressed by the cell associated with idiopathic pulmonary fibrosis is an alveolar cell. In some embodiments, the exogenous single-domain antibody or fragment thereof is specific to an antigen expressed by the cell associated with idiopathic pulmonary fibrosis is an alveolar epithelial cell (AEC). In some embodiments, the exogenous single-domain antibody or fragment thereof is specific to an antigen expressed by the cell associated with idiopathic pulmonary fibrosis is a bronchial cell. In some embodiments, the enucleated cell further comprises at least one additional exogenous therapeutic agent. In some embodiments, the enucleated cell further comprises a fusogenic moiety. In some embodiments, the fusogenic moiety comprises a viral fusogenic moiety. In some embodiments, the fusogenic moiety comprises an eukaryotic fusogenic moiety. In some embodiments, the enucleated cell further comprises an immune evasion moiety. In some embodiments, the immune evasion moiety comprises CD47, PD-L1, HLA-E, HLA-G, a fragment thereof, or a combination thereof. In some embodiments, the enucleated cell further comprises a targeting moiety. In some embodiments, the targeting moiety targets a biomarker of the cancer cell. In some embodiments, the exogenous single-domain antibody or fragment thereof is specific to an antigen expressed by a cancer cell, and wherein the biomarker is a separate and distinct entity from the antigen targeted by the exogenous single-domain antibody or fragment thereof. In some embodiments, the targeting moiety targets a biomarker of an immune cell within the microenvironment of the tumor. In some embodiments, the biomarker is expressed on surface of the immune cell. In some embodiments, the biomarker is released by the immune cell. In some embodiments, the targeting moiety comprises a chemokine. In some embodiments, the targeting moiety comprises a chemokine receptor. In some embodiments, the targeting moiety comprises an adhesion molecule. In some embodiments, the targeting moiety comprises an antigen. In some embodiments, the targeting moiety comprises an antigen that is a separate and distinct entity from an antigen expressed by the cancer cell. In some embodiments, the targeting moiety comprises an antibody that is not expressed by the cancer cell. In some embodiments, the target moiety comprises a membrane-bound antibody. In some embodiments, the membrane bound antibody is a membrane-bound single-domain antibody. In some embodiments, the enucleated cell has a diameter comprising between about 1 micrometers (μm) to about 100 μm. In some embodiments, the diameter comprises between about 1 μm to about 10 μm. In some embodiments, the diameter comprises between about 10 μm to about 100 μm. In some embodiments, the diameter is at least or about 1 μm, 5 μm, 8 μm, 10 μm, 20 μm, 30 μm, 40 μm, 50 μm, 60 μm, 70 μm, 80 μm, 90 μm, or 100 μm. In some embodiments, the diameter comprises about 8 μm. In some embodiments, the enucleated cell exhibits a diameter that is at least about 10%, 20%, 30%, 40%, 50%, 60%, 70%, 80%, 90%, 95%, or 99% smaller compared to the parent cell that is nucleated. In some embodiments, the parent cell is selected from the group consisting of: a stem cell, an induced pluripotent stem cell (iPSC), an adult stem cell, a mesenchymal stromal cell, an embryonic stem cell, a fibroblast, and a cell from a cell line. In some embodiments, the parent cell is mesenchymal stromal cell. In some embodiments, the enucleated cell exhibits viability after cryohibernation. In some embodiments, the enucleated cell exhibits the viability following the cryohibernation as measured at 24 hours following the cryohibernation that is equal to or greater than the viability of a comparable enucleated cell that is not cryohibernated. In some embodiments, the enucleated cell exhibits viability after cryopreservation. In some embodiments, the enucleated cell exhibits the viability following the cryopreservation as measured at 24 hours following the cryopreservation that is equal to or greater than the viability of a comparable enucleated cell that is not cryopreserved. In some embodiments, the enucleated cell is isolated. In some embodiments, the enucleated cell is purified. In some embodiments, the enucleated cell is lyophilized. In some embodiments, the enucleated cell comprises the exogenous single-domain antibody or fragment thereof comprising a neutralizing antibody. In some embodiments, the exogenous single-domain antibody or fragment thereof binds a VEGF. In some embodiments, the exogenous single-domain antibody or fragment thereof binds a VEGF-A. In some embodiments, the enucleated cell comprises a targeting moiety that targets an endothelial cell biomarker. In some embodiments, the endothelial cell biomarker is expressed by a vasculature cell. In some embodiments, the endothelial cell biomarker is expressed by a blood vessel cell. In some embodiments, the endothelial cell biomarker is expressed by a lymphatic vessel cell. In some embodiments, the enucleated cell comprises at least one additional exogenous agent comprising a polypeptide comprising a tumor necrosis factor (TNF) superfamily member polypeptide or a catalytically active fragment thereof. In some embodiments, the TNF superfamily member polypeptide or the catalytically active fragment thereof soluble in aqueous conditions, when solubility is measure in vitro by turbidimetric solubility assay or thermodynamic solubility assay. In some embodiments, the TNF superfamily member polypeptide comprises tumor necrosis factor superfamily member 14 (TNFSF14, also known as LIGHT). In some embodiments, the TNF superfamily member polypeptide comprises soluble LIGHT. In some embodiments, the at least one additional exogenous agent comprises a immune checkpoint molecule. In some embodiments, the at least one additional exogenous agent comprises a immune checkpoint inhibitor molecule. In some embodiments, the at least one additional exogenous agent comprises an angiogenesis inhibitor. In some embodiments, the angiogenesis inhibitor comprises a VEGF/VEGFR inhibitor. In some embodiments, the VEGF/VEGFR inhibitor comprises a VEGF-A inhibitor.

Aspects disclosed herein provide a cell line comprising the enucleated cell described herein.

Aspects disclosed herein provide a plurality of cells comprising the enucleated cell described herein.

Aspects disclosed herein provide pharmaceutical compositions comprising: the enucleated cell described herein; and a pharmaceutically acceptable: excipient, carrier, or diluent. In some embodiments, the pharmaceutical composition comprises a unit dose form. In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof. In some embodiments, the pharmaceutical composition is formulated for administering intravenously. In some embodiments, the pharmaceutical composition is formulated for administering intratumorally. In some embodiments, the pharmaceutical composition is formulated for administering pulmonarily. In some embodiments, the pharmaceutical is formulated for administering endotracheally. In some embodiments, the pharmaceutical composition is formulated for administering by inhaled nebulized form. In some embodiments, the pharmaceutical composition comprises at least one additional active agent. In some embodiments, the at least one additional active agent comprises a cytokine, a growth factor, a hormone, an antibody, an enzyme, a small molecule, a compound, or combinations thereof.

Aspects disclosed herein provide kits comprising: the enucleated cell described herein, the cell line described herein, the plurality of cells described herein, or the pharmaceutical composition described herein; and a container.

Aspects disclosed herein provide method of treating a disease or condition in a subject in need thereof, the method comprising: administering to the subject having the disease or the condition associated with a target cell in the subject a therapeutically effective amount of cell disclosed herein or the pharmaceutical composition disclosed herein, wherein the exogenous single-domain antibody or fragment thereof binds to an antigen expressed by the target cell in the subject, thereby treating the disease or the condition in the subject. In some embodiments, the enucleated cell is an autologous cell. In some embodiments, the enucleated cell is an allogenic cell. In some embodiments, the antigen comprises tumor-associated antigen (TAA). In some embodiments, the antigen comprises tumor-specific antigen (TSA). In some embodiments, the binding of the exogenous single-domain antibody or fragment thereof to the antigen directly kills the cancer cell. In some embodiments, the binding of the exogenous single-domain antibody or fragment thereof to the antigen disrupts cell cycle signaling of the cancer cell. In some embodiments, the binding of the exogenous single-domain antibody or fragment thereof to the antigen disrupts angiogenesis signaling of the cancer cell. In some embodiments, the binding of the exogenous single-domain antibody or fragment thereof to the antigen recruits an immune cell to the cancer cell. In some embodiments, the immune cell is a T cell. In some embodiments, the enucleated cell or the pharmaceutical composition is administered to the subject intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof. In some embodiments, the enucleated cell or the pharmaceutical composition is administered intravenously. In some embodiments, the enucleated cell or the pharmaceutical composition is administered intratumorally. In some embodiments, the enucleated cell or the pharmaceutical composition is administered pulmonarily. In some embodiments, the enucleated cell or the pharmaceutical composition is administered endotracheally. In some embodiments, the enucleated cell or the pharmaceutical composition is administered by inhaled nebulized form. In some embodiments, following administration of the enucleated cell or the pharmaceutical composition to the subject, the enucleated cell is viable fewer than or equal to 14 days in the subject. In some embodiments, following administration of the enucleated or the pharmaceutical composition to the subject, the enucleated cell is viable fewer than or equal to 4 days in the subject. In some embodiments, the target cell is a cancer cell. In some embodiments the disease or the condition is cancer or a neoplasm.

Aspects described herein provide an enucleated cell obtained from a parent cell with a nucleus, the enucleated cell comprising: one or more intracellular organelles for synthesis of an exogenous polypeptide comprising a tumor necrosis factor (TNF) superfamily member polypeptide or a catalytically active fragment thereof in absence of the nucleus. In some embodiments, the enucleated cell comprises at least one exogenous targeting moiety. In some embodiments, the exogenous polypeptide comprises a solubility of at least 0.0001 mg/ml, 0.0005 mg/ml, 0.001 mg/ml, 0.005 mg/ml, 0.01 mg/ml, 0.05 mg/ml, 0.1 mg/ml, 0.5 mg/ml, 1.0 mg/ml, 5.0 mg/ml, 10 mg/ml, 50 mg/ml, 100 mg/ml, 500 mg/ml 1,000 mg/ml 5,000 mg/ml, 10,000 mg/ml, 50,000 mg/ml, or 100,000 mg/ml in aqueous conditions when solubility is measured by turbidimetric solubility assay or thermodynamic solubility assay. In some embodiments, the exogenous polypeptide is expressed on an exoplasmic side of a cell membrane of the enucleated cell by the one or more intracellular organelles. In some embodiments, the exogenous polypeptide is released by the enucleated cell. In some embodiments, the enucleated cell further comprises an exogenous polynucleotide encoding the exogenous polypeptide. In some embodiments, the exogenous polypeptide comprises a sequence that is at least 75%, 80%, 85%, 90%, 95%, or 99% identical to SEQ ID NOs: 1501-1511. In some embodiments, the exogenous polypeptide comprises a sequence that is at least 75%, 80%, 85%, 90%, 95%, or 99% identical to SEQ ID NO: 1511. In some embodiments, the TNF superfamily member polypeptide is LIGHT. In some embodiments, the enucleated cell further comprises a second exogenous polypeptide. In some embodiments, the second exogenous polypeptide comprises an antibody, an immune checkpoint molecule, or a fragment thereof. In some embodiments, the second exogenous polypeptide comprises an antibody or an antigen-binding fragment thereof, or a single-domain antibody or an antigen-binding fragment thereof. In some embodiments, the antibody or the antigen-binding fragment thereof, or the single-domain antibody or the antigen-binding fragment thereof is a neutralizing antibody or neutralizing antigen-binding fragment thereof. In some embodiments, the neutralizing antibody or neutralizing antigen-binding fragment thereof targets an immune checkpoint molecule. In some embodiments, the neutralizing antibody or neutralizing antigen-binding fragment thereof targets Angiopoitin-1, Angiopoitin-2, Endostatin, FGF, MMP, DII4, Class 3 semaphorins, FGF, VEGFR, NRP-1, PDGF (BB-homodimer), PDGFR, TGF-β, endoglin, TGF-β receptors, CCL2, Integrins αVβ3, αVβ5, or α5β1, VE-cadherin, CD31, ephrin, plasminogen activator, plasminogen activator inhibitor-1, eNOS, COX-2, AC133, ID1/ID3, Class 3 semaphorin, or Nogo-A. In some embodiments, the neutralizing antibody or neutralizing antigen-binding fragment thereof targets VEGF. In some embodiments, the neutralizing antibody or neutralizing antigen-binding fragment thereof targets VEGF-A. In some embodiments, the immune checkpoint molecule comprises PD-1, PD-L1, CTLA-4, VISTA, PDCD1LG2 (CD273), B7-H3 (also called CD276), A2AR, CD27, LAG3, TIM-3, T cell immunoreceptor with Ig and ITIM domains (TIGIT), CD73, NKG2A, PVRIG, PVRL2, CEACAM1, CEACAM5, CEACAM6, FAK, CCR-2, CCL-2, LIF, CD47, SIRPα, M-CSF, CSF-1R, IL-3, IL-1RAP, IL-8, SEMA4D, Angiopoietin-2, CLEVER-1, Axl, phosphatidylserine or a fragment thereof. In some embodiments, the at least one exogenous targeting moiety comprises an antibody or an antigen-binding fragment thereof, or a single-domain antibody or an antigen-binding fragment thereof. In some embodiments, the antibody or the antigen-binding fragment thereof, or the single-domain antibody or the antigen-binding fragment thereof comprises an exogenous single-domain antibody or fragment thereof. In some embodiments, the antibody or the antigen-binding fragment thereof or the single-domain antibody or the antigen binding domain thereof targets a cancer cell marker. In some embodiments, the antibody or the antigen-binding fragment thereof, or the single-domain antibody or the antigen-binding fragment thereof targets an endothelial cell biomarker. In some embodiments, the endothelial cell biomarker is expressed by a vasculature cell. In some embodiments, the endothelial cell biomarker is expressed by a blood vessel cell. In some embodiments, the endothelial cell biomarker is expressed by a lymphatic vessel cell.

Aspects provided herein are a method of treating a disease or condition characterized, at least in part, by abnormal vasculature in a subject, the method comprising: administering to the subject having the disease or the condition an enucleated cell comprising one or more intracellular organelles that synthesizes or releases an exogenous polypeptide comprising a tumor necrosis factor (TNF) superfamily member polypeptide or a catalytically active fragment thereof in absence of the nucleus, wherein the exogenous polypeptide synthesized or released by the cell is therapeutically effective to normalize the abnormal vasculature in the subject. In some embodiments, the exogenous polypeptide comprises a soluble TNF superfamily member polypeptide. In some embodiments, the exogenous polypeptide is released by the enucleated cell. In some embodiments, the exogenous polypeptide comprises a polypeptide sequence that is at least 75%, 80%, 85%, 90%, 95%, or 99% identical to SEQ ID NOs: 1501-1511. In some embodiments, the exogenous polypeptide comprises a polypeptide sequence that is at least 75%, 80%, 85%, 90%, 95%, or 99% identical to SEQ ID NO: 1508. In some embodiments, the exogenous polypeptide comprises a polypeptide sequence that is at least 75%, 80%, 85%, 90%, 95%, or 99% identical to SEQ ID NO: 1511. In some embodiments, the TNF superfamily member is LIGHT. In some embodiments, the enucleated cell further comprises at least one exogenous targeting moiety comprising an antibody or an antigen-binding fragment thereof, or a single-domain antibody or an antigen-binding fragment thereof. In some embodiments, the antibody or the antigen-binding fragment thereof, or the single-domain antibody or the antigen-binding fragment thereof comprises an exogenous single-domain antibody or fragment thereof. In some embodiments, the antibody or the antigen-binding fragment thereof, or the single-domain antibody or the antigen-binding fragment thereof targets a cancer cell marker. In some embodiments, the antibody or the antigen-binding fragment thereof, or the single-domain antibody or the antigen-binding fragment thereof targets an endothelial cell biomarker. In some embodiments, the endothelial cell biomarker is expressed by a vasculature cell. In some embodiments, the endothelial cell biomarker is expressed by a blood vessel cell. In some embodiments, the endothelial cell biomarker is expressed by a lymphatic vessel cell. In some embodiments, the enucleated cell delivers the exogenous polypeptide to a cell within the abnormal vasculature of the subject. In some embodiments, the enucleated cell comprises at least one additional exogenous agent. In some embodiments, the at least one additional exogenous agent comprises an immune checkpoint molecule. In some embodiments, the at least one additional exogenous agent comprises an immune checkpoint molecule inhibitor. In some embodiments, the at least one additional exogenous agent comprises an angiogenesis inhibitor. In some embodiments, the angiogenesis inhibitor comprises a VEGF/VEGFR inhibitor. In some embodiments, the VEGF/VEGFR inhibitor comprises a VEGF-A inhibitor. In some embodiments, the at least one exogenous agent kills a cancer within the abnormal vasculature. In some embodiments, the at least one exogenous agent recruits an endogenous immune cell to the abnormal vasculature to kill a cancer within the abnormal vasculature. In some embodiments, the enucleated cell is administered to the subject intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof. In some embodiments, following administration of the enucleated cell to the subject, the enucleated cell is viable fewer than or equal to 14 days in the subject. In some embodiments, following administration of the enucleated cell to the subject, the enucleated cell is viable fewer than or equal to 4 days in the subject. In some embodiments, the disease or the condition is cancer or a neoplasm. In some embodiments, the abnormal vasculature is in the lung of the subject. In some embodiments, the method further comprises administering to the subject CPI-006, Monalizumab, COM701, CM24, NEO-201, Defactinib, PF-04136309, MSC-1, Hu5F9-G4 (5F9), ALX148, TTI-662, RRx-001, Lacnotuzumab (MCS110), LY3022855, SNDX-6352, Emactuzumab (RG7155), Pexidartinib (PLX3397), CAN04, Canakinumab (ACZ885), BMS-986253, Pepinemab (VX15/2503), Trebananib, FP-1305, Enapotamab vedotin (EnaV), Bavituximab, or a combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1 illustrates a process for generating the enucleated cells for the delivery of the single-domain antibody according to various embodiments described herein.

FIG. 2 illustrates a timeline for production of the enucleated cells for the delivery of the single-domain antibody according to various embodiments, as compared to a typical biological drug development timeline.

FIG. 3A is a representative graph showing the relative fold change in viable cells or enucleated cells (“cytoplasts”) over time.

FIG. 3B is a representative graph showing the viable cells and cytoplasts after recovery from frozen storage (cryopreservation).

FIG. 3C is a representative graph showing the relative viability of cytoplasts 24 hours after enucleation (fresh cytoplasts) or 24 hours after recovery from frozen storage (cryopreserved) following enucleation, where fresh and cryopreserved cytoplasts are compared to the viability of cytoplasts 4 hours after enucleation. Mean±SEM; n=10.

FIG. 4A is a representative line graph showing the viability of mesenchymal stem cell (MSC) and MSC-derived cytoplasts immediately after recovery from cryohibernation at 4 degrees Celsius for the indicated amounts of time. Viability was assessed in an automated cell count (Cell Countess) using Trypan blue dye exclusion and displayed as a ratio to the number of input cells.

FIG. 4B is a representative bar graph comparing the migrated MSC and MSC-derived cytoplasts in a Boyden chamber assay immediately after recovery from cryohibernation at 4 degrees Celsius for the indicated amounts of time. Cells and cytoplasts were allowed to migrate for 3 hours with either no serum (negative control) or 10% premium FBS (P-FBS) as a chemoattractant in the bottom chamber, and counts were normalized to loading controls.

FIG. 5A is a representative flow cytometry graphs showing the number of events counted over the signal strength of the cell surface C-X-C Motif Chemokine Receptor 4 (CXCR4) expression by fluorescent antibody on engineered cytoplasts and engineered parental MSCs as analyzed by FlowJo.

FIG. 5B is a representative bar graph showing the ratio of migrating cells or cytoplasts that migrated to the undersurface of the Boyden chamber membrane compared to the loading control. Mean±SEM; n=10. MSCs and MSC-derived cytoplasts with and without engineered CXCR4 receptors were allowed to migrate towards the indicated concentrations of stromal cell-derived factor 1α (SDF-1α) for 2 hours in a Boyden chamber assay.

FIG. 6A is a representative flow cytometry graph showing the number of events counted over the signal strength of the cell surface P-selectin glycoprotein ligand-1 (PSGL1) expression by fluorescent antibody on engineered cytoplasts and engineered parental MSCs as analyzed by FlowJo.

FIG. 6B is a representative graph showing cell surface binding of P-Selectin with engineered MSCs and MSC-derived cytoplasts as determined by flow cytometry. MSC control=parental MSCs. Engineered MSC=PSGL1/(Fucosyltransferase 7) Fut7 engineered MSC. Engineered cytoplast=PSGL1/Fut7 engineered MSC-derived cytoplasts.

FIG. 7A is a representative flow cytometry graph showing the number of events counted over the signal strength of the cell surface of mCD47 expression on engineered cytoplasts and MSCs as analyzed by FlowJo.

FIG. 7B is a representative bar graph showing the number of live cytoplasts (DiD+) that were not phagocytosed by macrophages (F4/80⁻ and CD11b⁻), indicating that cytoplasts escaped macrophage phagocytosis in the lung. Mean±SEM; n=3. DiD dye-labeled Control cytoplasts or engineered cytoplasts (mCD47 Cytoplasts) were retro-orbitally injected into the vasculature of mice. After 24 hours, tissues were harvested and stained with two different pan-macrophage markers (F4/80 and CD11b).

FIG. 7C is a representative bar graph showing live cytoplasts (DiD+) that were not phagocytosed by macrophages (F4/80⁻ and CD11b⁻), indicating that cytoplasts escaped macrophage phagocytosis in the liver. Mean±SEM; n=3. DiD dye-labeled Control cytoplasts or engineered cytoplasts (mCD47 Cytoplasts) were retro-orbitally injected into the vasculature of mice. After 24 hours, tissues were harvested and stained with two different pan-macrophage markers (F4/80 and CD11b).

FIG. 8A is a representative scatter plot showing the number of DiD-labeled MSCs or cytoplasts detected in the lung. MSCs were cultured under standard adherent conditions (2D) or in suspension by the handing drop method (3D) to generate 3D cytoplasts. MSCs and cytoplasts were labeled with Vybrant® DiD dye and retro-orbitally injected into the vasculature of C57BL/6 mice. Tissues were harvested after 24 hours and cell suspensions analyzed by flow cytometry. Mean±SEM; n=2.

FIG. 8B is a representative scatter plot showing the number of DiD-labeled MSCs or cytoplasts detected in the liver. MSCs were cultured under standard adherent conditions (2D) or in suspension by the handing drop method (3D) to generate 3D cytoplasts. MSCs and cytoplasts were labeled with Vybrant® DiD dye and retro-orbitally injected into the vasculature of C57BL/6 mice. Tissues were harvested after 24 hours and cell suspensions analyzed by flow cytometry. Mean±SEM; n=2.

FIG. 8C is a representative scatter plot showing the number of Vybrant® DiD-labeled MSCs or cytoplasts detected in the spleen. MSCs were cultured under standard adherent conditions (2D) or in suspension by the handing drop method (3D) to generate 3D cytoplasts. MSCs and cytoplasts were labeled with DiD dye and retro-orbitally injected into the vasculature of C57BL/6 mice. Tissues were harvested after 24 hours and cell suspensions analyzed by flow cytometry. Mean±SEM; n=2.

FIG. 9 illustrates cell surface staining of fluorescein isothiocyanate (FITC) labeled Annexin V on mesenchymal stromal cells (MSCs) or the cytoplasts analyzed by flow cytometry for cell viability analysis.

FIG. 10A is a representative graph showing the abundance of secreted single-domain antibody as measured by enzyme-linked immunoassay (ELISA) in conditioned media of non-transfected (hTERT) cell or cell transfected with a vector encoding the single-domain antibody (scFv). The transfected cell was enucleated and seeded in 6 well plates (0.5×10⁶/well) and the conditioned media was collected after 24 and 48 hours after enucleation for ELISA detection. Mean±SEM; n=3 biological replicates.

FIG. 10B is a representative graph showing the secreted anti-Programmed death-ligand 1 (PD-L1) nanobody, single-domain antibody, or scFv (NB) measured by ELISA in conditioned media of non-transfected (hTERT-MSCs only) or transfected (enucleated cells+NB αPD-L1) cells. Cells were seeded in 6 well plates (0.5×10⁶/well) and the conditioned media was collected after 24 and 48 hours after enucleation for ELISA detection. Mean±SEM; n=3 biological replicates.

FIG. 10C is a representative graph showing the secreted anti-cytotoxic T-lymphocyte-associated protein 4 (CTLA-4) NB measured by ELISA in conditioned media of non-transfected (hTERT-MSCs only) and transfected (enucleated cells+NB αCTLA-4) cells. Cells were seeded in 6 well plates (0.5×10⁶/well) and the conditioned media was collected after 24 and 48 hours after enucleation for ELISA detection. Mean±SEM; n=3 biological replicates.

### DETAILED DESCRIPTION

Disclosed herein are enucleated cells capable of being extensively engineered to express a single-domain antibody, or portion thereof, in the absence of a nucleus. Such enucleated cells may be used to express and deliver the single-domain antibody or portion thereof to a target cell or tissue in vivo even when administered systemically. The single-domain antibody or a portion thereof or a fragment thereof can exert therapeutic efficacy. For example, the single-domain antibody or a portion thereof or a fragment thereof can target and bind to an immune checkpoint molecule, thus reducing the expression of the immune checkpoint molecule in a subject and treating a disease or condition in the subject. In some aspects, the enucleated cell can be engineered to express an exogenous tumor necrosis factor (TNF) superfamily member polypeptide or a catalytically active fragment thereof. In some aspects, the enucleated cell can be engineered to express both the single-domain antibody and the exogenous TNF. In some aspects, the exogenous TNF normalizes abnormal vasculature in the subject. In some aspects, normalization of the abnormal vasculature in the subject increases therapeutic efficacy of the enucleated cell. For example, the normalized vasculature allows delivery of the single-domain antibody to a target site associated with the disease or condition.

The enucleated cells described herein may be engineered to express one or more targeting moieties that, when expressed by the enucleated cell (e.g., on its surface), guide the enucleated cell to the target cell or tissue in vivo. In order to avoid unintended clearance of the enucleated cells in vivo, the enucleated cell may also include an immune system evading moiety (a “don't eat me” signaling polypeptide) such as CD47, PD-L1, HLA-E, or HLA-G. In some embodiments, the enucleated cell described herein comprises an additional active agent such as a therapeutic agent.

There are two major problems for that arise with the systemic administration of conventional therapeutic cells. Firstly, most of the cells may be trapped in the small capillaries in the lung or other tissues, negatively impact biodistribution of the therapeutic cargo and may cause serious side effects as pulmonary embolism. Secondly, conventional therapeutic cells lacking the tissue-specific homing receptors and adhesion molecules (e.g., SDF-1α/CXCR4, CCL2/CCR2, PSGL-1) to target intended cells or tissue are unable to home to such intended cells or tissues efficiently.

The enucleated cells of the present disclosure solve such problems by, in some embodiments, being much smaller than traditional therapeutic cells and their parental cells (e.g., about 60% of the diameter of parental cells and ⅛ the volume) and do not have the rigid nucleus. Therefore, the enucleated cells may pass better through small capillaries and vessels than their conventional counterparts. In addition, the enucleated cells described herein may be engineered to express functional targeting moieties (e.g., homing receptors, adhesion molecules, or membrane-bound antibody such as membrane-bound single-domain antibody) to facilitate efficient homing to target cell or tissue, even when administered to a subject systemically. In some embodiments, the target cell or tissue is a cancer cell or tissue. In the case of enucleated cells that target a cancer cell or tissue, the enucleated cells may be engineered to express targeting moieties that recognize an antigen produced by the cancer cell or tissue. In some embodiments, the enucleated cells may be engineered to express other targeting moieties to guide the enucleated cells to the target tissue, including chemokines, integrins, adhesion molecules, membrane-bound antibody, or membrane-bound single-domain antibody.

In some embodiments, the single-domain antibody or portion thereof is exogenous to the enucleated cell or parent cell thereof. The exogenous single-domain antibody or portion thereof may confer a therapeutic effect such as to treat a disease or a condition described herein. In some embodiments, the single-domain antibody or portion thereof is fused with a plurality of antibodies or a plurality of single-domain antibodies that may target a single epitope or multiple epitopes (e.g., bispecific). In some embodiments, the single-domain antibody is conjugate to a small molecule such as an antibody-drug conjugate (ADC). In some embodiments, the small molecule is a cytotoxic drug or therapeutically effective portion thereof.

In some embodiments, the enucleated cell comprises polynucleotide encoding the single-domain antibody or portion thereof. In some embodiments, the polynucleotide is exogenous to the enucleated cell or the parent cell thereof. In some embodiments, the polynucleotide encodes the single-domain antibody fused with the plurality of antibodies or a plurality of single-domain antibodies described herein.

Also disclosed herein are methods of using the enucleated cells and compositions comprising the enucleated cells of the present disclosure. In some embodiments, methods for treating a disease or a condition by delivering the enucleated cell or compositions containing the enucleated cell to a subject are provided. In some embodiments, the disease or the condition is associated with the target cell or target tissue that the enucleated cell is engineered to target. In some embodiments, the target cell is a cancer cell. In some embodiments, the target cell is an epithelial cell. In some cases, the tissue is a tumor. In some embodiments, the tissue is lung tissue. In some embodiments, the disease or the condition comprises cancer. In some embodiments, the disease or the condition comprises idiopathic pulmonary fibrosis. In some embodiments, methods comprise using the enucleated cells of the present disclosure to transfer cargo (e.g., single domain antibody, therapeutic agent) to the target cell, such as, for example, with a fusogenic moiety or tunneling nanotubule.

Disclosed herein are methods of producing the enucleated cells described herein, by removing a nucleus from a nucleated parent cell such as, for example, without undergoing differentiation of the parent cell. For example, the parent cell containing a nucleus may be engineered to express the single-domain antibody, or portion thereof, targeting moiety or “don't eat me” signaling peptide; and subsequently, the nucleus of the parent cell may be removed. In another example, the parent cell containing the nucleus is enucleated, and the enucleated cell is engineered to express the domain antibody, or portion thereof, targeting moiety or “don't eat me” signaling peptide. In some embodiments, removal of the nucleus involves mechanically removing the nucleus.

Following enucleation of the parent cell, the enucleated cells described herein retain one or more intracellular organelles that are endogenous to the parent cell. In some embodiments, all of the one or more intracellular organelles are retained. In some embodiments, fewer than all of the one or more intracellular organelles are retained. In some embodiments, the Golgi apparatus and/or the endoplasmic reticulum are retained, which are involved in protein synthesis and secretion. Retention of the one or more intracellular organelles at least partially enables the enucleated cells to synthesize or release the biomolecule disclosed herein (e.g., single-domain antibody, or portion thereof, targeting moiety, immune-evading moiety, etc.) in the absence of the nucleus.

In some embodiments, the parent cell is any one of the nucleated cells described herein. In some embodiments, the parent cell is an adult stem cell. In some embodiments, the parent cell is a mesenchymal stromal cell (MSC). In some embodiments, the enucleated cell is derived from an inducible pluripotent stem cell (iPSC). In some embodiments, the parent cell is not an erythrocyte or erythroid precursor cell. In some embodiments, the parent cell is a platelet cell. In some embodiments, the parent cell is not an endothelial cell. In some embodiments, the parent cell is not an endothelial precursor cell nor an erythroid precursor cell. In some embodiments, the parent cell is not a platelet cell. In some embodiments, the parent cell is not an endothelial cell. In some embodiments, the parent cell is not an endothelial precursor cell. In some embodiments, the parent cell does not express complement receptor one (CR1). In some embodiments, the parent cell does not express CD44. In some embodiments, the parent cell does not express VLA-4. In some embodiments, the parent cell does not express BCAM. In some embodiments, the parent cell does not express ICAM. In some embodiments, the parent cell does not express a receptor for collagen. In some embodiments, the parent cell does not express a receptor for thrombopoietin. In some embodiments, the parent cell does not express a receptor for collagen. In some embodiments, the parent cell does not express a receptor for von Willebrand factor (VWF). In some embodiments, the parent cell does not express a receptor for fibrinogen. In some embodiments, the parent cell does not express GP1b-IX-V receptor. In some embodiments, the parent cell does not express GPIIb/IIIa receptor. In some embodiments, the parent cell does not express prostanoid receptor. In some embodiments, the parent cell does not express purinergic receptor. In some embodiments, the parent cell does not express thromboxane receptor.

### Compositions

Provided herein are cells comprising a single-domain antibody, or portion thereof, and compositions containing such enucleated cells. In some embodiments, the cells are enucleated. In some embodiments, the single-domain antibody is a therapeutic agent. In some embodiments, the enucleated cells are capable of expressing the single-domain antibody (e.g., therapeutic agent) in absence of a nucleus using one or more intracellular organelles retained by the enucleated cells from parent cells. In some embodiments, the single-domain antibody, or portion thereof (e.g., therapeutic agent) is exogenous to the enucleated cell or parent cell thereof. In some embodiments, the enucleated cell expresses the single-domain antibody, or portion thereof (e.g., therapeutic agent) at the surface of the enucleated cell. In some embodiments, the single-domain antibody, or portion thereof is secreted by the enucleated cell into extracellular space at a target tissue (e.g., a microenvironment). In some aspects, the composition comprises an enucleated cell described herein. In some aspects, the enucleated cell is obtained or derived from a nucleated cell (e.g., a parent cell). In some aspects, the enucleated cell comprises a transmembrane moiety. In some embodiments, the enucleated cells comprises a targeting moiety. In some embodiments, the enucleated cell comprises a therapeutic agent. In some embodiments, the enucleated cell comprises an antibody or antigen binding fragment thereof or a single-domain antibody or an antigen binding fragment thereof. In some aspects, the targeting moiety comprises an antibody or antigen binding fragment thereof or a single-domain antibody or an antigen binding fragment thereof. In some embodiments, the therapeutic agent comprises an antibody or antigen binding fragment thereof or a single-domain antibody or an antigen binding fragment thereof. In some embodiments, the enucleated cell is formulated into a pharmaceutical formulation described herein.

### Enucleated Cells

The enucleated cells of the present disclosure are obtained or derived from a corresponding nucleated cell (referred to herein as a “parent cell”). The parent cell may be derived from a variety of different cell types, including eukaryotic cells. For example, an enucleated cell may be derived from an adult stem cell, a mesenchymal stromal cell (MSC), a natural killer (NK) cell, a macrophage, a myoblast, a neutrophil, endothelial cell, endothelial precursor cell, and/or a fibroblast. In some embodiments, an enucleated cell is derived from a mesenchymal stromal cell. In some embodiments, the enucleated cell is derived from an inducible pluripotent stem cell (iPSC). In some embodiments, the parent cell is derived from a cell is immortalized using suitable methods. For example the, parent cell is immortalized by expressing human telomerase reverse transcriptase (hTERT), an oncogene, or a viral gene such as simian virus 40 (SV40). In some embodiments, the cytoplast is derived from a parent cell using suitable methods provided in U.S. Pat. No. 10,927,349, which is hereby incorporated by reference in its entirety. In some embodiments, the enucleated cell retains one or more intracellular organelles for synthesis of an exogenous single-domain antibody or fragment thereof in absence of the nucleus.

In some embodiments, the cell can originate from any organism having one or more cells. Non-limiting examples of cells include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell). In some embodiments, the cell is a somatic cell. In some embodiments, the cell is a stem cell or a progenitor cell. In some embodiments, the cell is a mesenchymal stem or progenitor cell. In some embodiments, the cell is a hematopoietic stem or progenitor cell. In some embodiments, the cell is a muscle cell, a skin cell, a blood cell, or an immune cell. Other non-limiting example of cells includes lymphoid cells such as B cell, T cell (Cytotoxic T cell, Natural Killer T cell, Regulatory T cell, T helper cell), Natural killer cell, cytokine induced killer (CIK) cells; myeloid cells such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

In some embodiments, the cell is a eukaryotic cell. Non-limiting examples of eukaryotic cells include mammalian (e.g., rodent, non-human primate, or human), non-mammalian animal (e.g., fish, bird, reptile, or amphibian), invertebrate, insect, fungal, or plant cells. In some embodiments, the eukaryotic cell is a yeast cell such as Saccharomyces cerevisiae. In some embodiments, the eukaryotic cell is a higher eukaryote such as mammalian, avian, plant, or insect cells. In some embodiments, the nucleated cell is a primary cell. In some embodiments, the nucleated cell is an immune cell (e.g., a lymphocyte (e.g., a T cell, a B cell), a macrophage, a natural killer cell, a neutrophil, a mast cell, a basophil, a dendritic cell, a monocyte, a myeloid-derived suppressor cell, an eosinophil). In some embodiments, the nucleated cell is a phagocyte or a leukocyte. In some embodiments, the nucleated cell is a stem cell (e.g., an adult stem cell (e.g., a hematopoietic stem cell, a mammary stem cell, an intestinal stem cell, mesenchymal stem cell, an endothelial stem cell, a neural stem cell, an olfactory adult stem cell, a neural crest stem cell, a testicular cell), an embryonic stem cell, an inducible pluripotent stem cell (iPS)). In some embodiments, the nucleated cell is a progenitor cell. In some embodiments, the nucleated cell is from a cell line. In some embodiments, the nucleated cell is a suspension cell. In some embodiments, the nucleated cell is an adherent cell. In some embodiments, the nucleated cell is a cell that has been immortalized by expression of an oncogene. In some embodiments, the nucleated cell is immortalized by the expression of human telomerase reverse transcriptase (hTERT) or any oncogene. In some embodiments, the nucleated cell is immortalized by the expression of a viral gene such as simian virus 40 (SV40). In some embodiments, the nucleated cell is a patient or subject derived cell (e.g., an autologous patient-derived cell, or an allogenic patient-derived cell). In some embodiments, the nucleated cell is transfected with a vector (e.g., a viral vector (e.g., a retrovirus vector (e.g., a lentivirus vector), an adeno-associated virus (AAV) vector, a vesicular virus vector (e.g., vesicular stomatitis virus (VSV) vector), or a hybrid virus vector), a plasmid) before the nucleated cell is enucleated using any of the enucleation techniques described herein.

In some embodiments, the cytoplast is derived from a cell autologous to the subject. In some embodiments, the cytoplast is derived from a cell allogenic to the subject.

In some embodiments, the cytoplast is derived from an immune cell. In some embodiments, the cytoplast is derived from a natural killer (NK) cell, a neutrophil, a macrophage, a lymphocyte, a fibroblast, an adult stem cell (e.g., hematopoietic stem cell, a mammary stem cell, an intestinal stem cell, a mesenchymal stem cell, a mesenchymal stromal cell, an endothelial stem cell, a neural stem cell, an olfactory adult stem cell, a neural crest stem cell, a skin stem cell, or a testicular cell), a mast cell, a basophil, an eosinophil, an endothelial cell, an endothelial cell precursor cell, or an inducible pluripotent stem cell.

In some embodiments, the parent cell is not an erythrocyte or erythroid precursor cell. In some embodiments, the parent cell is a platelet cell. In some embodiments, the parent cell is not an endothelial cell. In some embodiments, the parent cell is not an endothelial precursor cell. In some embodiments, the enucleated cell is not an erythrocyte or erythroid precursor cell. In some embodiments, the enucleated cell is not a platelet cell. In some embodiments, the enucleated cell is not an endothelial cell. In some embodiments, the enucleated cell is not an endothelial precursor cell. In some embodiments, the enucleated cell does not express complement receptor one (CR1). In some embodiments, the enucleated cell does not express CD44. In some embodiments, the enucleated cell does not express VLA-4. In some embodiments, the enucleated cell does not express BCAM. In some embodiments, the enucleated cell does not express ICAM. In some embodiments, the enucleated cell does not express a receptor for collagen. In some embodiments, the enucleated cell does not express a receptor for thrombopoietin. In some embodiments, the enucleated cell does not express a receptor for collagen. In some embodiments, the enucleated cell does not express a receptor for von Willebrand factor (VWF). In some embodiments, the enucleated cell does not express a receptor for fibrinogen. In some embodiments, the enucleated cell does not express GP1b-IX-V receptor. In some embodiments, the enucleated cell does not express GPIIb/IIIa receptor. In some embodiments, the enucleated cell does not express prostanoid receptor. In some embodiments, the enucleated cell does not express purinergic receptor. In some embodiments, the enucleated cell does not express thromboxane receptor.

In some embodiments, the parent cell may be enucleated and engineered for therapeutic use. In some embodiments, the parent cell is any one of the nucleated cells described herein. In some embodiments, the parent cell is an adult stem cell. In some embodiments, the parent cell is a mesenchymal stromal cell (MSC). In some embodiments, the enucleated cell is derived from an inducible pluripotent stem cell (iPSC). In some embodiments, the parent cell is not an erythrocyte or erythroid precursor cell. In some embodiments, the parent cell is a platelet cell. In some embodiments, the parent cell is not an endothelial cell. In some embodiments, the parent cell is not an endothelial precursor cell. A parent cell may be treated with cytochalasin to soften the cortical actin cytoskeleton. The nucleus may then physically extracted from the cell body by high-speed centrifugation in gradients of Ficoll to generate an enucleated cell. Because enucleate cells and intact nucleated cells sediment to different layers in the Ficoll gradient, enucleated cells may be easily isolated and prepared for therapeutic purposes or fusion to other cells (nucleated or enucleated). The enucleation process is clinically scalable to process tens of millions of cells. In some embodiments, enucleated cells may be used as a disease-homing vehicle to deliver clinically relevant cargos/payloads to treat various diseases.

In some embodiments, the enucleated cells contain one or more organelles or cytoskeleton to form a tunneling nanotube (TNT) or membrane nanotube. The inventors of the present disclosure discovered that enucleating a cell, can in some cases, increase the TNT in the cell, which may provide therapeutic advantages. In some embodiments, the increase in TNT of the enucleated cell is determined by the enucleated cell exhibiting an increased number of the TNT formation compared to TNT formation by an otherwise identical cell that has not been enucleated. In some embodiments, the increase in TNT of the enucleated cell is determined by the enucleated cell exhibiting an increased length of the TNT formation compared to a length of the TNT formation by an otherwise identical cell that has not been enucleated. In some embodiments, the increase in TNT of the enucleated cell is determined by the enucleated cell exhibiting an increased diameter of the TNT formation compared to a diameter of the TNT formation by an otherwise identical cell that has not been enucleated. In some embodiments, the tunneling nanotube delivers at least one exogenous agent described herein to a target cell. In some embodiments, the increased TNT formation, increased TNT length, or increased TNT diameter increases the efficacy of delivering of the at least one exogenous agent by the enucleated cell to the target cell. In some embodiments, the exogenous agent is a therapeutic agent. In some embodiments, the exogenous agent can normalize vasculature. In some embodiments, the tunneling nanotube is a protrusion that extend from plasma membrane of the enucleated cell, where the tunneling nanotube can comprise a length of about 1 μm to about 1000 μm. In some embodiments, the tunneling nanotube comprises a diameter of about 0.1 nm to about 10.0 μm. In some embodiments, the tunneling nanotube comprises actin. In some embodiments, the tunneling nanotube comprises both actin and microtubule. In some embodiments, the tunneling nanotube comprises content of the cytoplasm of the enucleated cell. For example, the tunneling nanotube can contain vesicles, organelles or the at least one exogenous agent described herein. In some embodiments, the tunneling nanotube can deliver the at least one exogenous agent by contacting with the target cell. In some embodiments, the tunneling nanotube can deliver the at least one exogenous agent to the target cell by forming a fluid communication between the enucleated cell and the target cell. In some embodiments, upon delivery of the at least one exogenous from the enucleated cell to the target cell, the target cell can form additional tunneling nanotube with other non-targeted cells. In such scenario (similar to bystander effect), the target cell can pass on the at least one exogenous agent and its effect to other non-targeted cells, thus propagating the therapeutic effect of the enucleated cell to other non-target cells.

Following enucleation of the parent cell, the enucleated cells described herein retain one or more intracellular organelles that are endogenous to the parent cell. In some embodiments, all of the one or more intracellular organelles are retained. In some embodiments, fewer than all of the one or more intracellular organelles are retained. In some embodiments, the Golgi apparatus and/or the endoplasmic reticulum are retained, which are involved in protein synthesis and secretion. Retention of the one or more intracellular organelles at least partially enables the enucleated cells to synthesize or release the biomolecule disclosed herein (e.g., single-domain antibody, or portion thereof, targeting moiety, immune-evading moiety, etc.) in the absence of the nucleus. In some embodiments, the enucleated cell retains cytoskeleton such as filament or tubules for formation of TNT.

Enucleated cells may be smaller than their nucleated counterparts (e.g., the nucleated parent cells), and for this reason may migrate better through small openings in the vasculature and tissue parenchyma. In addition, removing the large dense nucleus alleviates a major physical barrier allowing the cell to move freely through small openings in the vessels and tissue parenchyma. Therefore, enucleated cells have improved bio-distribution in the body and movement into target tissues. In some embodiments, an enucleated cell comprises at least 1 μm in diameter. In some embodiments, an enucleated cell is greater than 1 μm in diameter. In some embodiments, an enucleated cell is 1-100 μm in diameter (e.g., 1-90 μm, 1-80 μm, 1-70 μm, 1-60 μm, 1-50 μm, 1-40 μm, 1-30 μm, 1-20 μm, 1-10 μm, 1-5 μm, 5-90 μm, 5-80 μm, 5-70 μm, 5-60 μm, 5-50 μm, 5-40 μm, 5-30 μm, 5-20 μm, 5-10 μm, 10-90 μm, 10-80 μm, 10-70 μm, 10-60 μm, 10-50 μm, 10-40 μm, 10-30 μm, 10-20 μm, 10-15 μm 15-90 μm, 15-80 μm, 15-70 μm, 15-60 μm, 15-50 μm, 15-40 μm, 15-30 μm, 15-20 μm). In some embodiments, an enucleated cell is 10-30 μm in diameter. In some embodiments, the diameter of an enucleated cell is between 5-25 μm (e.g., 5-20 μm, 5-15 μm, 5-10 μm, 10-25 μm, 10-20 μm, 10-15 μm, 15-25 μm, 15-20 μm, or 20-25 μm). In some embodiments, the enucleated cell has a diameter that is about 8 μm. In some embodiments, some enucleated cells may advantageously be small enough to allow for better homing or delivery to a target site. For examples, the enucleated cells described herein may pass through passages in narrow lung tissues or lung structures such as alveolar duct or microcapillary that most cells such as the parent cells may not pass through.

In some embodiments, enucleated cells possess significant therapeutic value, because they remain viable, do not differentiate into other cell types, secrete bioactive molecules, and may physically migrate/home for fewer than or equal to about 5 days, may be extensively enucleated ex vivo to perform specific therapeutic functions, and may be fused to the same or other cell types to transfer desirable production, natural or enucleated. Therefore, enucleated cells have wide utility as a cellular vehicle to deliver therapeutically important biomolecules and disease-targeting cargos including genes, viruses, bacteria, mRNAs, shRNAs, siRNA, polypeptides (including antibodies and antigen binding fragments), plasmids, gene-editing machinery, or nanoparticles. The present disclosure enables the generation of safe (e.g., no unwanted DNA is transferred to the subject), and controllable (e.g., cell death occurs in 3-4 days) cell-based carrier that may be genetically enucleated to deliver specific disease-fighting and health promoting cargos to humans. In some embodiments, the enucleated cell remains viable and retain the function to migrate or home for greater than or equal to about 12 hours, 24 hours, 36 hours, 48 hours, 60 hours, 72 hours, 84 hours, 96 hours, 108 hours, or 5 days after being administered to the subject in need thereof.

In some embodiments, the enucleated cell is engineered to express at least one of an exogenous DNA molecule, an exogenous RNA molecule, an exogenous protein, or an exogenous protein, gene-editing machinery or combinations thereof. In some embodiments, the exogenous DNA molecule is a single-stranded DNA, a double-stranded DNA, an oligonucleotide, a plasmid, a bacterial DNA molecule, a DNA virus, or combinations thereof. In some embodiments, the exogenous RNA molecule is messenger RNA (mRNA), small interfering RNA (siRNA), microRNA (miRNA), short hairpin RNA (shRNA), a RNA virus, or combinations thereof. In some embodiments, the exogenous protein is a cytokine, a growth factor, a hormone, an antibody or the antigen-binding fragment thereof, an enzyme, or combinations thereof. In some embodiments, the antibody is a single-domain antibody or antigen-binding fragment thereof. In some embodiments, parental cells (e.g., nucleated cells) are genetically enucleated before enucleation (e.g., pre-enucleation). In some embodiments, the parent cell is genetically enucleated after enucleation (e.g., post-enucleation).

In some embodiments, the enucleated cell described herein comprises a transmembrane moiety. In some embodiments, the transmembrane moiety is genetically modified to be fused or complexed with the single-domain antibody or antigen-binding fragment thereof, or a therapeutic agent described herein, or a combination thereof. In some embodiments, the transmembrane moiety is genetically modified to fuse to the single-domain antibody or antigen-binding fragment thereof, or therapeutic agent described herein, or a combination thereof. In some embodiments, the enucleated cell comprises an immune-evading moiety. In some embodiments, the immune-evading comprises a “don't eat me” signaling peptide such as CD47, PD-L1, HLA-E, HLA-G, a fragment thereof, or a combination thereof.

In some embodiments, the enucleated cell or the composition comprising the enucleated cell may be cryopreserved (e.g. storing the enucleated cell or the composition comprising the enucleated cell at freezing temperature) or cryohibernated (e.g. storing the enucleated cell or the composition comprising the enucleated cell at a temperature that is between the ambient temperature and freezing temperature). The duration of cryopreservation or cryohibernation may be greater than or equal to about one hour, two hours, six hours, 12 hours, one day, two days, three days, four days, five days, six days, one week, two weeks, three weeks, four weeks, one month, two months, three months, or longer period of time. In some embodiments, the enucleated cell exhibits a viability after cryopreservation or cryohibernation that is greater than or equal to about 50%, 55%, 60%, 65%, 70%, 75%, 80%, 85%, 90%, 95%, or 99% similar to a comparable cell (e.g., a parent cell or an enucleated cell described herein that has not been cryopreserved or cryo-hibernated) after same the period of time of cryopreservation or cryohibernation. For example, in some embodiments, the viability is reduced by 50%, 55%, 60%, 65%, 70%, 75%, 80%, 85%, 90%, 95%, or 99% as compared with the comparable cell. In another example, in some embodiments, the viability is increased by 50%, 55%, 60%, 65%, 70%, 75%, 80%, 85%, 90%, 95%, or 99% as compared with the comparable cell. Viability in this context may be measured by Trypan blue dye exclusion as described herein. In some embodiments, the Trypan blue dye exclusion is performed by: (a) centrifuging an aliquot of a plurality of the cell without the nucleus in a suspension to create a cell pellet; (b) resuspending the cell pellet in serum-free medium to produce a serum-free cell suspension; (c) mixing 1 part Trypan blue dye and 1 part of the serum-free cell suspension; (d) counting the plurality of the cells without the nucleus within 3-5 minutes of (c), wherein at least some of the plurality of cells without the nucleus are unstained with the Trypan blue dye, which is indicative of viability. In some embodiments, the viability is measured using Annexin-V cell surface staining. In some embodiments, the viability is measured by expression of the exogenous polypeptide. For example, the viability of the enucleated cell can be determined by the expression of the exogenous antibody or single-domain antibody expressed by the enucleated cell. In some embodiments, the viability is measured by expression of cell surface markers of any one of the cell surface markers described herein such as CD105, CD90, CD45, CXCR4, PSGL-1, or CCR2. In some embodiments, the viability is measured by the cell activity of the enucleated cell. In some embodiments, the viability is measured by the homing capability of the enucleated cell as determined by the chemosensing or chemokine homing activity described herein.

In some embodiments, the enucleated cell or the composition comprising the enucleated cell may be lyophilized. In some embodiments, the enucleated cell exhibits a viability after being reconstituted from lyophilization that is greater than or equal to about 50%, 55%, 60%, 65%, 70%, 75%, 80%, 85%, 90%, 95%, or 99% similar to a comparable cell (e.g., a parent cell or an enucleated cell described herein that has not been lyophilized).

In some embodiments, the enucleated cell or the composition comprising the enucleated cell may be dehydrated. In some embodiments, the enucleated cell exhibits a viability after being rehydrated from lyophilization that is greater than or equal to about 50%, 55%, 60%, 65%, 70%, 75%, 80%, 85%, 90%, 95%, or 99% similar to a comparable cell (e.g., a parent cell or an enucleated cell described herein that has not been dehydrated).

In some embodiments, the enucleated cell or the composition comprising the enucleated cell is stable at 4° C. for greater than or equal to about one hour, two hours, six hours, 12 hours, one day, two days, three days, four days, five days, six days, one week, two weeks, three weeks, four weeks, one month, two months, three months, or longer period of time. In some embodiments, the composition is stable at room temperature for greater than or equal to about one hour, two hours, six hours, 12 hours, one day, two days, three days, four days, five days, six days, one week, two weeks, three weeks, four weeks, one month, two months, three months, or longer period of time. In some embodiments, the composition is stable at 37° C. for greater than or equal to about one hour, two hours, six hours, 12 hours, one day, two days, three days, four days, five days, six days, one week, two weeks, three weeks, four weeks, one month, two months, three months, or longer period of time. In some embodiments, the enucleated cell or the composition comprising the enucleated cell may remain viable after being administered to a subject in need thereof for treating the disease or condition described herein. In some embodiments, the enucleated cell or the composition comprising the enucleated cell may remain viable after being administered to the subject for greater than or equal to about one hour, two hours, six hours, 12 hours, one day, two days, three days, four days, five days, six days, one week, two weeks, three weeks, four weeks, one month, two months, three months, or longer period of time.

In some embodiments, the enucleated cell may be obtained from a parent cell that is autologous to the subject, who is in need of the treatment by the enucleated cell described herein. In some embodiments, the enucleated cell may be obtained from a parent cell that is allogenic to the subject, who is in need of the treatment by the enucleated cell described herein.

### Transmembrane Moiety

Described herein, in some embodiments, are cells or compositions comprising the enucleated cell comprising at least one transmembrane moiety. In some embodiments, the cells are enucleated, such as with the methods of enucleation disclosed herein. In some embodiments, transmembrane moiety is coupled to a polypeptide, such as a single-domain antibody or antigen-binding fragment thereof, a therapeutic agent disclosed here, or a combination thereof. In some embodiments, the transmembrane moiety is coupled by way of a covalent bond. In some embodiments, the transmembrane moiety is a fusion protein comprising the single-domain antibody or antigen-binding fragment thereof, a therapeutic agent disclosed here, or a combination thereof. In some embodiments, the exogenous polypeptide is complexed to the transmembrane moiety. In some embodiments, the transmembrane moiety comprises a full length protein or a variation thereof or a fragment thereof. In some embodiments, the transmembrane moiety is endogenous to the parent cell that is being enucleated for obtaining the enucleated cell. In some embodiments, the transmembrane moiety may be an exogenous transmembrane moiety to the parent cell or to the enucleated cell. In some embodiments, the transmembrane moiety comprises a single transmembrane α-helix (bitopic membrane protein. The transmembrane moiety comprises a polytopic transmembrane α-helical protein. In some embodiments, the transmembrane moiety comprises a polytopic transmembrane β-sheet protein. In some embodiments, the transmembrane moiety comprises a Type I, II, III, or IV transmembrane protein. Non-limiting examples of transmembrane protein may include CD4, CD14, glycophorin a (GPA), or any combination of integrins.

In some embodiments, the transmembrane moiety is added to the exogenous polypeptide by way of a modification. For example, a transmembrane moiety may be added to the N or C-terminus of the exogenous polypeptide to insert the exogenous polypeptide into the cell membrane of the enucleated cell described herein. Non-limiting examples of modifications that are made to the exogenous polypeptide to add the transmembrane moiety may include adding an anchor molecule. Anchor molecule can be any molecule (e.g., a glycolipid) that can be inserted and remain in the cellular membrane. In some embodiments, the anchor molecules comprises a glycosylphosphatidylinositol, a farnesyl, a palmitate, a myristate, or a combination thereof.

### Targeting Moiety

Described herein, in some embodiments, are cells comprising a targeting moiety. In some embodiments, the cells are enucleated, such as with the methods of enucleation disclosed herein. The targeting moiety described herein is designed to guide the enucleated cell to a target cell or target environment (e.g., tissue) in a subject following delivery (e.g., systemic delivery) of the enucleated cell to the subject. In some embodiments, the targeting moiety is expressed on the surface of the enucleated cell. In some embodiments, the targeting moiety is complexed with a transmembrane moiety described herein. In some embodiments, the targeting moiety is secreted by the enucleated cell. The enucleated cells comprising the targeting moiety localizes at the target cell or target environment in a subject with a 2 fold, 5 fold, 10 fold, 50 fold, 100 fold, 200 fold, 500 fold, 1,000 fold, 5,000 fold, or 10,000 fold increase as compared to localization of a comparable enucleated cell lacking the targeting moiety. In some embodiments, the enucleated cell comprising the targeting moiety localizes at the target cell or target environment in a subject with at least a 2 fold increased as compared to localization of a comparable enucleated cell lacking the targeting moiety. In some embodiments, the enucleated cell comprising the targeting moiety localizes at the target cell or target environment in a subject with at least a 5 fold increased as compared to localization of a comparable enucleated cell lacking the targeting moiety. In some embodiments, the enucleated cell comprising the targeting moiety localizes at the target cell or target environment in a subject with at least a 10 fold increased as compared to localization of a comparable enucleated cell lacking the targeting moiety. In some embodiments, the enucleated cell comprising the targeting moiety localizes at the target cell or target environment in a subject with at least a 20 fold increased as compared to localization of a comparable enucleated cell lacking the targeting moiety. In some embodiments, the enucleated cell comprising the targeting moiety localizes at the target cell or target environment in a subject with at least a 50 fold increased as compared to localization of a comparable enucleated cell lacking the targeting moiety. In some embodiments, the enucleated cell comprising the targeting moiety localizes at the target cell or target environment in a subject with at least a 5% increased as compared to localization of a comparable enucleated cell lacking the targeting moiety. In some embodiments, the enucleated cell comprising the targeting moiety localizes at the target cell or target environment in a subject with at least a 10% increased as compared to localization of a comparable enucleated cell lacking the targeting moiety. In some embodiments, the enucleated cell comprising the targeting moiety localizes at the target cell or target environment in a subject with at least a 20% increased as compared to localization of a comparable enucleated cell lacking the targeting moiety. In some embodiments, the enucleated cell comprising the targeting moiety localizes at the target cell or target environment in a subject with at least a 30% increased as compared to localization of a comparable enucleated cell lacking the targeting moiety. In some embodiments, the enucleated cell comprising the targeting moiety localizes at the target cell or target environment in a subject with at least a 40% increased as compared to localization of a comparable enucleated cell lacking the targeting moiety. In some embodiments, the enucleated cell comprising the targeting moiety localizes at the target cell or target environment in a subject with at least a 50% increased as compared to localization of a comparable enucleated cell lacking the targeting moiety. In some embodiments, the enucleated cell comprising the targeting moiety localizes at the target cell or target environment in a subject with at least a 60% increased as compared to localization of a comparable enucleated cell lacking the targeting moiety. In some embodiments, the enucleated cell comprising the targeting moiety localizes at the target cell or target environment in a subject with at least a 70% increased as compared to localization of a comparable enucleated cell lacking the targeting moiety. In some embodiments, the enucleated cell comprising the targeting moiety localizes at the target cell or target environment in a subject with at least a 80% increased as compared to localization of a comparable enucleated cell lacking the targeting moiety. In some embodiments, the enucleated cell comprising the targeting moiety localizes at the target cell or target environment in a subject with at least a 90% increased as compared to localization of a comparable enucleated cell lacking the targeting moiety. In some embodiments, the enucleated cell comprising the targeting moiety localizes at the target cell or target environment in a subject with at least a 100% increased as compared to localization of a comparable enucleated cell lacking the targeting moiety.

In some embodiments, the targeting moiety comprises an exogenous antibody or an exogenous antigen-binding fragment for targeting a biomarker described herein. In some embodiments, the targeting moiety comprises an exogenous antibody or an exogenous antigen-binding fragment for targeting a chemokine receptor or a chemokine ligand, or portion thereof, involved in chemokine signaling. In some embodiments, the exogenous antibody is an exogenous single-domain antibody or fragment thereof.

In some embodiments, the targeting moiety targets the biomarker expressed by, or associated with, a target cell or with a microenvironment. In some embodiments, the biomarker may be released by the target cell. The biomarker may indicate the presence of the disease or the condition. In some embodiments, the biomarker is expressed by immune cells responding to the target cell or the microenvironment associated with the disease or the condition. In some embodiments, the biomarker may be an epitope or antigen. In some embodiments, the biomarker comprising the epitope may be bound by an antibody that is different from the antibody or the antigen-binding fragment thereof that confers therapeutic property (e.g., the therapeutic agent).

In some embodiments, the targeting moiety targets a biomarker expressed or released by a lung cell or a lung cancer cell. Non-limiting example of cancer cell biomarkers includes carbonic anhydrase 9 (CA9), carbonic anhydrase 12 (CA12), Kita-Kyushu Lung Cancer Antigen 1 (CXorf61), desmoglein 3 (DSG3), FAT Atypical Cadherin 2 (FAT2), G-protein coupled receptor 87 (GPR87), Kisspeptin receptor (KISS1R), LY6/PLAUR Domain Containing 3 (LYPD3), solute carrier family 7 member 11 (SLC7A11), transmembrane serine protease 4 (TMPRSS4), tissue factor pathway inhibitor (TFPI), midkine (MDK), secreted phosphoprotein 1 (OPN), matrix metallopeptidase 2 (MMP2), TIMP metallopeptidase inhibitor 1 (TIMP1), Carcinoembryonic antigen (CEA), Keratin 19 (CYFRA 21-1), SCC, advanced glycosylation end-product specific receptor (AGER), adipogenesis regulatory factor (C10orf116), adducin 2 (ADD2), periaxin (PRX), laminin subunit beta 3 (LAMB3), synemin (SYNM), spectrin alpha, erythrocytic 1 (SPTA1), ankyrin 1 (ANK1), hemoglobin subunit epsilon 1 (HBE1), hemoglobin subunit gamma 1 (HBG1), carbonic anhydrase 1 (CA1), tenascin XB (TNXB), multimerin 2 (MMRN2), hemoglobin subunit alpha 1 (HBA1), caveolin 1 (CAV1), hemoglobin subunit beta (HBB), collagen type VI alpha 6 chain (COL6A6), chromosome 1 open reading frame 198 (C1orf198), chloride intracellular channel 2 (CLIC2), serum deprivation-response protein (SDPR), EH domain containing 2 (EHD2), apolipoprotein A2 (APOA2), NADH: ubiquinone oxidoreductase subunit B7 (NDUFB7), protein kinase C delta binding protein (PRKCDBP), laminin subunit alpha 3 (LAMA3), EvC ciliary complex subunit 2 (LBN), actin-like protein (ACT), insulin like growth factor binding protein 3 (IGFBP3), prostaglandin D2 synthase (L-PGDS), hapless (HAP), hepatocyte growth factor (HGF), eukaryotic translation initiation factor 4 gamma 2 (AAG1/2), clusterin (CLU), calreticulin (SSA), tRNA suppressor anticodon 2-1 (TTA), apolipoprotein A4 (APOA4), fibrinogen alpha chain (FIBA), serum amyloid A cluster (SAA), ceruloplasmin (CP), haptoglobin (HP), transthyretin (TTR), keratin 2 (KRT2A), solute carrier family 1 (GLT1B), casein kinase 1 (CK1), serine/threonine kinase 1 (AKT), MBL2 (mannose binding lectin 2), fibrinogen alpha chain (FGA), gelsolin (GSN), ficolin 3 (FCN3), carnosine dipeptidase 1 (CNDP1), calcitonin related polypeptide alpha (CALCA), carbamoyl-phosphate synthase 1 (CPS1), chromogranin B (CHGB), involucrin (IVL), anterior gradient 2, protein disulphide isomerase family member (AGR2), nuclear autoantigenic sperm protein (NASP), phosphofructokinase, platelet (PFKP), thrombospondin 2 (THBS2), thioredoxin domain containing 17 (TXNDC17), proprotein convertase subtilisin/kexin type 1 (PCSK1), cellular retinoic acid binding protein 2 (CRABP2), acyl-CoA binding domain containing 3 (ACBD3), desmoglein 2 (DSG2), LPS responsive beige-like anchor protein (LRBA), serine/threonine kinase receptor associated protein (STRAP), VGF nerve growth factor inducible (VGF), NOP2 nucleolar protein (NOP2), lipocalin 2 (LCN2), creatine kinase, mitochondrial 1B (CKMT1B), aldo-keto reductase family 1 member B10 (AKR1B10), carboxypeptidase D (CPD), proteasome activator subunit 3 (PSME3), villin 1 (VIL1), serpin family B member 5 (SERPINB5), ribosomal protein L5 (RPL5), plakophilin 1 (PKP1), ribosomal protein L10 (RPL10), aldo-keto reductase family 1 member B10 (AKR1B10), aldo-keto reductase family 1 member C1 (AKR1C1), proliferating cell nuclear antigen (PCNA), ribosomal protein S2 (RPS2), aldo-keto reductase family 1 member C3 (AKR1C3), acyl-CoA binding domain containing 3 (ACBD3), visinin like 1 (VSNL1), adenosylhomocysteinase (AHCY), stromal interaction molecule 1 (STIM1 or IMMP10), p21 (RAC1) activated kinase 2 (PAK2), involucrin (IVL), isoleucine-tRNA synthetase (TARS), proteasome 26S subunit ubiquitin receptor, non-ATPase 2 (PSMD2), guanylate binding protein 5 (GBPS), minichromosome maintenance complex component 6 (MCM6), N-myc downstream regulated 1 (NDRG1), NOP58 ribonucleoprotein (NOP58), 5100 calcium binding protein A2 (S100A2), neuregulin 1 (NRG1-2), carnosine dipeptidase 1 (CNDP1), ubiquitin cross-reactive protein (UCRP), cerberus (CER), plasminogen activator, urokinase (UPA or PLAU), matrix metallopeptidase 14 (MT1-MMP), stratifin (SFN), transferrin (TF), albumin (ALB), 5100 calcium binding protein A9 (S100A9), stathmin 1 (STMN), enolase (ENO), insulin like growth factor binding protein 7 (IGFBP7), matrix metallopeptidase 14 (MMP14), thrombospondin 1 (THBS1), and thrombospondin 2 (THBS2).

In some embodiments, the targeting moiety targets a biomarker expressed or released by a cancer cell that has metastasized. For example, the cancer cell may arise from one tissue and subsequently metastasizes to a different location. In some embodiments, the metastasized cancer cell expresses the non-limiting example of cancer biomarker described herein. In some embodiments, the metastasized cancer cell expresses cancer biomarker includes Melanoma Associated Antigen (MAGE-A3), Membrane associated glycoprotein (MUC-1), glycoproteine-epithelial cell adhesion molecule (EpCAM), KRAS Proto-Oncogene (KRAS), Anaplastic lymphoma kinase (ALK), Cytotoxic T-Lymphocyte Associated Protein 4 (CTLA-4), Programmed cell death protein 1 (PD-1), Epidermal growth factor (EGF), Serine protease easter (EA), Telomerase reverse transcriptaseh (TERT), PRAME Nuclear Receptor Transcriptional Regulator (PRAME), Receptor tyrosine-protein kinase erbB-2 (HER), or Vascular endothelial growth factor (VEGF), Carcinoembryonic antigen (CEA), MAGE-AL MAGE-A4, Survivin, Six Transmembrane Epithelial Antigene of the Prostate 1 (STEAP1), SRY (sex determining region Y)-box 2 (SOX2), or Cancer/testis antigen 1 (CTAG1B).

In some embodiments, the targeting moiety targets a biomarker expressed or released by an endothelial cell. In some embodiments, the endothelial cells are a blood vessel cell. In some embodiments, the endothelial cell is a lymphatic vessel cell. In some embodiments, the biomarker is expressed or released by a blood vessel cell. In some embodiments, the biomarker is expressed or released by a lymphatic vessel cell. Non-limiting examples of the endothelial cell biomarker include angiotensin I converting enzyme (ACE or CD143), C1qR1/CD93, VE-Cadherin, CC Chemokine Receptor D6, CD31/PECAM-1, CD34, CD36/SR-B3, CD151, CD160, CD300g/Nepmucin, CL-K1/COLEC11, CL-P1/COLEC12, Coagulation Factor III/Tissue Factor, DC-SIGNR/CD299, DCBLD2/ESDN, ECSCR, EMMPRIN/CD147, Endoglin/CD105, Endomucin, Endosialin/CD248, EPCR, Erythropoietin R, endothelial cell adhesion molecule (ESAM), fatty acid binding protein 5 (FABP5 or E-FABP), fatty acid binding protein 6 (FABP6), ICAM-1/CD54, ICAM-2/CD102, IL-1 RI, IL-13 R alpha 1, Integrin alpha 4/CD49d, Integrin alpha 4 beta 1, Integrin alpha 4 beta 7/LPAM-1, Integrin beta 2/CD18, Kruppel like factor 4 (KLF4), lymphatic vessel endothelial hyaluronan receptor 1 (LYVE-1), MCAM/CD146, Nectin-2/CD112, PD-ECGF/Thymidine Phosphorylase, Podocalyxin, Podoplanin, sphingosine-1-phosphate receptor 1 (S1P1 or EDG-1), sphingosine-1-phosphate receptor 2 (S1P2 or EDG-5), sphingosine-1-phosphate receptor 3 (S1P3 or EDG-3), sphingosine-1-phosphate receptor 4 (S1P4 or EDG-6), sphingosine-1-phosphate receptor 5 (SIPS or EDG-8), E-Selectin/CD62E, P-Selectin/CD62P, SLAM/CD150, Stabilin-1, Stabilin-2, plexin domain containing 1 (TEM7 or PLXDC1), ANTXR cell adhesion molecule 1 (TEM8 or ANTXR1), Thrombomodulin/BDCA-3, thrombospondin type 1 domain containing 1 (THSD1), thrombospondin type 1 domain containing 7A (THSD7A), TEK receptor tyrosine kinase (Tie-2), TNF RI/TNFRSF1A, TNF RII/TNFRSF1B, TRA-1-85/CD147, TRAIL R2/TNFRSF10B, TRAILR1/TNFRSF10A, VCAM-1/CD106, VE-Statin, VEGFR1/Flt-1, VEGFR2/KDR/Flk-1, VEGFR3/Flt-4, angiogenic factor with G-patch and FHA domains 1 (VGSQ), or von Willebrand factor (vWF-A2).

In some embodiments, the targeting moiety comprises a chemokine receptor or a chemokine ligand, or portion thereof, involved in chemokine signaling such as, for example, SDF-1a/CXCR4, CCL2/CCR2, or adhesion molecules such, as for example, PSGL-1. As shown herein, the enucleated cell may be enucleated to express functional CXCR4, CCR2 as well as glycosylated PSGL-1, which may greatly promote the specific targeting of the enucleated cell. In some embodiments, the targeting moiety such as, CXCR4, CCR2 or PSGL-1 may be expressed on the surface of the enucleated cell. Non-limiting examples of cell surface proteins that may be expressed on the cell surface of the enucleated cell as the targeting moiety include chemokines such as CXCR4, CCR2, CCR1, CCR5, CXCR7, CXCR2, and CXCR1. In some embodiments, the enucleated cell may be enucleated to secrete the targeting moiety or is tethered to the extracellular matrix, e.g., SDF1α or CCL2. Non-limiting examples of targeting moiety that may be secreted by the enucleated cell include SDF1α, CCL2, CCL3, CCL5, CCL8, CCL1, CXCL9, CXCL10, CCL11 and CXCL12. In some embodiments, the enucleated cell comprises cell-matrix receptors and cell-cell adhesion molecules include integrins, cadherins, glycoproteins, and heparin sulfate proteoglycans.

In some embodiments, the enucleated cells may further include (e.g., by engineering or from the cell from which they were obtained) a surface marker that aids in their evasion of the subject immune system. For example, in some embodiments, the enucleated cells may include a CD47, PD-L1, HLA-E, HLA-G, a fragment thereof, or a combination thereof. Without being bound by any particular theory, it is believed that a CD47, PD-L1, HLA-E, HLA-G, a fragment thereof, or a combination thereof helps to prevent the enucleated cells from being phagocytosed by macrophages. Non-limiting examples of cell-matrix receptors and cell-cell adhesion molecules include integrins, cadherins, glycoproteins, or heparin sulfate proteoglycans. In some embodiments, the cell-matrix receptors or cell-cell adhesion molecules include PD-L1, HLA-E, or HLA-G. Non-limiting examples of therapeutic molecules include tumor antigens and immunomodulatory peptides, polyamines, and ATP. In some embodiments, the therapeutic molecules can be recognized by immune cells and can induce immune response. For example, the therapeutic molecules can be 4-1BB or any one of the cytokine described herein to induce immune response.

### Therapeutic Agent

In some embodiments, the cells of the present disclosure comprise at least one therapeutic agent. In some embodiments, the cells are enucleated, such as with the methods of enucleation disclosed herein. In some embodiments, the therapeutic agent comprises an active agent In some embodiments, an active agent comprises at least one of a DNA molecule, a RNA molecule, a protein (e.g., an enzyme, an antibody, an antigen, a toxin, cytokine, a protein hormone, a growth factor, a cell surface receptor, or a vaccine), a peptide (e.g., a peptide hormone or an antigen), a small molecule (e.g., a steroid, a polyketide, an alkaloid, a toxin, an antibiotic, an antiviral, a colchicine, a taxol, a mitomycin, or emtansine), a gene editing factor, a nanoparticle, or another active agent (e.g., bacteria, bacterial spores, bacteriophages, bacterial components, viruses (e.g., oncolytic viruses), exosomes, lipids, or ions). Non-limiting examples of RNA molecules include messenger RNA (mRNA), short hairpin RNA (shRNA), small interfering RNA (siRNA), microRNA, long non-coding RNA (lncRNA) and a RNA virus. Non-limiting examples of DNA molecules include a single-stranded DNA, double-stranded DNA, an oligonucleotide, a plasmid, a bacterial DNA molecule and a DNA virus. Non-limiting examples of proteins include a cytokine, a growth factor, a hormone, an antibody or an antigen-binding fragment thereof, a single-domain antibody or antigen binding fragment thereof, a small-peptide based drug, oand an enzyme. Non-limiting examples of oncolytic viruses include Talimogene laherparepvec, Onyx-015, GL-ONC1, CV706, Voyager-V1, and HSV-1716. Some wild-type viruses also show oncolytic behavior such as Vaccinia virus, Vesicular stomatitis virus, Poliovirus, Reovirus, Senecavirus, ECHO-7, or Semliki Forest virus.

In some embodiments, an enucleated cell is engineered to produce (e.g., express, and in some cases, release or secrete) the therapeutic agent. In some embodiments, the parent cell from which the enucleated cell was obtained may be engineered to produce the therapeutic agent prior to enucleation to produce the enucleated cell. In some embodiments, the enucleated cell is engineered to produce the therapeutic agent after enucleation (in absence of the nucleus). In some embodiments, the therapeutic agent is exogenous to the enucleated cell or parent cell thereof. In some embodiments, the therapeutic agent is endogenous to the enucleated cell or parent cell thereof. In some embodiments, the enucleated cell of the present disclosure comprises at least two, three, four, five, six, seven, eight, nine, ten, or more therapeutic agents.

In some embodiments, the therapeutic agent comprises modified version of the DNA molecule, the RNA molecule, the protein, the peptide, the small molecule active agent, and/or the gene-editing factor as compared to a naturally occurring version. In some embodiments, the therapeutic agent is a corrected, a truncated, or a non-mutated version and/or copy of the DNA molecule, the RNA molecule, the protein, the peptide, the small molecule active agent, and/or the gene-editing factor. For example, the therapeutic agent can correct a mutated tumor protein p53 (p53) or epidermal growth factor receptor (EGFR) in the target cell as part of the treatment for lung cancer.

The therapeutic agent may be, or include, a targeting moiety described herein. Non-limiting example of the targeting moieties that may be produced by or contained in a enucleated cell includes chemokine receptors, adhesion molecules, and antigen-binding polypeptides (e.g. single-domain antibodies and antigen-binding fragments thereof), or a portion thereof. In some embodiments, the therapeutic agent may be, or include, a transmembrane moiety described herein.

In some embodiments, the therapeutic agent is recombinantly expressed by the enucleated cell or parent cell thereof. In some embodiments, the parent cell from which the enucleated cell is derived or obtained is engineered to produce or express the therapeutic agent. In some embodiments, expression of the therapeutic agent is stable (e.g., permanent). In some embodiments, the expression of the therapeutic agent by the parent cell is transient (e.g., non-permanent). In some embodiments, the parent cell is enucleated prior to engineering the enucleated cell to recombinantly express the therapeutic agent. In some embodiments, the parent cell is engineered to recombinantly express the therapeutic agent prior to enucleation.

In some embodiments, the therapeutic agent is not naturally expressed (e.g., in the absence of engineering) in the cell from which the enucleated cell was derived or obtained (e.g., the therapeutic agent is exogenous to the parent cell). In some embodiments, the therapeutic agent is not naturally expressed in the subject (e.g., the therapeutic agent is exogenous to the subject). In some embodiments, the therapeutic agent is not naturally expressed in the subject at the intended site of therapy (e.g., a tumor, or a particular tissue such as the brain, the intestine, the lungs, the heart, the liver, the spleen, the pancreas, muscles, eyes, and the like) (e.g., the therapeutic agent is exogenous to the intended site of therapy). In some embodiments, the level of the therapeutic agent is not naturally occurring in the enucleated cell of the parent cell, such as over expression or under expression of the therapeutic agent.

In some embodiments, the therapeutic agent is derived from a synthetic cell and loaded into the enucleated cell. For example, the therapeutic agent may be endocytosed into the cell prior to or after enucleation of the cell. Alternatively, the therapeutic agent may be synthesized by the cell and subsequently delivered to a target cell.

In some embodiments, therapeutic agent comprises at least 2 (e.g., at least 2, 3, 4, 5, or more) different DNA molecules, RNA molecules, proteins, peptides, small molecule active agents, or gene-editing factors, in any combination. For example, in some embodiments, a therapeutic agent comprises a DNA molecule and a small molecule active agent. For example, in some embodiments, the therapeutic agent comprises two different small molecule active agents. For example, in some embodiments, the therapeutic agent comprises a chemokine receptor (e.g., for targeting) and a small molecule active agent.

In some embodiments, the therapeutic agent comprises a polypeptide. In some embodiments, the polypeptide is exogenous. In some embodiments, the polypeptide is encoded by an exogenous polynucleotide delivered into the parent cell or the enucleated cell. In some embodiments, the polypeptide is synthesized or released by at least one intracellular organelle of the enucleated cell. In some embodiments, the polypeptide is released by the enucleated cell. In some embodiments, the polypeptide is expressed on the cell surface or the enucleated cell. In some embodiments, the enucleated cell delivers the polypeptide to a target cell. In some embodiments, the target cell is a cancer cell. In some embodiments, the cancer cell expresses the cancer biomarker of any cancer described herein. In some embodiments, the target cell is an endothelial cell. In some embodiments, the endothelial cell expresses an endothelial biomarker described herein. In some embodiments, the endothelial cell is a blood vessel cell. In some embodiments, the endothelial cell is a lymphatic vessel cell.

In some embodiments, the exogenous polypeptide comprises a cytokine of any one of the cytokine described herein. In some embodiments, the exogenous polypeptide comprises a soluble cytokine. For example, the exogenous polypeptide can comprise an extracellular domain or fragment of the cytokine. In some embodiments, the exogenous polypeptide comprises a solubility as determined by turbidimetric solubility assay or thermodynamic solubility assay by dissolving the exogenous polypeptide in solvent such as organic solvent, including dimethyl sulfoxide (DMSO), dimethylformamide (DMF), acetonitrile, etc., or inorganic solvent, including water or phosphate-buffered saline (PBS). In some embodiments, the exogenous polypeptide comprises a solubility that is at least 0.0001 mg/ml, 0.0005 mg/ml, 0.001 mg/ml, 0.005 mg/ml, 0.01 mg/ml, 0.05 mg/ml, 0.1 mg/ml, 0.5 mg/ml, 1.0 mg/ml, 5.0 mg/ml, 10 mg/ml, 50 mg/ml, 100 mg/ml, 500 mg/ml 1,000 mg/ml 5,000 mg/ml, 10,000 mg/ml, 50,000 mg/ml, or 100,000 mg/ml.

In some embodiments, the exogenous polypeptide comprises a tumor necrosis factor (TNF) superfamily member polypeptide or a catalytically active fragment thereof. Non-limiting examples of the TNF superfamily member polypeptide include Lymphotoxin alpha (TNFβ), Tumor necrosis factor (TNFα), Lymphotoxin beta (TNFγ), OX40 ligand (CD252, Gp34, or CD134L), CD40 ligand (CD154, TRAP, Gp39, or T-BAM), Fas ligand (CD178, APTL, or CD95L), CD27 ligand (CD70), CD30 ligand (CD153), CD137 ligand (4-1 BBL), TNF-related apoptosis-inducing ligand (CD253 or APO-2L), Receptor activator of nuclear factor kappa-B ligand (CD254, OPGL, TRANCE, or ODF), TNF-related weak inducer of apoptosis (APO-3L or DR3L), a proliferation-inducing ligand (CD256, TALL-2, or TRDL1), B-cell activating factor (CD257, BLyS, TALL-1, or TNFSF20), LIGHT (CD258 or HVEML), Vascular endothelial growth inhibitor (TL1 or TL-1A), TNF superfamily member 18 (GITRL, AITRL, or TL-6), or Ectodysplasin A (ED1-A1 or ED1-A2). In some embodiments, the exogenous polypeptide comprises a peptide sequence that is at least 70%, 75%, 80%, 85%, 90%, 95%, 99%, or more identical to SEQ ID NOs: 1501, 1504, 1505, or 1508. In some embodiments, the exogenous polypeptide comprises LIGHT or a catalytically active fragment thereof. In some embodiments, the exogenous polypeptide comprises a peptide sequence that is at least 70%, 75%, 80%, 85%, 90%, 95%, 99%, or more identical to SEQ ID NO: 1508. In some embodiments, the exogenous polypeptide comprises LIGHT or a catalytically active fragment thereof. In some embodiments, the exogenous polypeptide comprises a peptide sequence that is at least 70%, 75%, 80%, 85%, 90%, 95%, 99%, or more identical to SEQ ID NO: 1511.

In some embodiments, the exogenous polypeptide comprises a soluble member of the TNF superfamily or a soluble catalytic active fragment thereof. In some embodiments, the soluble TNF superfamily member polypeptide or the catalytic active fragment thereof comprises a peptide sequence that is at least 70%, 75%, 80%, 85%, 90%, 95%, 99%, or more identical to SEQ ID NOs: 1501-1511. In some embodiments, the soluble TNF superfamily member polypeptide or the catalytic active fragment thereof comprises a peptide sequence that is at least 70%, 75%, 80%, 85%, 90%, 95%, 99%, or more identical to SEQ ID NOs: 1508-1510. In some embodiments, the soluble TNF superfamily member polypeptide or the catalytic active fragment thereof comprises a peptide sequence that is at least 70%, 75%, 80%, 85%, 90%, 95%, 99%, or more identical to SEQ ID NO: 1511.

In some embodiments, the therapeutic agent comprises any one of the immune checkpoint molecule described herein or an immune checkpoint molecule inhibitor for inhibiting any one of the immune checkpoint molecule described herein. Non-limiting examples of the immune checkpoint molecule include PD-1, PD-L1, CTLA-4, VISTA, PDCD1LG2 (CD273), B7-H3 (also called CD276), A2AR, CD27, LAG3, TIM-3, T cell immunoreceptor with Ig and ITIM domains (TIGIT), CD73, NKG2A, PVRIG, PVRL2, CEACAM1, CEACAM5, CEACAM6, FAK, CCR-2, CCL-2, LIF, CD47, SIRPα, M-CSF, CSF-1R, IL-3, IL-1RAP, IL-8, SEMA4D, Angiopoietin-2, CLEVER-1, Axl, phosphatidylserine or a fragment thereof.

In some embodiments, the therapeutic agent comprises an antibody such as a single-domain antibody described herein. In some embodiments, the antibody or the single-domain antibody binds to an immune checkpoint molecule. In some embodiments, the single-domain antibody bind to, and modulates the expression or the activity of the immune checkpoint molecule. In some embodiments, the single-domain antibody is an inhibitor of the activity or expression of the immune checkpoint molecule. In some embodiments, the single-domain antibody is an activator of the activity or expression of the immune checkpoint molecule. In some embodiments, the antibody or single-domain antibody binds to PD-1, PD-L1, CTLA-4, VISTA, PDCD1LG2 (CD273), B7-H3 (also called CD276), A2AR, CD27, LAG3, TIM-3, T cell immunoreceptor with Ig and ITIM domains (TIGIT), CD73, NKG2A, PVRIG, PVRL2, CEACAM1, CEACAM5, CEACAM6, FAK, CCR-2, CCL-2, LIF, CD47, SIRPα, M-CSF, CSF-1R, IL-3, IL-1RAP, IL-8, SEMA4D, Angiopoietin-2, CLEVER-1, Axl, phosphatidylserine or a fragment thereof. In some embodiments, the therapeutic agent comprising the antibody or the single-domain antibody binds to PD-L1. In some embodiments, the therapeutic agent comprising the antibody or the single-domain antibody binds to CTLA-4. In some embodiments, the immune checkpoint molecule comprises an amino acid sequence that is greater than or equal to about 80% identical to any one of SEQ ID NOs: 155-164, 203, 204, 315-322, 511, 531-535, 551-554, 571, 594, 611-619, or 711. In some embodiments, the single-domain antibody or fragment thereof is encoded by a deoxyribonucleic acid (DNA) sequence that is greater than or equal to about 50%, 55%, 60%, 65%, 70%, 75%, 80%, 85%, 90%, 95%, or 99% identical to SEQ ID NO: 801. In some embodiments, the single-domain antibody or fragment thereof comprises an amino acid sequence that is greater than or equal to about 50%, 55%, 60%, 65%, 70%, 75%, 80%, 85%, 90%, 95%, or 99% identical to SEQ ID NO: 851. In some embodiments, the single-domain antibody or fragment thereof is encoded from a DNA sequence that is greater than or equal to about 50%, 55%, 60%, 65%, 70%, 75%, 80%, 85%, 90%, 95%, or 99% identical to SEQ ID NO: 901. In some embodiments, the single-domain antibody or fragment thereof comprises an amino acid sequence that is greater than or equal to about 50%, 55%, 60%, 65%, 70%, 75%, 80%, 85%, 90%, 95%, or 99% identical to SEQ ID NO: 951.

In some embodiments, therapeutic agent comprises antibody such as single-domain antibody binding a connective tissue growth factor (CTGF). In some embodiments, the single-domain antibody or fragment thereof comprises a polypeptide sequence that is greater than or equal to about 50%, 55%, 60%, 65%, 70%, 75%, 80%, 85%, 90%, 95%, or 99% identical to SEQ ID NO: 1701. In some embodiments, the single-domain antibody or fragment thereof binds to an amino acid sequence of CTGF, wherein the amino acid sequence of CTGF comprises SEQ ID NO: 1601 or SEQ ID NO: 1602.

In some embodiments, the therapeutic agent comprises an exogenous agent comprising an angiogenesis inhibitor. In some embodiments, the angiogenesis inhibitor comprises an inhibitor that inhibits vascular endothelial growth factor (VEGF) receptor (VEGFR), or the combination thereof (VEGF/VEGFR). In some embodiments, the VEGF/VEGFR inhibitor comprises a compound, small molecule, peptide, antibody, or a combination thereof. In some embodiments, the VEGF inhibitor inhibits VEGF-A, VEGF-B, VEGF-C, VEGF-D, placental growth factor (PIGF), or a combination thereof. In some embodiments, the VEGF inhibitor is a VEGF-A inhibitor. In some embodiments, the VEGF inhibitor comprises an antibody or a single-domain antibody for targeting and inhibiting VEGF-A. In some embodiments, the VEGF inhibitor comprises a peptide such as a KDR or FLT1 peptide. In some embodiments, the VEGF inhibitor comprises an antibody or a single-domain antibody for targeting and inhibiting VEGF-A receptor.

### Antibody and Single-Domain Antibodies

Described herein, in some embodiments, are enucleated cells comprising an antibody or an antigen-binding fragment. In some embodiments, the enucleated cells comprise a polynucleotide encoding the antibody or the antigen-binding fragment thereof. In a particular embodiment, the antibody or the antigen-binding fragment thereof of the disclosure may be neutralizing antibody, non-neutralizing antibody, or a combination thereof. In some embodiments, the antibody or antigen-binding fragment is a single-domain antibody. The utility and advantages of single-domain antibodies (sdAbs) include, but are not limited to, their smaller size, larger number of accessible epitopes, relatively low production costs and improved robustness, as compared with their full-length antibodies.

In some embodiments, the antibody or the antigen-binding fragment thereof described herein is a humanized antibody, a variant, or a derivative thereof, that may, for example, be formulated for administration to a human. In some embodiments, the humanized antibody is chimeric humanized antibody or fully human antibody, for example, comprising an amino acid sequence from or with similarity to a human antibody amino acid sequence, and a non-human amino acid sequence. For example, a portion of the heavy and/or light chain of a chimeric humanized antibody may be identical to or similar to a corresponding sequence in a human antibody, while the remainder of the chain(s) may be non-human, for example, identical or similar to a corresponding sequence in an antibody derived from another species or belonging to another antibody class or subclass. The non-human sequence may be humanized to reduce the likelihood of immunogenicity while preserving target specificity, for example, by incorporation of human DNA to the genetic sequence of the genes that produce the antibodies in the non-human animal. Humanized antibody may be fully human antibody, for example, containing an amino acid sequence that is a human antibody amino acid sequence.

The antibody or the antigen-binding fragment thereof of the present disclosure may comprise a basic four chain antibody unit. The basic four chain antibody unit comprises two heavy chain (H) polypeptide sequences and two light chain (L) polypeptide sequences. Each of the heavy chains comprises one N-terminal variable (VH) region and three or four C-terminal constant (CH1, CH2, CH3, and CH4) regions. Each of the light chains comprises one N-terminal variable (VL) region and one C-terminal constant (CL) region. The light chain variable region is aligned with the heavy chain variable region and the light chain constant region is aligned with first heavy chain constant region CH1. The pairing of a heavy chain variable region and light chain variable region together forms a single antigen-binding site. Each light chain is linked to a heavy chain by one covalent disulfide bond. The two heavy chains are linked to each other by one or more disulfide bonds depending on the heavy chain isotype. Each heavy and light chain may also comprise regularly-spaced intrachain disulfide bridges. The C-terminal constant regions of the heavy chains comprise the Fc region of the antibody, which may mediate effector functions, for example, through interactions with Fc receptors or complement proteins.

The light chain may be designated kappa or lambda based on the amino acid sequence of the constant region. The heavy chain may be designated alpha, delta, epsilon, gamma, or mu based on the amino acid sequence of the constant region. Antibodies are categorized into five immunoglobulin classes, or isotypes, based on the heavy chain. IgA comprises alpha heavy chains, IgD comprises delta heavy chains, IgE comprises epsilon heavy chains, IgG comprises gamma heavy chains, and IgM comprises mu heavy chains. Antibodies of the IgG, IgD, and IgE classes comprise monomers of the four chain unit described above (two heavy and two light chains), while the IgM and IgA classes comprises multimers of the four chain unit. The alpha and gamma classes may further be divided into subclasses on the basis of differences in the sequence and function of the heavy chain constant region. Subclasses of IgA and IgG expressed by humans include IgG1, IgG2, IgG3, IgG4, IgA1 and IgA2. An antibody of the disclosure comprises a human light chain constant domain sequence, e.g., a kappa (IgK) or lambda (IgL) chain. In some embodiments, the antibody comprises a human IgK constant domain, variant, derivative, or fragment thereof. In some embodiments, the antibody comprises a human IgL constant domain, variant, derivative, or fragment thereof.

In some embodiments, an antibody or an antigen-binding fragment thereof, or a single-domain antibody or an antigen-binding fragment thereof described herein comprises a signal peptidase. Signal peptides may result in higher protein expression and/or secretion by a cell. Signal peptidases may cleave a signal peptide off the antibody or the antigen-binding fragment thereof, or the single-domain antibody or the antigen-binding fragment thereof, for example, during a secretion process, generating a mature antibody that does not comprise the signal peptide sequence.

The constant regions of the antibody or the antigen-binding fragment thereof, or the single-domain antibody or the antigen-binding fragment thereof may mediate various effector functions and may be minimally involved in antigen binding. Different IgG isotypes or subclasses may be associated with different effector functions or therapeutic characteristics, for example, because of interactions with different Fc receptors and/or complement proteins. Antibodies comprising Fc regions that engage activating Fc receptors can, for example, participate in antibody-dependent cell-mediated cytotoxicity (ADCC), antibody-dependent cellular phagocytosis (ADCP), complement-dependent cytotoxicity (CDC), induction of signaling through immunoreceptor tyrosine-based activation motifs (ITAMs), and induction of cytokine secretion. Antibodies comprising Fc regions that engage inhibitory Fc receptors can, for example, induce signaling through immunoreceptor tyrosine-based inhibitory motifs (ITIMs).

Different antibody subclasses comprise different abilities to elicit immune effector functions. For example, IgG1 and IgG3 may effectively recruit complement to activate CDC, IgG2 elicits minimal ADCC. IgG4 has a lesser ability to trigger immune effector functions. Modifications to the constant regions may also affect antibody characteristics, for example, enhancement or reduction of Fc receptor ligation, enhancement or reduction of ADCC, enhancement or reduction of ADCP, enhancement or reduction of CDC, enhancement or reduction of signaling through ITAMs, enhancement or reduction of cytokine induction, enhancement or reduction of signaling through ITIMs, enhancement or reduction of half-life, or enhancement or reduction of co-engagement of antigen with Fc receptors. Modifications may include, for example, amino acid mutations, altering post-translational modifications (e.g., glycosylation), combining domains from different isotypes or subclasses, or a combination thereof.

Antibodies or the antigen-binding fragments thereof of the disclosure comprises constant regions or Fc regions that are selected or modified to provide suitable antibody characteristics, for example, suitable characteristics for treating a disease or condition as disclosed herein. In some embodiments, IgG1 may be used, for example, to promote inflammation, immune activation, and immune effector functions for the treatment of an infection. In some embodiments, IgG4 may be used, for example, in cases where antagonistic property of the antibody with reduced immune effector functions are desired (e.g., to neutralize coronavirus antigens and inhibit viral entry into cells without promoting inflammation and immune activation).

The variable (V) regions may mediate antigen binding and define the specificity of a particular antibody for an antigen. The variable region comprises relatively invariant sequences called framework regions, and hypervariable regions, which differ considerably in sequence among antibodies of different binding specificities. The variable region of each antibody heavy or light chain comprises four framework regions separated by three hypervariable regions. The variable regions of heavy and light chains fold in a manner that brings the hypervariable regions together in close proximity to create an antigen binding site. The four framework regions largely adopt an f3-sheet configuration, while the three hypervariable regions form loops connecting, and in some cases forming part of, the f3-sheet structure.

Within hypervariable regions are amino acid residues that primarily determine the binding specificity of the antibody. Sequences comprising these residues are known as complementarity determining regions (CDRs). One antigen binding site of an antibody comprises six CDRs, three in the hypervariable regions of the light chain, and three in the hypervariable regions of the heavy chain. The CDRs in the light chain may be designated LCDR1, LCDR2, LCDR3, while the CDRs in the heavy chain may be designated HCDR1, HCDR2, and HCDR3.

In some embodiments, antibodies or the antigen-binding fragments thereof of the disclosure include variants or derivatives thereof. For example, a non-human animal may be genetically modified to produce antibody variants or derivatives. In some embodiments, an antibody may be a single-domain antibody (sdAb), for example, a heavy chain only antibody (HCAb) VHH, or nanobody. Non-limiting examples of antigen-binding fragments include Fab, Fab′, F(ab′)2, dimers and trimers of Fab IL-6Rs, Fv, scFv, minibodies, dia-, tria-, and tetrabodies, and linear antibodies. Fab and Fab′ are antigen-binding fragments that comprise the VH and CH1 domains of the heavy chain linked to the VL and CL domains of the light chain via a disulfide bond. A F(ab′)2 comprises two Fab or Fab′ that are joined by disulfide bonds. A Fv comprises the VH and VL domains held together by non-covalent interactions. A scFv (single-chain variable fragment) is a fusion protein that comprises the VH and VL domains connected by a peptide linker. Manipulation of the orientation of the VH and VL domains and the linker length may be used to create different forms of molecules that may be monomeric, dimeric (diabody), trimeric (triabody), or tetrameric (tetrabody). Minibodies are scFv-CH3 fusion proteins that assemble into bivalent dimers.

In other embodiments, the antibody is a binding fragment thereof. In some cases, the antibody is a humanized antibody or binding fragment thereof, a chimeric antibody or binding fragment thereof, a monoclonal antibody or binding fragment thereof, a multi-specific antibody or binding fragment thereof, a bispecific antibody or binding fragment thereof, or a single-domain antibody (e.g. Nanobody®) thereof. In some cases, the antibody is monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (“dsFv”), single-domain antibody (sdAb), Ig NAR, camelid antibody or binding fragment thereof, or a chemically modified derivative thereof. In some embodiments, the antibody may be a multi-specific antibody. In some cases, the multi-specific antibody comprises two or more target binding moieties in which each of the two or more target binding moieties binds specifically to an antigen, and the two or more antigens are different. In some cases, the multi-specific antibody comprises target binding moieties that specifically bind to three or more different antigens, four or more different antigens, or five or more different antigens. In some embodiments, the antibody may be a bispecific antibody. In some cases, the bispecific antibody or binding fragment includes a Knobs-into-Holes (KiH), Asymmetric Re-engineering Technology-immunoglobulin (ART-Ig), Triomab quadroma, bispecific monoclonal antibody (BiMAb, BsmAb, BsAb, bsMab, BS-Mab, or Bi-MAb), FcAAdp, XmAb, Azymetric, Bispecific Engagement by Antibodies based on the T-cell receptor (BEAT), Bispecific T-cell Engager (BiTE), Biclonics, Fab-scFv-Fc, Two-in-one/Dual Action Fab (DAF), FinomAb, scFv-Fc-(Fab)-fusion, Dock-aNd-Lock (DNL), Adaptir (previously SCORPION), Tandem diAbody (TandAb), Dual-affinity-ReTargeting (DART), or nanobody. In some embodiments, the bispecific antibody is a trifunctional antibody or a bispecific mini-antibody. In some cases, the bispecific antibody is a trifunctional antibody. The trifunctional antibody may be a full length monoclonal antibody comprising binding sites for two different antigens.

In some cases, the bispecific antibody is a bispecific mini-antibody. In some cases, the bispecific mini-antibody comprises divalent Fab2, F(ab)′3 fragments, bis-scFv, (scFv)2, diabody, minibody, triabody, tetrabody or a bi-specific T-cell engager (BiTE). In some embodiments, the bi-specific T-cell engager is a fusion protein that contains two single-chain variable fragments (scFvs) in which the two scFvs target epitopes of two different antigens.

In some embodiments, the antibody described herein comprises an IgG framework, an IgA framework, an IgE framework, or an IgM framework. In some cases, the antibody comprises an IgG framework (e.g., IgG1, IgG2, IgG3, or IgG4). In some cases, the antibody comprises an IgG1 framework. In some cases, the antibody comprises an IgG2 (e.g., an IgG2a or IgG2b) framework. In some cases, the antibody comprises an IgG2a framework. In some cases, the antibody comprises an IgG2b framework. In some cases, the antibody comprises an IgG3 framework. In some cases, the antibody comprises an IgG4 framework.

In some cases, the antibody described herein comprises one or more mutations in a framework region, e.g., in the CH1 domain, CH2 domain, CH3 domain, hinge region, or a combination thereof. In some cases, the one or more mutations are to stabilize the antibody and/or to increase half-life. In some cases, the one or more mutations are to modulate Fc receptor interactions, to reduce or eliminate Fc effector functions such as FcyR, antibody-dependent cell-mediated cytotoxicity (ADCC) or complement-dependent cytotoxicity (CDC). In additional cases, the one or more mutations are to modulate glycosylation.

In some embodiments, the antibody comprises a humanized antibody or binding fragment thereof or a chimeric antibody or binding fragment thereof. In some embodiments, the comprises a multi-specific antibody or binding fragment thereof. In some embodiments, the antibody comprises a bispecific antibody or binding fragment thereof. In some embodiments, the antibody may be an IgG-scFv, nanobody, BiTE, diabody, DART, TandAb, scDiabody, scDiabody-CH3, triple body, mini-antibody, minibody, TriBi minibody, scFv-CH3 KIH, Fab-scFv-Fc KIH, Fab-scFv, scFv-CH-CL-scFv, F(ab′)2, F(ab′)2-scFv2. scFv-KIH, Fab-scFv-Fc, tetravalent HCAb, scDiabody-Fc, diabody-Fc, tandem scFv-Fc, or intrabody. In some cases, the antibody is monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (“dsFv”), single-domain antibody (e.g., a scFv), Ig NAR, camelid antibody, or binding fragment thereof, or a chemically modified derivative thereof.

In some embodiments, the antibody or the antigen-binding fragment thereof, or single-domain antibody or the antigen-binding fragment thereof binds to an epitope expressed by the target cell associated with the disease or condition described herein. In some embodiments, the antibody or the antigen-binding fragment thereof, or the single-domain antibody or the antigen-binding fragment thereof binds to an epitope associated with the microenvironment described herein. Non-limiting examples of epitopes include peptide fragment of cytokine, immune checkpoint molecule, or any other protein associated with the disease or the condition. Non-limiting examples of cytokine may include 4-1BBL, acylation stimulating protein, adipokine, albinterferon, APRIL, Arh, BAFF, Bcl-6, CCL1, CCL1/TCA3, CCL11, CCL12/MCP-5, CCL13/MCP-4, CCL14, CCL15, CCL16, CCL17/TARC, CCL18, CCL19, CCL2, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCLS, CCL6, CCL7, CCL8, CCL9, CCR10, CCR3, CCR4, CCR5, CCR6, CCR7, CCR8, CD153, CD154, CD178, CD40LG, CD70, CD95L/CD178, Cerberus (protein), chemokines, CLCF1, CNTF, colony-stimulating factor, common b chain (CD131), common g chain (CD132), CX3CL1, CX3CR1, CXCL1, CXCL10, CXCL11, CXCL12, CXCL13, CXCL14, CXCL15, CXCL16, CXCL17, CXCL2, CXCL2/MIP-2, CXCL3, CXCL4, CXCL5, CXCL6, CXCL7, CXCL9, CXCR3, CXCR4, CXCR5, EDA-A1, Epo, erythropoietin, FAM19A1, FAM19A2, FAM19A3, FAM19A4, FAM19A5, Flt-3L, FMS-like tyrosine kinase 3 ligand, Foxp3, GATA-3, GcMAF, G-CSF, GITRL, GM-CSF, granulocyte colony-stimulating factor, granulocyte-macrophage colony-stimulating factor, hepatocyte growth factor, IFNA1, IFNA10, IFNA13, IFNA14, IFNA2, IFNA4, IFNA5/IFNaG, IFNA7, IFNA8, IFNB1, IFNE, IFNG, IFNZ, IFN-α, IFN-β, IFN-γ, IFNω/IFNW1, IL-1, IL-10, IL-10 family, IL-10-like, IL-11, IL-12, IL-13, IL-14, IL-15, IL-16, IL-17, IL-17 family, IL-17A-F, IL-18, IL-18BP, IL-19, IL-1A, IL-1B, IL-1F10, IL-1F3/IL-1RA, IL-1F5, IL-1F6, IL-1F7, IL-1F8, IL-1F9, IL-1-like, IL-1RA, IL-1RL2, IL-1α, IL-1β, IL-2, IL-20, IL-21, IL-22, IL-23, IL-24, IL-28A, IL-28B, IL-29, IL-3, IL-31, IL-33, IL-35, IL-4, IL-5, IL-6, IL-6-like, IL-7, IL-8/CXCL8, IL-9, inflammasome, interferome, interferon, interferon beta-1a, interferon beta-1b, interferon gamma, interferon type I, interferon type II, interferon type III, interferons, interleukin, interleukin 1 receptor antagonist, Interleukin 8, IRF4, Leptin, leukemia inhibitory factor (LIF), leukocyte-promoting factor, LIGHT, LTA/TNFB, LT-β, lymphokine, lymphotoxin, lymphotoxin alpha, lymphotoxin beta, macrophage colony-stimulating factor, macrophage inflammatory protein, macrophage-activating factor, M-CSF, MHC class III, miscellaneous hematopoietins, monokine, MSP, myokine, myonectin, nicotinamide phosphoribosyltransferase, oncostatin M (OSM), oprelvekin, OX40L, platelet factor 4, promegapoietin, RANKL, SCF, STAT3, STAT4, STAT6, stromal cell-derived factor 1, TALL-1, TBX21, TGF-α, TGF-β, TGF-β1, TGF-β2, TGF-β3, TNF, TNFSF10, TNFSF11, TNFSF12, TNFSF13, TNFSF14, TNFSF15, TNFSF4, TNFSF8, TNF-α, TNF-β, Tpo, TRAIL, TRANCE, TWEAK, vascular endothelial growth inhibitor, XCL1, or XCL2. Example of immune checkpoint molecule may include VISTA, PDCD1LG2 (CD273), PD-L1, CTLA-4, PD-L2, B7-1 (CD80), B7-2 (CD86), B7-H3 (CD276), B7-H2, B7-H4 (VTCN1), HVEM (CD270, TNFRSF14), Galectin 9, Galectin3, CEACAM1 (CD66a), OX-2 (CD200), PVR (CD155), PVRL2 (Nectin-2, CD112), FGL-1, PECAM-1, TSG-6, CD47, Stabilin-1 (Clever-1), Neuropilin 1, Neuropilin 2, CD158 (family), IGSF2 (CD101), CD155, GITRL, CD137L, OX40L, LIGHT, CD70, PD-1, RGMB, CTLA-4 (CD152), BTLA, CD160, Tim-3, CD200R, TIGIT, CD112R (PVRIG), LAG-3 (CD223), PECAM-1, CD44, SIRP alpha (CD172a), or IGSF11. In some embodiments, the epitope is a peptide sequence selected from LAG-3, P2X7, or albumin, or any combination or portions thereof. In some embodiments, the antibody or the antigen-binding fragment thereof (e.g., single-domain antibody) binds to an epitope expressed by the target cell associated with idiopathic pulmonary fibrosis. In some embodiments, the antibody or the antigen-binding fragment thereof (e.g., single-domain antibody) binds to an epitope comprising a peptide sequence encoding CTGF. In some embodiments, the exogenous single-domain antibody or fragment thereof is specific to an antigen comprising at least one peptide sequence in SEQ ID NOs: 1601-1602. In some embodiments, the exogenous single-domain antibody or fragment comprises a peptide sequence that is greater than or equal to about 50%, 55%, 60%, 65%, 70%, 75%, 80%, 85%, 90%, 95%, or 99% identical to SEQ ID NO: 1701.

In some embodiments, the exogenous antibody or the single-domain antibody functions as an agonist or an antagonist, where upon binding to any one of the epitope described herein, the binding of the antibody or the single-domain antibody induces agonist or antagonist effect. For example, the exogenous antibody or the single-domain antibody, upon binding to immune checkpoint inhibitor such as PD-L1/PD-1 or CTLA-4, exerts agonistic or antagonistic effect on the immune checkpoint signaling pathway (SEQ ID NOs: 801, 851, 901, and 951). In some embodiments, the exogenous antibody or the single-domain antibody expressed by the enucleated cell is encoded from a nucleic acid sequence that is greater than or equal to about 50%, 55%, 60%, 65%, 70%, 75%, 80%, 85%, 90%, 95%, or 99% identical to SEQ ID NO: 801. In some embodiments, the exogenous single-domain antibody or fragment thereof comprises a polypeptide sequence that is greater than or equal to about 50%, 55%, 60%, 65%, 70%, 75%, 80%, 85%, 90%, 95%, or 99% identical to SEQ ID NO: 851. In some embodiments, the exogenous single-domain antibody or fragment thereof is encoded from a nucleic acid sequence that is greater than or equal to about 50%, 55%, 60%, 65%, 70%, 75%, 80%, 85%, 90%, 95%, or 99% identical to SEQ ID NO: 901. In some embodiments, the exogenous single-domain antibody or fragment thereof comprises a polypeptide sequence that is greater than or equal to about 50%, 55%, 60%, 65%, 70%, 75%, 80%, 85%, 90%, 95%, or 99% identical to SEQ ID NO: 951.

In some cases, the epitope may include a peptide sequence of a pathogen protein. For example, the epitope may be a viral protein or a fragment thereof. In some embodiments, the epitope may be a viral protein of a coronavirus. In some embodiments, the coronavirus may be severe acute respiratory syndrome-related virus (SARS-CoV). In some embodiments, the SARS-CoV is SARS-CoV-2. In some embodiments, the epitope may be a viral protein selected from orf1a, orf1ab, spike protein (S protein), 3a, 3b, envelope protein (E protein), matrix protein (M protein), p6, 7a, 7b, 8b, 9b, nucleocapsid protein (N protein), orf14, nsp1 (leader protein), nsp2, nsp3, nsp4, nsp5 (3C-like proteinase), nsp6, nsp7, nsp8, nsp9, nsp10 (growth-factor-like protein), nsp12 (RNA-dependent RNA polymerase, or RdRp), nsp13 (RNA 5′-triphosphatase), nsp14 (3′-to-5′ exonuclease), nsp15 (endoRNAse), and nsp16 (2′-O-ribose methyltransferase), a portion thereof, or combinations thereof.

Another example of an epitope may include a peptide sequence of a viral protein of an influenza virus. In some embodiments, the influenza virus is selected from the genera consisting of Influenza virus A, Influenza virus B, Influenza virus C and Influenza virus D. In further embodiments, the influenza A virus is of the subtype H1N1, H2N2, H3N2, H5N1, H7N7, H1N2, H9N2, H7N2, H7N3, H10N7, H7N9, or H6N1. In further embodiments, the influenza B virus of the B/Yamagata/16/88-like lineage or the B/Victoria/2/87-like lineage. In some embodiments, the influenza may be any strain of the influenza virus or any serotypes within a stain of influenza virus. In some cases, the influenza virus comprises any combination viral surface glycoproteins haemagluttinin (H or HA) and neuraminidase (N or NA).

In some embodiments, the epitope is encoded from a nucleic acid sequence provided in SEQ ID NOs: 131-134, 142-152, 201, 202, 301-312, 501, 521-526, 541-545, 561, 584, 591-601, and 701-705. In some embodiments, the epitope is encoded from a nucleic acid sequence that is greater than or equal to about 50%, 55%, 60%, 65%, 70%, 75%, 80%, 85%, 90%, 91%, 92%, 93%, 94%, 95%, 96%, 97%, 98%, 99%, or more identical to the nucleic acid sequences found in SEQ ID NOs: 131-134, 142-152, 201, 202, 301-312, 501, 521-526, 541-545, 561, 584, 591-601, and 701-705.

In some embodiments, the epitope comprises a peptide sequence provided in SEQ ID NOs: 155-164, 203, 204, 315-322, 511, 531-535, 551-554, 571, 594, 611-619, and 711. In some embodiments, the epitope comprises a peptide sequence that is greater than or equal to about 50%, 55%, 60%, 65%, 70%, 75%, 80%, 85%, 90%, 91%, 92%, 93%, 94%, 95%, 96%, 97%, 98%, 99%, or more identical to the peptide sequences found in SEQ ID NOs: 155-164, 203, 204, 315-322, 511, 531-535, 551-554, 571, 594, 611-619, and 711.

In some cases, the antibody or the antigen-binding fragment thereof (e.g., single-domain antibody) is encoded from polynucleotide sequence described herein. In some embodiments, the polynucleotide sequence is exogenous to the enucleated cell or parent cell. In some embodiments, the polynucleotide comprises a nucleic acid sequence that is greater than or equal to about 50%, 55%, 60%, 65%, 70%, 75%, 80%, 85%, 90%, 91%, 92%, 93%, 94%, 95%, 96%, 97%, 98%, 99%, or more identical to the mRNA or the cDNA sequence encoding the antibody or the antigen-binding fragment thereof described herein.

In some embodiments, the enucleated cell comprises a polynucleotide comprising a nucleic acid sequence encoding the antibody or antigen binding fragment thereof (e.g., single-domain antibody). Non-limiting examples of antibody or antigen binding fragment thereof encoded by the polynucleotide herein may be found in SEQ ID NOs: 1-36, 101-111, 121-123, 165-192, 195, 205, 206, 211-213, 221-231, 241-245, 325-331, and 401-404.

In some embodiments, the enucleated cell comprises an antibody or antigen-binding fragment (e.g., single-domain antibody) comprising an amino acid sequence provided in SEQ ID NOs: 1-36, 101-111, 121-123, 165-192, 195, 205, 206, 211-213, 221-231, 241-245, 325-331, and 401-404. In some embodiments, the single-domain antibody comprises an amino acid sequence that is greater than or equal to about 50%, 55%, 60%, 65%, 70%, 75%, 80%, 85%, 90%, 91%, 92%, 93%, 94%, 95%, 96%, 97%, 98%, 99%, or more identical to the amino acid sequences listed in SEQ ID NOs: 1-36, 101-111, 121-123, 165-192, 195, 205, 206, 211-213, 221-231, 241-245, 325-331, and 401-404.

In some embodiments, the antibody or the antigen-binding fragment thereof, or the single-domain antibody, described herein may bind to tumor necrosis factor (TNF). Examples of the peptide sequence of the antibody or the antigen-binding fragment thereof or the single-domain antibody for TNF may be found in SEQ ID NOs: 1-36.

In some embodiments, the antibody or the antigen-binding fragment thereof, or the single-domain antibody, described herein may bind to albumin. Non-limiting examples of the peptide sequence of the antibody or the single-domain antibody for albumin may be found in SEQ ID NOs: 101-111. In some embodiments, the exogenous single-domain antibody or fragment comprises a peptide sequence that is greater than or equal to about 50%, 55%, 60%, 65%, 70%, 75%, 80%, 85%, 90%, 95%, 96%, 97%, 98% or 99% identical to SEQ ID NOs: 101-111.

In some embodiments, the antibody or the antigen-binding fragment thereof or the single-domain antibody described herein may bind to purinergic receptor P2X 7 (P2X7). Examples of the peptide sequence of the antibody or the antigen-binding fragment thereof or the single-domain antibody for P2x7 may be found in SEQ ID NOs: 121-123. In some embodiments, the exogenous single-domain antibody or fragment comprises a peptide sequence that is greater than or equal to about 50%, 55%, 60%, 65%, 70%, 75%, 80%, 85%, 90%, 95%, 96%, 97%, 98% or 99% identical to SEQ ID NOs 121-123.

In some embodiments, the antibody or the antigen-binding fragment thereof or the single-domain antibody described herein may bind to human PD-L1 encoded by a nucleic acid that is at least 75%, 80%, 85%, 90%, 95%, 96%, 97%, 98%, or 99% identical to any one of SEQ ID NOs: 131-152. In some embodiments, the antibody or the antigen-binding fragment thereof or the single-domain antibody described herein may bind to human PD-L1 comprising a polypeptide sequence that is at least 75%, 80%, 85%, 90%, 95%, 96%, 97%, 98%, or 99% identical to any one of SEQ ID NOs: 155-164. Examples of the peptide sequence of the antibody or the antigen-binding fragment thereof or the single-domain antibody for human PD-L1 may be found in SEQ ID NOs: 165-192. In some embodiments, the exogenous single-domain antibody or fragment comprises a peptide sequence that is greater than or equal to about 50%, 55%, 60%, 65%, 70%, 75%, 80%, 85%, 90%, 95%, 96%, 97%, 98% or 99% identical to SEQ ID NOs: 165-192.

In some embodiments, the antibody or the antigen-binding fragment thereof or the single-domain antibody described herein may bind to mouse CD274 antigen (PD-L1). Examples of the peptide sequence of the antibody or the antigen-binding fragment thereof or the single-domain antibody for mouse PD-L1 that is at least 75%, 80%, 85%, 90%, 95%, 96%, 97%, 98%, or 99% identical to SEQ ID NO: 195.

In some embodiments, the antibody or the antigen-binding fragment thereof or the single-domain antibody described herein may bind to human CTLA-4 encoded by a nucleic acid that is at least 75%, 80%, 85%, 90%, 95%, 96%, 97%, 98%, or 99% identical to SEQ ID NO: 201 or SEQ ID NO: 202. In some embodiments, the antibody or the antigen-binding fragment thereof or the single-domain antibody described herein may bind to human CTLA-4 comprising a polypeptide sequence that is at least 75%, 80%, 85%, 90%, 95%, 96%, 97%, 98%, or 99% identical to SEQ ID NO: 203 or SEQ ID NO: 204. Examples of the peptide sequence of the antibody or the antigen-binding fragment thereof or the single-domain antibody for human CTLA-4 may be found in SEQ ID NO: 205 and 206. In some embodiments, the exogenous single-domain antibody or fragment comprises a peptide sequence that is greater than or equal to about 50%, 55%, 60%, 65%, 70%, 75%, 80%, 85%, 90%, 95%, 96%, 97%, 98% or 99% identical to SEQ ID NOs: 205 or 206.

In some embodiments, the antibody or the antigen-binding fragment thereof or the single-domain antibody described herein may bind to mouse CTLA-4. Examples of the peptide sequence of the antibody or the antigen-binding fragment thereof or the single-domain antibody for mouse CTLA-4 may be found in SEQ ID NOs: 211-213. In some embodiments, the exogenous single-domain antibody or fragment comprises a peptide sequence that is greater than or equal to about 50%, 55%, 60%, 65%, 70%, 75%, 80%, 85%, 90%, 95%, 96%, 97%, 98% or 99% identical to SEQ ID NOs: 211-213.

In some embodiments, the antibody or the antigen-binding fragment thereof, or the single-domain antibody, described herein may bind to interleukin-6 receptor (IL-6R). Examples of the peptide sequence of the antibody or the antigen-binding fragment thereof or the single-domain antibody for IL-6R may be found in SEQ ID NOs: 221-231 and 241-245. In some embodiments, the exogenous single-domain antibody or fragment comprises a peptide sequence that is greater than or equal to about 50%, 55%, 60%, 65%, 70%, 75%, 80%, 85%, 90%, 95%, 96%, 97%, 98% or 99% identical to SEQ ID NOs: 221-231 and 241-245.

In some embodiments, the antibody or the antigen-binding fragment thereof or the single-domain antibody described herein may bind to human Lymphocyte-activation gene 3 (LAG-3) encoded by a nucleic acid that is at least 75%, 80%, 85%, 90%, 95%, 96%, 97%, 98%, or 99% identical to any one of SEQ ID NOs: 301-312. In some embodiments, the antibody or the antigen-binding fragment thereof or the single-domain antibody described herein may bind to human LAG-3 comprising a polypeptide sequence that is at least 75%, 80%, 85%, 90%, 95%, 96%, 97%, 98%, or 99% identical to any one of SEQ ID NOs: 315-321. Examples of the peptide sequence of the antibody or the antigen-binding fragment thereof or the single-domain antibody for human LAG-3 may be found in SEQ ID NOs: 325-331. In some embodiments, the exogenous single-domain antibody or fragment comprises a peptide sequence that is greater than or equal to about 50%, 55%, 60%, 65%, 70%, 75%, 80%, 85%, 90%, 95%, 96%, 97%, 98% or 99% identical to SEQ ID NOs: 325-331.

In some embodiments, the antibody or the antigen-binding fragment thereof or the antigen-binding fragment thereof or the single-domain antibody described herein may bind to Spike protein as, for example, the spike glycoprotein of a coronavirus. Examples of the peptide sequence of the antibody or the antigen-binding fragment thereof or the single-domain antibody or antigen-binding fragment thereof for Spike protein may be found in SEQ ID NOs: 401-404. In some embodiments, the exogenous single-domain antibody or fragment comprises a peptide sequence that is greater than or equal to about 50%, 55%, 60%, 65%, 70%, 75%, 80%, 85%, 90%, 95%, 96%, 97%, 98% or 99% identical to SEQ ID NOs: 401-404.

In some embodiments, the antibody or the antigen-binding fragment thereof or the single-domain antibody or antigen-binding fragment thereof described herein may bind to an epitope of any one of the pathogens described herein. Non-limiting example of pathogens can be found in Table 1.

In some embodiments, the antibody or the antigen-binding fragment thereof or the single-domain antibody or antigen-binding fragment thereof described herein may bind to programmed cell death 1 ligand 2 (PDCD1LG2) encoded by a nucleic acid that is at least 75%, 80%, 85%, 90%, 95%, 96%, 97%, 98%, or 99% identical to SEQ ID NO: 501. In some embodiments, the antibody or the antigen-binding fragment thereof or the single-domain antibody described herein may bind to PDCD1LG2 comprising a polypeptide sequence that is at least 75%, 80%, 85%, 90%, 95%, 96%, 97%, 98%, or 99% identical to SEQ ID NO: 511.

In some embodiments, the antibody or the antigen-binding fragment thereof or the single-domain antibody described herein may bind to Programmed cell death protein 1 (PDCD-1 or PD-1) encoded by a nucleic acid that is at least 75%, 80%, 85%, 90%, 95%, 96%, 97%, 98%, or 99% identical to any one of SEQ ID NO: 521-526. In some embodiments, the antibody or the antigen-binding fragment thereof or the single-domain antibody described herein may bind to PDCD-1 comprising a polypeptide sequence that is at least 75%, 80%, 85%, 90%, 95%, 96%, 97%, 98%, or 99% identical to any one of SEQ ID NO: 531-535.

In some embodiments, the antibody or the antigen-binding fragment thereof or the single-domain antibody described herein may bind to PD-1 encoded by a nucleic acid that is at least 75%, 80%, 85%, 90%, 95%, 96%, 97%, 98%, or 99% identical to any one of SEQ ID NO: 541-545. In some embodiments, the antibody or the antigen-binding fragment thereof or the single-domain antibody described herein may bind to PD-1 comprising a polypeptide sequence that is at least 75%, 80%, 85%, 90%, 95%, 96%, 97%, 98%, or 99% identical to any one of SEQ ID NO: 551-554. In some embodiments, the antibody or the antigen-binding fragment thereof or the single-domain antibody described herein that may bind to PD-1 encoded from a nucleic acid sequence that is at least 75%, 80%, 85%, 90%, 95%, 96%, 97%, 98%, or 99% identical to SEQ ID NO: 561.

In some embodiments, the antibody or the antigen-binding fragment thereof or the single-domain antibody described herein may bind to LAG3-Associated protein (LAG3P) encoded by a nucleic acid that is at least 75%, 80%, 85%, 90%, 95%, 96%, 97%, 98%, or 99% identical to any one of SEQ ID NO: 561. In some embodiments, the antibody or the antigen-binding fragment thereof or the single-domain antibody described herein may bind to LAG3P comprising a polypeptide sequence that is at least 75%, 80%, 85%, 90%, 95%, 96%, 97%, 98%, or 99% identical to any one of SEQ ID NO: 571.

In some embodiments, the antibody or the antigen-binding fragment thereof or the single-domain antibody described herein may bind to T-cell immunoglobulin and mucin domain-containing protein 3 (TIM3) encoded by a nucleic acid that is at least 75%, 80%, 85%, 90%, 95%, 96%, 97%, 98%, or 99% identical to SEQ ID NO: 581. In some embodiments, the antibody or the antigen-binding fragment thereof or the single-domain antibody described herein may bind to TIM3 comprising a polypeptide sequence that is at least 75%, 80%, 85%, 90%, 95%, 96%, 97%, 98%, or 99% identical to any one of SEQ ID NO: 594.

In some embodiments, the antibody or the antigen-binding fragment thereof or the single-domain antibody described herein may bind T cell immunoreceptor with Ig and ITIM domains (TIGIT) encoded by a nucleic acid that is at least 75%, 80%, 85%, 90%, 95%, 96%, 97%, 98%, or 99% identical to any one of SEQ ID NO: 591 to SEQ ID NO: 601. In some embodiments, the antibody or the antigen-binding fragment thereof or the single-domain antibody described herein may bind to TIGIT comprising a polypeptide sequence that is at least 75%, 80%, 85%, 90%, 95%, 96%, 97%, 98%, or 99% identical to any one of SEQ ID NO: 611 to SEQ ID NO: 619.

In some embodiments, the antibody or the antigen-binding fragment thereof or the single-domain antibody described herein may bind V-domain Ig suppressor of T cell activation (VISTA) encoded by a nucleic acid that is at least 75%, 80%, 85%, 90%, 95%, 96%, 97%, 98%, or 99% identical to any one of SEQ ID NO: 701 to SEQ ID NO: 705. In some embodiments, the antibody or the antigen-binding fragment thereof or the single-domain antibody described herein may bind to VISTA comprising a polypeptide sequence that is at least 75%, 80%, 85%, 90%, 95%, 96%, 97%, 98%, or 99% identical to SEQ ID NO: 711.

In some embodiments, the antibody or the antigen-binding fragment thereof or the single-domain antibody described herein may bind to human Brain-Derived Neurotrophic Factor (BDNF) to exert an antagonist effect against human BDNF. In some embodiments, the antibody or the antigen-binding fragment thereof or the single-domain antibody described herein may bind to mouse BDNF to exert an antagonist effect against mouse BDNF. In some embodiments, the antibody or the antigen-binding fragment thereof or the single-domain antibody described herein may bind to human Tropomyosin receptor kinase B (TrkB) to exert an antagonist effect against human TrkB. In some embodiments, the antibody or the antigen-binding fragment thereof or the single-domain antibody described herein may bind to mouse TrkB to exert an antagonist effect against mouse TrkB. In some embodiments, the antibody or the antigen-binding fragment thereof or the single-domain antibody described herein may bind to human TrkB to exert an agonist effect against human TrkB. In some embodiments, the antibody or the antigen-binding fragment thereof or the single-domain antibody described herein may bind to mouse TrkB to exert an agonist effect against mouse TrkB. In some embodiments, the antibody or the antigen-binding fragment thereof or the single-domain antibody described herein may bind to human PD-1 to exert an agonist effect against human PD-1. In some embodiments, the antibody or the antigen-binding fragment thereof or the single-domain antibody described herein may bind to mouse PD-1 to exert an agonist effect against mouse PD-1. In some embodiments, the antibody or the antigen-binding fragment thereof or the single-domain antibody described herein may bind to human PD-L1 to exert an antagonist effect against human PD-L1. In some embodiments, the antibody or the antigen-binding fragment thereof or the single-domain antibody described herein may bind to mouse PD-L1 to exert an antagonist effect against mouse PD-L1.

In some embodiments, the antibody or the antigen-binding fragment thereof or the single-domain antibody described herein may bind to CTLA-4 such as Yervoy (ipilimumab). In some embodiments, the antibody or the antigen-binding fragment thereof or the single-domain antibody described herein may bind to PD-1 such as Nivolumab (Opdivo), Pembrolizumab (Keytruda), or Cemiplimab (Libtayo). In some embodiments, the antibody or the antigen-binding fragment thereof or the single-domain antibody described herein may bind to PD-L1 such as Avelumab (Bavencio), durvalumab (Imfinzi), or atezolizumab (Tecentriq). In some embodiments, the antibody or the antigen-binding fragment thereof or the single-domain antibody described herein may bind to HER2 such as Herceptin (trastuzumab), margetuximab-cmkb (MARGENZA), or Pertuzumab (Perjeta). In some embodiments, the antibody or the antigen-binding fragment thereof or the single-domain antibody described herein may bind to TROP-2 such as Sacituzumab (Trodelvy). In some embodiments, the antibody or the antigen-binding fragment thereof or the single-domain antibody described herein may bind to IL-6 such as Siltuximab (Sylvant). In some embodiments, the antibody or the antigen-binding fragment thereof or the single-domain antibody described herein may bind to IL-6R such as Tocilizumab (Actemra). In some embodiments, the antibody or the antigen-binding fragment thereof or the single-domain antibody described herein may bind to CD20 such as Rituximab (MabThera).

In some embodiments, the antibody or the antigen-binding fragment thereof or the single-domain antibody or antigen-binding fragment thereof described herein may be conjugated with a drug to form an antibody-drug conjugate (ADC). Examples of the drug or compound that may be part of the ADC may include a chemotherapeutic agent, cytotoxic agent, cytokine, growth-inhibitory agent, anti-hormonal agent, anti-angiogenic agent, cardio protectant, and/or checkpoint inhibitor. Non-limiting checkpoint inhibitor includes IMP321/Eftilagimod alpha (Immutep), Relatlimab BMS-986016, Ipilimumab (Yervoy), Pembrolizumab (Keytruda), Nivolumab (Opdivo), Cemiplimab (Libtayo), Atezolizumab (Tecentriq), Avelumab (Bavencio), Durvalumab (Imfinzi), Ipilimumab (Yervoy), LAG525, MK-4280, Irinotecan, Oxaliplatin, REGN3767, TSR-033, BI754111, Sym022, FS118 (a bi-specific anti-LAG3/PD-L1 antagonistic mAb), MGD013 (a bi-specific anti-LAG3/PD-1 antagonistic mAb), TSR-022, Niraparib, Bevacizumab, MBG453, Decitabine, Spartalizumab, Sym023, INCAGN2390, LY3321367, Ramucirumab, Abemaciclib, Merestinib, BMS-986258, SHR-1702, Camrelizumab, MK-7684, Etigilimab/OMP-313 M32, Tiragolumab/MTIG7192A/RG-6058, BMS-986207, AB-154, ASP-8374, JNJ-61610588, CA-170d, Enoblituzumab/MGA271, MGD009, I-8H9/omburtamab, Trastuzumab, MGD013 (Anti-PD-1, anti-LAG-3 dual checkpoint inhibitor), BGB-A1217, CM-24 (MK-6018), BMS 986178, MEDI6469, PF-04518600, GSK3174998, MOXR0916, Utomilimab (PF-05082566), Urelumab (BMS-663513) ES101, BMS-986156, TRX-518, AMG 228, JTX-2011, GSK3359609, BMS-986226, MEDI-570, or Varlilumab (CDX-1127). Such compounds or drugs may be present in combination in amounts that are effective for the purpose intended. In some embodiments, the ADC comprises molecule for treating idiopathic pulmonary fibrosis. In some embodiments, the ADC comprises nintedanib or pirfenidone.

In some embodiments, the antibody or the antigen-binding fragment thereof or the single-domain antibody described herein may bind to VEGF, VEGFR, or a combination thereof (VEGF/VEGFR). In some embodiments, the antibody or the antigen-binding fragment thereof or the single-domain antibody described herein may bind to VEGF-A, VEGF-B, VEGF-C, VEGF-D, placental growth factor (PIGF), Angiopoitin-1, Angiopoitin-2, Endostatin, FGF, MMP, DII4, Class 3 semaphorins, FGF, VEGFR, NRP-1, PDGF (BB-homodimer), PDGFR, TGF-β, endoglin, TGF-β receptors, CCL2, Integrins αVβ3, αVβ5, or α5β1, VE-cadherin, CD31, ephrin, plasminogen activator, plasminogen activator inhibitor-1, eNOS, COX-2, AC133, ID1/ID3, Class 3 semaphorin, or Nogo-A, or a combination thereof. In some embodiments, the antibody or the antigen-binding fragment thereof or the single-domain antibody described herein may bind to VEGF-A receptor, VEGF-B receptor, VEGF-C receptor, VEGF-D receptor, placental growth factor receptor (PIGF), or a combination thereof.

### Pharmaceutical Formulations

Described herein are pharmaceutical formulations comprising the enucleated cells or the compositions described herein. In some embodiments, the pharmaceutical formulations further comprise a pharmaceutically acceptable: carrier, excipient, diluent, or nebulized inhalant.

In some embodiments, the pharmaceutical formulations include two or more active agents, or two or more therapeutic agents as disclosed herein. In some embodiments, the two or more active agents are contained in a single dosage unit such as, for example, when the enucleated cell comprises two or more therapeutic agents. In embodiments, the two or more active agents are contained in separate dosage units such as when the enucleated cell is administered separately from an additional therapeutic agent or adjuvant. In some embodiments, the active agents that may be, in some embodiments, the additional therapeutic agent include a chemotherapeutic agent, cytotoxic agent, cytokine, growth-inhibitory agent, anti-hormonal agent, anti-angiogenic agent, cardio protectant, and/or checkpoint inhibitor. Non-limiting checkpoint inhibitor includes IMP321/Eftilagimod alpha (Immutep), Relatlimab BMS-986016, Ipilimumab (Yervoy), Pembrolizumab (Keytruda), Nivolumab (Opdivo), Cemiplimab (Libtayo), Atezolizumab (Tecentriq), Avelumab (Bavencio), Durvalumab (Imfinzi), Ipilimumab (Yervoy), LAG525, MK-4280, Irinotecan, Oxaliplatin, REGN3767, TSR-033, BI754111, Sym022, FS118 (a bi-specific anti-LAG3/PD-L1 antagonistic mAb), MGD013 (a bi-specific anti-LAG3/PD-1 antagonistic mAb), TSR-022, Niraparib, Bevacizumab, MBG453, Decitabine, Spartalizumab, Sym023, INCAGN2390, LY3321367, Ramucirumab, Abemaciclib, Merestinib, BMS-986258, SHR-1702, Camrelizumab, MK-7684, Etigilimab/OMP-313 M32, Tiragolumab/MTIG7192A/RG-6058, BMS-986207, AB-154, ASP-8374, JNJ-61610588, CA-170d, Enoblituzumab/MGA271, MGD009, I-8H9/omburtamab, Trastuzumab, MGD013 (Anti-PD-1, anti-LAG-3 dual checkpoint inhibitor), BGB-A1217, CM-24 (MK-6018), BMS 986178, MEDI6469, PF-04518600, GSK3174998, MOXR0916, Utomilimab (PF-05082566), Urelumab (BMS-663513) ES101, BMS-986156, TRX-518, AMG 228, JTX-2011, GSK3359609, BMS-986226, MEDI-570, or Varlilumab (CDX-1127). Such compounds or drugs may be present in combination in amounts that are effective for the purpose intended. Non-limiting examples of active agents that may be, in some embodiments, the additional therapeutic agent include CPI-006 (for inhibiting CD73 and allowing T cell and APC activation); Monalizumab (for inhibiting NKG2A); COM701 (for inhibiting PVRIG/PVRL2 and activating T cell); CM24 (for inhibiting CEACAM1 and allowing T and NK cells activation); NEO-201 (for inhibiting CEACAM5 and CEACAM6 which allows T cell activation while interfering with tumor cell growth); Defactinib (for inhibiting FAK and interfering with tumor growth); PF-04136309 (for inhibiting CCR-2 and CCL-2 and allowing T cell recruitment and activation); MSC-1 (for inhibiting LIF and allowing T cell and APC activation while interfering with cancer growth); Hu5F9-G4 (5F9), ALX148, TTI-662, and RRx-001 (for inhibiting CD47 or SIRPα and allowing T cell and APC activation); Lacnotuzumab (MCS-110), LY3022855, SNDX-6352, Emactuzumab (RG7155), and Pexidartinib (PLX3397) (for inhibiting M-CSF or CSF-1R and allowing APC activation); CAN04 and Canakinumab (ACZ885) (for inhibiting IL-3 or IL-1RAP and allowing T cell and APC activation); BMS-986253 (for inhibiting IL-8 and decreasing immunosuppressive tumor microenvironment while interfering with tumor growth); Pepinemab (VX15/2503) (for inhibiting SEMA4D and decreasing immunosuppressive tumor microenvironment while interfering with tumor growth); Trebananib (for inhibiting Angiopoietin-2 and allowing APC activation while interfering with cancer growth); FP-1305 (for inhibiting CLEVER-1 and allowing APC activation); Enapotamab vedotin (EnaV) (for inhibiting Axl and allowing APC activation while interfering with cancer growth); or Bavituximab (for inhibiting phosphatidylserine and allowing T cell and APC activation while interfering with cancer growth).

In practicing the methods of treatment or use provided herein, therapeutically effective amounts of pharmaceutical formulations described herein are administered to a mammal having a disease, disorder, or condition to be treated, e.g., cancer. In some embodiments, the mammal is a human. A therapeutically effective amount may vary widely depending on the severity of the disease, the age and relative health of the subject, the potency of the therapeutic agent used and other factors. The therapeutic agents, and in some cases, pharmaceutical formulations described herein, may be used singly or in combination with one or more therapeutic agents as components of mixtures.

The pharmaceutical formulations described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical formulations including a therapeutic agent may be manufactured in a conventional manner such as, by way of example only, by means of conventional mixing, dissolving, granulating, dragee-making, levigating, emulsifying, encapsulating, entrapping or compression processes.

The pharmaceutical formulations may include at least an exogenous therapeutic agent as an active ingredient in free-acid or free-base form, or in a pharmaceutically acceptable salt form. In addition, the methods and compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In certain embodiments, pharmaceutical formulations provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, pharmaceutical formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, I about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

The pharmaceutical formulations described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents such as parabens, chlorobutanol, phenol, sorbic acid, and the like. In some cases, it is desirable to include isotonic agents such as sugars, sodium chloride, and the like. Prolonged absorption of the injectable pharmaceutical form may be brought about by the use of agents delaying absorption such as aluminum monostearate and gelatin.

For intravenous injections or drips or infusions, a pharmaceutical formulations described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Pharmaceutical formulations for injection may be presented in unit dosage form, e.g., in ampoules or in multi dose containers, with an added preservative. The composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical formulations described herein are conveniently delivered in the form of an aerosol spray presentation from pressurized packs or nebulizers, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of such as, by way of example only, gelatin for use in an inhaler or insufflator may be formulated containing a powder mix of the therapeutic agent described herein and a suitable powder base such as lactose or starch. Formulations that include a composition are prepared as solutions in saline, employing benzyl alcohol or other suitable preservatives, fluorocarbons, and/or other solubilizing or dispersing agents known in the art. Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the compositions described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, the pharmaceutical formulations of the exogenous therapeutic agents are in the form of a capsules, including push fit capsules made of gelatin, as well as soft, sealed capsules made of gelatin and a plasticizer such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

Pharmaceutical formulations for oral administration are in dosages suitable for such administration. In one aspect, solid oral dosage forms are prepared by mixing a composition with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the composition is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating may provide a delayed release of a therapeutic agent from the formulation. In other embodiments, the film coating aids in patient compliance. Film coatings typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

Buccal formulations are administered using a variety of formulations known in the art. In addition, the buccal dosage forms described herein may further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a pharmaceutical formulations is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a composition described herein is in a form suitable for parenteral injection as a sterile suspensions, solutions or emulsions in oily or aqueous vehicles, and contain formulatory agents such as suspending, stabilizing and/or dispersing agents. The compositions for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

In some embodiments, the compositions are provided that include particles of a therapeutic agent and at least one dispersing agent or suspending agent for oral administration to a subject. The formulations may be a powder and/or granules for suspension, and upon admixture with water, a substantially uniform suspension is obtained.

Furthermore, the pharmaceutical formulations optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the pharmaceutical formulations optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other the pharmaceutical formulations optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

An aerosol formulation for nasal administration is generally an aqueous solution designed to be administered to the nasal passages in drops or sprays. Nasal solutions may be similar to nasal secretions in that they are generally isotonic and slightly buffered to maintain a pH of about 5.5 to about 6.5, although pH values outside of this range may additionally be used. Antimicrobial agents or preservatives may also be included in the formulation.

An aerosol formulation for inhalations and inhalants may be designed so that the agent or combination of agents is carried into the respiratory tree of the subject when administered by the nasal or oral respiratory route. Inhalation solutions may be administered, for example, by a nebulizer. Inhalations or insufflations, comprising finely powdered or liquid drugs, may be delivered to the respiratory system as a pharmaceutical aerosol of a solution or suspension of the agent or combination of agents in a propellant, e.g., to aid in disbursement. Propellants may be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants may include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Hydrocarbon propellants useful include, for example, propane, isobutane, n-butane, pentane, isopentane and neopentane. A blend of hydrocarbons may also be used as a propellant. Ether propellants include, for example, dimethyl ether as well as the ethers. An aerosol formulation may also comprise more than one propellant. For example, the aerosol formulation comprises more than one propellant from the same class such as two or more fluorocarbons; or more than one, more than two, more than three propellants from different classes such as a fluorohydrocarbon and a hydrocarbon. The compositions of the present disclosure may also be dispensed with a compressed gas, e.g., an inert gas such as carbon dioxide, nitrous oxide or nitrogen.

Aerosol formulations may also include other components, for example, ethanol, isopropanol, propylene glycol, as well as surfactants or other components such as oils and detergents. These components may serve to stabilize the formulation and/or lubricate valve components.

The aerosol formulation may be packaged under pressure and may be formulated as an aerosol using solutions, suspensions, emulsions, powders and semisolid preparations. For example, a solution aerosol formulation comprises a solution of an agent such as a transporter, carrier, or ion channel inhibitor in (substantially) pure propellant or as a mixture of propellant and solvent. The solvent may be used to dissolve the agent and/or retard the evaporation of the propellant. Solvents may include, for example, water, ethanol and glycols. Any combination of suitable solvents may be use, optionally combined with preservatives, antioxidants, and/or other aerosol components.

An aerosol formulation may be a dispersion or suspension. A suspension aerosol formulation comprises a suspension of an agent or combination of agents, e.g., a transporter, carrier, or ion channel inhibitor, and a dispersing agent. Dispersing agents may include, for example, sorbitan trioleate, oleyl alcohol, oleic acid, lecithin and corn oil. A suspension aerosol formulation may also include lubricants, preservatives, antioxidant, and/or other aerosol components.

An aerosol formulation may similarly be formulated as an emulsion. An emulsion aerosol formulation may include, for example, an alcohol such as ethanol, a surfactant, water and a propellant, as well as an agent or combination of agents, e.g., a transporter, carrier, or ion channel. The surfactant used may be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

### Methods

Disclosed herein, in some embodiments, are methods of making and using the cells of the present disclosure. Methods disclosed herein comprise methods of producing an enucleated cell from a nucleated cell (parent cell) utilizing high-speed centrifugation. In some embodiments, the methods of producing an enucleated cell of the present disclosure do not include differentiation of the nucleated cell. In some embodiments, the enucleated cell is stored under conditions that slow or suspend biological activity of the cell, such as cryopreservation, lyophilization or cryohybernation. In some embodiments, the biological activity of the enucleated cell that is stored under such conditions may be restored at the point of need, and optionally further engineered as needed. Methods of delivering the enucleated cell to a subject disclosed herein are also provided. In some embodiments, the delivering comprising administering the enucleated cell or a composition comprising the enucleated cell to the subject, such as to treat a disease or a condition or the subject disclosed herein. In some embodiments, the method described herein is for treating a disease or condition characterized, at least in part, by abnormal vasculature in a subject, the method comprising: administering to the subject having the disease or the condition an enucleated cell comprising one or more intracellular organelles that synthesizes or releases an exogenous polypeptide comprising a tumor necrosis factor (TNF) superfamily member polypeptide or a catalytically active fragment thereof in absence of the nucleus, wherein the exogenous polypeptide synthesized or released by the cell is therapeutically effective to normalize the abnormal vasculature in the subject. In some embodiments, the method described herein comprises treating a disease or condition by administering the enucleated cell expressing the single-domain antibody or the antigen-binding fragment thereof to the subject, where the single-domain antibody or the antigen-binding fragment thereof targets an immune checkpoint molecule. In some embodiments, the method described herein comprises treating a disease or condition by administering the enucleated cell expressing the single-domain antibody or the antigen-binding fragment thereof to the subject, where the single-domain antibody or the antigen-binding fragment thereof targets an immune checkpoint molecule described herein. In some embodiments, the method described herein comprises treating a disease or condition by administering the enucleated cell expressing the single-domain antibody or the antigen-binding fragment thereof to the subject, where the single-domain antibody or the antigen-binding fragment thereof targets CTGF.

### Methods of Producing an Enucleated Cell

Disclosed herein are methods of producing an enucleated cell described herein by removing a nucleus of a nucleated cell (parent cell). In some embodiments, methods of producing the enucleated cell do not require differentiation of the parent cell. In some embodiments, the parent cell containing a nucleus is engineered to express the single-domain antibody or antigen-binding fragment thereof, therapeutic agent, transmembrane moiety, immune-evading moiety, and/or targeting moiety described herein; and subsequently, the nucleus of the parent cell is removed. In some embodiments, the parent cell containing the nucleus is enucleated, and the enucleated cell is engineered to express single-domain antibody or antigen-binding fragment thereof, therapeutic agent, transmembrane moiety, immune-evading moiety, and/or targeting moiety described herein. In some embodiments, the parent cell is engineered to express one or more of the biomolecules above (e.g., immune-evading moiety and/or targeting moiety), and the resulting enucleated cell (e.g., already expressing the immune-evading moiety and/or targeting moiety) is further engineered to express a second of the biomolecules above (e.g., a therapeutic agent). In this manner, the enucleated cells of the present disclosure can be extensively engineered prior to enucleation, stored for long periods of time as needed (through for e.g., lyophilization, cryohibernation, cryopreservation), and quickly engineered to express a therapeutic agent closer to the time of need.

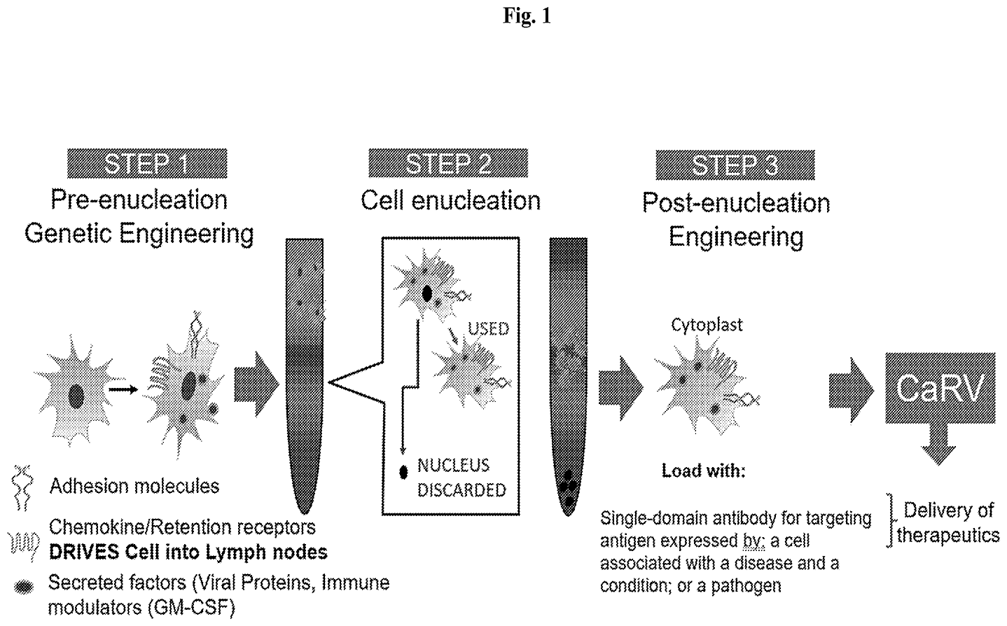

As shown in FIG. 1, the parent cell may be engineered prior to enucleation to express adhesion molecules, chemokine or retention receptors or both, that target the lymph tissue (e.g., lymph nodes) in a subject. In addition, or alternatively, the resulting enucleated cell is engineered to express and, in some cases, secrete the therapeutic agent such as, for example, an antibody or an antigen-binding fragment thereof (e.g., single-domain antibody). In some embodiments, the enucleated cell may be administered to a subject in need thereof to treat a disease or a condition in the subject.

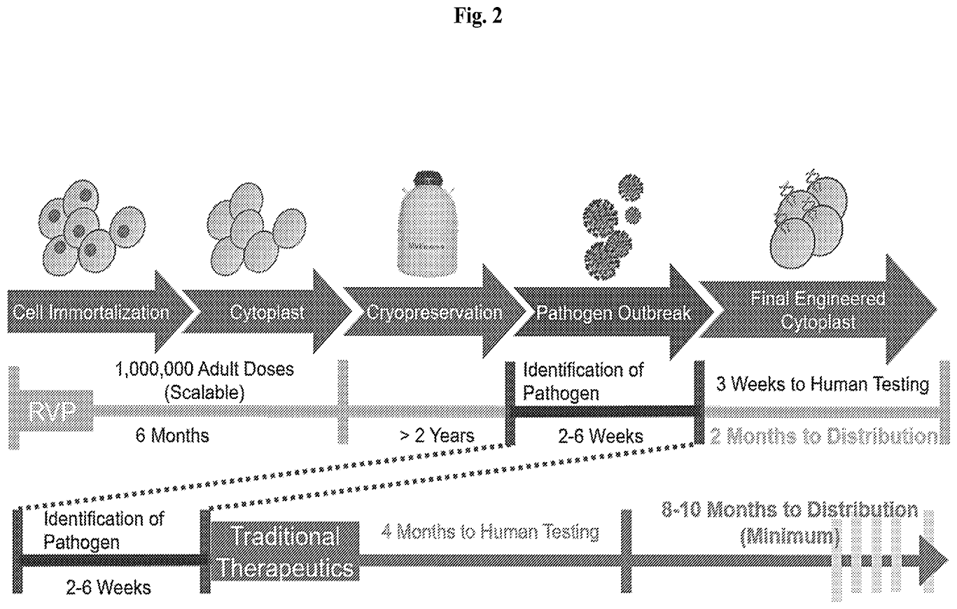

The ability to extensively engineer the enucleated cells described herein before and after enucleation streamlines the manufacturing process considerably as compared with comparable biological drug development timelines. The process of manufacturing the enucleated cells of the present disclosure is roughly 2 months, as compared with conventional biological drug development timelines, which is 12 months or longer. Now referring to FIG. 2, the enucleated cell of the present disclosure may be prepared in advance and cryopreserved for a length of time. This means, the enucleated cell of the present disclosure (e.g., engineered to express the homing receptors, immune activators, etc.) may be rapidly deployed. Such technical aspect is particular important when the enucleated cell is used to treat a disease or a condition stemmed from an outbreak of pathogen exposure or infection.

In some embodiments, removal of the nucleus involves mechanically removing the nucleus. The parent cell may be treated with cytochalasin to soften the cortical actin cytoskeleton. The nucleus is then physically extracted from the cell body by high-speed centrifugation in gradients of Ficoll to generate an enucleated cell. Because enucleate cells and intact nucleated cells sediment to different layers in the Ficoll gradient, enucleated cells may be easily isolated and prepared for therapeutic purposes or fusion to other cells (nucleated or enucleated). The enucleation process is clinically scalable to process tens of millions of cells. In some embodiments, enucleated cells may be used as a disease-homing vehicle to deliver clinically relevant cargos/payloads to treat various diseases.

Various methods may be used to introduce a biomolecule (e.g., the therapeutic agent, transmembrane moiety, immune-evading moiety, and/or targeting moiety described herein) into the parent cell or the enucleated cell described herein. Non-limiting examples of methods that may be used to introduce a biomolecule into the parent cell or the enucleated cell include: liposome mediated transfer, an adenovirus, an adeno-associated virus, a herpes virus, a retroviral based vector, a lentiviral vector, electroporation, microinjection, lipofection, transfection, calcium phosphate transfection, dendrimer-based transfection, cationic polymer transfection, cell squeezing, sonoporation, optical transfection, impalection, hydrodynamic delivery, magnetofection, nanoparticle transfection, or combinations thereof. In some embodiments of any of the compositions and methods provided herein, a therapeutic agent, a virus, an antibody, or a nanoparticle may be introduced into the enucleated cells.

In some embodiments, the enucleated cell is preserved via cryopreservation, cryohibernation, or lyophilization. Cryopreservation comprises freezing the enucleated cell, while cryohibernation comprises storing the enucleated cell at a temperature that is below room temperature but without freezing the enucleated cell. In some embodiments, the enucleated cell is lyophilized. In some embodiments, the lyophilized enucleated cell can be reconstituted, and the reconstituted enucleated cell exhibits comparable viability to the enucleated cell that has not been lyophilized. In some embodiments, the lyophilization comprises components: freezing the cell; subjecting the cell to drying under a very low pressure (e.g., <3000 mTorr) using vacuum. The drying component can lead to sublimation and dehydrate the cell while maintaining cellular viability and biologic function. In some embodiments, the freezing phase comprises balancing the duration and temperature of the freezing to for maintaining cell viability and stability, appropriate crystal formation, and the speed of reconstitution. The triple point of a substance is the temperature and the pressure at which the sublimation curve, fusion curve and vaporization curve meet. Achievement of the triple point which varies for different substances ensures that sublimation rather than melting will occur in the following drying steps. To facilitate faster and more efficient freeze-drying, larger ice crystals are preferred, because they form a network within the product that promotes faster removal of water vapor during sublimation. To produce larger crystals, the product should be frozen slowly or the temperature can be cycled up and down in a process called annealing. Fresh or frozen living tissue or cells do not have a single homogeneous melting point (eutectic point) and consequently the freezing stage of the material (cells or tissue) is cooled below its triple point which represents the temperature and pressure at which the solid, liquid and gas phases of the material can coexist. Living cells do have a critical point on a phase diagram at which both the liquid and the gas phase of an object or substance have the same density and are therefore indistinguishable. The product critical point temperature must be maintained to prevent melt-back or cake collapse occurring during primary and secondary drying which reflects incomplete sublimation. In the case of substances where preservation of structure is required like living cells, large ice crystals maybe detrimental and may break the cell walls which can result in increasingly poor texture and loss of nutritive content. In this case, the freezing should be done rapidly, in order to lower the material to below its critical point quickly, thus avoiding the formation of large ice crystals. The freezing temperatures for cells or tissue can vary but ranges in general between −50° C. (−58° F.) and −80° C. (−112° F.).

During the drying phase, the ambient pressure is lowered to the range of a few millibars, and then heat is supplied by conduction or radiation to the material for the ice to sublime. The amount of heat necessary can be calculated using the sublimating molecules' latent heat of sublimation. In this initial drying phase, about 95% of the water in the material or substance is sublimated. This phase is often slow and can even last for several days depending on the substance and technology employed but if too much heat is added to quickly the material's structure could be altered. In this phase, pressure is controlled through the application of a partial vacuum. The vacuum speeds up the sublimation, making it useful as a deliberate drying process. A cold condenser chamber and/or condenser plates are used as a surface(s) for the water vapor to re-liquify and solidify on. It is important to note that in this range of pressure, the heat cannot be provided by a convection effect because of the low air density. The drying phase also aims to remove remaining unfrozen water molecules since the ice induced with freezing should be removed during the primary drying phase. This part of the freeze-drying process is governed by the material's adsorption isotherms. In this phase, the temperature is raised higher than in the primary drying phase and can even be above 0° C. (32° F.), to break any physico-chemical interactions that have formed between the water molecules and the frozen material. Usually, during this phase the pressure is also lowered in this stage to encourage desorption. However, there are products that benefit from increased pressure as well. After the freeze-drying process is complete, the vacuum is usually broken with an inert gas such as nitrogen before the material is sealed. At the end of the operation, the residual water content in the product is extremely low and should range from <1% to 4% of the original concentration.

In some embodiments, the lyophilization of the enucleated cell comprises the use of lyoprotectants for retaining cell viability and biologic function. Lyoprotectant comprises addition of reagents, salts, or additives that protects cell during the desiccation process. Common lyoprotectants include trehalose, DMSO, methylcellulose, sucrose, antioxidants, human or animal serum proteins, and cellular stress proteins. Additionally, methods for increasing the transport of lyoprotectants inside the cells in suspension can be utilized as a way of improving the viability and function of cells after lyophilization. These methods include electroporation, addition of reagents that enhance intracellular transport, genetic modification of cells to upregulate the expression of pores on cell membranes, and mechanical microfluidic devices that partially disrupt cell membrane integrity and potentially promote intracellular transport of lyoprotectants.

### Methods of Use

Disclosed herein, in some embodiments, are methods of using the compositions described herein. In some embodiments, the methods include treating a disease or a condition of a subject by administering a composition described herein (e.g., a pharmaceutical composition containing enucleated cells engineered to express a therapeutic agent) to the subject. In some embodiments, the method utilizes an enucleated cell described herein for generating tissues or organs ex vivo (e.g., via 3D bioprinting for whole tissue and organ development for transplantation, skin grafting, development of synthetic meat sources, bioscaffolds, etc). In some embodiments, the method utilizes an enucleated cell described herein for propagating and manufacturing cytoplasmic replicating viruses such as VSV, rabies, etc. In some embodiments, the method utilizes an enucleated cell described herein as a diagnostic tool to detect disease or disease location in the body. In some embodiments, the method utilizes an enucleated cell described herein for cosmetic applications. In some embodiments, the method utilizes an enucleated cell described herein for as a source for purification of proteins, membranes, lipids, various RNAs, organelles, or any cellular component that needs to be free of nuclear DNA. In some embodiments, the method utilizes an enucleated cell described herein as a fusogen, where the enucleated cell can be an ex vivo source to transfer gene editing modalities to various cell types in vitro. In some embodiments, the method utilizes an enucleated cell described herein for promoting wound healing or as a regenerative medicine.

The present disclosure also provides methods for the use of enucleated cells (natural or enucleated) as fusion partners to other cells (therapeutic or natural) to enhance and/or transfer biomolecules described herein such as, for example, a single-domain antibody or antigen-binding fragment thereof and/or therapeutic agent. In some embodiments, the biomolecules include, DNA/genes, RNA (mRNA, shRNA, siRNA, miRNA), nanoparticles, peptides, proteins, and plasmids, bacteria, viruses, small molecule drugs, ions, cytokines, growth factors, and hormones. In some embodiments, the enucleated cell is engineered to express a fusogenic moiety. The fusogenic moiety can be any biomolecule (e.g., sugar, lipid, or protein) that promotes fusion of the membrane. In some embodiments, the fusogenic moiety is a fusogenic protein. A fusogenic protein allows the enucleated cell expressing the fusogenic protein to fuse with a target cell. In some embodiments, the fusogenic protein facilitates the merging of an enucleated cell expressing the fusogenic protein with a target cell, allowing the contents of the enucleated cell to enter into the target cell. In some embodiments, the fusogenic protein is heterotypic such as viral classes I-III or Hapless 2 precursor HAP2 or SNARE. In some embodiments, the fusogenic protein is homoleptic such as EFF-1/AFF-1. Other non-limiting examples of the fusogenic protein is Izumol or Syncytin. In some embodiments, the fusogenic protein is a viral protein. In some embodiments, the fusogenic protein from a virus is VSV-g, hERV-W-ENV (Syncytin), or MV-Ed-F+MV-Ed-H (Hemagglutinin). Unlike nucleated cells, the fusion of enucleated cells to the same or another cell type of similar or different origin generates a unique cell hybrid that lacks problematic nuclear transfer, while maintaining desirable therapeutic attributes including, but not limited to, cell surface proteins, signal transduction molecules, secreted proteins, and epigenetic changes. In some embodiments, the fusogenic protein can be expressed on the TNT, thereby facilitating the fusion between the enucleated cell and the target cell when the TNT is contacted with the target cell.

In some embodiments, the fusogenic protein is encoded from a polynucleotide that is greater than or equal to about 50%, 55%, 60%, 65%, 70%, 75%, 80%, 85%, 90%, 95%, or 99% identical to any one of the nucleic acid sequences in SEQ ID NOs: 1001-1106. In some embodiments, the fusogenic protein comprises a polypeptide sequence that is greater than or equal to about 50%, 55%, 60%, 65%, 70%, 75%, 80%, 85%, 90%, 95%, or 99% identical any one of the peptide sequences in SEQ ID NOs: 1201-1205, 1207, 1209-1211, 1213-1229, 1231-1233, 1237-1247, 1249, 1251-1257, and 1259-1305. In some embodiments, the fusogenic protein is encoded from a polynucleotide that is greater than or equal to about 50%, 55%, 60%, 65%, 70%, 75%, 80%, 85%, 90%, 95%, or 99% identical to any one of SEQ ID NO: 1001-1106. In some embodiments, the fusogenic protein comprises a polypeptide sequence that is greater than or equal to about 50%, 55%, 60%, 65%, 70%, 75%, 80%, 85%, 90%, 95%, or 99% identical any one of SEQ ID NO: 1201-1305.

In some embodiments, immortalized cells, such as Human telomerase reverse transcriptase (hTERT) cells or cancer cells, when enucleated, can be administered to a subject in need thereof and to vaccinate the subject against a disease or a condition disclosed herein. Non-limiting examples of human immortalized cells include HeLa (human epithelial) cells, 293/293T/HEK-293T (human embryonic kidney) cells, SH-SY5Y (human neuroblastoma, cloned from bone marrow) cells, and the like. Non-limiting examples of mammalian immortalized cells include 3T3 (mouse embryonic fibroblast) cells. COS (monkey kidney) cells; MDCK (dog kidney epithelial) cells, CHO (Chinese hamster ovary) cells, PC12 (Rat pheochromacytoma chromaffin) cells, and Neuro-2a/N2a (mouse neuroblastoma) cells. For example, once enucleated, cancer cells obtained from the subject can be administered to the subject to induce immunity against a tumor without risk of metastasis. Also, the enucleated cancer cell can be allogenic, thus does not trigger other harmful immune response to the subject. In some embodiments, the enucleated cell in this context may not have a therapeutic payload (e.g., a single-domain antibody or therapeutic agent). In some embodiments, the enucleated cell may comprise one or more of a targeting moiety, immune system evading moiety, therapeutic agent (e.g., mRNA encoding an immune checkpoint molecule, an immune checkpoint molecule inhibitor, a single-domain antibody or an antigen binding fragment thereof, or an oncolytic virus), or a combination thereof. In some embodiments, the enucleated cull can activate the innate or adaptive immunity of the subject by either immunizing the subject with a pathogen or an antigen. In some embodiments, the enucleated cell can activate the innate or adaptive immunity of the subject by delivering, a therapeutic agent that can activate the immune response of the subject.

### Methods of Treatment

Provided herein are methods of treating a disease or a condition in a subject by administering compositions described herein to the subject. In some embodiments, administration is by any suitable mode of administration, including systemic administration (e.g., intravenous, inhalation, etc.). In some embodiments, the subject is human. In some embodiments, the disease or the condition comprises a disease or a condition of lung tissue. In some embodiments, the disease or the condition comprises cancer. In some embodiments, the disease or the condition comprises idiopathic pulmonary fibrosis. In some embodiments, the composition comprises an enucleated cell disclosed herein that has been engineered to express, and in some cases, secrete a therapeutic agent comprising a single-domain antibody or antigen-binding fragment that binds to an immune checkpoint molecule, such as PD-L1. In some embodiments, the composition comprises an enucleated cell disclosed herein that has been engineered to express, and in some cases, secrete a therapeutic agent comprising a single-domain antibody or antigen-binding fragment that binds to an epithelial biomarker, such as CTGF.

In some embodiments, the disease or the condition comprises an infection (e.g., human immunodeficiency virus (HIV)-infection, Chagas disease, tuberculosis), a neurological disease (e.g., Parkinson's Disease, Huntington's Disease, Alzheimer's Disease) an autoimmune disease (e.g., diabetes, Crohn's disease, multiple sclerosis, sickle cell anemia), a cardiovascular disease (e.g., acute myocardial infarction, heart failure, refractory angina), a ophthalmologic disease, a skeletal disease, a metabolic disease (e.g., phenylketonuria, glycogen storage deficiency type 1A, Gaucher disease), an inflammatory disease (e.g., cancer, inflammatory bowel disease), or a disease caused by external pathogen or toxin in a subject. In some embodiments, the disease or the condition comprises idiopathic pulmonary fibrosis. In some embodiments, the subject is in need of, or has been determined to be in need of such an enucleated cell treatment.

In some embodiments, the enucleated cell described herein comprises an antibody or an antigen-binding fragment thereof (e.g., single-domain antibody) that binds to an epitope expressed by a cancer cell or an epitope associated with a tumor microenvironment. In some embodiments, the binding of the antibody or the antigen-binding fragment thereof to the epitope provides a therapeutic effect to treat cancer in a subject. In some embodiments, the binding of the antibody or the antigen-binding fragment thereof to the epitope recruits immune cells to activate immune response against the cancer. In some embodiments, the cancer comprises a cancer in lung tissue. In some embodiments, the cancer is lung cancer. Non-limiting examples of cancer may include Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the target cancer cell represents a subpopulation within a cancer cell population such as a cancer stem cell.

In some embodiments, the cancer may be lung cancer, including non-small cell lung cancer (NSCLC), small cell lung cancer (SCLC), or any other lung cancer type. For example, the lung cancer may include adenocarcinoma, squamous carcinoma, large cell (undifferentiated) carcinoma, large cell neuroendocrine carcinoma, adenosquamous carcinoma, sarcomatoid carcinoma, lung carcinoid tumor, or adenoid cystic carcinoma. Other non-limiting examples of lung cancer includes lymphoma, sarcoma, benign lung tumor, or hamartoma. In some embodiments, the cancer is metastatic cancer. In some embodiments, the cancer metastasized to the lung from a different tissue or source. For example, the metastatic cancer that may be found in the lung may include breast cancer, colon cancer, prostate cancer, sarcoma, bladder cancer, neuroblastoma, and Wilm's tumor.

In some embodiments, described herein are enucleated cells and methods of using these enucleated cells to treat a lung disease or condition. In some embodiments, the lung disease or condition comprises asthma, collapse of part or all of the lung (pneumothorax or atelectasis), swelling and inflammation in the main passages (bronchial tubes) that carry air to the lungs (bronchitis), chronic obstructive pulmonary disease (COPD), lung cancer described herein, lung infection (e.g., pneumonia) abnormal buildup of fluid in the lungs (pulmonary edema), blocked lung artery (pulmonary embolus), or idiopathic pulmonary fibrosis (IPF).

In some embodiments, described herein are enucleated cells and methods of using these enucleated cells to treat a disease or condition associated with abnormal vasculature in a subject. Abnormal vasculature can be associated with disease or condition such as inflammation (e.g., IPF) and cancer (e.g., any one of the cancers described herein). In some embodiments, the enucleated cells described herein, when contacted with the abnormal vasculature, increases the normalization of the abnormal vasculature, where the adhesion between endothelial cells is increased to prevent leakage of intravascular factors out of the vasculature. In some embodiments, the normalization of the abnormal vasculature includes decreasing of damages such as cell dead of the endothelial cells of the vasculature. In some embodiments, the normalization of the abnormal vasculature includes angiogenesis of immature or leaky vessels. In some embodiments, the normalization exerted by the enucleated cells can include normalization of blood vessel, lymphatic vessel, or a combination thereof.

In some embodiments, the normalization of the blood vessel or lymphatic vessel allows delivery of the therapeutics to the target cell. For example, the enucleated cell described herein can deliver exogenous agent to first normalize the abnormal blood vessel or lymphatic vessel, where the abnormal blood vessel or lymphatic vessels: allows a cell associated with a disease or condition (e.g., a cancer cell) to receive nutrient from the blood flow; but are sufficiently small or leaky to prevent effectively therapeutic delivery to the cell associated with a disease or condition. The normalization of the blood vessel or lymphatic vessel then allows the delivery of therapeutics for treating the cell associated with the disease or condition. In some embodiments, the enucleated cell described herein comprises an exogenous agent for normalizing the abnormal blood vessel or lymphatic vessel. In some embodiments, the same enucleated cell comprising the exogenous agent for normalizing the abnormal blood vessel or lymphatic vessel can further comprise a second exogenous agent to be delivered to the target cell for treatment of a disease or a condition. In some embodiments, the disease or the condition comprises cancer or IPF. For example, an enucleated cell can first deliver a soluble form of the TNF member (e.g., a soluble form of LIGHT) to normalize the abnormal blood vessel or lymphatic vessel. After normalization, the same enucleated cell can deliver anticancer therapeutic such as immune checkpoint molecule inhibitor to treat the cell associated with the disease or condition.

In some embodiments, the disease or condition may be caused by a pathogen. In some embodiments, the enucleated cell described herein comprises an antibody or an antigen-binding fragment thereof or single-domain antibody that binds to an epitope expressed by the pathogen or an epitope associated with a microenvironment associated with the pathogen. In some cases, the binding of the antibody or the antigen-binding fragment thereof or single-domain antibody to the epitope confers therapeutic property against the pathogen. In some embodiments, the binding of the antibody or the antigen-binding fragment thereof or single-domain antibody to the epitope recruits immune cells to activate immune response to confer therapeutic property against the pathogen. For example, the disease or condition may be caused by virus, bacterium, fungus, parasite, or molecule resulted from detoxification. In some embodiments, the pathogens: may be easily disseminated or transmitted from person to person; result in high mortality rates and have the potential for major public health impact; may cause public panic and social disruption; and require special action for public health preparedness. Example of these pathogens may include Anthrax (Bacillus anthracis), Botulism (Clostridium botulinum toxin), Plague (Yersinia pestis), Smallpox (variola major), Tularemia (Francisella tularensis), or Viral hemorrhagic fevers, including Filoviruses (Ebola, Marburg) and Arenaviruses (Lassa, Machupo).

In some embodiments, the pathogen: may be moderately easy to disseminate; result in moderate morbidity rates and low mortality rates; and require specific enhancements of diagnostic capacity and enhanced disease surveillance. Example of these pathogens may include Brucellosis (Brucella species), Epsilon toxin of Clostridium perfringens, Food safety threats (e.g., Salmonella species, Escherichia coli 0157:H7, or Shigella), Glanders (Burkholderia mallei), Melioidosis (Burkholderia pseudomallei), Psittacosis (Chlamydia psittaci), Q fever (Coxiella burnetii), Ricin toxin from Ricinus communis (castor beans), Staphylococcal enterotoxin B, Typhus fever (Rickettsia prowazekii), Viral encephalitis (alphaviruses such as eastern equine encephalitis, Venezuelan equine encephalitis, and western equine encephalitis), or Water safety threats (e.g., Vibrio cholerae and Cryptosporidium parvum).

In some embodiments, the pathogen may include emerging pathogen that has a high potential for mortality and morbidity, but the extend of which is not fully understood. Non-limiting examples of these pathogens may include Nipah virus and hantavirus.

In some embodiments, the pathogen comprises a toxin. In some cases, the toxin may be secreted by any one of the pathogens described herein. Non-limiting example of pathogens and the diseases or conditions associated with these pathogens that may be treated with the composition described herein may be found in Table 1.

TABLE 1
Non-Limiting Examples of Pathogen Targets and Associated Disease or Conditions

| Target | Disease |
|---|---|
| Viral Target | Associated Disease or Condition |
| Respiratory syncytial virus (RSV) | RSV Infection |
| Rhesus monkey rotavirus (RV) | RV-induced diarrhea |
| Rhesus monkey RV serotype G3, strain RRV | RV-induced diarrhea |
| P domain VP1 capsid protein | Norovirus |
| H5 hemagglutinin | H5N1 influenza |
| HA1 hemagglutinin | H5N2 influenza |
| Nucleoprotein | Influenza A |
| Nsp9 | Porcine reproductive and respiratory syndrome virus |
| | (PRRSV) |
| Hepatitis C (HCV) E2 glycoprotein | HCV |
| NS3/4A | HCV genotype 3a |
| Retrovirus (Rev) | HIV-1 |
| CXCR4 | HIV-1 |
| Human glycophorin A | HIV (diagnostics) |
| HIV-1 Nef | HIV-1 |
| Nucleoprotein | Ebolavirus (diagnostics biothreat assays: MARSA) |
| Nucleoprotein prNΔ85 | Hantavirus (diagnostics) |
| H5N1 Influenza | H5N1 Influenza (diagnostics) |
| H3N2 | H3N2 Influenza (diagnostics) |
| HIV-1 virion infectivity factor (Vil) | HIV monitoring (diagnostics) |
| Bacterial Target | Associated Disease or Condition |
| Lectin domain F18 fimbriae | ETEC and STEC |
| F4 fimbriae | ETEC |
| FeaGac Adhesin of F4 fimbriae | ETEC |
| Flagella | Campylobacter jejuni |
| Flagella | Pseudomonas aeruginosa |
| Biofilm-associated protein | Acinetobacter baemannii |
| Streptococcus mutans strain HG982 | S. mutans |
| TssM protein of type VI secretion system | Gram-negative bacteria |
| TEM-1 and Bcll β-lactamase | β-lactamase-resistant bacterial strains |
| UreC subunit of urease | Helicobacter pylori |
| Parasite or Fungus | Associated Disease or Condition |
| VSG | Trypanosoma brucei |
| VSG | Human African Trypanosoma |
| Paraflagellar rod protein | Detection of all trypanosoma species (diagnostics) |
| Cell wall protein Malf1 | Malassezia furfur |
| Myosin tail interaction protein | Plasmodium falciparum |
| Toxin | Associated Disease or Condition |
| Toxic venom fractions: Aahl′ and Aahll | Androctonus australis hector (Aah) scorpion venom |
| HNc | Hemiscorpius lepturus scorpion venom |
| α-Cobratoxin | Naja kaouthia venom |
| RTA/RTB subunits | Ricin |
| CDTa toxin | Clostridium difficile |
| CDTa/CDTb toxin | C. difficile |
| LPS derived from Neisseria meningitidis | N. meningitidis |
| Staphylococcal enterotoxin B | Toxin of Cholera |
| Anthrax | Bacillus anthracis |
| Shiga toxin 1 and 2 | Shiga toxin-producing Escherichia coli |
| Botulinum neurotoxin A and E | Clostridium botulinum |
| ADP-ribosylating toxin | Salmonella typhimurium |
| Tetanus toxin and CD11b/CD18 (mac-1) | Clostridium tetani |

### Doses and Frequency of Administration

The enucleated cells described herein, or the composition containing such enucleated cells (referred to in this section as “composition”) may be administered to a subject in a suitable dose, mod of administration, and frequency, which depends on the intended effect.

In some embodiments, the composition is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60, 70, 80, 90, 100 times) during a period of time.

In some embodiments, the composition is administered in a therapeutically-effective amount by various forms and routes including, for example, oral, or topical administration. In some embodiments, a composition may be administered by parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition may be administered by absorption through epithelial or mucocutaneous linings (e.g., oral mucosa, rectal and intestinal mucosa administration). In some embodiments, the composition is delivered via multiple administration routes.

In some embodiments, the composition is administered by intravenous infusion. In some embodiments, the composition is administered by slow continuous infusion over a long period such as more than 24 hours. In some embodiments, the composition is administered as an intravenous injection or a short infusion.

A composition may be administered in a local manner, for example, via injection of the agent directly into an organ, optionally in a depot or sustained release formulation or implant. A composition may be provided in the form of a rapid release formulation, in the form of an extended release formulation, or in the form of an intermediate release formulation. A rapid release form may provide an immediate release. An extended release formulation may provide a controlled release or a sustained delayed release. In some embodiments, a pump may be used for delivery of the composition. In some embodiments, a pen delivery device may be used, for example, for subcutaneous delivery of a composition of the disclosure.

The composition provided herein may be administered in conjunction with other therapies disclosed herein, including, for example, an antiviral therapy, a chemotherapy, an antibiotic, a cell therapy, a cytokine therapy, or an anti-inflammatory agent. In some embodiments, a circular polyribonucleotide or the antibody or the antigen-binding fragment thereof described herein may be used singly or in combination with one or more therapeutic agents as a component of mixtures. In some embodiments, a linear polyribonucleotide or the antibody or the antigen-binding fragment thereof described herein may be used singly or in combination with one or more therapeutic agents as a component of mixtures.

The compositions (e.g., enucleated cells or pharmaceutical formulation comprising the enucleated cell described herein) may be administered before, during, or after the occurrence of a disease or condition, and the timing of administering the composition containing a therapeutic agent may vary. In some cases, the composition may be used as a prophylactic and may be administered continuously to subjects (e.g., the subject for immunization or the subject for treatment) with a susceptibility to a coronavirus or a propensity to a condition or disease associated with a coronavirus. Prophylactic administration may lessen a likelihood of the occurrence of the infection, disease or condition, or may reduce the severity of the infection, disease or condition.

The composition may be administered to a subject before the onset of the symptoms. The composition may be administered to a subject (e.g., the subject for immunization or the subject for treatment) after (e.g., as soon as possible after) a test result, for example, a test result that provides a diagnosis, a test that shows the presence of a coronavirus in a subject (e.g., the subject for immunization or the subject for treatment), or a test showing progress of a condition, e.g., decreased blood oxygen levels. A therapeutic agent may be administered after (e.g., as soon as is practicable after) the onset of a disease or condition is detected or suspected. A therapeutic agent may be administered after (e.g., as soon as is practicable after) a potential exposure to a coronavirus, for example, after a subject (e.g., the subject for immunization or the subject for treatment) has contact with an infected subject, or learns they had contact with an infected subject that may be contagious.

Actual dosage levels of an agent of the disclosure (e.g., antibody or antigen-binding fragment thereof, or therapeutic agent) may be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions of the present invention employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear polyribonucleotide or antibody or antigen-binding fragment thereof.

A composition described herein may be in a unit dosage form suitable for a single administration of a precise dosage. In unit dosage form, the formulation may be divided into unit doses containing appropriate quantities of the compositions. In unit dosage form, the formulation may be divided into unit doses containing appropriate quantities of one or more linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

A dose may be based on the amount of the agent per kilogram of body weight of a subject (e.g., the subject for immunization or the subject for treatment). A dose of an agent (e.g., antibody) is in the range of 10-3000 mg/kg, e.g., 100-2000 mg/kg, e.g., 300-500 mg/kg/day for 1-10 or 1-5 days; e.g., 400 mg/kg/day for 3-6 days; e.g., 1 g/kg/d for 2-3 days In some embodiments, a dose may be based on the number of the enucleated cells per kilogram of body weight of a subject. In some embodiments, a dose may be is administered in a dosage amount of between about 1000 cells/kg body weight to about 1,000,000,000,000 cells/kg body weight, about 1,000 cells/kg body weight to about 1,000,000,000,000 cells/kg body weight. In some embodiments, a dose may be is administered in a dosage amount of between about 1000 cells/kg body weight to about 1,000,000,000,000 cells/kg body weight. about 1,000 cells/kg body weight to about 10,000 cells/kg body weight, about 1,000 cells/kg body weight to about 100,000 cells/kg body weight, about 1,000 cells/kg body weight to about 1,000,000 cells/kg body weight, about 1,000 cells/kg body weight to about 10,000,000 cells/kg body weight, about 1,000 cells/kg body weight to about 100,000,000 cells/kg body weight, about 1,000 cells/kg body weight to about 1,000,000,000 cells/kg body weight, about 1,000 cells/kg body weight to about 10,000,000,000 cells/kg body weight, about 1,000 cells/kg body weight to about 100,000,000,000 cells/kg body weight, about 1,000 cells/kg body weight to about 1,000,000,000,000 cells/kg body weight, about 10,000 cells/kg body weight to about 100,000 cells/kg body weight, about 10,000 cells/kg body weight to about 1,000,000 cells/kg body weight, about 10,000 cells/kg body weight to about 10,000,000 cells/kg body weight, about 10,000 cells/kg body weight to about 100,000,000 cells/kg body weight, about 10,000 cells/kg body weight to about 1,000,000,000 cells/kg body weight, about 10,000 cells/kg body weight to about 10,000,000,000 cells/kg body weight, about 10,000 cells/kg body weight to about 100,000,000,000 cells/kg body weight, about 10,000 cells/kg body weight to about 1,000,000,000,000 cells/kg body weight, about 100,000 cells/kg body weight to about 1,000,000 cells/kg body weight, about 100,000 cells/kg body weight to about 10,000,000 cells/kg body weight, about 100,000 cells/kg body weight to about 100,000,000 cells/kg body weight, about 100,000 cells/kg body weight to about 1,000,000,000 cells/kg body weight, about 100,000 cells/kg body weight to about 10,000,000,000 cells/kg body weight, about 100,000 cells/kg body weight to about 100,000,000,000 cells/kg body weight, about 100,000 cells/kg body weight to about 1,000,000,000,000 cells/kg body weight, about 1,000,000 cells/kg body weight to about 10,000,000 cells/kg body weight, about 1,000,000 cells/kg body weight to about 100,000,000 cells/kg body weight, about 1,000,000 cells/kg body weight to about 1,000,000,000 cells/kg body weight, about 1,000,000 cells/kg body weight to about 10,000,000,000 cells/kg body weight, about 1,000,000 cells/kg body weight to about 100,000,000,000 cells/kg body weight, about 1,000,000 cells/kg body weight to about 1,000,000,000,000 cells/kg body weight, about 10,000,000 cells/kg body weight to about 100,000,000 cells/kg body weight, about 10,000,000 cells/kg body weight to about 1,000,000,000 cells/kg body weight, about 10,000,000 cells/kg body weight to about 10,000,000,000 cells/kg body weight, about 10,000,000 cells/kg body weight to about 100,000,000,000 cells/kg body weight, about 10,000,000 cells/kg body weight to about 1,000,000,000,000 cells/kg body weight, about 100,000,000 cells/kg body weight to about 1,000,000,000 cells/kg body weight, about 100,000,000 cells/kg body weight to about 10,000,000,000 cells/kg body weight, about 100,000,000 cells/kg body weight to about 100,000,000,000 cells/kg body weight, about 100,000,000 cells/kg body weight to about 1,000,000,000,000 cells/kg body weight, about 1,000,000,000 cells/kg body weight to about 10,000,000,000 cells/kg body weight, about 1,000,000,000 cells/kg body weight to about 100,000,000,000 cells/kg body weight, about 1,000,000,000 cells/kg body weight to about 1,000,000,000,000 cells/kg body weight, about 10,000,000,000 cells/kg body weight to about 100,000,000,000 cells/kg body weight, about 10,000,000,000 cells/kg body weight to about 1,000,000,000,000 cells/kg body weight, or about 100,000,000,000 cells/kg body weight to about 1,000,000,000,000 cells/kg body weight. In some embodiments, a dose may be is administered in a dosage amount of between about 1000 cells/kg body weight to about 1000000000000 cells/kg body weight. about 1,000 cells/kg body weight, about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, about 100,000,000,000 cells/kg body weight, or about 1,000,000,000,000 cells/kg body weight. In some embodiments, a dose may be is administered in a dosage amount of between about 1000 cells/kg body weight to about 1000000000000 cells/kg body weight. at least about 1,000 cells/kg body weight, about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, or about 100,000,000,000 cells/kg body weight. In some embodiments, a dose may be is administered in a dosage amount of between about 1000 cells/kg body weight to about 1000000000000 cells/kg body weight. at most about 10,000 cells/kg body weight, about 100,000 cells/kg body weight, about 1,000,000 cells/kg body weight, about 10,000,000 cells/kg body weight, about 100,000,000 cells/kg body weight, about 1,000,000,000 cells/kg body weight, about 10,000,000,000 cells/kg body weight, about 100,000,000,000 cells/kg body weight, or about 1,000,000,000,000 cells/kg body weight. In some embodiments, the cell without the nucleus is administered to the subject twice within at least an hour, 2 hours, 4 hours, 6 hours, 8 hours, 12 hours, 1 day, 2 days, a week, 2 weeks, 3 weeks, a month, 2 months, 3 months, 4 months, 5 months, 6 months, 7 months, 8 months, 9 months, 10 months, 11 months, a year, 2 years, 3 years, or 4 years.

In some embodiments, the composition or pharmaceutical composition comprising the same enucleated cell can be repeatedly administered to the subject in need thereof. In some embodiments, the first administrations of the composition or pharmaceutical composition comprising the enucleated cell normalizes blood vessel or lymphatic vessel. In some embodiments, the composition or pharmaceutical composition comprising the same enucleated cell can be subsequently administered to the subject for: maintaining the normalization of the blood vessel or lymphatic vessel; and delivering the exogenous agent for treating a disease or condition described herein.

### Kits

Disclosed herein, in some embodiments, are kits for using the compositions described herein. In some embodiments, the kits disclosed herein may be used to treat a disease or condition in a subject. In some embodiments, the kits comprise an assemblage of materials or components apart from the composition.

In some embodiments, the kits described herein comprise a pharmaceutical formulation disclosed herein, comprising the enucleated cells engineered to express (and in some cases secrete) a single-domain antibody or antigen-binding fragment disclosed herein. In some embodiments, the single-domain antibody or antigen binding fragment is specific to an immune checkpoint molecule, such as PD-L1. In some embodiments, the enucleated cells are further engineered to express a targeting moiety, such as a chemokine receptor, an integrin signaling molecule or an antibody or antigen-binding fragment thereof that enables the enucleated cells to efficiently migrate to the target tissue in a subject, once administered. In some embodiments, the target tissue comprises lung tissue. In some embodiments, the kits further comprise an additional therapeutic agent, such as those disclosed herein. In some embodiments, the kit further comprises instructions for administering the pharmaceutical formulation and/or additional therapeutic agent to the subject to treat a disease or a condition in the subject such as cancer. In some embodiments, the cancer comprises cancer of the lung tissue. In some embodiments, the cancer is lung cancer.

In some embodiments, the kits described herein comprise a pharmaceutical formulation disclosed herein, comprising the enucleated cells engineered to express (and in some cases secrete) a single-domain antibody or antigen-binding fragment specific to an epithelial marker, such as CTGF. In some embodiments, the enucleated cells are further engineered to express a targeting moiety, such as a chemokine receptor, an integrin signaling molecule or an antibody or antigen-binding fragment thereof that enables the enucleated cells to efficiently migrate to the target tissue in a subject, once administered. In some embodiments, the target tissue comprises lung tissue. In some embodiments, the kits further comprise an additional therapeutic agent, such as those disclosed herein. In some embodiments, the kit further comprises instructions for administering the pharmaceutical formulation and/or additional therapeutic agent to the subject to treat a disease or a condition in the subject such pulmonary fibrosis. In some embodiments, the pulmonary fibrosis comprises idiopathic pulmonary fibrosis;

In some embodiments, the kits described herein comprise components for selecting for a homogenous population of the enucleated cells. In some embodiments, the kits described herein comprise components for selecting for a heterogenous population of the enucleated cells. In some embodiments, the kit comprises the components for assaying the number of units of a biomolecule (e.g., a therapeutic agent) synthesized, and/or released or expressed on the surface by the enucleated cell. In some embodiments, the kit comprises components for performing assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., cancer) in a subject. In some embodiments, the kit is configured particularly for the purpose of treating mammalian subjects. In some embodiments, the kit is configured particularly for the purpose of treating human subjects.

Instructions for use may be included in the kit. In some embodiments, the kit comprises instructions for administering the composition to a subject in need thereof. In some embodiments, the kit comprises instructions for further engineering the composition to express a biomolecule (e.g., a therapeutic agent). In some embodiments, the kit comprises instructions thawing or otherwise restoring biological activity of the composition, which may have been cryopreserved, lyophilized, or cryo-hibernated during storage or transportation. In some embodiments, the kit comprises instructions for measure viability of the restored compositions, to ensure efficacy for its intended purpose (e.g., therapeutic efficacy if used for treating a subject).

Optionally, the kit also contains other useful components such as diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components are typically contained in suitable packaging material(s).

### Definitions

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease may be, for example, at least 10%, at least 20%, at least 30%, at least 40% or more, and is preferably down to a level accepted as within the range of normal for an individual without a given disease. Other examples of “decrease” include a decrease of at least 2-fold, at least 5-fold, at least 10-fold, at least 20-fold, at least 50-fold, at least 100-fold, at least 1000-fold or more as compared to a reference level.

The terms “individual” or “subject” are used interchangeably and encompass mammals. Non-limiting examples of mammal include any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents such as rats, mice and guinea pigs, and the like. The mammal may be a human. The term “animal” as used herein comprises human beings and non-human animals. In one embodiment, a “non-human animal” is a mammal, for example a rodent such as rat or a mouse. A “patient,” as used herein refers to a subject that has, or has been diagnosed with, a disease or a condition described herein.

The term “immune-evading moiety,” as used herein, refers to a signaling peptide, or portion thereof, that reduces cellular phagocytosis through its interaction with a signal receptor protein expressed by phagocytic cells such as macrophages and dendritic cells. In some embodiments, the immune-evading moiety blocks immune cell recognition or immune cell activation.

The term “targeting moiety,” as used herein, refers to an entity that guides a cell such as for example, an enucleated cell, to a target tissue or cell. The targeting moiety can be virtually any biomolecule, including a protein, polypeptide, a sugar, a nucleic acid, or a small molecule, or portions thereof. In some embodiments, the targeting moiety disclosed herein comprise a chemokine receptor, a chemokine, an integrin signaling molecule, an antibody or antigen-binding fragment thereof, or a single-domain antibody or antigen-binding fragment thereof.

The term “transmembrane moiety,” as used herein, refers to an entity that spans (at least partially) the cell membrane of a cell (e.g., enucleated cell).

The terms “expression” or “expressing” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a control, e.g., wild-type state while “down-regulated” generally refers to a decreased expression level of a polynucleotide (e.g., RNA such as mRNA) and/or polypeptide sequence relative to its expression in a control, e.g., a wild-type state.

As used herein, a “cell” generally refers to a biological cell.

As used herein, “enucleation” is the rendering of a cell to a non-replicative state such as, for example, through removal of the nucleus

As used herein, the term “cytoplast,” “cell without a nucleus,” or “enucleated cell” are used interchangeably to refer to a nucleus-free cell that was obtained from a previously nucleated cell (e.g., any cell described herein). In some embodiments, the nucleated cell comprises cell organelles and the cytoplast derived from the nucleated cell retains such organelles, which in some cases, enables cellular functions such as cell motility, protein synthesis, protein secretion, formation of tunneling nanotube, and the like. In some embodiments “obtaining” refers to a process encompassing enucleation of a nucleated cell. In some embodiments, enucleation does not involve differentiating the nucleated cell into an enucleated cell using natural processes or otherwise.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. The term “gene” is to be interpreted broadly, and may encompass mRNA, cDNA, cRNA and genomic DNA forms of a gene. In some uses, the term “gene” encompasses the transcribed sequences, including 5′ and 3′ untranslated regions (5′-UTR and 3′-UTR), exons and introns. In some genes, the transcribed region may contain “open reading frames” that encode polypeptides. In some uses of the term, a “gene” comprises only the coding sequences (e.g., an “open reading frame” or “coding region”) necessary for encoding a polypeptide. In some aspects, genes do not encode a polypeptide, for example, ribosomal RNA genes (rRNA) and transfer RNA (tRNA) genes. In some aspects, the term “gene” includes not only the transcribed sequences, but in addition, also includes non-transcribed regions including upstream and downstream regulatory regions, enhancers and promoters. The term “gene” may encompass mRNA, cDNA and genomic forms of a gene.

The term “packaging material” refers to one or more physical structures used to house the contents of the kit such as compositions and the like. The packaging material is constructed by well-known methods, preferably to provide a sterile, contaminant-free environment. The packaging materials employed in the kit are those customarily utilized in gene expression assays and in the administration of treatments.

As used herein, the term “package” refers to a suitable solid matrix or material such as glass, plastic, paper, foil, and the like, capable of holding the individual kit components. For example, a package may be a glass vial or prefilled syringes used to contain suitable quantities of the pharmaceutical. The packaging material has an external label which indicates the contents and/or purpose of the kit and its components.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three dimensional structure, and may perform any function, known or unknown. A polynucleotide comprises one or more analogs (e.g., altered backbone, sugar, or nucleobase). Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

As used herein, the terms “polypeptide,” “peptide” and “protein” may be used interchangeably herein in reference to a polymer of amino acid residues. A protein may refer to a full-length polypeptide as translated from a coding open reading frame, or as processed to its mature form, while a polypeptide or peptide may refer to a degradation fragment or a processing fragment of a protein that nonetheless uniquely or identifiably maps to a particular protein. A polypeptide may be a single linear polymer chain of amino acids bonded together by peptide bonds between the carboxyl and amino groups of adjacent amino acid residues. Polypeptides may be modified, for example, by the addition of carbohydrate, phosphorylation, etc.

As used herein, the terms “fragment,” or “portion,” or equivalent terms may refer to a portion of a protein that has less than the full length of the protein and optionally maintains the function of the protein.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g., thermodynamically more stable under a given set of conditions such as stringent conditions commonly used in the art) to hybridization with non-target sequences during a hybridization reaction. Typically, hybridizable sequences share a degree of sequence complementarity over all or a portion of their respective lengths such as between 25%-100% complementarity, including greater than or equal to about 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity such as for the purpose of assessing percent complementarity, may be measured by any suitable alignment algorithm, including but not limited to the Needleman-Wunsch algorithm (see e.g., the EMBOSS Needle aligner available at www.ebi.ac.uk/Tools/psa/emboss needle/nucleotide.html, optionally with default settings), the BLAST algorithm. Optimal alignment may be assessed using any suitable parameters of a chosen algorithm, including default parameters.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (e.g., gaps may be introduced in one or both of the candidate and reference sequences for optimal alignment and non-homologous sequences may be disregarded for comparison purposes). Alignment, for purposes of determining percent identity, may be achieved in various ways that are commonly known. Percent identity of two sequences may be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “in vivo” may be used to describe an event that takes place in an organism such as a subject's body.

As used herein, the term “ex vivo” may be used to describe an event that takes place outside of an organism such as subject's body. An “ex vivo” assay cannot be performed on a subject. Rather, it may be performed upon a sample separate from a subject. Ex vivo may be used to describe an event occurring in an intact cell outside a subject's body.

As used herein, the term “in vitro” may be used to describe an event that takes places contained in a container for holding laboratory reagent such that it is separated from the living biological source organism from which the material is obtained. In vitro assays may encompass cell-based assays in which cells alive or dead are employed. In vitro assays may also encompass a cell-free assay in which no intact cells are employed.

“Treat, “treating,” or “treatment,” as used herein, refers to alleviating or abrogating a disorder, disease, or condition; or one or more of the symptoms associated with the disorder, disease, or condition; or alleviating or eradicating a cause of the disorder, disease, or condition itself. Desirable effects of treatment may include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “effective amount” and “therapeutically effective amount,” as used interchangeably herein, generally refer to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle such as a liquid or solid filler, diluent, excipient, solvent, or encapsulating material. A component may be “pharmaceutically acceptable” in the sense of being compatible with the other ingredients of a pharmaceutical formulation. It may also be suitable for use in contact with the tissue or organ of humans and animals without excessive toxicity, irritation, allergic response, immunogenicity, or other problems or complications, commensurate with a reasonable benefit/risk ratio.

As used herein, the term “administration,” “administering” and variants thereof means introducing a composition or agent into a subject and includes concurrent and sequential introduction of a composition or agent. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and administration by another. A suitable route of administration allows the composition or the agent to perform its intended function. For example, if a suitable route is intravenous, the composition is administered by introducing the composition or agent into a vein of the subject. Administration may be carried out by any suitable route. In some embodiments, the administering is intravenous administration. In some embodiments, the administering is pulmonary administration. In some embodiments, the administering is inhalation.

The term “pharmaceutical formulation” refers to a mixture of a composition disclosed herein with other chemical components such as diluents or carriers (e.g., pharmaceutically acceptable inactive ingredients) such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical formulation may facilitate administration of the composition to an organism. Multiple techniques of administering a compound exist in the art including, but not limited to, oral, injection, aerosol, parenteral, and topical administration.

The term “fusogenic protein”, as used herein, refers to a polypeptide that, when expressed on the surface of a cell such as the enucleated cell, facilitates fusion of the cell comprising the fusogenic protein and a target cell.

The term, “anchor molecule” comprises a molecule that can be inserted and remaining in a cellular membrane. For example, glycosylphosphatidylinositol (GPI) is an anchor molecule that can be complexed with a protein, where the GPI can be inserted and remaining in the cellular membrane, thereby anchoring the protein to the cellular membrane.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EMBODIMENTS

Embodiment 1. An enucleated cell obtained from a parent cell with a nucleus, the enucleated cell comprising: one or more intracellular organelles for synthesis of an exogenous single-domain antibody or fragment thereof in absence of the nucleus.

Embodiment 2. The enucleated cell of Embodiment 1, wherein the exogenous single-domain antibody or fragment thereof is encapsulated in the enucleated cell.

Embodiment 3. The enucleated cell of Embodiment 1, wherein the exogenous single-domain antibody or fragment thereof is expressed on an exoplasmic side of a cell membrane of the enucleated cell by the one or more intracellular organelles.

Embodiment 4. The enucleated cell of Embodiment 1, wherein the exogenous single-domain antibody or fragment thereof is expressed on a cytosolic side of a cell membrane of the enucleated cell by the one or more intracellular organelles.

Embodiment 5. The enucleated cell of Embodiment 1, wherein the exogenous single-domain antibody or fragment thereof is complexed with a transmembrane moiety.

Embodiment 6. The enucleated cell of Embodiment 5, wherein the transmembrane moiety comprises a transmembrane polypeptide.

Embodiment 7. The enucleated cell of Embodiment 6, wherein the exogenous single-domain antibody or fragment thereof is complexed with N-terminus of the transmembrane polypeptide.

Embodiment 8. The enucleated cell of Embodiment 6, wherein the exogenous single-domain antibody or fragment thereof is complexed with C-terminus of the transmembrane polypeptide.

Embodiment 9. The enucleated cell of Embodiment 1, wherein the exogenous single-domain antibody or fragment thereof comprises a modification relative to an otherwise identical reference single-domain antibody or fragment thereof, wherein the modification anchors the exogenous single-domain antibody or fragment thereof to an exoplasmic or a cytosolic side of a cell membrane of the enucleated cell.

Embodiment 10. The enucleated cell of Embodiment 9, wherein the modification comprises complexing the exogenous single-domain antibody or fragment thereof to an anchor molecule comprising glycosylphosphatidylinositol, farnesyl, palmitate, myristate, or a combination thereof.

Embodiment 11. The enucleated cell of Embodiment 1, wherein the exogenous single-domain antibody or fragment thereof is released by the enucleated cell by secreting the exogenous single-domain antibody or fragment thereof from the enucleated cell.

Embodiment 12. The enucleated cell of Embodiment 10, wherein the exogenous single-domain antibody or fragment thereof is released upon death of the enucleated cell.

Embodiment 13. The enucleated cell of Embodiment 11, wherein the exogenous single-domain antibody or fragment thereof is released upon rupture of the enucleated cell.

Embodiment 14. The enucleated cell of Embodiment 11, wherein the exogenous single-domain antibody or fragment thereof is transferred from the enucleated cell to another cell by fusing the enucleated cell with the another cell.

Embodiment 15. The enucleated cell of any one of preceding Embodiments, wherein the exogenous single-domain antibody or fragment thereof is conjugated to a cytotoxic drug.

Embodiment 16. The enucleated cell of Embodiment 1, wherein the enucleated cell comprises an exogenous polynucleotide encoding a polypeptide sequence that corresponds to the exogenous single-domain antibody or fragment thereof.

Embodiment 17. The enucleated cell of Embodiment 16, wherein the polypeptide sequence comprises a sequence provided in SEQ ID NOs: 1-36, 101-111, 121-123, 165-192, 195, 205, 206, 211-213, 221-231, 241-245, 325-331, and 401-404.

Embodiment 18. The enucleated cell of Embodiment 1, wherein the exogenous single-domain antibody or fragment thereof is specific to an antigen encoded by at least one nucleic acid in SEQ ID NOs: 131-134, 142-152, 201-202, 301-312, 501, 521-526, 541-545, 561, 584, 591-601, and 701-705.

Embodiment 19. The enucleated cell of Embodiment 1, wherein the exogenous single-domain antibody or fragment thereof is specific to an antigen comprising a peptide sequence encoding PD-L1.

Embodiment 20. The enucleated cell of Embodiment 1, wherein the exogenous single-domain antibody or fragment thereof is specific to an antigen comprising at least one peptide sequence in SEQ ID NOs: 155-164, 203, 204, 315-322, 511, 531-535, 551-554, 571, 594, 611-619, and 711.

Embodiment 21. The enucleated cell of Embodiment 1, wherein the exogenous single-domain antibody or fragment thereof is specific to an antigen associated with at least one pathogen in Table 1.

Embodiment 22. The enucleated cell of Embodiment 1, wherein the exogenous single-domain antibody or fragment thereof is specific to an antigen comprising at least one peptide sequence encoding Connective tissue growth factor (CTGF).

Embodiment 23. The enucleated cell of Embodiment 1, wherein the exogenous single-domain antibody or fragment thereof is specific to an antigen comprising at least one peptide sequence in SEQ ID NOs: 1601-1602.

Embodiment 24. The enucleated cell of Embodiment 1, wherein the exogenous single-domain antibody or fragment thereof is encoded from a nucleic acid sequence that is greater than or equal to about 50%, 55%, 60%, 65%, 70%, 75%, 80%, 85%, 90%, 95%, or 99% identical to SEQ ID NO: 801.

Embodiment 25. The enucleated cell of Embodiment 1, wherein the exogenous single-domain antibody or fragment thereof comprises a polypeptide sequence that is greater than or equal to about 50%, 55%, 60%, 65%, 70%, 75%, 80%, 85%, 90%, 95%, or 99% identical to SEQ ID NO: 851.

Embodiment 26. The enucleated cell of Embodiment 1, wherein the exogenous single-domain antibody or fragment thereof is encoded from a nucleic acid sequence that is greater than or equal to about 50%, 55%, 60%, 65%, 70%, 75%, 80%, 85%, 90%, 95%, or 99% identical to SEQ ID NO: 901.

Embodiment 27. The enucleated cell of Embodiment 1, wherein the exogenous single-domain antibody or fragment thereof comprises a polypeptide sequence that is greater than or equal to about 50%, 55%, 60%, 65%, 70%, 75%, 80%, 85%, 90%, 95%, or 99% identical to SEQ ID NO: 951.

Embodiment 28. The enucleated cell of Embodiment 1, wherein the exogenous single-domain antibody or fragment thereof comprises a polypeptide sequence that is greater than or equal to about 50%, 55%, 60%, 65%, 70%, 75%, 80%, 85%, 90%, 95%, or 99% identical to SEQ ID NO: 1701.

Embodiment 29. The enucleated cell of Embodiment 1, wherein the exogenous single-domain antibody or fragment thereof is specific to an antigen expressed by a cancer cell in lung tissue.

Embodiment 30. The enucleated cell of Embodiment 29, wherein the cancer cell is a non-small cell lung cancer (NSCLC) cell.

Embodiment 31. The enucleated cell of Embodiment 29, wherein the cancer cell is a cell of adenocarcinoma, squamous carcinoma, large cell (undifferentiated) carcinoma, large cell neuroendocrine carcinoma, adenosquamous carcinoma, or sarcomatoid carcinoma.

Embodiment 32. The enucleated cell of Embodiment 29, wherein the cancer cell is a cell of small cell lung cancer (SCLC).

Embodiment 33. The enucleated cell of Embodiment 29, wherein the cancer cell is a cell of lung carcinoid tumor.

Embodiment 34. The enucleated cell of Embodiment 29, wherein the cancer cell is a cell of adenoid cystic carcinoma.

Embodiment 35. The enucleated cell of Embodiment 29, wherein the cancer cell is a cell of lymphoma.

Embodiment 36. The enucleated cell of Embodiment 29, wherein the cancer cell is a cell of sarcoma.

Embodiment 37. The enucleated cell of Embodiment 29, wherein the cancer cell is a cell of benign lung tumor.

Embodiment 38. The enucleated cell of Embodiment 37, wherein the cancer cell is a cell of hamartoma.

Embodiment 39. The enucleated cell of Embodiment 1, wherein the exogenous single-domain antibody or fragment thereof is specific to an antigen expressed by a cell associated with idiopathic pulmonary fibrosis.

Embodiment 40. The enucleated cell of Embodiment 39, wherein the exogenous single-domain antibody or fragment thereof is specific to an antigen expressed by the cell associated with idiopathic pulmonary fibrosis is a lung cell.

Embodiment 41. The enucleated cell of Embodiment 39, wherein the exogenous single-domain antibody or fragment thereof is specific to an antigen expressed by the cell associated with idiopathic pulmonary fibrosis is an immune cell.

Embodiment 42. The enucleated cell of Embodiment 39, wherein the exogenous single-domain antibody or fragment thereof is specific to an antigen expressed by the cell associated with idiopathic pulmonary fibrosis is an alveolar cell.

Embodiment 43. The enucleated cell of Embodiment 39, wherein the exogenous single-domain antibody or fragment thereof is specific to an antigen expressed by the cell associated with idiopathic pulmonary fibrosis is an alveolar epithelial cell (AEC).

Embodiment 44. The enucleated cell of Embodiment 39, wherein the exogenous single-domain antibody or fragment thereof is specific to an antigen expressed by the cell associated with idiopathic pulmonary fibrosis is a bronchial cell.

Embodiment 45. The enucleated cell of any one of preceding Embodiments, wherein the enucleated cell further comprises at least one additional exogenous agent.

Embodiment 46. The enucleated cell of Embodiment 1, wherein the enucleated cell further comprises a fusogenic moiety.

Embodiment 47. The enucleated cell of Embodiment 46, wherein the fusogenic moiety comprises a viral fusogenic moiety.

Embodiment 48. The enucleated cell of Embodiment 46, wherein the fusogenic moiety comprises an eukaryotic fusogenic moiety.

Embodiment 49. The enucleated cell of Embodiment 1, wherein the enucleated cell further comprises an immune evasion moiety.

Embodiment 50. The enucleated cell of Embodiment 49, wherein the immune evasion moiety comprises CD47, PD-L1, HLA-E, HLA-G, a fragment thereof, or a combination thereof.

Embodiment 51. The enucleated cell of Embodiment 1, wherein the enucleated cell further comprises a targeting moiety.

Embodiment 52. The enucleated cell of Embodiment 51, wherein the targeting moiety targets a biomarker of the cancer cell.

Embodiment 53. The enucleated cell of Embodiment 52, wherein the exogenous single-domain antibody or fragment thereof is specific to an antigen expressed by a cancer cell, and wherein the biomarker is a separate and distinct entity from the antigen targeted by the exogenous single-domain antibody or fragment thereof.

Embodiment 54. The enucleated cell of Embodiment 51, wherein the targeting moiety targets a biomarker of an immune cell within the microenvironment of the tumor.

Embodiment 55. The enucleated cell of Embodiment 54, wherein the biomarker is expressed on surface of the immune cell.

Embodiment 56. The enucleated cell of Embodiment 54, wherein the biomarker is released by the immune cell.

Embodiment 57. The enucleated cell of any one of Embodiments 51-56, wherein the targeting moiety comprises a chemokine.

Embodiment 58. The enucleated cell of any one of Embodiments 51-56, wherein the targeting moiety comprises a chemokine receptor.

Embodiment 59. The enucleated cell of any one of Embodiments 41-56, wherein the targeting moiety comprises an adhesion molecule.

Embodiment 60. The enucleated cell of any one of Embodiments 51-56, wherein the targeting moiety comprises an antigen.

Embodiment 61. The enucleated cell of any one of Embodiments 51-56, wherein the targeting moiety comprises an antigen that is a separate and distinct entity from an antigen expressed by the cancer cell.

Embodiment 62. The enucleated cell of any one of Embodiments 51-56, wherein the targeting moiety comprises an antibody that is not expressed by the cancer cell.

Embodiment 63. The enucleated cell of any one of Embodiments 51-56, wherein the targeting moiety comprises a membrane-bound antibody.

Embodiment 64. The enucleated cell of Embodiment 63, wherein the membrane bound antibody is a membrane-bound single-domain antibody.

Embodiment 65. The enucleated cell of Embodiment 1, wherein the enucleated cell has a diameter comprising between about 1 micrometers (μm) to about 100 μm.

Embodiment 66. The enucleated cell of Embodiment 65, wherein the diameter comprises between about 1 μm to about 10 μm.

Embodiment 67. The enucleated cell of Embodiment 65, wherein the diameter comprises between about 10 μm to about 100 μm.

Embodiment 68. The enucleated cell of Embodiment 65, wherein the diameter is at least or about 1 μm, 5 μm, 8 μm, 10 μm, 20 μm, 30 μm, 40 μm, 50 μm, 60 μm, 70 μm, 80 μm, 90 μm, or 100 μm.

Embodiment 69. The enucleated cell of Embodiment 68, wherein the diameter comprises about 8 μm.

Embodiment 70. The enucleated cell of Embodiment 1, wherein the enucleated cell exhibits a diameter that is at least about 10%, 20%, 30%, 40%, 50%, 60%, 70%, 80%, 90%, 95%, or 99% smaller compared to the parent cell that is nucleated.

Embodiment 71. The enucleated cell of Embodiment 1, wherein the parent cell is selected from the group consisting of: a stem cell, an induced pluripotent stem cell (iPSC), an adult stem cell, a mesenchymal stromal cell, an embryonic stem cell, a fibroblast, and a cell from a cell line.

Embodiment 72. The enucleated cell of Embodiment 71, wherein the parent cell is mesenchymal stromal cell.

Embodiment 73. The enucleated cell of Embodiment 1, wherein the enucleated cell exhibits viability after cryohibemation.

Embodiment 74. The enucleated cell of Embodiment 73, wherein the enucleated cell exhibits the viability following the cryohibemation as measured at 24 hours following the cryohibemation that is equal to or greater than the viability of a comparable enucleated cell that is not cryohibernated.

Embodiment 75. The enucleated cell of Embodiment 1, wherein the enucleated cell exhibits viability after cryopreservation.

Embodiment 76. The enucleated cell of Embodiment 75, wherein the enucleated cell exhibits the viability following the cryopreservation as measured at 24 hours following the cryopreservation that is equal to or greater than the viability of a comparable enucleated cell that is not cryopreserved.

Embodiment 77. The enucleated cell of any one of preceding Embodiments, wherein the enucleated cell is isolated.

Embodiment 78. The enucleated cell of any one of Embodiments 1-77, wherein the enucleated cell is purified.

Embodiment 79. The enucleated cell of any one of Embodiments 1-77, wherein the enucleated cell is lyophilized.

Embodiment 80. A cell line comprising the enucleated cell of any one of preceding Embodiments.

Embodiment 81. A plurality of cells comprising the enucleated cell of any one of preceding Embodiments.

Embodiment 82. A pharmaceutical composition comprising: the enucleated cell of any one of Embodiments 1-80; and a pharmaceutically acceptable: excipient, carrier, or diluent.

Embodiment 83. The pharmaceutical composition of Embodiment 82 comprises a unit dose form.

Embodiment 84. The pharmaceutical composition of Embodiment 82 or 83, which is formulated for administering intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof.

Embodiment 85. The pharmaceutical composition of any one of Embodiments 82-84, which is formulated for administering intravenously.

Embodiment 86. The pharmaceutical composition of any one of Embodiments 82-84, which is formulated for administering intratumorally.

Embodiment 87. The pharmaceutical composition of any one of Embodiments 82-84, which is formulated for administering pulmonarily.

Embodiment 88. The pharmaceutical composition of any one of Embodiments 82-84, which is formulated for administering endotracheally.

Embodiment 89. The pharmaceutical composition of any one of Embodiments 82-84, which is formulated for administering by inhaled nebulized form.

Embodiment 90. The pharmaceutical composition of any one of Embodiments 82-84, further comprising at least one additional active agent.

Embodiment 91. The pharmaceutical composition of Embodiment 90, wherein the at least one additional active agent comprises a cytokine, a growth factor, a hormone, an antibody, an enzyme, a small molecule, a compound, or combinations thereof.

Embodiment 92. A kit comprising: the enucleated cell of any one of Embodiments 1-80, the cell line of Embodiment 81, the plurality of cells of Embodiment 71, or the pharmaceutical composition of any one of Embodiments 82-91; and a container.

Embodiment 93. A method of treating a disease or condition in a subject in need thereof, the method comprising: administering to the subject having the disease or the condition associated with a target cell in the subject a therapeutically effective amount of cell of any one of Embodiments 1-80 or the pharmaceutical composition of any one of Embodiments 82-91, wherein the exogenous single-domain antibody or fragment thereof binds to an antigen expressed by the target cell in the subject, thereby treating the disease or the condition in the subject.

Embodiment 94. The method of Embodiment 93, wherein the enucleated cell is an autologous cell.

Embodiment 95. The method of Embodiment 93, wherein the enucleated cell is an allogenic cell.

Embodiment 96. The method of Embodiment 93, wherein the antigen comprises tumor-associated antigen (TAA).

Embodiment 97. The method of Embodiment 93, wherein the antigen comprises tumor-specific antigen (TSA).

Embodiment 98. The method of any one of Embodiments 93-97, wherein the binding of the exogenous single-domain antibody or fragment thereof to the antigen directly kills the cancer cell.

Embodiment 99. The method of any one of Embodiments 93-97, wherein the binding of the exogenous single-domain antibody or fragment thereof to the antigen disrupts cell cycle signaling of the cancer cell.

Embodiment 100. The method of any one of Embodiments 93-97, wherein the binding of the exogenous single-domain antibody or fragment thereof to the antigen disrupts angiogenesis signaling of the cancer cell.

Embodiment 101. The method of any one of Embodiments 93-97, wherein the binding of the exogenous single-domain antibody or fragment thereof to the antigen recruits an immune cell to the cancer cell.

Embodiment 102. The method of Embodiment 83, wherein the immune cell is a T cell.

Embodiment 103. The method of any one of Embodiments 93-102, wherein the enucleated cell of any one of Embodiments 1-80 or the pharmaceutical composition of any one of Embodiments 82-91 is administered to the subject intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof.

Embodiment 104. The method of Embodiment 87, wherein the enucleated cell of any one of Embodiments 1-80 or the pharmaceutical composition of any one of Embodiments 82-91 is administered intravenously.

Embodiment 105. The method of Embodiment 86, wherein the enucleated cell of any one of Embodiments 1-80 or the pharmaceutical composition of any one of Embodiments 82-91 is administered intratumorally.

Embodiment 106. The method of Embodiment 86, wherein the enucleated cell of any one of Embodiments 1-80 or the pharmaceutical composition of any one of Embodiments 82-91 is administered pulmonarily.

Embodiment 107. The method of Embodiment 86, wherein the enucleated cell of any one of Embodiments 1-80 or the pharmaceutical composition of any one of Embodiments 82-91 is administered endotracheally.

Embodiment 108. The method of Embodiment 86, wherein the enucleated cell of any one of Embodiments 1-80 or the pharmaceutical composition of any one of Embodiments 82-91 is administered by inhaled nebulized form.

Embodiment 109. The method of any one of Embodiments 93-108, wherein, following administration of the enucleated cell of any one of Embodiments 1-80 or the pharmaceutical composition of any one of Embodiments 82-91 to the subject, the enucleated cell is viable fewer than or equal to 14 days in the subject.

Embodiment 110. The method of any one of Embodiments 93-109, wherein, following administration of the enucleated cell of any one of Embodiments 1-90 or the pharmaceutical composition of any one of Embodiments 92-101 to the subject, the enucleated cell is viable fewer than or equal to 4 days in the subject.

Embodiment 111. The method of any one of Embodiments 93-110, wherein the target cell is a cancer cell.

Embodiment 112. The method of any one of Embodiments 93-111, wherein the disease or the condition is cancer or a neoplasm.

Embodiment 113. The enucleated cell of any one of Embodiments 1-79, wherein the exogenous single-domain antibody or fragment thereof comprises a neutralizing antibody.

Embodiment 114. The enucleated cell of any one of Embodiments 1-79 or 113, wherein the exogenous single-domain antibody or fragment thereof binds a VEGF.

Embodiment 115. The enucleated cell of Embodiment 114, wherein the exogenous single-domain antibody or fragment thereof binds a VEGF-A.

Embodiment 116. The enucleated cell of any one of Embodiments 51-64, wherein the targeting moiety targets an endothelial cell biomarker.

Embodiment 117. The enucleated cell of Embodiment 116, wherein the endothelial cell biomarker is expressed by a vasculature cell.

Embodiment 118. The enucleated cell of Embodiment 116 wherein the endothelial cell biomarker is expressed by a blood vessel cell.

Embodiment 119. The enucleated cell of Embodiment 116, wherein the endothelial cell biomarker is expressed by a lymphatic vessel cell.

Embodiment 120. The enucleated cell of Embodiment 45, wherein the at least one additional exogenous agent comprises a polypeptide comprising a tumor necrosis factor (TNF) superfamily member polypeptide or a catalytically active fragment thereof.

Embodiment 121. The enucleated cell of Embodiment 120, wherein the TNF superfamily member polypeptide or the catalytically active fragment thereof soluble in aqueous conditions, when solubility is measure in vitro by turbidimetric solubility assay or thermodynamic solubility assay.

Embodiment 122. The enucleated cell of Embodiment 120 or 121, wherein the TNF superfamily member polypeptide comprises LIGHT.

Embodiment 123. The enucleated cell of any one of Embodiments 120-122, wherein the TNF superfamily member polypeptide comprises soluble LIGHT.

Embodiment 124. The enucleated cell of Embodiment 45, wherein the at least one additional exogenous agent comprises an immune checkpoint molecule.

Embodiment 125. The enucleated cell of Embodiment 45, wherein the at least one additional exogenous agent comprises an immune checkpoint inhibitor molecule.

Embodiment 126. The enucleated cell of Embodiment 45, wherein the at least one additional exogenous agent comprises an angiogenesis inhibitor.

Embodiment 127. The enucleated cell of Embodiment 126, wherein the angiogenesis inhibitor comprises a VEGF/VEGFR inhibitor.

Embodiment 128. The enucleated cell of Embodiment 126, wherein the VEGF/VEGFR inhibitor comprises a VEGF-A inhibitor.

Embodiment 129. An enucleated cell obtained from a parent cell with a nucleus, the enucleated cell comprising: one or more intracellular organelles for synthesis of an exogenous polypeptide comprising a tumor necrosis factor (TNF) superfamily member polypeptide or a catalytically active fragment thereof in absence of the nucleus.

Embodiment 130. The enucleated cell of Embodiment 129, further comprising at least one exogenous targeting moiety.

Embodiment 131. The enucleated cell of Embodiment 129 or 130, wherein the exogenous polypeptide comprises a solubility of at least 0.0001 mg/ml, 0.0005 mg/ml, 0.001 mg/ml, 0.005 mg/ml, 0.01 mg/ml, 0.05 mg/ml, 0.1 mg/ml, 0.5 mg/ml, 1.0 mg/ml, 5.0 mg/ml, 10 mg/ml, 50 mg/ml, 100 mg/ml, 500 mg/ml 1,000 mg/ml 5,000 mg/ml, 10,000 mg/ml, 50,000 mg/ml, or 100,000 mg/ml in aqueous conditions when solubility is measured by turbidimetric solubility assay or thermodynamic solubility assay.

Embodiment 132. The enucleated cell of Embodiment 129 or 130, wherein the exogenous polypeptide is expressed on an exoplasmic side of a cell membrane of the enucleated cell by the one or more intracellular organelles.

Embodiment 133. The enucleated cell of Embodiment 129 or 130, wherein the exogenous polypeptide is released by the enucleated cell.

Embodiment 134. The enucleated cell of any one of Embodiments 129-133, further comprising an exogenous polynucleotide encoding the exogenous polypeptide.

Embodiment 135. The enucleated cell of any one of Embodiments 129-134, wherein the exogenous polypeptide comprises a sequence that is at least 75%, 80%, 85%, 90%, 95%, or 99% identical to SEQ ID NOs: 1501-1511.

Embodiment 136. The enucleated cell of any one of Embodiment 135, wherein the exogenous polypeptide comprises a sequence that is at least 75%, 80%, 85%, 90%, 95%, or 99% identical to SEQ ID NO: 1511.

Embodiment 137. The enucleated cell of any one of Embodiments 129-136, wherein the TNF superfamily member polypeptide is LIGHT.

Embodiment 138. The enucleated cell of any one of Embodiments 129-136, further comprising a second exogenous polypeptide.

Embodiment 139. The enucleated cell of Embodiment 138, wherein the second exogenous polypeptide comprises an antibody, an immune checkpoint molecule, or a fragment thereof.

Embodiment 140. The enucleated cell of Embodiment 138, wherein the second exogenous polypeptide comprises an antibody, or antigen-binding fragment thereof.

Embodiment 141. The enucleated cell of Embodiment 140, wherein the antibody or antigen-binding fragment thereof is a neutralizing antibody or neutralizing antigen-binding fragment thereof.

Embodiment 142. The enucleated cell of Embodiment 141, wherein the neutralizing antibody or neutralizing antigen-binding fragment thereof targets.

Embodiment 143. The enucleated cell of Embodiment 141, wherein the neutralizing antibody or neutralizing antigen-binding fragment thereof targets Angiopoitin-1, Angiopoitin-2, Endostatin, FGF, MMP, DII4, Class 3 semaphorins, FGF, VEGFR, NRP-1, PDGF (BB-homodimer), PDGFR, TGF-β, endoglin, TGF-β receptors, CCL2, Integrins αVβ3, αVβ5, or α5β1, VE-cadherin, CD31, ephrin, plasminogen activator, plasminogen activator inhibitor-1, eNOS, COX-2, AC133, ID1/ID3, Class 3 semaphorin, or Nogo-A.

Embodiment 144. The enucleated cell of Embodiment 141, wherein the neutralizing antibody or neutralizing antigen-binding fragment thereof targets VEGF.

Embodiment 145. The enucleated cell of Embodiment 144, wherein the neutralizing antibody or neutralizing antigen-binding fragment thereof targets VEGF-A.

Embodiment 146. The enucleated cell of Embodiment 139, wherein the immune checkpoint molecule comprises PD-1, PD-L1, CTLA-4, VISTA, PDCD1LG2 (CD273), B7-H3 (also called CD276), A2AR, CD27, LAG3, TIM-3, T cell immunoreceptor with Ig and ITIM domains (TIGIT), CD73, NKG2A, PVRIG, PVRL2, CEACAM1, CEACAM5, CEACAM6, FAK, CCR-2, CCL-2, LIF, CD47, SIRPα, M-CSF, CSF-1R, IL-3, IL-1RAP, IL-8, SEMA4D, Angiopoietin-2, CLEVER-1, Axl, phosphatidylserine or a fragment thereof.

Embodiment 147. The enucleated cell of Embodiment 130, wherein the at least one exogenous targeting moiety comprises an antibody or an antigen-binding fragment thereof.

Embodiment 148. The enucleated cell of Embodiment 140, wherein the antibody or the antigen-binding fragment thereof comprises an exogenous single-domain antibody or fragment thereof.

Embodiment 149. The enucleated cell of Embodiment 148, wherein the antibody or the antigen-binding fragment thereof targets a cancer cell marker.

Embodiment 150. The enucleated cell of Embodiment 148, wherein the antibody or the antigen-binding fragment thereof targets an endothelial cell biomarker.

Embodiment 151. The enucleated cell of Embodiment 150, wherein the endothelial cell biomarker is expressed by a vasculature cell.

Embodiment 152. The enucleated cell of Embodiment 150, wherein the endothelial cell biomarker is expressed by a blood vessel cell.

Embodiment 153. The enucleated cell of Embodiment 150, wherein the endothelial cell biomarker is expressed by a lymphatic vessel cell.

Embodiment 154. A method of treating a disease or condition characterized, at least in part, by abnormal vasculature in a subject, the method comprising: administering to the subject having the disease or the condition an enucleated cell comprising one or more intracellular organelles that synthesizes or releases an exogenous polypeptide comprising a tumor necrosis factor (TNF) superfamily member polypeptide or a catalytically active fragment thereof in absence of the nucleus, wherein the exogenous polypeptide synthesized or released by the cell is therapeutically effective to normalize the abnormal vasculature in the subject.

Embodiment 155. The method Embodiment 154, wherein the exogenous polypeptide comprises a soluble TNF superfamily member polypeptide.

Embodiment 156. The method Embodiment 154, wherein the exogenous polypeptide is released by the enucleated cell.

Embodiment 157. The method of any one of Embodiments 154-156, wherein the exogenous polypeptide comprises a polypeptide sequence that is at least 75%, 80%, 85%, 90%, 95%, or 99% identical to SEQ ID NOs: 1501-1511.

Embodiment 158. The method of any one of Embodiment 157 wherein the exogenous polypeptide comprises a polypeptide sequence that is at least 75%, 80%, 85%, 90%, 95%, or 99% identical to SEQ ID NO: 1508.

Embodiment 159. The method of any one of Embodiment 157, wherein the exogenous polypeptide comprises a polypeptide sequence that is at least 75%, 80%, 85%, 90%, 95%, or 99% identical to SEQ ID NO: 1511.

Embodiment 160. The method of any one of Embodiments 154-159, wherein the TNF superfamily member polypeptide is LIGHT.

Embodiment 161. The method of Embodiment 154, wherein the enucleated cell further comprises at least one exogenous targeting moiety comprising an antibody or an antigen-binding fragment.

Embodiment 162. The method of Embodiment 161, wherein the antibody or the antigen-binding fragment comprises an exogenous single-domain antibody or fragment thereof.

Embodiment 163. The method of Embodiment 162, wherein the antibody or the antigen-binding fragment targets a cancer cell marker.

Embodiment 164. The method of Embodiment 162, wherein the antibody or the antigen-binding fragment targets an endothelial cell biomarker.

Embodiment 165. The method of Embodiment 164, wherein the endothelial cell biomarker is expressed by a vasculature cell.

Embodiment 166. The method of Embodiment 164, wherein the endothelial cell biomarker is expressed by a blood vessel cell.

Embodiment 167. The method of Embodiment 164, wherein the endothelial cell biomarker is expressed by a lymphatic vessel cell.

Embodiment 168. The method of any one of Embodiments 154-167, wherein the enucleated cell delivers the exogenous polypeptide to a cell within the abnormal vasculature of the subject.

Embodiment 169. The method of nay one of Embodiments 154-168, wherein the enucleated cell comprises at least one additional exogenous agent.

Embodiment 170. The method of Embodiment 169, wherein the at least one additional exogenous agent comprises an immune checkpoint molecule.

Embodiment 171. The method of Embodiment 169, wherein the at least one additional exogenous agent comprises an immune checkpoint inhibitor molecule.

Embodiment 172. The method of Embodiment 169, wherein the at least one additional exogenous agent comprises an angiogenesis inhibitor.

Embodiment 173. The method of Embodiment 172, wherein the angiogenesis inhibitor comprises a VEGF/VEGFR inhibitor.

Embodiment 174. The method of Embodiment 173, wherein the VEGF/VEGFR inhibitor comprises a VEGF-A inhibitor.

Embodiment 175. The method of Embodiment 169, wherein the at least one exogenous agent kills a cancer within the abnormal vasculature.

Embodiment 176. The method of Embodiment 169, wherein the at least one exogenous agent recruits an endogenous immune cell to the abnormal vasculature to kill a cancer within the abnormal vasculature.

Embodiment 177. The method of any one of Embodiments 154-176, wherein the enucleated cell is administered to the subject intrathecally, intraocularly, intravitreally, retinally, intravenously, intramuscularly, intraventricularly, intracerebrally, intracerebellarly, intracerebroventricularly, intraperenchymally, subcutaneously, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof.

Embodiment 178. The method of any one of Embodiments 154-177, wherein, following administration of the enucleated cell to the subject, the enucleated cell is viable fewer than or equal to 14 days in the subject.

Embodiment 179. The method of any one of Embodiments 154-178, wherein, following administration of the enucleated cell to the subject, the enucleated cell is viable fewer than or equal to 4 days in the subject.

Embodiment 180. The method of any one of Embodiments 154-179, wherein the disease or the condition is cancer or a neoplasm.

Embodiment 181. The method of any one of Embodiments 154-180, wherein the abnormal vasculature is in the lung of the subject.

Embodiment 182. The method of any one of Embodiments 154-181 furthermore comprises administering to the subject CPI-006, Monalizumab, COM701, CM24, NEO-201, Defactinib, PF-04136309, MSC-1, Hu5F9-G4 (5F9), ALX148, TTI-662, RRx-001, Lacnotuzumab (MCS110), LY3022855, SNDX-6352, Emactuzumab (RG7155), Pexidartinib (PLX3397), CAN04, Canakinumab (ACZ885), BMS-986253, Pepinemab (VX15/2503), Trebananib, FP-1305, Enapotamab vedotin (EnaV), Bavituximab, or a combination thereof.

### EXAMPLES

The following illustrative examples are representative of embodiments of the stimulation, systems, and methods described herein and are not meant to be limiting in any way.

### Example 1. Successful Enucleation and Survival of Mammalian Cells

The enucleation efficiency and recovery rate of various types of mammalian cells (e.g., mesenchymal stem cells, neutrophils, fibroblast, and natural killer cells) was determined. After removal of the mammalian cells from the cell culture plates, the mammalian cells were enucleated by density gradient centrifugation using discontinuous Ficoll gradients, high-speed centrifugation (FIG. 2A-FIG. 2D). Table 2 summarizes the results of enucleation using a suspension protocol. Enucleation efficiency and cell viability was the highest in both hTERT transformed and primary mesenchymal stem cells (MSCs), as well as in fibroblasts and neutrophils. Table 3 summarizes the results of enucleation using an adherent protocol. Enucleation efficiency was greater than 70% in both mesenchymal stem cells and macrophages. This experiment showed that various types of mammalian cells could undergo enucleation using any of the methods described herein.

TABLE 2
Enucleation efficiency and viability determinations of mammalian cells

| | | Enucleation | Recovery | Viability after | Yield per |
|---|---|---|---|---|---|
| Cell type | | Efficiency | Rate | 24 hours | run |
| MSC cells | AD-MSC (hTERT) | 90%-95% | 60%-90% | 80%-95% | 12-15M |
| | UC-MSC (primary) | 85%-90% | 60%-80% | 80%-95% | 10-15M |
| | BM-MSC (primary) | 80%-90% | 40%-50% | 80%-90% | ~8M |
| NK cells | NKL | 50%-85% | 20%-50% | 50%-75% | ~8M |
| | NK-92 | 70%-90% | 20%-40% | 20%-40% | ~5M |
| Macrophages | RAW | 85%-95% | 40%-70% | 20%-40% | ~15M |
| | 264.7 | | | | |
| Neutrophils | HL-60 | 60%-98% | 20%-40% | 60%-80% | ~15M |
| Fibroblasts | L929 | 70%-90% | 50%-70% | 70%-90% | ~15M |
| | NIH3T3 | 70%-80% | 40%-50% | 70%-80% | ~9M |

Enucleation efficiency = enucleated cells versus total recovered cells;
Recovery rate = recovered cells versus total input cells used for enucleation.
Viability after 24 hours = live cells measured by Trypan blue staining versus total cells;
Yield per run = the number of cytoplasts harvested for each run;
M = million cells
AD-MSC (hTERT) = human hTERT immortalized adipose-derived mesenchymal stem cells;
BM-MSC (primary) = human primary bone marrow-derived mesenchymal stem cells;
NK = natural killer cells.

TABLE 3
Enucleation efficiencies and viability determinations of mammalian cells

| | | Enucleation | Recovery | Viability after | Yield |
|---|---|---|---|---|---|
| Cell type | | Efficiency | Rate | 24 hours | per run |
| MSC cells | AD-MSC | 70%-95% | 40%-60% | 80%-95% | 1M |
| | (hTERT) | | | | |
| Macrophages | RAW | 85%-95% | 40%-70% | 10%-30% | ~1M |
| | 264.7 | | | | |

Enucleation efficiency = enucleated cells versus total recovered cells;
Recovery rate = recovered cells versus total input cells used for enucleation.
Viability after 24 hours = live cells measured by Trypan blue staining versus total cells;
Yield per run = the number of cytoplasts harvested for each run;
M = million cells

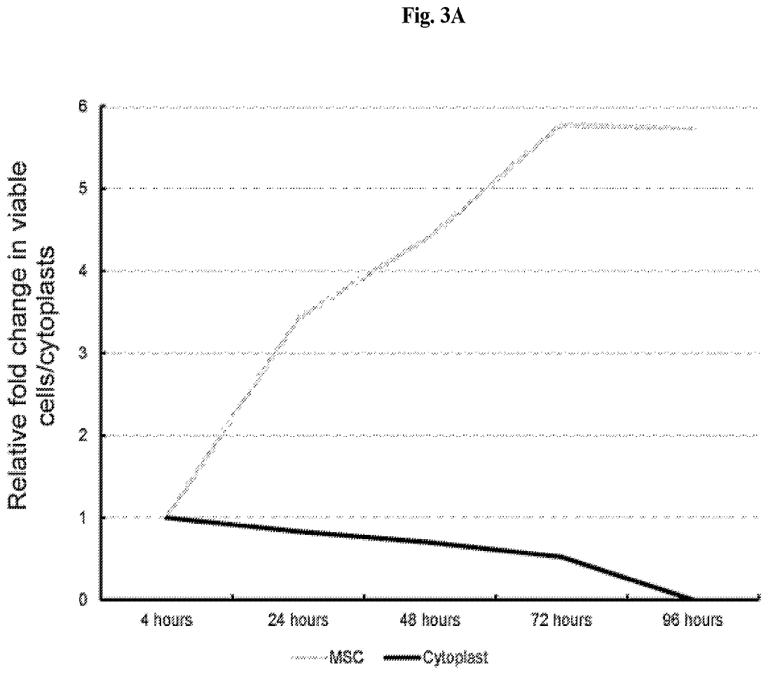

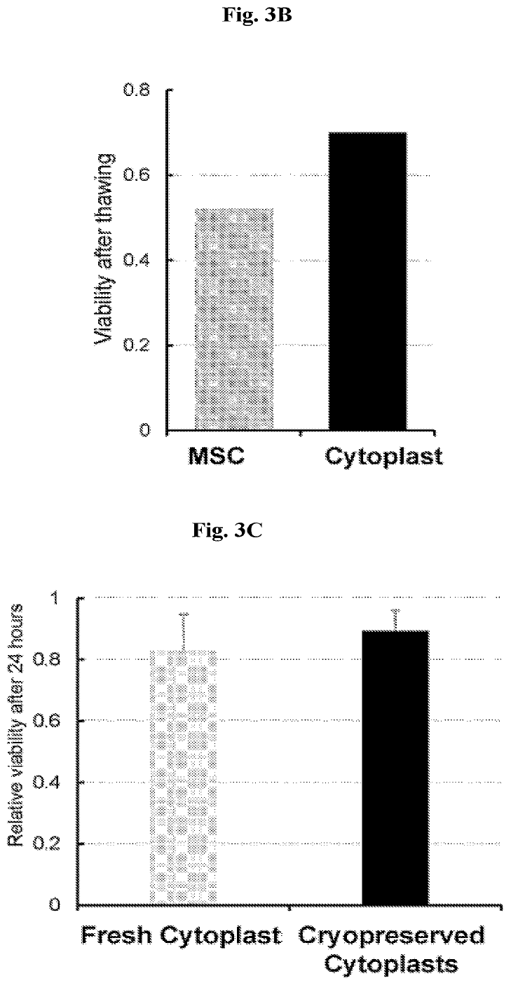

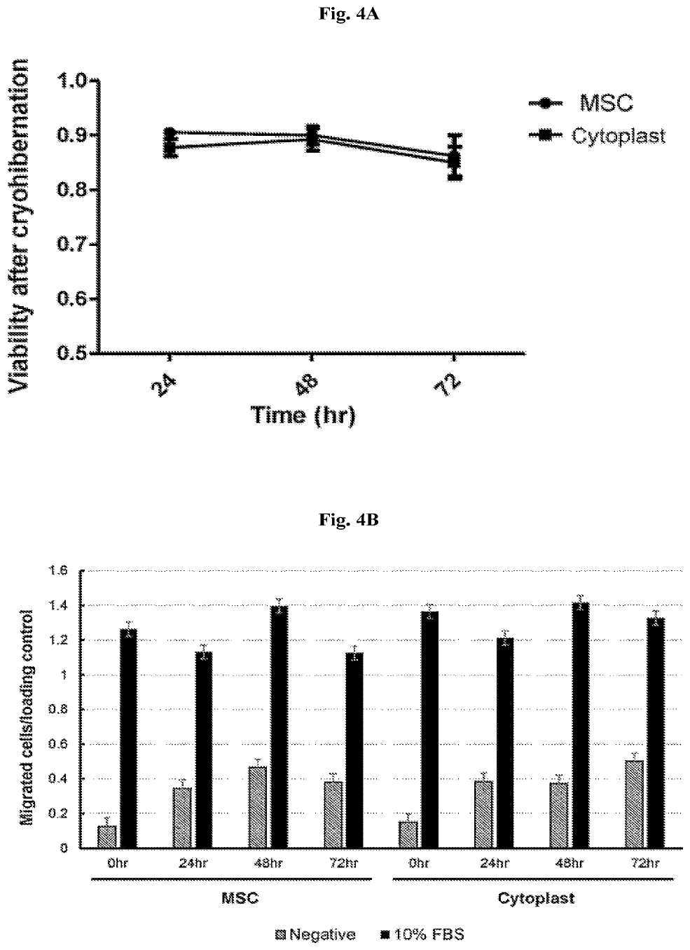

Next, the survival of cytoplasts was determined across 96 hours (FIG. 3A). Whereas MSC proliferated over-time, cytoplasts did not. Instead, the relative fold change in viable cytoplasts remained fairly constant for 72 hours before declining at 96 hours. Thus, cytoplast survival spanned 3-4 days. As most cell-based therapies are not used immediately, the viability of cytoplasts after cryopreservation was determined. Surprisingly, the viability of cytoplast after cryopreservation was greater than the viability of MSC following cryopreservation (FIG. 3B). Cytoplasts plated immediately after enucleation and cytoplasts recovered from cryopreservation displayed similar relative cell viability after 24 hours (FIG. 3C). This experiment showed that cytoplasts survival was not affected by cryopreservation. Additionally, the viability of cytoplasts after cryohibernation was similar to the viability of MSC following cryohibernation (FIG. 4A). Cytoplasts recovered after cryohibernation for various lengths of time were able to undergo induced migration in a Boyden chamber assay similar to MSCs recovered after cryohibernation, (FIG. 4B).

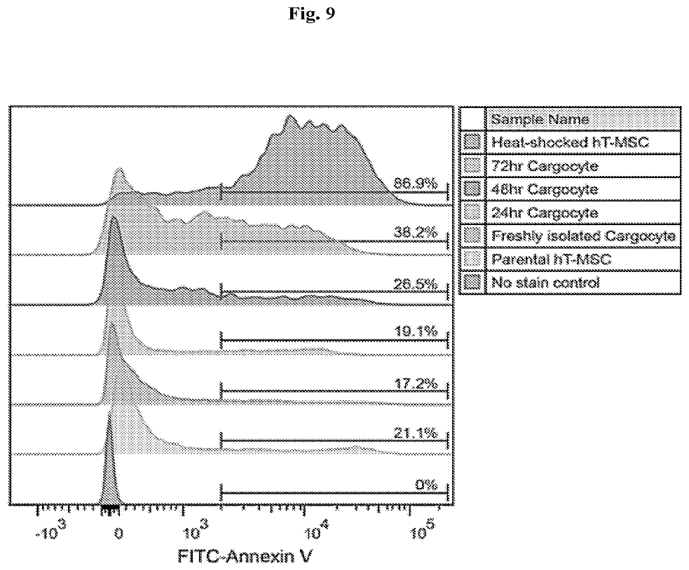

An additional viability study of the cytoplast was performed. FIG. 9 illustrates a cell surface staining of FITC-labeled Annexin V on MSCs or cytoplasts analyzed by flow cytometry. Data were analyzed in Flowjo and normalized to mode. Parental MSC═Non-engineered MSC; Isotype control=MSC stained with isotype matching IgG. 2 hr (hour)/24 hr/48 hr/72 hr Cytoplast=MSC-derived cytoplast analyzed at indicated time-point post-enucleation; Heat-shocked cells served as a positive control for apoptotic MSC cell death. Representative results from 3 independent experiments shown. After 3 Days post enucleation cytoplasts exhibited apoptosis as indicated by Annexin V Staining and FACS.

Next, a large-scale production of cells was set up ex vivo, followed by large-capacity density gradient centrifugation and enucleation, which lead to the generation of a therapeutic cytoplast. In one embodiment, the therapeutic cytoplast is loaded with therapeutic cargo (e.g., mRNA, drugs, peptides, etc.) for disease treatment. In another embodiment, the therapeutic cytoplast is prepared for immediate use (e.g., for intravenous injection (IV), intraperitoneal injection (IP), tissue, or in vitro applications).

### Example 2. Enucleated Cells Retain Intact and Functional Organelles

After determining whether cytoplasts could retain viability after cryopreservation, flow cytometry analysis were performed in order to determine whether the cell surface marker profile of MSC-derived cytoplasts differed from bone-marrow derived MSC. Both MSC-derived cytoplasts and bone-marrow derived MSCs maintained cell surface expression of CD45, CD90, CD44, CD146, and CD166. The cytoplasts reorganized the cytoskeleton, spread on matrix proteins in 2D and 3D culture systems, and formed tunneling nanotubes, which can transfer bioproducts between cells of the same or different origin. Organelle-staining indicated that Golgi, ER, F-actin cytoskeleton, lysosomes, endosomes, microtubules, and mitochondria remain intact in cytoplasts. Furthermore, cytoplasts exhibited homing potential in vitro. Cytoplasts readily migrated on extracellular matrix proteins and migrated directionally towards soluble chemokine gradients (e.g., via chemosensing). Notably, cytoplasts transfected exogenously with purified mRNAs produced functional intracellular proteins, which could mimic therapeutic mRNA applications being developed for a variety of clinical uses and disease-states. This also demonstrates that the machineries for mRNA translation and protein synthesis operate normally in cytoplasts in the absence of a nucleus, and thus can be used to produce bioactive molecules with therapeutic value.

Cytoplasts transfected exogenously with purified mRNA encoding known secreted proteins produce functional extracellular proteins in conditioned culture media, indicating that the ER/Golgi and secretory pathways operate normally in cytoplasts in the absence of a nucleus. In addition, treatment of macrophages and endothelial cells with cytoplast-conditioned media containing secreted proteins activated key signal transduction responses in these cells. These results show proof of concept that cytoplasts could be used as novel vehicles to produce and deliver secreted proteins and biomolecules with therapeutic value. Cytoplasts can be loaded with various cargo including, but not limited to, siRNA, shRNA, mRNA, DNA plasmids, peptides, and chemotherapeutic agents.

### Example 3. Enucleated Cells Can Express Functional Cell Surface Proteins

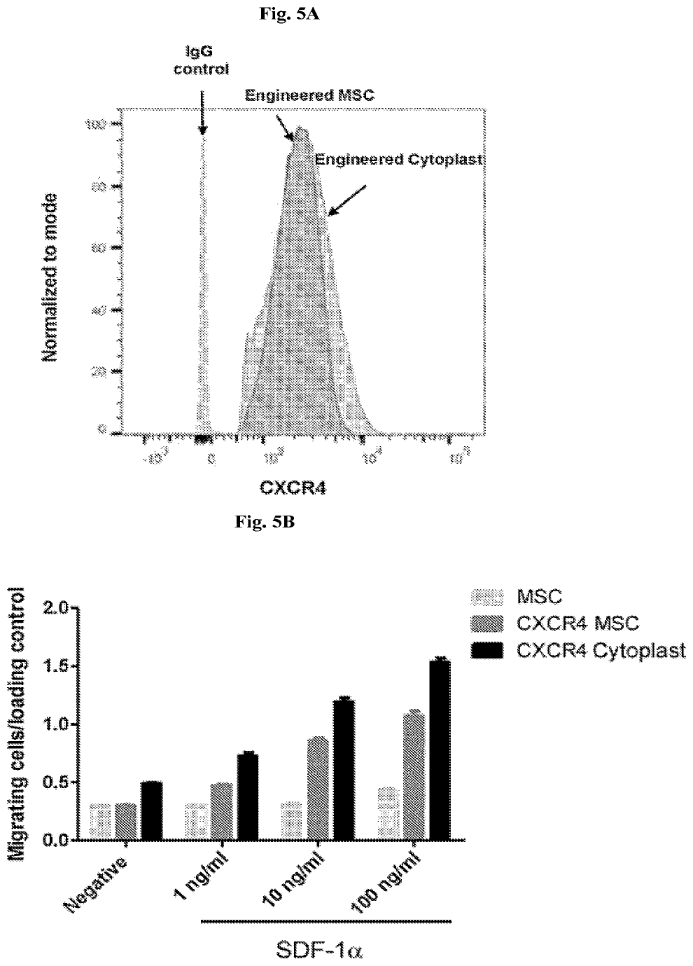

As shown in FIG. 5A, the engineered MSCs may be engineered to express, and MSCs expressing CXCR4 and engineered MSC-derived cytoplasts expressing CXCR4 express comparable levels of CXCR4, as determined by flow cytometry. To determine whether engineered cytoplasts can express functional cell surface proteins, MSCs and MSC-derived cytoplasts expressing CXCR4 receptors were allowed to migrate towards various concentrations of SDF-1a. As shown in FIG. 5B, MSC-derived cytoplasts engineered to express functional CXCR4 can migrate towards SDF-1a, and cell migration increases with increasing concentrations of SDF-1a. Furthermore, the number of migrating MSC-derived cytoplasts was greater than the number of migrating MSCs expressing CXCR4 (FIG. 5).

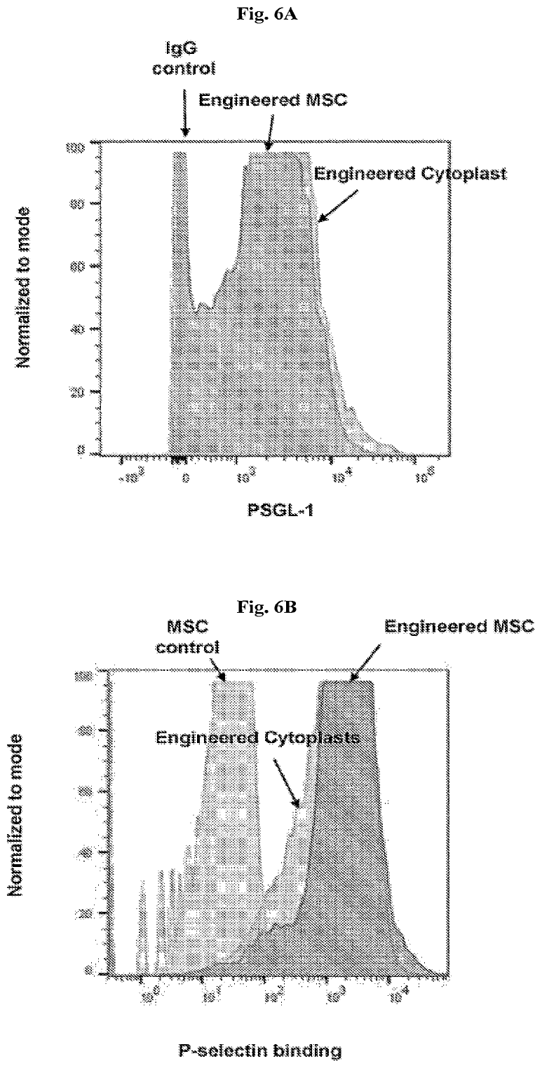

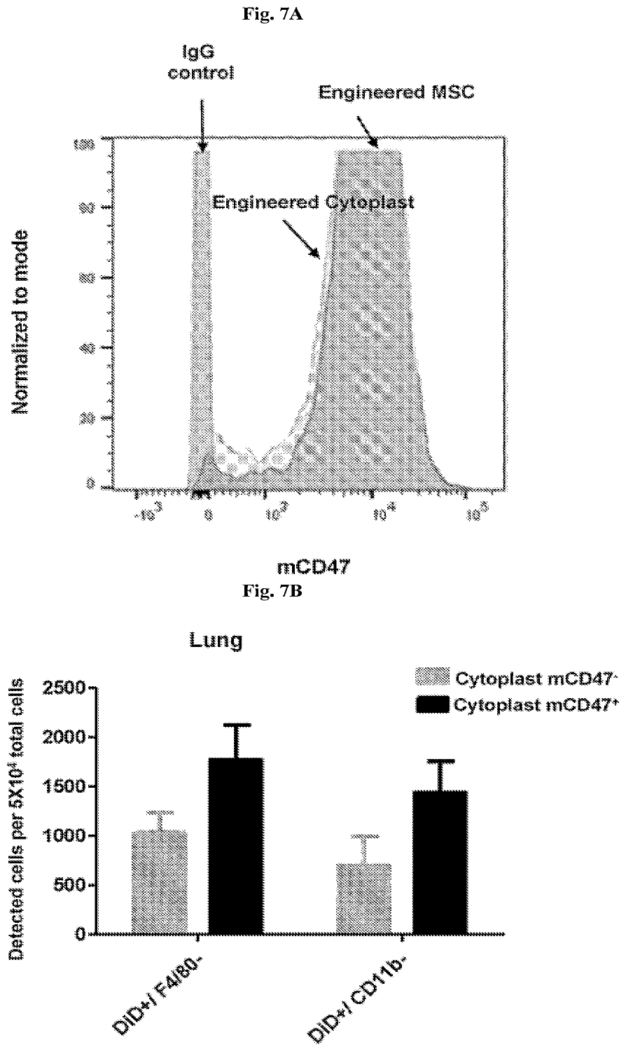

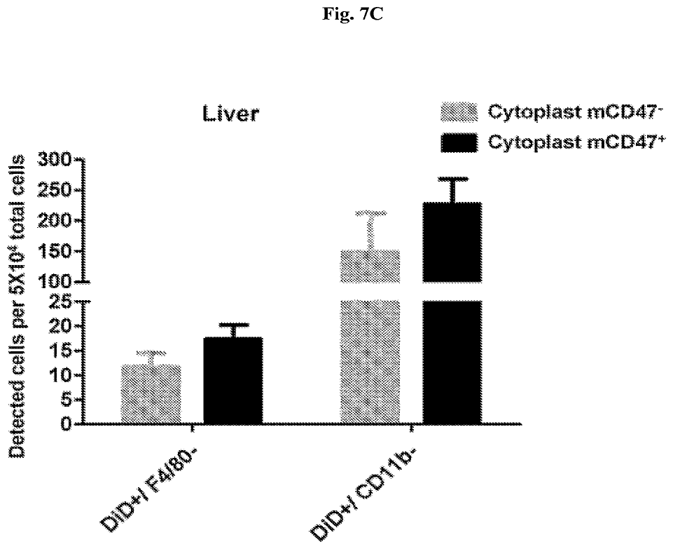

FIGS. 6A-B show that MSC-derived cytoplasts can be engineered to express functional cell adhesion proteins known to mediate cell adhesion to the inflamed vasculature. FIG. 7A-C show that MSC-derived cytoplasts can be engineered to express cell proteins known to modulate macrophage interactions and phagocytosis of therapeutic cells.

### Example 4. 3D-cultured Enucleated Cells Show Better Biodistribution In Vivo

MSCs were cultured in 3D-hanging drops (3D MSCs) then enucleated to generate 3D cytoplasts. The 3D culture protocol of MSC by hanging drops is modified from Curr Protoc Stem Cell Biol. 2014 Feb. 6; 28: Unit-2B.6. (Thomas J. Bartoshl and Joni H. Ylostalo).

Healthy MSCs were harvested from 2D-cultured plates by Trypsin and resuspended in fresh α-MEM (ThermoFisher 12561056) full medium (16.5% Premium FBS, 1% Antibiotic-Antimycotic, 1% Glutamax, 1% HEPES) at 1.43 million cells/ml. The lid of a 15 cm plate was opened completely and 20 ml PBS was added to the plate. A multichannel pipette was used to make droplets on the lid of the plate at 35 μl per droplet (approx. 50,000 cells/droplet). About 100-120 droplets were placed on each lid. The lid was closed and the plate was placed back into the incubator. Droplets were cultured for 2 days, then harvested by cell lifter and collected into 15 ml tubes (approx. 300 droplets per tube). The tubes were centrifuged for 5 minutes at 1,200 rpm. The supernatant was removed and the tubes were washed twice with PBS. All P BS was then removed and 7.5 ml of freshly thawed 0.25% Trypsin-EDTA (ThermoFisher 25200114) was added to each tube. The tubes were incubated in a water bath for 4 minutes. The droplets were gently pipetted with 1 ml pipettes with low-retention tips about 10-20 times and incubated in the water bath for another 4 minutes. The droplets were again gently pipetted with 1 ml pipettes with low-retention tips about 10-20 times until most of the droplets were dissociated. 7.5 ml of full serum medium (GlutaMAX Supplement (Gibco 35050061); Fetal Bovine Serum-Premium Select (Atlanta Biologicals S11550); HEPES (1 M) (Gibco 15630080); antibiotic-Antimycotic (100×) (Gibco 15240062)) was added to each tube and the tubes were centrifuged for 10 minutes at 1,200 rpm. The dissociated cells were washed with 10 ml of full serum medium and the cells were resuspended with 5 ml full serum medium. The cells were passed through a 70 μm cell filter and then the filter was washed with 5 ml full serum medium. The cells were counted and resuspended with pre-treated 12.5% Ficoll at more than 10M/ml. 30-40M cells were used for each enucleation tube. Subsequently, the protocol for enucleation described above was followed.

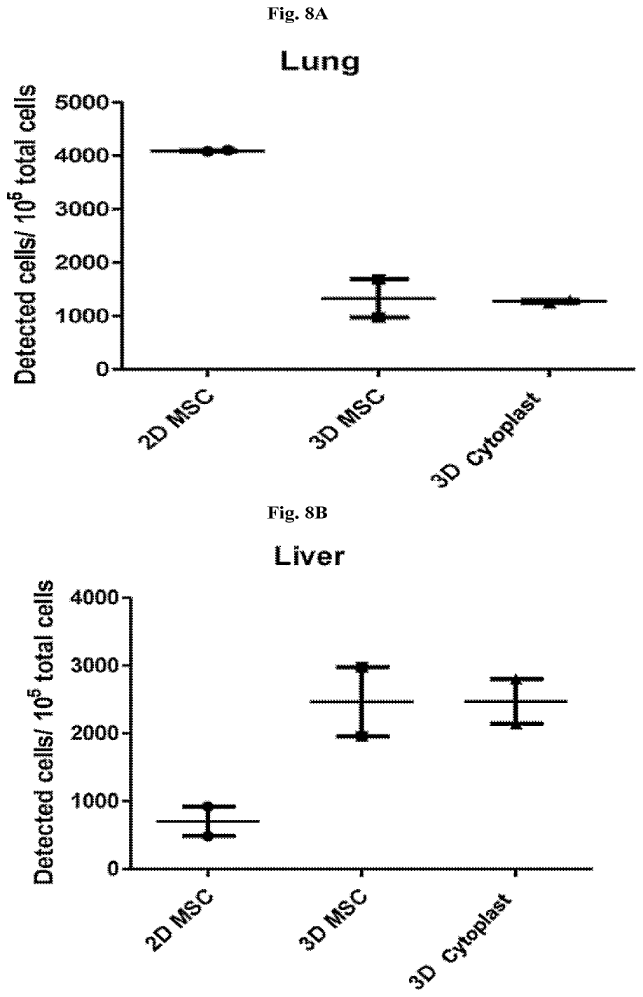

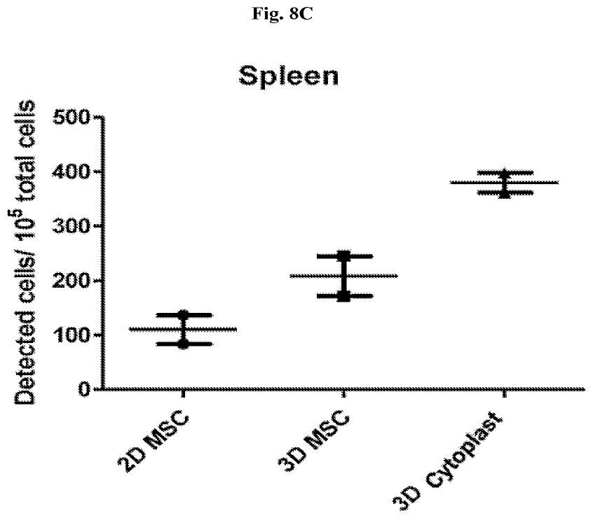

DiD labeled normal 2D-cultured MSCs (2D MSC), 3D MSCs or 3D cytoplasts were retro-orbitally injected into BalB/C mice respectively. Indicated tissues were harvested 24 hours after injection and DiD labeled cells analyzed by FACS. FIG. 8A-FIG. 8C show the successful generation of 3D-derived cytoplasts from 3D-cultured MSCs and also shows the 3D-derived cytoplasts have less lung trapping and better biodistribution to peripheral organs than 2D-cultured cells after injection into the circulation. This is expected to greatly improve their therapeutic ability to locate and deliver cargo to tissues.

### Example 5. Generating the Enucleated Cells

### Enucleation of Mesenchymal Stromal Cells (MSC)

Preparation of 50% Ficoll solution: In a glass beaker shielded from light, grams of Ficoll (PM400, GE Healthcare 17-0300-500) were dissolved in an equivalent number of milliliters ultrapure water (Invitrogen 10977-015) by continual magnetic stirring for 24 hours at room temperature. The mixture was then autoclaved for 30 minutes. Once the mixture was cooled, it was stirred again to ensure uniform consistency. The refractive index was measured on a refractometer (Reichert 13940000), and was in the range of 1.4230-1.4290. Aliquots were stored at −20 degrees Celsius.

Preparation of 2×MEM: For each 50 ml quantity, 10 mL 10×MEM (Gibco, 11430-030), 2.94 mL exactly Sodium Bicarbonate (7.5%, Gibco, 25080-094), 1 mL 100×Pen-Strep (Gibco 15140-122) and 36 mL ultrapure water (Invitrogen 10977-015) was used. The solution was then filtered through 0.22 μm membrane flask (Olympus 25-227) and stored at 4 degrees Celsius.

On the day before enucleation, MSCs were seeded at 2.5 M per 15 cm plate (Olympus 25-203) in 20 mL MSC medium DMEM 1× (Gibco 12561-056); 16.5% premium FBS (Atlanta Biologics S1150); 1% HEPES 1M (Gibco 15630-80); 1% Anti-Anti 100× (Gibco 15240-062); 1% Glutamax 100× (Gibco 35050-061)]. Next, Cytochalasin B (Sigma Aldrich C6762) was added to the 2×MEM (2 μM/mL final concentration).

Preparation of Ficoll gradients: 2× CytoB was added to 50% Ficoll aliquots at 1:1 dilution to make 25% Ficoll stock concentration. Next, 17%, 16%, 15% and 12.5% Ficoll were made by diluting 25% Ficoll with the appropriate volume of 1×MEM buffer (2×MEM containing Cytochalasin B added to ultrapure water at 1:1 dilution). The dilutions were equilibrated in a CO₂ incubator for at least 1 hour covered with loose cap. The Ficoll gradients were then poured into 13.2 mL ultra-clear tubes (Beckman, 344059), and incubated overnight (6-18 hours) in the CO₂ incubator.

On the day of enucleation, 12-25M MSC (ideally 20M) were collected into each tube for enucleation. Media was aspirated, and the cells washed once with phosphate buffered saline (PBS) (GIBCO 14190-144). Five mL of TrypLE-Select (Gibco, 12563011) was added to each plate, and incubated up to 5 minutes. When 90% of the cells were detached, 5 mL full MSC media was added, and the cells were collected into 50 ml tubes (3-4 plates/tube). The tubes were then centrifuged at 1, 200 rpm for 5 minutes. The pellet was resuspended in 10 mL PBS. Cells were counted, pelleted, and re-suspended with 12.5% Ficoll. Next, the cell-Ficoll mixture was dropwise passed through a 40 μm cell strainer (Falcon 352340) into a new 50 mL tube. Using a syringe, 3.2 mL of cell suspension was slowly loaded onto the pre-made gradients. One mL of 1×MEM buffer was added at the final (top) layer with syringe. The tubes were then loaded into rotor buckets, balanced, and run in the ultracentrifuge (Beckman, L8M) for 60 minutes, 26,000 rpm, 31° C., Accel 7, Deccel 7. At the end of the centrifugation, there were three layers: one near the top of the 12.5% (cytoplasts and debris), one near the 12.5/15% interface (cytoplasts), and a pellet at the bottom of the 25% (karyoplasts). The layers above 15% Ficoll solution were collected into 15 ml conical tubes. The collected layers are then diluted with more than 4 volumes warm serum-free MSC medium (i.e. 3 mL of Ficoll and filled with up to 15 mL media). After gently mixing, the mixture was pelleted for 10 minutes at 1,200 rpm. Following three washes with warm serum-free MSC medium, the cells were resuspended in media according to the experimental protocol, e.g., transfection media vs. migration media vs. serum free media vs. full media. Efficiency of enucleation was determined in a 12-well plate by adding full MSC media with 1:2000 dilution Vybrant® Dyecycle Green (Molecular Probes V35004) or 1:5000 dilution Hoechst 33342. A small volume of each layer was added to each well and allowed to attach/stain for 10 minutes in the incubator. The percentage of negative cytoplasts per population was determined by epifluorescent microscopy.

Cytoplast mRNA Transfection

1 M cytoplasts were suspended with warm 1 ml amino acid-free α-MEM full medium (ThermoFisher 12561056; 16.5% Premium fetal bovine serum (FBS), 1% Glutamax (Gibco 35050061), 1% HEPES (Gibco 15630080)). 1 μg mRNA was diluted with warm opti-MEM and mixed with pipet at least 20 times. 4 μl lipofectamine-3000 (ThermoFisher L300015) was added to 46 μl warm opti-MEM (ThermoFisher 31985062) and mixed with pipet for at least 20 times. The ratio of mRNA and lipofectamine-3000 was 1:4 (w/v). The mRNA and lipofectamine-3000 dilutions were mixed with pipet for at least 20 times and incubated at room temperature for 15 minutes. The mRNA and lipofectamine-3000 mixture was added to the cytoplast suspension, mixed well and incubated at 37° C. for 30 minutes. The suspension was shaken every 5 minutes to prevent cell clumping. After incubation, the cells were centrifuged, and re-suspended in normal α-MEM full medium (16.5% Premium FBS, 1% Antibiotic-Antimycotic, 1% Glutamax, 1% HEPES) or PBS.

Cytoplast siRNA Transfection

1 M cytoplasts were suspended with warm 1 ml A/A free α-MEM full medium (16.5% Premium FBS, 1% Glutamax, 1% HEPES). Two μl siRNA was diluted with warm opti-MEM and mixed with pipet at least 20 times. Eight μl lipofectamine-3000 was diluted with 92 μl warm opti-MEM and mixed with pipet at least 20 times. The ratio of siRNA and lipofectamine-3000 was 1:4 (v/v). The siRNA and lipofectamine-3000 dilutions were mixed with pipet at least 20 times and incubated at room temperature for 15 minutes. The siRNA and lipofectamine-3000 mixture was added to the cytoplast suspension, mixed well and incubated at 37° C. for 20 minutes. The suspension was shaken every 5 minutes to prevent cell clumping. After a 20 minute incubation, the cells were centrifuged, and re-suspended with normal α-MEM full medium (16.5% Premium FBS, 1% Antibiotic-Antimycotic, 1% Glutamax, 1% HEPES).

### Generation of Oncolytic Virus Infected Cytoplasts

One day before enucleation (usually 18 hrs before enucleation), 2.5*10\hat{ }6 hTERT-MSCs were seeded on a 15-cm dish. Roughly two hours after seeding, the cells were washed once with PBS. Cells were then infected with oHSV-GFP (Imanis OV3001) at different MOIs (0.05 or 0.5 for example) with 8 mL serum free opti-MEM. Next, cells were incubated at 37° C. for 2 hours with occasionally shaking. The virus inoculum was then discarded. 20 mL pre-warmed full culture medium (α-MEM, 16.5% Premium FBS, 1% Antibiotic-Antimycotic, 1% Glutamax, 1% HEPES) was added to each well. The cells were incubated at 37° C. until enucleation.

### Lentivirus Overexpressing Functional Proteins in Cytoplasts

Target cells were plated in one well of 6-well plate at density of 1-2×10⁵ cells/well, or 10 cm plate with 0.5-1 M MSCs. The next day, the concentrated recombinant lentivirus was thawed in a 37° C. water bath and removed from the bath immediately once thawed. The cells were then washed with PBS 3 times. 200 μL serum free medium or 2 mL serum free medium (1:1250 SureENTRY) was added. The target cells were infected in a 6-well plate with MOI 10:1. The next day, the viral supernatant was removed and the appropriate complete growth medium was added to the cells. After 72 hours incubation, the cells were subcultured into 2×100 mm dishes. The appropriate amount of selection drug (i.e., puromycin) was added for stable cell-line generation. 10-15 days after selection, clones were picked for expansion and were screened for positive ones. The selected positive clones were expanded for enucleation. Engineered cytoplasts were prepared as outlined above. The target protein expression on cytoplasts was determined by ordinary biochemical methods or functional assays, e.g., fluorescent activated cell sorting (FACS), western blot, or Boyden chamber assay.

Peptide Loading into Cytoplasts

1×10⁵/ml per well were plated onto a 4-chamber glass slide (LabTek II 4-chamber glass slide, 155383) in full MSC media DMEM 1× (Gibco 12561-056); 16.5% premium FBS (Atlanta Biologics S1150); 1% HEPES 1M (Gibco 15630-80); 1% Anti-Anti 100× (Gibco 15240-062); 1% Glutamax 100× (Gibco 35050-061)]. Cells were allowed to attach for at least 1 hour or overnight. Cells were then rinsed with PBS (Gibco 14190-144). Arg9(FAM) (10 mM, Anaspec, AS-61207) was diluted in full media to a total concentration of 1:100 (100 uM). Cytoplasts were then incubated for 1 to 2 hours and rinsed 3 times with PBS. Hoechst 33342 (Invitrogen) was added at a 1:5000 dilution in full media for at least 10 minutes. Cells were then washed with PBS and imaged by epifluorescent microscopy.

### Example 6. Producing a Pharmaceutical Formulation for Treatment of Cancer

Described herein are pharmaceutical formulations for treatment of cancer, where the pharmaceutical formulations comprise an enucleated cell described herein. The pharmaceutical formulation can comprise a pharmaceutically acceptable excipient, carrier, or diluent described herein. The pharmaceutical formulation can comprise an adjuvant. The pharmaceutical formulation can comprise an additional therapeutic agent such as immune checkpoint inhibitor (e.g., IMP321/Eftilagimod alpha (Immutep), Relatlimab BMS-986016, Ipilimumab (Yervoy), Pembrolizumab (Keytruda), Nivolumab (Opdivo), Cemiplimab (Libtayo), Atezolizumab (Tecentriq), Avelumab (Bavencio), Durvalumab (Imfinzi), Ipilimumab (Yervoy), LAG525, MK-4280, Irinotecan, Oxaliplatin, REGN3767, TSR-033, BI754111, Sym022, FS118 (a bi-specific anti-LAG3/PD-L1 antagonistic mAb), MGD013 (a bi-specific anti-LAG3/PD-1 antagonistic mAb), TSR-022, Niraparib, Bevacizumab, MBG453, Decitabine, Spartalizumab, Sym023, INCAGN2390, LY3321367, Ramucirumab, Abemaciclib, Merestinib, BMS-986258, SHR-1702, Camrelizumab, MK-7684, Etigilimab/OMP-313 M32, Tiragolumab/MTIG7192A/RG-6058, BMS-986207, AB-154, ASP-8374, JNJ-61610588, CA-170d, Enoblituzumab/MGA271, MGD009, I-8H9/omburtamab, Trastuzumab, MGD013 (Anti-PD-1, anti-LAG-3 dual checkpoint inhibitor), BGB-A1217, CM-24 (MK-6018), BMS 986178, MEDI6469, PF-04518600, GSK3174998, MOXR0916, Utomilimab (PF-05082566), Urelumab (BMS-663513) ES101, BMS-986156, TRX-518, AMG 228, JTX-2011, GSK3359609, BMS-986226, MEDI-570, or Varlilumab (CDX-1127)).

The pharmaceutical formulation can be formulated for administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

### Example 7. Treating Cancer with Enucleated Cell

A subject diagnosed with cancer such as triple negative breast cancer, is administered a pharmaceutical formulation comprising a plurality of cytoplasts engineered to contain or express an anti-cancer therapeutic agent such as immune checkpoint inhibitor (e.g., anti-PD-1/PD-L1 monoclonal antibody or single-domain antigen-binding fragment thereof, or an antibody-drug conjugate thereof) or single-domain antibody or antigen binding fragment thereof. The pharmaceutical formulation is formulated for intravenous administration or administration by inhalation. The pharmaceutical formulation is administered once to the subject intravenously or by inhalation (induction phase). The pharmaceutical formulation is thereafter administered to the subject with a frequency of at least weekly, once every two weeks, once every three weeks, once every four weeks, once every five weeks, once every six weeks, once every seven weeks, once every two months, once every six months, or once per any optimal time interval or cycle. In some cases, the pharmaceutical formulation is administrated at a time interval or cycle for maintenance phase. Reduction in tumor mass and/or reduction in tumor biomarker levels (carcinoembryonic antigen (CEA), cytokeratin fragment 19 (CYFRA21-1) and neuron-specific enolase (NSE)) is monitored during the treatment course. Following induction of the treatment, tumor mass and biomarker levels decrease, and continues to decrease in a dose-dependent manner, illustrating that the pharmaceutical formulation is therapeutically effective to treat the cancer.

In this example, the plurality of cytoplasts are generated from mesenchymal stromal cell (MSC) or inducible pluripotent stem cell (iPSC) and are genetically engineered to express a homing receptor specific to a ligand expressed on cancer cells (e.g., Carbonic Anhydrase 9 (CA9), Carbonic Anhydrase 12 (CA12), Cancer/Testis Antigen 83 (CT83), Desmoglein (DSG3), FAT Atypical Cadherin 2 (FAT2), Probable G-protein coupled receptor 87 (GPR87), KISS1 Receptor (KISS1R), LY6/PLAUR Domain Containing 3 (LYPD3), Solute Carrier Family 7 Member 11 (SLC7A11), and Transmembrane Serine Protease 4 (TMPRSS4)). The MSC or iPSC are also engineered to express a “don't eat me” signaling peptide such as CD47, PD-L1, HLA-E, HLA-G, a fragment thereof, or a combination thereof. Engineering of the MSC and iPSC to expression of the homing receptor and the “don't eat me” signaling peptide is performed using suitable methods described herein before enucleation of the MSC or iPSC to generate the cytoplasts having the homing receptors and “don't eat me” signaling peptide expressed at the cell surface of the MSC or iPSC.

The MSC or iPSC expressing the homing receptor and the “don't eat me” signaling peptide are enucleated using suitable methods described herein, and are optionally, preserved using lyophilization, cryohibernation, or cryopreservation. At the point of need, the MSC or iPSC engineered to express the homing receptor and the “don't eat me” signaling peptide are further engineered to express the anti-cancer therapeutic agent using suitable methods described herein (e.g., transfection of mRNA encoding the anti-PD-1 antibody or single-domain antibody). The anti-cancer therapeutic agent is expressed in the cytoplasts, along with the homing receptor and the “don't eat me” signaling peptide. The cytoplasts are formulated for human administration.

### Example 8. Treating Anthrax Infection with Enucleated Cell

A subject has been exposed to anthrax (Bacillus anthracis) and is administered a pharmaceutical formulation comprising a plurality of cytoplasts engineered to contain or express an antibody or single-domain antibody, or antigen binding fragment thereof, that binds to an epitope of Bacillus anthracis to neutralize the infection. The pharmaceutical formulation is formulated for intravenous administration or administration by inhalation. The pharmaceutical formulation is administered once to the subject intravenously or by inhalation (induction phase). The pharmaceutical formulation is thereafter administered to the subject with a frequency of at least weekly, once every two weeks, once every three weeks, once every four weeks, once every five weeks, once every six weeks, once every seven weeks, once every two months, once every six months, or once per any optimal time interval or cycle. Following administration of the pharmaceutical formulation, a reduction in the Bacillus anthracis in the subject is observed, demonstrating that the pharmaceutical formulation is therapeutically effective to treat a disease or a condition caused by an infection by Bacillus anthracis.

In this example, the plurality of cytoplasts are generated from mesenchymal stromal cell (MSC) or inducible pluripotent stem cell (iPSC) and are genetically engineered to express antibody or single-domain antibody, or antigen binding fragment thereof either before, or after enucleation to produce the plurality of cytoplasts. The binding of the antibody or the antigen-binding fragment thereof or the single-domain antibody to the epitope expressed by Bacillus anthracis or the epitope of the spore of Bacillus anthracis either directly confers therapeutic property by targeting the Bacillus anthracis or the spore of Bacillus anthracis for degradation or recruits and activates immune cell to the Bacillus anthracis or the spore of Bacillus anthracis. In some cases, the enucleated cell is manufactured and stored in large quantity in anticipation of anthrax infection outbreak. Storage may be cryopreservation, cryohibernation, or lyophilization of the cytoplasts. When there is an urgent need, the biological activity of the cytoplasts may be revived, and the cytoplasts may be prepared in a pharmaceutical formulation for administration.

### Example 9. Producing Enucleated Cell Expressing a Single-Chain Antibody

MSCs are genetically engineered to express one or more exogenous single-chain antibodies. The nucleic acid encoding the single-chain antibody includes histidine tag (6×His tag) (SEQ ID NO: 1702). In some embodiments, the nucleic acid encoding the single-chain antibody is flanked by 5′- and 3′-untranslated regions (UTRs) from mouse α-globin. Full substitution of pseudouridine is used to synthesize transcripts. After adding 5′ cap structure (CleanCap® AG) and 3′ poly(A) tail (120A) (SEQ ID NO: 1703), the synthesized nucleic acid (e.g., mRNA) is purified with silica membrane. The pre-made mRNAs from TriLink is directly used for mRNA transfection of lung homing cytoplasts (1 μg/1×10⁶) using Lipofectamine 3000 (ThermoFisher, #L3000008) for 30 minutes. Transfected cytoplasts were rinsed then plated in a 24-well-plate (25,000 per well, 1 ml media); the conditioned media is collected at 24, 48, 72, and 96 hours; and antibody levels are quantified by ELISA. 96 well ELISA plates are coated with anti 6×His antibody (“6×His” disclosed as SEQ ID NO: 1702) (Cell Signaling, #2365) followed by binding to cytoplast conditioned media containing 6×His-antibodies (“6×His” disclosed as SEQ ID NO: 1702). An anti-camelid VHH conjugated to peroxidase antibody is used as a secondary antibody (Jackson Immunoresearch, 128-035-232) followed by TMB incubation, stop solution (Biolegend) and read with a μQuant plate reader (Biotek) at a wavelength of 450 nm. The standard curve is based on bacterial purified 6×His-antibodies (“6×His” disclosed as SEQ ID NO: 1702).

Experiments are conducted in triplicate and results are reported as the mean ng/ml and significant differences determined with Students' t test. The ability of antibodies produced by cytoplasts to modulate target activity in vitro is tested using cell-based assays that express the target antigen and respond to antibody binding by FACS or a detectable cellular change such as proliferation, death, immune suppression, cytokine secretion, etc. For example, macrophage migration inhibitory factor (MIF) antibody bioactivity experiments are performed by using the murine macrophage cell line, RAW264.7 (ATCC) with or without LPS stimulation. Cells are incubated in a Nunc Maxisorp 96-well flat-bottom tissue culture plate either alone or in combination with 10 ng LPS, with or without 125, 250, or 500 nM of cytoplasts or bacteria purified anti-MIF antibodies. Cells are incubated for 18 h at 37° C., after which the supernatant is collected and tested in a mouse TNF-α ELISA (R&D Systems). Experiments are conducted in triplicate and results are reported as IC50s for condition and significant differences determined with Students' t test.

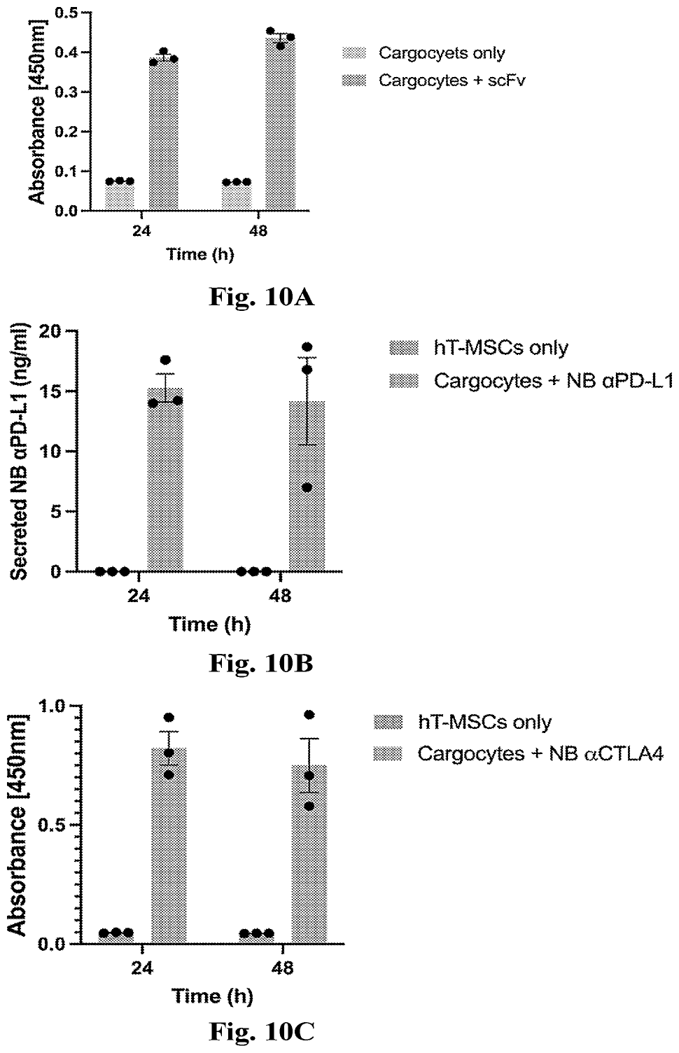

FIGS. 10A-C illustrate exemplary experimental observations of the enucleated cells engineered cells expressing an antibody described herein (e.g., nanobody, single-domain antibody, or scFv). For cell enucleation, human telomerase reverse transcriptase (hTERT)-immortalized, adipose-derived mesenchymal stromal cells were loaded onto a Ficoll gradient (GE Healthcare, #17-0300-500) with Cytochalasin B (Sigma Aldrich, #C6762) at a final concentration of 10 μg/ml. Cells were spun down for 60 minutes at 26,000 rpm and 31° C. with minimal braking. Enucleation efficiency was examined by epifluorescence microscope (Nikon Eclipse Ti) after staining with Vybrant Dyecycle Green (Invitrogen, #V35004). For scFv secretion, lentivirus pLV-EF1A mouse CTLA-4 scFv was purchased from VectorBuilder, and hTERT MSCs were transduced at 2 to 5 MOI in Opti-MEM medium (ThermoFisher, #31985088) with 8 μg/ml SureENTRY transduction reagent (Qiagen, #336921). After 4 hours of co-incubation, transduction complex was replaced with CCM. Cells were seeded at 5E5 cells per 6 well and the conditioned media was collected after 24 and 48 hours. scFv detection was done by coating a high absorbing ELISA plate (Biolegend, #423501) with anti CH3 (BioRad, #MCA878G) antibody. The Plate was incubated over night at 4° C. Then, blocked and incubated with conditioned media for 2 h at room temperature with shaking. scFv was detected with purified anti-human IgG Fc (BioLegend, #409302) and the signals were developed with TMD substrate (Biolegend, #421101). Absorbance was read on the μQuant plate reader (Biotek) at 450 nm, and the background was measured at 570 nm.

For scFv secretion, 1 μg of mRNA encoding anti PD-L1 NB (nanobody or single-domain antibody or scFv) or anti CTLA-4 NB (nanobody or single-domain antibody or scFv) were added to 49 μl of pre-warmed opti-MEM (ThermoFisher, #31985088) and separately 4 μl Lipofectamine 3000 (ThermoFisher, #L3000008) was added to 46 μl opti-MEM. The Lipofectamine and mRNA solutions were mixed together and incubated for 15 minutes at room temperature. MSCs or enucleated cells were suspended in αMEM without antibiotics at 1E6 cells/ml. 100 μl of mixed mRNA+lipofectamine-3000 solution was added to 1 ml MSC or enucleated cells suspension, mixed thoroughly and incubated at 37° C. for 30 minutes. Cells were washed and 2.5E4 cells were seeded in each 24 well, conditioned media was collected every 24 hours. scFv levels were determined by coating a high absorbing ELISA plate (Biolegend, #423501) with anti-His tag antibody (ThermoFisher, #MA121315) for detection of anti PD-L1 NB and with anti-FLAG tag antibody (Sigma, #F3165) for detection of anti CLTA4 NB. Plates were incubated overnight at 4° C. Then, plates were blocked and incubated with conditioned medias for 2 h at room temperature with shaking. scFv was detected with Peroxidase Goat Anti-Alpaca IgG, VHH domain (Jackson Immunoreasearch, #128-035-232) and the signal were developed with TMD substrate (Biolegend, #421101). Absorbance was read on the μQuant plate reader (Biotek) at 450 nm and the background was measured at 570 nm. FIG. 10A is a representative graph showing the secreted scFv measured by ELISA in conditioned media of non-transfected (hTERT) and transfected (scFv) cells. Cells were enucleated (enucleated cells only and enucleated cells+scFv) and seeded in 6 well plates (0.5×10⁶/well) and the conditioned media was collected after 24 and 48 hours after enucleation for ELISA detection. Mean±SEM; n=3 biological replicates. FIG. 10B is a representative graph showing the secreted anti PD-L1 NB measured by ELISA in conditioned media of non-transfected (hTERT-MSCs only) and transfected (enucleated cells+NB αPD-L1) cells. Cells were seeded in 6 well plates (0.5×10⁶/well) and the conditioned media was collected after 24 and 48 hours after enucleation for ELISA detection. Mean±SEM; n=3 biological replicates. FIG. 10C is a representative graph showing the secreted anti CTLA-4 NB measured by ELISA in conditioned media of non-transfected (hTERT-MSCs only) and transfected (enucleated cells+NB αCTLA-4) cells. Cells were seeded in 6 well plates (0.5×10⁶/well) and the conditioned media was collected after 24 and 48 hours after enucleation for ELISA detection. Mean±SEM; n=3 biological replicates. FIG. 10A-C demonstrate that the enucleated cells expressed and secreted (as shown by detecting the antibodies in the conditioned media) scFv or single-domain antibody.

### Example 10. Producing a Pharmaceutical Formulation for Treatment of Idiopathic Pulmonary Fibrosis (IPF)

Described herein are pharmaceutical formulations for treatment of IPF, where the pharmaceutical formulations comprise an enucleated cell described herein. The pharmaceutical formulation can comprise a pharmaceutically acceptable excipient, carrier, or diluent described herein. The pharmaceutical formulation can comprise an adjuvant. The pharmaceutical formulation can comprise an additional therapeutic agent such as nintedanib or pirfenidone.

The pharmaceutical formulation can be formulated for administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

### Example 11. Treating Idiopathic Pulmonary Fibrosis (IPF)

A subject diagnosed with IPF is administered a pharmaceutical formulation comprising a plurality of cytoplasts engineered to contain or express a therapeutic agent such as an antibody-drug conjugate thereof or a single-domain antibody or an antigen binding fragment thereof targeting CTGF. The pharmaceutical formulation can comprise at least one additional therapeutic such as nintedanib or pirfenidone. The pharmaceutical formulation is formulated for intravenous administration or administration by inhalation. The pharmaceutical formulation is administered once to the subject intravenously or by inhalation (induction phase). The pharmaceutical formulation is thereafter administered to the subject with a frequency of at least weekly, once every two weeks, once every three weeks, once every four weeks, once every five weeks, once every six weeks, once every seven weeks, once every two months, once every six months, or once per any optimal time interval or cycle. In some cases, the pharmaceutical formulation is administrated at a time interval or cycle for maintenance phase.

In this example, the plurality of cytoplasts are generated from mesenchymal stromal cell (MSC) or inducible pluripotent stem cell (iPSC) and are genetically engineered to express a homing receptor specific to a ligand expressed by a lung cell described herein. The MSC or iPSC are also engineered to express a “don't eat me” signaling peptide such as CD47, PD-L1, HLA-E, HLA-G, a fragment thereof, or a combination thereof. Engineering of the MSC and iPSC to expression of the homing receptor and the “don't eat me” signaling peptide is performed using suitable methods described herein before enucleation of the MSC or iPSC to generate the cytoplasts having the homing receptors and “don't eat me” signaling peptide expressed at the cell surface of the MSC or iPSC. The MSC or iPSC expressing the homing receptor and the “don't eat me” signaling peptide are enucleated using suitable methods described herein, and are optionally, preserved using lyophilization, cryohibernation, or cryopreservation. At the point of need, the MSC or iPSC engineered to express the homing receptor and the “don't eat me” signaling peptide are further engineered to express the anti-cancer therapeutic agent using suitable methods described herein (e.g., transfection of mRNA encoding the anti-CTGF antibody or single-domain antibody). The anti-cancer therapeutic agent is expressed in the cytoplasts, along with the homing receptor and the “don't eat me” signaling peptide. The cytoplasts are formulated for human administration.

### Example 12. Lyophilization of Enucleated Cell

Mesenchymal stromal cells (MSCs) have been shown to have therapeutic. The use of MSCs cell therapy has been limited due to the costly and demanding storage and transfer cryopreservation conditions. Described herein are lyophilization techniques to ease this process by preserving cell stability and viability over time in lower temperatures (e.g., −80° C. or −20° C.). The optimization of enucleated cell lyophilization, while keeping the viability, activity, and protein expression of the enucleated cell intact, can make therapy with the enucleated more accessible. Prolonging enucleated cell viability in higher temperatures compared to liquid nitrogen can make it an off-the-shelf product and reduce transport, delivery and storage costs. Moreover, lyophilized enucleated cell will be less sensitive to transient warming events than enucleated cell resuspended in freezing media and stored in liquid nitrogen. In addition, lyophilization does not include the defrosting process to which cell damage is attributed, particularly at large volumes (e.g., from recrystallization).

### Experimental Procedure: Cell Viability Assay

In order to determine if and how the lyophilization process effects enucleated viability, human hTERT MSCs are grown in complete αMEM media (16.6% FBS, 1×Glutamax, 1×AA and HEPES). 24 hours before enucleation, cells are incubated with 0, 100 mM, or 250 mM trehalose in complete media in 37° C. After enucleation, the enucleated cells are resuspended in PBS containing appropriate concentrations of trehalose to a final concentration of 5×10⁶ cells/ml in 500 μl. The groups list in Table 4 are tested.

TABLE 4
Lyophilization of enucleated cell

| Cell type | Enucleation | trehalose | Storage temp | Time points |
|---|---|---|---|---|
| 3D hTERT | Yes | 0, 100 or 250 mM | −20° C. or −80° C. | 1, 7, 14, 28 days |
| 3D hTERT | No | 0 | — | |
| 3D triple hTERT | Yes | 0, 100 or 250 mM | −20° C. or −80° C. | 1, 7, 14, 28 days |
| 3D triple hTERT | No | 0 | — | |
| 3D triple NB hTERT | Yes | 0, 100 or 250 mM | −20° C. or −80° C. | 1, 7, 14, 28 days |
| 3D triple NB hTERT | No | 0 | — | |

Lyophilization can done overnight, and samples can be stored at −20° C. or −80° C. for 1, 7, 14, or 28 days. For each time point the cell can be resuspended in 0.5 ml of PBS with appropriate trehalose concentration for 5 minutes followed by 4.5 ml complete pre warmed αMEM. Cells can be counted and their size measured using automated cell counter comparing cell number and morphology before and after lyophilization. 7,000 cells of each condition can be seeded in a 96-well plate and analyzed for viability using XTT after 1, 24, 48, 72, and 96 hours comparing to nucleated cells as controls. The remaining cells can be seeded for cell surface expression, cargo expression and cell activity.

The enucleated cell incubated with trehalose and stored at −80° C. is more viable than enucleated cell that is incubated without trehalose and at −20° C. Longer storage period can lead to the less viable enucleated cell with potentially up to 25%-30% dead enucleated cell.

### Experimental Procedure: Surface Markers Expression

100,000 cells from each sample that described in Table 4 are analyzed for cell surface expression by FACS in different time points (1, 24, 48, and 96 hours). Each sample is incubated with antibodies against MSCs surface markers (CD105+, CD90+, or CD45−) and engineered overexpressed markers (CXCR4, PSGL-1, or CCR2). The lyophilized enucleated cell is going to recover their membranes lipid structure and express native and transfected receptors.

### Experimental Procedure: Cargo Expression

700,000 cells of 3D triple hTERT and 3D triple NB hTERT groups are seeded on a 6-well plate. The supernatant from each sample is collected at different time points (1, 24, 48 and 96 hour) and analyzed for anti-human PD-L1 singe-domain antibody concentration using ELISA. Experiments that were done on mRNA stability after lyophilization showed that the addition of trehalose maintained mRNA stability and protein expression for up to 3 months. As such, by using trehalose, the enucleated cell is able to keep the same level of antibody expression as freshly prepared enucleated cell.

### Experimental Procedure: Enucleated Cell Activity

Boyden chamber migration assay can be used to assess in vitro enucleated cell activity. 50,000 cells of each group in Table 4 are seeded on fibronectin coasted 8 μm pore inserts for 2 hours. The lower chambers are filled with serum free αMEM with 0.25% BSA as a negative control or 10% FBS as a positive control. To test migration towards chemokines, the lower chambers have SDF1α (100 ng/ml) or CCL2 (100 ng/ml). The inserts are removed and stained with crystal violet. Migrated cells are imaged and analyzed with ImageJ. If the enucleated cell maintains its viability, cargo expression, and receptor expression, the enucleated cell should exhibit homing capabilities and subsequently migrate.

While the foregoing disclosure has been described in some detail for purposes of clarity and understanding, it will be clear to one skilled in the art from a reading of this disclosure that various changes in form and detail may be made without departing from the true scope of the disclosure. For example, all the techniques and apparatus described above may be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed:

1. An enucleated cell, comprising:
(a) an immune checkpoint inhibitor; and
(b) one or more intracellular organelles configured to express the immune checkpoint inhibitor or release the immune checkpoint inhibitor from the enucleated cell.

2. The enucleated cell of claim 1, wherein the immune checkpoint inhibitor comprises a programmed cell death protein 1 (PD-1) inhibitor.

3. The enucleated cell of claim 1, wherein the immune checkpoint inhibitor comprises a programmed death ligand 1 (PD-L1) inhibitor.

4. The enucleated cell of claim 3, wherein the PD-L1 inhibitor comprises a single-domain antibody or antigen-binding fragment thereof.

5. The enucleated cell of claim 4, wherein the single-domain antibody or antigen-binding fragment thereof comprises an amino acid sequence that is greater than or equal to about 80% identical to SEQ ID NO: 851.

6. The enucleated cell of claim 3, wherein the PD-L1 inhibitor blocks binding between PD-L1 and PD-1.

7. The enucleated cell of claim 1, further comprising an exogenous nucleic acid encoding the immune checkpoint inhibitor.

8. The enucleated cell of claim 1, further comprising a virus, wherein the virus encodes the immune checkpoint inhibitor.

9. The enucleated cell of claim 1, wherein the immune checkpoint inhibitor comprises small interfering RNA (siRNA), microRNA (miRNA), short hairpin RNA (shRNA), RNA virus, or a combination thereof.

10. The enucleated cell of claim 1, wherein the immune checkpoint inhibitor comprises a small molecule.

11. The enucleated cell of claim 1, wherein the immune checkpoint inhibitor is coupled to a cytotoxic drug.

12. The enucleated cell of claim 1, further comprising a fusion protein at a cell surface of the enucleated cell, wherein the fusion protein is configured to transfer the immune checkpoint inhibitor from the enucleated cell to a target cell.

13. The enucleated cell of claim 1, wherein the enucleated cell is an enucleated stem cell.

14. The enucleated cell of claim 13, further comprising a stem cell marker, wherein the stem cell marker comprises CD90, CD146, CD166, or any combination thereof.

15. The enucleated cell of claim 1, wherein the enucleated cell is not (i) an erythrocyte, (ii) a platelet cell, or (iii) an enucleated hematopoietic stem cell.

16. The enucleated cell of claim 1, wherein the enucleated cell does not comprise complement receptor one (CR1), VLA-4, BCAM. ICAM, or any combination thereof.

17. The enucleated cell of claim 1, wherein the enucleated cell does not comprise GP1b-IX-V receptor, GPIIb/IIIa receptor, prostanoid receptor, purinergic receptor thromboxane receptor, or any combination thereof.

18. The enucleated cell of claim 1, wherein the enucleated cell does not comprise a receptor for: collagen, thrombopoietin, von Willebrand factor (VWF), fibrinogen, or any combination thereof.

19. The enucleated cell of claim 1, further comprising an exogenous tumor necrosis factor (TNF) superfamily member polypeptide or a catalytically active fragment thereof.

20. The enucleated cell of claim 19, wherein the exogenous TNF superfamily member polypeptide comprises TNF superfamily member 14 (LIGHT) or catalytically active fragment thereof.

21. The enucleated cell of claim 1, further comprising a targeting moiety.

22. The enucleated cell of claim 21, wherein the targeting moiety comprises a cytokine or a cytokine binding antibody or fragment therefor.

23. The enucleated cell of claim 21, wherein the targeting moiety comprises a homing receptor specific to a ligand or receptor expressed by a target cell.

24. The enucleated cell of claim 23, wherein the target cell is a cancer cell.

25. The enucleated cell of claim 24, wherein the cancer cell is a cell of non-small cell lung cancer (NSCLC), small cell lung cancer (SCLC), adenocarcinoma, squamous carcinoma, large cell (undifferentiated) carcinoma, large cell neuroendocrine carcinoma, adenosquamous carcinoma, sarcomatoid carcinoma, benign lung tumor, or hamartoma.

26. The enucleated cell of claim 1, further comprising an immune evasion moiety, wherein the immune evasion moiety comprises (i) cluster of differentiation (CD47), (ii) PD-L1, (iii) major histocompatibility complex, class I, E (HLA-E), (iv) major histocompatibility complex, class I, G (HLA-G), (v) a fragment of any one of (i) to (iv), or (vi) any combination thereof.

27. The enucleated cell of claim 1, wherein the enucleated cell has a diameter comprising between about 5 micrometer (μm) to 25 μm.

28. The enucleated cell of claim 1, wherein the enucleated cell is isolated.

29. The enucleated cell of claim 1, wherein the enucleated cell is cryopreserved, cryohibernated, or lyophilized.

30. A pharmaceutical formulation, comprising: the enucleated cell of claim 1; and a pharmaceutically acceptable: excipient, carrier, or diluent.


SEQUENCE LISTING (1703 sequences)

SEQ ID NO: 1 - protein, 157 aa, synthetic construct [sequence omitted]

SEQ ID NO: 2 - protein, 115 aa, synthetic construct
  REGION 1..115: note = Synthetic polypeptide
QVQLVESGGGLVQPGGSLRLSCAASGFTFSNYWMYWVRQAPGKGLEWVSEINTNGLITKYPDSVKGRFTISRDNAKNTLYLQMNSLKPEDTALYYCARSPSGFNRGQGTQVTVSS

SEQ ID NO: 3 - protein, 157 aa, synthetic construct [sequence omitted]

SEQ ID NO: 4 - protein, 129 aa, synthetic construct
  REGION 1..129: note = Synthetic polypeptide
QVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSS

SEQ ID NO: 5 - protein, 121 aa, synthetic construct
  REGION 1..121: note = Synthetic polypeptide
QVQLVESGGGLVQAGGSLRLSCTASGQTSSTADMGWFRQPPGKGREFVARISGIDGTTYYDEPVKGRFTISRDKAQNTVYLQMDSLKPEDTAVYYCRSPRYADQWSAYDYWGQGTQVTVSS

SEQ ID NO: 6 - protein, 115 aa, synthetic construct
  REGION 1..115: note = Synthetic polypeptide
QVQLVESGGGLVQPGGSLRLSCAASGFTFSDYWMYWVRQAPGKGLEWVSEINTNGLITKYPDSVKGRFTISRDNAKNTLYLQMNSLKPEDTALYYCARSPSGFNRGQGTQVTVSS

SEQ ID NO: 7 - protein, 123 aa, synthetic construct
  REGION 1..123: note = Synthetic polypeptide
EVQLVESGGGLVQAGGSLSLSCSASGRSLSNYYMGWFRQAPGKERELLGNISWRGYNIYYKDSVKGRFTISRDDAKNTIYLQMNRLKPEDTAVYYCAASILPLSDDPGWNTYWGQGTQVTVSS

SEQ ID NO: 8 - protein, 115 aa, synthetic construct
  REGION 1..115: note = Synthetic polypeptide
EVQLVESGGGLVQPGGSLRLSCAASGFTFSDYWMYWVRQAPGKGLEWVSEINTNGLITKYPDSVKGRFTISRDNAKNTLYLQMNSLKPEDTAVYYCARSPSGFNRGQGTQVTVSS

SEQ ID NO: 9 - protein, 115 aa, synthetic construct
  REGION 1..115: note = Synthetic polypeptide
EVQLVESGGGLVQPGGSLRLSCAASGFTFSDYWMYWVRQAPGKGLEWVSEINTNGLITKYPDSVKGRFTISRDNAKNTLYLQMNSLKPEDTALYYCARSPSGFNRGQGTLVTVSS

SEQ ID NO: 10 - protein, 115 aa, synthetic construct
  REGION 1..115: note = Synthetic polypeptide
EVQLVESGGGLVQPGGSLRLSCAASGFTFSDYWMYWVRQAPGKGLEWVSEINTNGLITKYPDSVKGRFTISRDNAKNTLYLQMNSLKPEDTAVYYCARSPSGFNRGQGTLVTVSS

SEQ ID NO: 11 - protein, 115 aa, synthetic construct
  REGION 1..115: note = Synthetic polypeptide
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 12 - protein, 363 aa, synthetic construct
  REGION 1..363: note = Synthetic polypeptide
QVQLVESGGGLVQPGGSLRLSCAASGFTFSDYWMYWVRQAPGKGLEWVSEINTNGLITKYPDSVKGRFTISRDNAKNTLYLQMNSLKPEDTALYYCARSPSGFNRGQGTQVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLKPEDTALYYCARSPSGFNRGQGTQVTVSS

SEQ ID NO: 13 - protein, 379 aa, synthetic construct
  REGION 1..379: note = Synthetic polypeptide
EVQLVESGGGLVQAGGSLSLSCSASGRSLSNYYMGWFRQAPGKERELLGNISWRGYNIYYKDSVKGRFTISRDDAKNTIYLQMNRLKPEDTAVYYCAASILPLSDDPGWNTYWGQGTQVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGSEVQLVESGGGLVQAGGSLSLSCSASGRSLSNYYMGWFRQAPGKERELLGNISWRGYNIYYKDSVKGRFTISRDDAKNTIYLQMNRLKPEDTAVYYCAASILPLSDDPGWNTYWGQGTQVTVSS

SEQ ID NO: 14 - protein, 384 aa, synthetic construct
  REGION 1..384: note = Synthetic polypeptide
QVQLVESGGGLVQPGGSLRLSCAASGFTFSDYWMYWVRQAPGKGLEWVSEINTNGLITKYPDSVKGRFTISRDNAKNTLYLQMNSLKPEDTALYYCARSPSGFNRGQGTQVTVSSGGGGSGGGGSGGGGSGGGGSGGGGSGGGGSEVQLVESGGGLVQPGGSLRLSCAASGFTFSDYWMYWVRQAPGKGLEWVSEINTNGLITKYPDSVKGRFTISRDNAKNTLYLQMNSLKPEDTALYYCARSPSGFNRGQGTQVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSS

SEQ ID NO: 15 - protein, 400 aa, synthetic construct
  REGION 1..400: note = Synthetic polypeptide
EVQLVESGGGLVQAGGSLSLSCSASGRSLSNYYMGWFRQAPGKERELLGNISWRGYNIYYKDSVKGRFTISRDDAKNTIYLQMNRLKPEDTAVYYCAASILPLSDDPGWNTYWGQGTQVTVSSGGGGSGGGGSGGGGSGGGGSGGGSSGGGGSEVQVVESGGGLVQAGGSLSLSCSASGRSLSNYYMGWFRQAPGKERELLGNISWRGYNIYYKDSVKGRFTISRDDAKNTIYLQMNRLKPEDTAVYYCAASILPLSDDPGWNTYWGQGTQVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSS

SEQ ID NO: 16 - protein, 363 aa, synthetic construct
  REGION 1..363: note = Synthetic polypeptide
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSS

SEQ ID NO: 17 - protein, 379 aa, synthetic construct
  REGION 1..379: note = Synthetic polypeptide
EVQLVESGGGLVQPGGSLRLSCAASGRSLSNYYMGWFRQAPGKGRELLGNISWRGYNIYYKDSVKGRFTISRDDSKNTIYLQMNSLRPEDTAVYYCAASILPLSDDPGWNTYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRSLSNYYMGWFRQAPGKGRELLGNISWRGYNIYYKDSVKGRFTISRDDSKNTIYLQMNSLRPEDTAVYYCAASILPLSDDPGWNTYWGQGTLVTVSS

SEQ ID NO: 18 - protein, 129 aa, synthetic construct
  REGION 1..129: note = Synthetic polypeptide
EVQLVESGGGLVQPGGSLRLSCAASGFTFSEPSGYTYTIGWFRQAPGKGREFVARIYWSSGLTYYADSVKGRFTISRDIAKNTVDLQMNSLRPEDTAVYYCAARDGIPTSRSVGSYNYWGQGTLVTVSS

SEQ ID NO: 19 - protein, 123 aa, synthetic construct
  REGION 1..123: note = Synthetic polypeptide
EVQLVESGGGLVQPGGSLRLSCAASGRSLSNYYMGWFRQAPGKGRELLGNISWRGYNIYYKDSVKGRFTISRDDSKNTIYLQMNSLRPEDTAVYYCAASILPLSDDPGWNTYWGQGTLVTVSS

SEQ ID NO: 20 - protein, 123 aa, synthetic construct
  REGION 1..123: note = Synthetic polypeptide
EVQLVESGGGLVQPGGSLRLSCAASGRSLSNYYMGWFRQAPGKGRELLGNISWRGYNIYYKDSVKGRFTISRDDSKNTIYLQMNSLKPEDTAVYYCAASILPLSDDPGWNTYWGQGTQVTVSS

SEQ ID NO: 21 - protein, 123 aa, synthetic construct
  REGION 1..123: note = Synthetic polypeptide
EVQLVESGGGLVQPGGSLRLSCAASGFTFSNYYMGWFRQAPGKGRELLGNISWRGYNIYYKDSVKGRFTISRDDSKNTIYLQMNSLKPEDTAVYYCAASILPLSDDPGWNTYWGQGTQVTVSS

SEQ ID NO: 22 - protein, 123 aa, synthetic construct
  REGION 1..123: note = Synthetic polypeptide
EVQLVESGGGLVQPGGSLRLSCAASGRSLSNYYMGWFRQAPGKGRELLGNISWRGYNIYYKDSVKGRFTISRDDSKNTIYLQMNSLKTEDTAVYYCAASILPLSDDPGWNTYWGQGTQVTVSS

SEQ ID NO: 23 - protein, 123 aa, synthetic construct
  REGION 1..123: note = Synthetic polypeptide
EVQLVESGGGLVQPGGSLRLSCAASGRSLSNYYMGWFRQAPGKGRELLGNISWRGYNIYYKDSVKGRFTISRDDSKNTIYLQMNSLKPEDTAVYYCAASILPLSDDPGWNTYWGQGTLVTVSS

SEQ ID NO: 24 - protein, 115 aa, synthetic construct [sequence omitted]

SEQ ID NO: 25 - protein, 115 aa, synthetic construct
  REGION 1..115: note = Synthetic polypeptide
EVQLVESGGGLVQPGGSLRLSCATSGFDFSVSWMYWVRQAPGKGLEWVSEINTNGLITKYVDSVKGRFTISRDNAKNTLYLQMDSLIPEDTALYYCARSPSGSFRGQGTQVTVSS

SEQ ID NO: 26 - protein, 129 aa, synthetic construct
  REGION 1..129: note = Synthetic polypeptide
QVQLVESGGGLVQAGGSLRLSCAASGRTFSEPSGYTYTIGWFRQAPGKEREFVARIYWSSGLTYYADSVKGRFTISRDIAKNTVDLLMNSLKPEDTAVYYCAARDGIPTSRSVGSYNYWGQGTQVTVSS

SEQ ID NO: 27 - protein, 129 aa, synthetic construct
  REGION 1..129: note = Synthetic polypeptide
QVKLEESGGGLVQPGDSLRLSCAASGRTFSDYSGYTYTVGWFRQAPGKEREFVARIYWSSGNTYYADSVKGRFTISRDIAKNTVDLLMNNLEPEDTAVYYCAARDGIPTSRSVESYNYWGQGTQVTVSS

SEQ ID NO: 28 - protein, 129 aa, synthetic construct
  REGION 1..129: note = Synthetic polypeptide
AVQLVESGGGLVQPGDSLRLSCAASGRTFSDYSGYTYTVGWFRQAPGKEREFVARIYWSSGNTYYADSVKGRFTISRDIAKNTVDLLMNNLEPEDTAVYYCAARDGIPTSRSVESYNYWGQGTQVTVSS

SEQ ID NO: 29 - protein, 127 aa, synthetic construct
  REGION 1..127: note = Synthetic polypeptide
AVQLVDSGGGLVQAGGSLRLSCAASGRTFSAHSVYTMGWFRQAPGKEREFVARIYWSSANTYYADSVKGRFTISRDNAKNTVDLLMNCLKPEDTAVYYCAARDGIPTSRSVEAYNYWGQGTQVTVSS

SEQ ID NO: 30 - protein, 123 aa, synthetic construct
  REGION 1..123: note = Synthetic polypeptide
QVQLVESGGGLVQAGGSLSLSCAASGRSFTGYYMGWFRQAPGKERQLLASISWRGDNTYYKESVKGRFTISRDDAKNTIYLQMNSLKPEDTAVYYCAASILPLSDDPGWNTNWGQGTQVTVSS

SEQ ID NO: 31 - protein, 117 aa, synthetic construct
  REGION 1..117: note = Synthetic polypeptide
AVQLVESGGGLVQGGGSLRLACAASERIFDLNLMGWYRQGPGNERELVATCITVGDSTNYADSVKGRFTISMDYTKQTVYLHMNSLRPEDTGLYYCKIRRTWHSELWGQGTQVTVSS

SEQ ID NO: 32 - protein, 116 aa, synthetic construct
  REGION 1..116: note = Synthetic polypeptide
EVQLVESGGGLVQEGGSLRLACAASERIWDINLLGWYRQGPGNERELVATITVGDSTSYADSVKGRFTISRDYDKNTLYLQMNSLRPEDTGLYYCKIRRTWHSELWGQGTQVTVSS

SEQ ID NO: 33 - protein, 115 aa, synthetic construct [sequence omitted]

SEQ ID NO: 34 - protein, 116 aa, synthetic construct
  REGION 1..116: note = Synthetic polypeptide
AVQLVDSGGGLVQPGGSLRLSCAASGFSFGSFGMSWVRQYPGKEPEWVSSINGRGDDTRYADSVKGRFSISRDNAKNTLYLQMNSLKPEDTAEYYCTIGRSVSRSRTQGTQVTVSS

SEQ ID NO: 35 - protein, 114 aa, synthetic construct
  REGION 1..114: note = Synthetic polypeptide
AVQLVESGGGLVQPGGSLRLTCTASGFTFRSFGMSWVRQAPGKDQEWVSAISADSSTKNYADSVKGRFTISRDNAKKMLYLEMNSLKPEDTAVYYCVIGRGSPSSPGTQVTVSS

SEQ ID NO: 36 - protein, 114 aa, synthetic construct
  REGION 1..114: note = Synthetic polypeptide
QVQLAESGGGLVQPGGSLRLTCTASGFTFGSFGMSWVRQAPGEGLEWVSAISADSSDKRYADSVKGRFTISRDNAKKMLYLEMNSLKSEDTAVYYCVIGRGSPASQGTQVTVSS

SEQ ID NO: 37-100 - skipped (64 entries)

SEQ ID NO: 101 - protein, 115 aa, synthetic construct; identical to SEQ ID NO: 6
  REGION 1..115: note = Synthetic polypeptide

SEQ ID NO: 102 - protein, 129 aa, synthetic construct; identical to SEQ ID NO: 28
  REGION 1..129: note = Synthetic polypeptide

SEQ ID NO: 103 - protein, 127 aa, synthetic construct; identical to SEQ ID NO: 29
  REGION 1..127: note = Synthetic polypeptide

SEQ ID NO: 104 - protein, 123 aa, synthetic construct; identical to SEQ ID NO: 30
  REGION 1..123: note = Synthetic polypeptide

SEQ ID NO: 105 - protein, 123 aa, synthetic construct; identical to SEQ ID NO: 7
  REGION 1..123: note = Synthetic polypeptide

SEQ ID NO: 106 - protein, 117 aa, synthetic construct; identical to SEQ ID NO: 31
  REGION 1..117: note = Synthetic polypeptide

SEQ ID NO: 107 - protein, 116 aa, synthetic construct; identical to SEQ ID NO: 32
  REGION 1..116: note = Synthetic polypeptide

SEQ ID NO: 108 - protein, 115 aa, synthetic construct [sequence omitted]

SEQ ID NO: 109 - protein, 116 aa, synthetic construct; identical to SEQ ID NO: 34
  REGION 1..116: note = Synthetic polypeptide

SEQ ID NO: 110 - protein, 585 aa, synthetic construct [sequence omitted]

SEQ ID NO: 111 - protein, 120 aa, synthetic construct
  REGION 1..120: note = Synthetic polypeptide
QVQLQESGGGLVQAGGSLRLSCAASGYISDAYYMGWYRQAPGKEREFVATITHGTNTYYADSVKGRFTISRDNAKNTVYLQMNSLKPEDTAVYYCAVLETRSYSFRYWGQGTQVTVSSLE

SEQ ID NO: 112-120 - skipped (9 entries)

SEQ ID NO: 121 - protein, 592 aa, synthetic construct
  REGION 1..592: note = Synthetic polypeptide
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVHEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFK

SEQ ID NO: 122 - protein, 270 aa, synthetic construct
  REGION 1..270: note = Synthetic polypeptide
IFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVHEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEK

SEQ ID NO: 123 - protein, 296 aa, synthetic construct
  REGION 1..296: note = Synthetic polypeptide
IFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVHEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQ

SEQ ID NO: 124-130 - skipped (7 entries)

SEQ ID NO: 131 - DNA, 360 bp, Homo sapiens
  source 1..360: mol_type = genomic DNA
atggcaaatcctcctacattctccccggcactgctggttgttaccgaaggcgataatgcgacctttacctgtagtttctccaatacgagcgaatcgtttgtcctgaactggtatcgtatgagcccgtctaatcagaccgataaactggcggccttcccggaagatcgctctcagccgggccaagacagccgttttcgcgttacgcaactgccgaacggtcgtgatttccatatgagtgtggttcgcgcccgtcgcaatgactccggcacctacctgtgtggtgcaatttcactggctccgaaagcccaaatcaaagaatcgctgcgtgcggaactgcgtgttaccgaacgtcgtgccgaa

SEQ ID NO: 132 - DNA, 633 bp, Homo sapiens
  source 1..633: mol_type = genomic DNA
tttacggttacggttccgaaagacctgtatgtggttgaatacggctctaatatgacgattgaatgcaaattcccggttgaaaaacaactggatctggcggccctgattgtgtattgggaaatggaagacaaaaacatcatccaattcgtgcatggcgaagaagatctgaaagttcagcacagctcttaccgtcaacgcgcacgtctgctgaaagaccagctgagcctgggcaatgcagctctgcagatcacggatgttaaactgcaagacgccggtgtctatcgctgcatgatttcttatggcggtgcagactacaaacgtatcaccgtcaaagtgaacgctccgtacaacaaaattaatcagcgcatcctggtggttgatccggttacgtccgaacatgaactgacctgtcaagcggaaggctatccgaaagccgaagtcatttggaccagttccgatcaccaggtgctgtcaggtaaaaccacgaccacgaactcgaaacgcgaagaaaaactgtttaatgtcacgagcaccctgcgtattaacaccacgaccaatgaaatcttctactgcacctttcgtcgtctggacccggaagaaaatcatacggcggaactggttatcccggaactg

SEQ ID NO: 133 - DNA, 360 bp, Homo sapiens; identical to SEQ ID NO: 131
  source 1..360: mol_type = genomic DNA

SEQ ID NO: 134 - DNA, 633 bp, Homo sapiens; identical to SEQ ID NO: 132
  source 1..633: mol_type = genomic DNA

SEQ ID NO: 135-141 - skipped (7 entries)

SEQ ID NO: 142 - DNA, 531 bp, Homo sapiens
  source 1..531: mol_type = genomic DNA
atgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 143 - DNA, 537 bp, Homo sapiens
  source 1..537: mol_type = genomic DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggagattag

SEQ ID NO: 144 - DNA, 873 bp, Homo sapiens
  source 1..873: mol_type = genomic DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 145 - DNA, 531 bp, Homo sapiens; identical to SEQ ID NO: 142
  source 1..531: mol_type = genomic DNA

SEQ ID NO: 146 - DNA, 738 bp, Homo sapiens
  source 1..738: mol_type = genomic DNA
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccaggtaatattctgaatgtgtccattaaaatatgtctaacactgtcccctagcacctag

SEQ ID NO: 147 - DNA, 873 bp, Homo sapiens; identical to SEQ ID NO: 144
  source 1..873: mol_type = genomic DNA

SEQ ID NO: 148 - DNA, 3461 bp, Homo sapiens
  source 1..3461: mol_type = genomic DNA
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggagattagatcctgaggaaaaccatacagctgaattggtcatcccagaactacctctggcacatcctccaaatgaaaggactcacttgggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 149 - DNA, 873 bp, Homo sapiens; identical to SEQ ID NO: 144
  source 1..873: mol_type = genomic DNA

SEQ ID NO: 150 - DNA, 873 bp, Homo sapiens; identical to SEQ ID NO: 144
  source 1..873: mol_type = genomic DNA

SEQ ID NO: 151 - DNA, 873 bp, Homo sapiens; identical to SEQ ID NO: 144
  source 1..873: mol_type = genomic DNA

SEQ ID NO: 152 - DNA, 873 bp, Homo sapiens; identical to SEQ ID NO: 144
  source 1..873: mol_type = genomic DNA

SEQ ID NO: 153-154 - skipped (2 entries)

SEQ ID NO: 155 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 156 - protein, 178 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGD

SEQ ID NO: 157 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 158 - protein, 176 aa, Homo sapiens; identical to SEQ ID NO: 155

SEQ ID NO: 159 - protein, 245 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 160 - protein, 290 aa, Homo sapiens; identical to SEQ ID NO: 157

SEQ ID NO: 161 - protein, 290 aa, Homo sapiens; identical to SEQ ID NO: 157

SEQ ID NO: 162 - protein, 290 aa, Homo sapiens; identical to SEQ ID NO: 157

SEQ ID NO: 163 - protein, 290 aa, Homo sapiens; identical to SEQ ID NO: 157

SEQ ID NO: 164 - protein, 290 aa, Homo sapiens; identical to SEQ ID NO: 157

SEQ ID NO: 165 - protein, 120 aa, synthetic construct
  REGION 1..120: note = Synthetic polypeptide
QVQLQESGGGLVQPGGSLRLSCAASGFTFSSYAMSWVRQAPGKTLEWVSDINTGGDTSDYADSVKGRFTISRDNAKNIVYLQMNSLKAEDTAVYYCANVPKELVLSFGSWGQGTQVTVSS

SEQ ID NO: 166 - protein, 120 aa, synthetic construct
  REGION 1..120: note = Synthetic polypeptide
QVQLQESGGGLVQPGGSLRLSCAASGFTFSSFAMSWVRQAPGKGLEWVSDIDTTGRTTDYADSVKGRFTISRDNAENTLYLQMNDLKPEDTAVYYCANVPKELVLSFGSWGPGTQVTVSS

SEQ ID NO: 167 - protein, 120 aa, synthetic construct
  REGION 1..120: note = Synthetic polypeptide
QVQLQESGGGSVQPGGSLRLSCEASGFTFSSYAMSWVRQAPGEGLEWVSDINTGGDNTDYAGSVKGRFTISRDNAKNTLYLQMDSLKPEDTAKYYCANVPKELVHSFNSWGQGTQVTVSS

SEQ ID NO: 168 - protein, 120 aa, synthetic construct
  REGION 1..120: note = Synthetic polypeptide
QVQLQESGGGLVQPGGSLRLSCAASGFTFSSFAMSWVRQVPGRTLEWVSDINTGGDSTDYANSVKGRFTISRDNAKNTLYLQMNSLKPDDTAVYYCANVPKELVFSFASWGRGTQVTVSS

SEQ ID NO: 169 - protein, 146 aa, synthetic construct
  REGION 1..146: note = Synthetic polypeptide
TGQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSGSMDPGGSHHHHHHHH

SEQ ID NO: 170 - protein, 124 aa, synthetic construct
  REGION 1..124: note = Synthetic polypeptide
MAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNALEHHHHHH

SEQ ID NO: 171 - protein, 119 aa, synthetic construct
  REGION 1..119: note = Synthetic polypeptide
EVQLVESGGGLVQAGGSLRLSCAASGSIASIHAMGWFRQAPGKEREFVAVITWSGGITYYADSVKGRFTISRDNAKNTVYLQMNSLKPEDTAIYYCAGDKHQSSWYDYWGQGTQVTVSS

SEQ ID NO: 172 - protein, 119 aa, synthetic construct
  REGION 1..119: note = Synthetic polypeptide
EVQLVESGGGLVQAGGSLRLSCAASGSIASIHAMGWFRQAPGKEREFVAVITWSGGITYYADSVKGRFTISRDNAKNTVYLQMNSLKPEDTAIYYCAGDKHQSSWYDYWGQGTLVTVSS

SEQ ID NO: 173 - protein, 119 aa, synthetic construct
  REGION 1..119: note = Synthetic polypeptide
EVQLVESGGGLVQAGGSLRLSCAASGSIASIHAMGWFRQAPGKEREFVAVITWSGGITYYADSVKGRFTISRDNAKNTVYLQMNSLKPEDTATYYCAGDKHQSSWYDYWGQGTLVTVSS

SEQ ID NO: 174 - protein, 119 aa, synthetic construct
  REGION 1..119: note = Synthetic polypeptide
EVQLVESGGGVVQAGGSLRLSCAASGSIASIHAMGWFRQAPGKEREFVAVITWSGGITYYADSVKGRFTISRDNAKNTVYLQMNSLKPEDTAIYYCAGDKHQSSWYDYWGQGTLVKVSS

SEQ ID NO: 175 - protein, 120 aa, synthetic construct
  REGION 1..120: note = Synthetic polypeptide
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSA

SEQ ID NO: 176 - protein, 119 aa, synthetic construct
  REGION 1..119: note = Synthetic polypeptide
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 177 - protein, 119 aa, synthetic construct
  REGION 1..119: note = Synthetic polypeptide
EVQLVESGGGLVQPGGSLRLSCAASGSIASIHAMGWFRQAPGKEREFVAVITWSGGITYYADSVKGRFTISRDNSKNTVYLQMNSLRPEDTAIYYCAGDKHQSSWYDYWGQGTLVTVSS

SEQ ID NO: 178 - protein, 119 aa, synthetic construct
  REGION 1..119: note = Synthetic polypeptide
EVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSS

SEQ ID NO: 179 - protein, 270 aa, synthetic construct
  REGION 1..270: note = Synthetic polypeptide
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 180 - protein, 424 aa, synthetic construct
  REGION 1..424: note = Synthetic polypeptide
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSGGGGSGGGGSGGGGSGGGGSGGGGSGGGGSGGGGSEVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 181 - protein, 119 aa, synthetic construct
  REGION 1..119: note = Synthetic polypeptide
DVQLVESGGGVVQPGGSLRLSCAASGSIASIHAMGWFRQAPGKEREFVAVITVSGGITYYADSVKGRFTISRDNSKNTVYLQMNSLRPEDTALYYCAGDKHQSSFYDYWGQGTLVTVSS

SEQ ID NO: 182 - protein, 119 aa, synthetic construct
  REGION 1..119: note = Synthetic polypeptide
DVQLVESGGGVVQPGGSLRLSCAASGSIASIHAMGWFRQAPGKEREFVAVITVSGGITYYADSVKGRFTISRDQSKNTVYLQMNSLRPEDTALYYCAGDKHQSSFYDYWGQGTLVTVSS

SEQ ID NO: 183 - protein, 119 aa, synthetic construct
  REGION 1..119: note = Synthetic polypeptide
DVQLVESGGGVVQPGGSLRLSCAASGSIASIHAMGWFRQAPGKEREFVAVITVSGGITYYADSVKGRFTISRDPSKNTVYLQMNSLRPEDTALYYCAGDKHQSSFYDYWGQGTLVTVSS

SEQ ID NO: 184 - protein, 749 aa, synthetic construct
  REGION 1..749: note = Synthetic polypeptide
EVQLVESGGGLVQPGGSLRLSCAASGSIASIHAMGWFRQAPGKEREFVAVITWSGGITYYADSVKGRFTISRDNSKNTVYLQMNSLRPEDTAIYYCAGDKHQSSWYDYWGQGTLVTVSSGGGGSGGGGSGGGGSGGGGSGGGGSGGGGSGGGGSEVQLVESGGGLVQPGGSLRLSCAASGSIASIHAMGWFRQAPGKEREFVAVITWSGGITYYADSVKGRFTISRDNSKNTVYLQMNSLRPEDTAIYYCAGDKHQSSWYDYWGQGTLVTVSSGGGGSGGGGSGGGGSGGGGSGGGGSGGGGSGGGGSEVQLVESGGGLVQAGGSLRLSCAASGRTFSDYVMGWFRQARGNEREFVAAISESGGRTHYADAVKGRFTISRDNAKNTLYLQMNSLKPEDTAVYYCATTLLWWTSEYAPIKANDYDYWGQGTLVTVSSGGGGSGGGGSGGGGSGGGGSGGGGSGGGGSGGGGSEVQLVESGGGLVQAGGSLRLSCAASGRTFSDYVMGWFRQARGNEREFVAAISESGGRTHYADAVKGRFTISRDNAKNTLYLQMNSLKPEDTAVYYCATTLLWWTSEYAPIKANDYDYWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 185 - protein, 749 aa, synthetic construct
  REGION 1..749: note = Synthetic polypeptide
DVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSGGGGSGGGGSGGGGSGGGGSGGGGSGGGGSGGGGSEVQLVESGGGVVQPGGSLRLSCAASGSIASIHAMGWFRQAPGKEREFVAVITWSGGITYYADSVKGRFTISRDNSKNTVYLQMNSLRPEDTALYYCAGDKHQSSWYDYWGQGTLVTVSSGGGGSGGGGSGGGGSGGGGSGGGGSGGGGSGGGGSEVQLVESGGGVVQPGGSLRLSCAASGRTFSDYVMWFRQAPGKEREFVAAISESGGRTHYADSVKGRFTISRDNSKNTLYLQMNSLRPEDTALYYCATTLLWWTSEYAPIKANDYDYWGQGTLVTVSSGGGGSGGGGSGGGGSGGGGSGGGGSGGGGSGGGGSEVQLVESGGGVVQPGGSLRLSCAASGRTFSDYVMGWFRQAPGKEREFVAAISESGGRTHYADSVKGRFTISRDNSKNTLYLQMNSLRPEDTALYYCATTLLWWTSEYAPIKANDYDYWGQGTLVTVSSGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 186 - DNA, 321 bp, synthetic construct
  misc_feature 1..321: note = Synthetic polynucleotide
  source 1..321: mol_type = other DNA
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccattcactttcggccctgggaccaaagtggatatcaaa

SEQ ID NO: 187 - DNA, 318 bp, synthetic construct
  misc_feature 1..318: note = Synthetic polynucleotide
  source 1..318: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcccaccttcggccaagggacacgactggagattaaa

SEQ ID NO: 188 - DNA, 318 bp, synthetic construct
  misc_feature 1..318: note = Synthetic polynucleotide
  source 1..318: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctttcggcggagggaccaaggtggagatcaaa

SEQ ID NO: 189 - DNA, 318 bp, synthetic construct
  misc_feature 1..318: note = Synthetic polynucleotide
  source 1..318: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgacgttcggccaagggaccaaggtggaaatcaaa

SEQ ID NO: 190 - DNA, 318 bp, synthetic construct; identical to SEQ ID NO: 189
  misc_feature 1..318: note = Synthetic polynucleotide
  source 1..318: mol_type = other DNA

SEQ ID NO: 191 - DNA, 321 bp, synthetic construct
  misc_feature 1..321: note = Synthetic polynucleotide
  source 1..321: mol_type = other DNA
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtataatagttacccgtacacttttggccaggggaccaagctggagatcaaa

SEQ ID NO: 192 - DNA, 324 bp, synthetic construct
  misc_feature 1..324: note = Synthetic polynucleotide
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 193-194 - skipped (2 entries)

SEQ ID NO: 195 - protein, 130 aa, synthetic construct
  REGION 1..130: note = Synthetic polypeptide
MAQVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGGHHHHHH

SEQ ID NO: 196-200 - skipped (5 entries)

SEQ ID NO: 201 - DNA, 672 bp, Homo sapiens
  source 1..672: mol_type = genomic DNA
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattga

SEQ ID NO: 202 - DNA, 672 bp, Homo sapiens; identical to SEQ ID NO: 201
  source 1..672: mol_type = genomic DNA

SEQ ID NO: 203 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 204 - protein, 223 aa, Homo sapiens; identical to SEQ ID NO: 203

SEQ ID NO: 205 - protein, 144 aa, synthetic construct
  REGION 1..144: note = Synthetic polypeptide
QVQLVESGGGLVQPGGSLRLSCAASGYIYSAYCMGWFRQAPGKGLEGVAAIYIGGGSTYYADSVKGRFTISRDNSKNTLYLQMNSLRAEDTAVYYCAADVIPTETCLGGSWSGPFGYWGQGTLVTVSSGSMDPGGSHHHHHHHH

SEQ ID NO: 206 - protein, 125 aa, synthetic construct
  REGION 1..125: note = Synthetic polypeptide
AMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 207-210 - skipped (4 entries)

SEQ ID NO: 211 - protein, 117 aa, synthetic construct
  REGION 1..117: note = Synthetic polypeptide
IQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDP

SEQ ID NO: 212 - protein, 129 aa, synthetic construct
  REGION 1..129: note = Synthetic polypeptide
MAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGHHHHHH

SEQ ID NO: 213 - protein, 129 aa, synthetic construct; identical to SEQ ID NO: 212
  REGION 1..129: note = Synthetic polypeptide

SEQ ID NO: 214-220 - skipped (7 entries)

SEQ ID NO: 221 - protein, 121 aa, synthetic construct [sequence omitted]

SEQ ID NO: 222 - protein, 245 aa, synthetic construct
  REGION 1..245: note = Synthetic polypeptide
EVQLVESGGGLVQPGGSLRLSCAASGSVFKINVMAWYRQAPGKGRELVAGIISGGSTSYADSVKGRFTISRDNAKNTLYLQMNSLRPEDTAVYYCAFITTESDYDLGRRYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 223 - protein, 121 aa, synthetic construct
  REGION 1..121: note = Synthetic polypeptide
EVQLVESGGGLVQPGGSLRLSCAASGTTFRINVMAWYRQAPGKGRELVAGIITNGSTSYADSVKGRFTISRDNAKNTLYLQMNSLRPEDTAVYYCAFVTTNSDYDLGRDYWGQGTLVTVSS

SEQ ID NO: 224 - protein, 121 aa, synthetic construct
  REGION 1..121: note = Synthetic polypeptide
EVQLVESGGGLVQPGGSLRLSCAASGSIFRVNVMAWYRQAPGKGRELVAAVINGGTTTYADSVKGRFTISRDNAKNTLYLQMNSLRPEDTAVYYCAFVTTNSDYDLGRDYWGQGTLVTVSS

SEQ ID NO: 225 - protein, 121 aa, synthetic construct
  REGION 1..121: note = Synthetic polypeptide
EVQLVESGGGLVQPGGSLRLSCAASGTTFKINIMAWYRQAPGKGRELVAGVITGGNTTYADSVKGRFTISRDNAKNTLYLQMNSLRPEDTAVYYCAFVTTNSDYDLGRDYWGQGTLVTVSS

SEQ ID NO: 226 - protein, 121 aa, synthetic construct
  REGION 1..121: note = Synthetic polypeptide
EVQLVESGGGLVQPGGSLRLSCAASGSTFRINVMAWYRQAPGKGRELVAGVINDGSTTYADSVKGRFTISRDNAKNTLYLQMNSLRPEDTAVYYCAFVTTNSDYDLGRDYWGQGTLVTVSS

SEQ ID NO: 227 - protein, 121 aa, synthetic construct
  REGION 1..121: note = Synthetic polypeptide
EVQLVESGGGLVQPGGSLRLSCAASGSVFKINVMAWYRQAPGKGRELVAGIVSGGSTSYADSVKGRFTISRDNAKNTLYLQMNSLRPEDTAVYYCAFITTNSDYDLGRRYWGQGTLVTVSS

SEQ ID NO: 228 - protein, 121 aa, synthetic construct
  REGION 1..121: note = Synthetic polypeptide
EVQLVESGGGLVQPGGSLRLSCAASGTTFKVNVMAWYRQAPGKGRELVAGIINGGSTTYADSVKGRFTISRDNAKNTLYLQMNSLRPEDTAVYYCAFVTTNSDYDLGRDYWGQGTLVTVSS

SEQ ID NO: 229 - protein, 121 aa, synthetic construct
  REGION 1..121: note = Synthetic polypeptide
EVQLVESGGGLVQPGGSLRLSCAASGSVFKINVMAWYRQAPGKGRELVAGIVSGGSTSYADSVKGRFTISRDNAKNTLYLQMNSLRPEDTAVYYCAFITTESDYDLGRRYWGQGTLVTVSS

SEQ ID NO: 230 - protein, 121 aa, synthetic construct
  REGION 1..121: note = Synthetic polypeptide
EVQLVESGGGLVQPGGSLRLSCAASGSIFKINVMAWYRQAPGKGRELVAGIVTGGSTSYADSVKGRFTISRDNAKNTLYLQMNSLRPEDTAVYYCAFITTESDYDLGRRYWGQGTLVTVSS

SEQ ID NO: 231 - protein, 121 aa, synthetic construct
  REGION 1..121: note = Synthetic polypeptide
EVQLVESGGGLVQPGGSLRLSCAASGSVFKINVMAWYRQAPGKGRELVAGIVTGGSTSYADSVKGRFTISRDNAKNTLYLQMNSLRPEDTAVYYCAFITTESDYDLGRRYWGQGTLVTVSS

SEQ ID NO: 232-240 - skipped (9 entries)

SEQ ID NO: 241 - protein, 115 aa, synthetic construct [sequence omitted]

SEQ ID NO: 242 - protein, 449 aa, synthetic construct
  REGION 1..449: note = Synthetic polypeptide
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 214 aa, synthetic construct [sequence omitted]

SEQ ID NO: 244 - protein, 222 aa, synthetic construct
  REGION 1..222: note = Synthetic polypeptide
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 245 - protein, 214 aa, synthetic construct [sequence omitted]

SEQ ID NO: 246-300 - skipped (55 entries)

SEQ ID NO: 301 - DNA, 1081 bp, Homo sapiens
  source 1..1081: mol_type = genomic DNA
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacaggtcagcctcaggtgggaaaggagt

SEQ ID NO: 302 - DNA, 1578 bp, Homo sapiens
  source 1..1578: mol_type = genomic DNA
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctgctgtttctcatccttggtgtcctttctctgctccttttggtgactggagcctttggctttcacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctctga

SEQ ID NO: 303 - DNA, 1335 bp, Homo sapiens
  source 1..1335: mol_type = genomic DNA
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacaggtgcccaacgctctgggagagccccaggtgccctcccagcaggccacctcctgctgtttctcatccttggtgtcctttctctgctccttttggtgactggagcctttggctttcacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctctga

SEQ ID NO: 304 - DNA, 1373 bp, Homo sapiens
  source 1..1373: mol_type = genomic DNA
tgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctgctgtttctcatccttggtgtcctttctctgctccttttggtgactggagcctttggctttcacctttggagaagacag

SEQ ID NO: 305 - DNA, 1083 bp, Homo sapiens
  source 1..1083: mol_type = genomic DNA
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacaggtcagcctcaggtgggaaaggagtag

SEQ ID NO: 306 - DNA, 1575 bp, Homo sapiens
  source 1..1575: mol_type = genomic DNA
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctgctgtttctcatccttggtgtcctttctctgctccttttggtgactggagcctttggctttcacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctc

SEQ ID NO: 307 - DNA, 1578 bp, Homo sapiens
  source 1..1578: mol_type = genomic DNA
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtgggtacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctgctgtttctcacccttggtgtcctttctctgctccttttggtgactggagcctttggctttcacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctctga

SEQ ID NO: 308 - DNA, 707 bp, Homo sapiens
  source 1..707: mol_type = genomic DNA
cacgaggctgctctccgccacggccctgctctgttccctgggacacccccgcccccacctcctcaggctgcctgatctgcccagctttccagctttcctctggattccggcctctggtcatccctccccaccctctctccaaggccctctcctggtctcccttcttctagaaccccttcctccacctccctctctgcagaacttctcctttaccccccaccccccaccactgccccctttccttttctgacctccttttggagggctcagcgctgcccagaccataggagagatgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgngggcccaggccccgccgctacacggtgctgagcgtgggtcccggaggcctgcgcagcgggaggctgcccctgcagccccgcgtccagctggatgaagcgcgccggcagcgcggggacttctcgctatggctgcgcccag

SEQ ID NO: 309 - DNA, 5253 bp, Homo sapiens
  source 1..5253: mol_type = genomic DNA
tgacctggagctgaggcagccagcagatctcagcagcccagtccaaataaactccctgtcagcagcaagcctgcgtctggctctttcttgtccaagtgaaggttcccgtctctccccagcctctgagggctcccttctcccaccctagcctcctaacctactttccccagccagcctccctcaccttccccttttctgtttctggctgcttcgtgtttctttcttggtctctctctctcttgcgctcactctctctttctctctcctgggtaatcatttctctgaagctcccccgaaaaagcccttgccccttgcctccgcagctctctcctccgtccttccacccaccttccacccacagtgcgctggctgccctggtttccttgatattttgtcctttgggtcacccacccacactcagctgcacttcagcacttgtccttggccctccggctgcagcagatgcgcacatatgctcgcgcgcacgtgcacacacacatgcacacacacgcgcgcgcgcacacgcacacacacacacacacacacacacgcgcgcggcgcgcagaggactgccttgtcaagtcttccaggagatccggtctggaaacctctctgggcagccggctggggctcctacctctgccccaccccccacgttcctcagcccttggaggcttttctggtcccaggcatgagaacacactgccacccagtggtcactgcttgtcacaccccaacggaggtcactgggccggctgcggcaggcctgaggagttgagcagtgcagtggacaaggcagggcacagactgctgtgaatgcaggcacgcggtttgtattatgcacgttctatgtgcagcatatgagtatgtgcccggtgactgtcacatgtcctatgcacccttcaggagagggactgaacaatgcatgtctcaggagatacagcctggctggcctctcaagggtgtaatctatgtctgacaaaggggaaggctgtgaacaaaggcctgtgcgtagagcaaaacagccagtgcagctgtgggagtcgggagccccggcttctcattccagctttgctgatgattctgtagtcaatctccctgagcctcagtttcctcacctattaaatgggataatgataataacccaccaaaatgggttgttgtgaatattggataataaagggctttggaaactgtgaagtgctctgtgtgtgtggagtacttgtctgattatccattagtgaaggagaaagcaggcctgctctggtcatgctaagaatgatattgaagaacccaagtcggcctgcgccctgctctgtctgtctgtcttggagcattcacctgggccatgtcaatggtcctactcagcagagcaaacacggcctcagcacatggcatcttccagtctggcatgggacagagacttctgcctgcaaataggtgtggggccctggactttggccacagatgactctgtcaggggactttaaccccatacaggagaacaaaatattgaaagcgagtgggtgagtgggagtgaaactgctggagcaggaggggcatggtggcatgtgtgagtggcatcccactgggtctgtgtgccgtgtgcaaaaagacacaaaaagacagacctgaggacatgtccctgcagagacacaatagtcactcattagagacagacgatgaagaaggtgacacctttgttctgtgcagagccttaggtagaggggggaccctctgaagcctgaacaatgaaccatttacagcaatctgaggttctgagcctcaggatggtgatgactcttgccttgtcactggcagagaaacactggcacaggaaggaggaagctctggccctgccagagatgatcacactgcaaaaggaagtcttgaagtccttttagaagcttccaggccaggtgtcatggcttatgcctgtaatctcagcactttgggaggcggaggcagaaggattgcttgagcccaggagttcgagaccagcctgggcagtatagtgagacctcatctctaaaaaaaaaaaaaaagttagttgggcatgctggtacgcccctgtggtcccagctatttgggaggttgaggtgggaggatcacctaascctgggaggttgaggctgcagtgagccataatcgtgccactatactccagcctgggtgacagagtgagatcctgtctcaaaaacaaaaaacaagaagcctccaaagcatcgttcctacccttcgctgctgctcagagactcctcagctccagcccaggaccctgctccccacagccttagtggaagccctgatattttaatagggcagggtttcctggaaagtctgtgggtggcatttttccttttactagctarcatcctatggccctttgggcttatgagagtagtcatcgaagatcactgagctatgcgggactccccatgctctttcataaaggggacagggaggggacagaggcaacgaagggcctcctttttcctttaccccttagagaagagcaaagggcagaaggaagaaagggcatatgggctgcattccgtgagtgcttctctgtggggtctctcctcagggtgacattctgctcccactcctcttcccacagccccccactctccgtctggcaggataatctccagcccttttcctgtcctccaaaggcccctagggagcttggttaattcatttggcttctccaggttgatctgtaaaccagtgttccaggctgcggtcctggaggggaaggagcatcaggaggccacgagctcagggagcttcagtgggtgggtgggtgggaatcatctcagctccttgttaagaaggaaggggggtgcgtgagttacctattggcgtataacaaacaaattaccctaaaacttagtaatttaaaataataagctgggtgtggtggctcacactggtaatcccaacactttggaagactgaggcaggaggattgcttgaggccgggagtgtgagaccagcctgggtgacttagtgagatcacacttctacttttttttttttttgagacagagtctcactctgtcacccaggctggagtgcagtggcttgatctcggctcactgcaacctccatctgggctcaagcgattctcctgccacagcctcccaagtagctgggactacaggtgcataccaccacaccctgctaatttttgaatttttgtattttaattaataattaataaattatttttgagacagagttttgcttttgtcgcccaggctggagtgcagtttggtgccatctcggctcactgcaagctccgcctctcgggttcaagcaattctcctgcctcagcctctggagtagctgggactacaggcgccagccaccacgcccggctaactttctgtgtttttagtagagacggggtttcactgtgttagctaggatgggtctggatctcctgacctcgtgatccgcctgcctcggcctcccaaagtgctgggattacaggcgtgaaccaccacacccagcctaccatttttttttctttttgaatttaaaaatgagctgggcacggtggcatgcacctgtggtcctagttattcaggaggctgagggagaagaatcactggaggctaggatttccaggctgtagtgagccatgattgtgctatttcactccagcttgggtgacagggtgagatcctgtgtctaaaaagaataataacaataataataaacattatctcacacaacgaggaatccaggagcagtcttgctgagtagttctggcacagggttgctcatgaggttgctgctgaggtgagggtcaggtctggagccatctgaaggctgggatggggctggagattctgcctccaagacagctcactcgggaggctgtcggccagaggcttaactcctccctgtgtgggtgtctttatggggcctctcacaacacgacacagcaactggcttcccccagaggacagcgtggaaaagacaaaagctaccacatctttatttcaagattagatttgatcttgaaagcaacgtgccataatttctgccctattctattgatcacagagaccaacctggtgcaagcaggagggggcaatgcaagggtgtgaactgagggagacaggagtcactgggggccatcttgcagcctggctgtcagagtggggagcagtgcctgaaacagagataggccaagagacgggaagggacgtctctgagcatctccatccccagccctcttctcttgtccactctccagaagttctggaagccaagtgaggtccagggccttggcaagtcgagtgtgtgaggtaggaggcgatactgtgcacattcagccttggagtcgggtttgccagcggttcccctgacccctacgtggtcttctcagctccatagtgcagaggaggagcctctgcaggcccagaaatttccctgcccacctcccctaccaggtagagatgttagggttcaggcctgagcccctgctgctatccctccttcaaaggaggagatcaaggagcttagggtcccccgcacaggccaccccagggtggggttcttcctttgaagggaattgctttggggtggggtcggttctatctgctcactctgtggctgacagtttctccaaggggctgcaggtgtcagctgtctgagcccgggctgagctctgaaacgtgcctactcaaacttcccgtggggtaggggaggcccagaaccaccctctgagagtggcaaaaagtggtcctggagccaggggaaatgtggatggggtagagatggcatcgatgctcctagactttgcttcccagagaacagccccaaagacccctcccctgctccccatcctcaaaccctgcccccagccccctttgcagagggcgtcagtgctgccaaagatacttctggtaaggaggccaccgcagagttgcacctggggctttcaactttcctgcaaccctggctgccctaggcctctcagatctgtaactttaaacagagagcaccagttgggcaatagaatgagcatttcatactgcataaaaattagtgaggtggtaagaaccatcgactatctcaggcattactacatggcgtttccaattttttttttttaatatacatgcaaggcacattgatataaaaatagatctctggccaacaaatatattaaataagcaaattaaaattttggctttgcgatattggcaacatcaaaatataaccagatagctttgg

SEQ ID NO: 310 - DNA, 193 bp, Homo sapiens
  source 1..193: mol_type = genomic DNA
gaggaggagaacggggctgaggaggaagaagaagaaactgccgaggatggagaggaggaagatgaaggggaagaagaaggatgaggaagaagaagaagaggatgatgaagggcccgcgctgaagagagctgccgaagaggaggatgaagcggatcccaaacggcagaagacagaaaatggggcatcggcgtga

SEQ ID NO: 311 - DNA, 2456 bp, Homo sapiens
  source 1..2456: mol_type = genomic DNA
ggatcccaaacggcagaagacagaaaatggggcatcggcgtgagcccctgccaacaggctggggttgggaggcctctctgggcctggaggtgggggtgggggcagccaagtccagccactcttcacctggctccctgctctgggccctgcaccagagctgccaccctcttctttctccccagccttctcatttccgcctctccagacactgcgccctccaccctcactctgccattgttccacctcctgacctgctccatctgagctctccagctggcccccaattgctcctctctctctttgctctctttctccctcccctaccagcctcattcttcctccggtagcctctcccacctaacctctgcatcccccagcctcatgtcctgccccatccctatcctgcctgatccctggatctccctcagatcccctcttctcaaacagcgccaggccggggtggggccggggttggggccgagccccacagctgcccccctcccctccctttttgtataatttaataaagaaatggtcgcgcttctgtttttaacctgtctcctgctttcccgggggctccagtcagtgcgacaaaagggtagagagggaggagggtggctgacctcccattctgccaggacccttccctttgatacaaaagcagcagcacactcctaccacccacgactcaagaagacaagatcagccctccaggggtggcctggagcccagctcttctgagagctgacttatgtcctgttttggattctcagggatgctcctccccagcaatacacacttccctgctaacccacacaagacctagacactgctgagcccagtggaaagctaccagctctattgtgctgaaccaggcacagagaaacagcccagaaacaggaagtcctgcccctgagctgggagagggctggtagtcctgggtctcagcctgctaggatctggagtgagctgcctggcatcagaaatgccctctgcctggcatggaagcgagctccttccagtcaagcagggaaatcctgggtctcgtgtcatccccaggtcggctgtatgtttcgtcacctgccctccaactctccctgggctgggctggggagcgggagttgtatgactcttgtcagtcttgggccttccctagtcccactgcctctgtttcccctgagtttgatctctgttaggagatttctaagatttctgcatttctccatctccgcatggcccttgccttctcctcttagttcggaacctctggactggaggatgcttctgagggcatggggacctgagttggagtggggaggggtgttgactcatcaacagcatcatttctcccaggagacgcccgtaactcatccaaggtcaggactacacactgtcacgcacacaggtacactcacagcttttatcttcacgctccctaaccttggcaaattgtacaatttctttgaagctcagtttcctcatctgtacaatggggaaaagcattagatttcatgaattactataagtaaagtgtccaatacagtgcttagcacgtaatgaagcctcaatacaatgttagttattctccatgccccacaaagctggcatgcctagcctcagacctaccattttttggggtgcagtaaggcttcctgtccaccatgttcccagggacattgtactgatgggtggaaaggcaggtctaaaggggtcacgaagttctgggaggttaagggaacgaggaaggagattgagcaacaaggaaagagcttgccaagaaggaggtgtgaatattgggactgaggaggcagcttagagatgggcaagggggcagttccaggcagaaatggttcgtggaggcagaaggtccctgggagagggagcagtctggagggtggggcaggggcgaggagggggaggtggggagacccaggactgaggaagtaaacaaggggagcgccaccacagaggtggagaggtggagggtgctgctgctgggaatcaaccccctcagactttccactgcgaagcgaaaccgtaagccctggggtgcggggggcgggccgggaggaggggaagtggggaaggtggagggaaggccgggcacaggggtgaaggcccagagaccagcagaacggcatcccagccacgacggccactttgctctgtctgctgtccgccacggccctgctctgttccctgggacacccccgcccccacctcctcaggctgcctgatctgcccagctttccagctttcctctggattccggcctctggtcatccctccccaccctctctccaaggccctctcctggtctcccttcttctagaaccccttcctccacctccctctctgcagaacttctcctttccccccaccccccaccactgccccctttccttttctgacctccttttggagggctcagcgctgcccagaccataggagagatg

SEQ ID NO: 312 - DNA, 1578 bp, Homo sapiens
  source 1..1578: mol_type = genomic DNA
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctgctgtttctcacccttggtgtcctttctctgctccttttggtgactggagcctttggctttcacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctctga

SEQ ID NO: 313-314 - skipped (2 entries)

SEQ ID NO: 315 - protein, 360 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITGQPQVGKE

SEQ ID NO: 316 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 317 - protein, 444 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 318 - protein, 360 aa, Homo sapiens; identical to SEQ ID NO: 315

SEQ ID NO: 319 - protein, 525 aa, Homo sapiens; identical to SEQ ID NO: 316

SEQ ID NO: 320 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLTLGVLSLLLLVTGAFGFHLWRRQWRPRRFSALEQGIHPPQAQSKIEELEQEPEPEPEPEPEPEPEPEPEQL

SEQ ID NO: 321 - protein, 63 aa, Homo sapiens
RRRTGLRRKKKKLPRMERRKMKGKKKDEEEEEEDDEGPALKRAAEEEDEADPKRQKTENGASA

SEQ ID NO: 322 - protein, 525 aa, unidentified; identical to SEQ ID NO: 320
  source 1..525: note = LAG-3 polypeptide sequence

SEQ ID NO: 323-324 - skipped (2 entries)

SEQ ID NO: 325 - protein, 128 aa, synthetic construct
  REGION 1..128: note = Synthetic polypeptide
EVQLVESGGGLVQAGGSLRLSCAASGRTFSDYVMGWFRQARGNEREFVAAISESGGRTHYADAVKGRFTISRDNAKNTLYLQMNSLKPEDTAVYYCATTLLWWTSEYAPIKANDYDYWGQGTLVTVSS

SEQ ID NO: 326 - protein, 127 aa, synthetic construct
  REGION 1..127: note = Synthetic polypeptide
EVQLVESGGGWQPGGSLRLSCAASGRTFSDYVMGWFRQAPGKEREFVAAISESGGRTHYADSVKGRFTISRDNSKNTLYLQMNSLRPEDTALYYCATTLLWWTSEYAPIKANDYDYWGQGTLVTVSS

SEQ ID NO: 327 - protein, 441 aa, synthetic construct
  REGION 1..441: note = Synthetic polypeptide
EVQLVESGGGLVQAGGSLRLSCAASGRTFSDYVMGWFRQARGNEREFVAAISESGGRTHYADAVKGRFTISRDNAKNTLYLQMNSLKPEDTAVYYCATTLLWWTSEYAPIKANDYDYWGQGTLVTVSSGGGGSGGGGSGGGGSGGGGSGGGGSGGGGSGGGGSEVQLVESGGGLVQAGGSLRLSCAASGRTFSDYVMGWFRQARGNEREFVAAISESGGRTHYADAVKGRFTISRDNAKNTLYLQMNSLKPEDTAVYYCATTLLWWTSEYAPIKANDYDYWGQGTLVTVSSGGGGSGGGGSGGGGSGGGGSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 328 - protein, 279 aa, synthetic construct
  REGION 1..279: note = Synthetic polypeptide
DVQLVESGGGVVQPGGSLRLSCAASGRTFSDYVMGWFRQAPGKEREFVAAISESGGRTHYADSVKGRFTISRDNSKNTLYLQMNSLRPEDTALYYCATTLLWWTSEYAPIKANDYDYWGQGTLVTVSSGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 329 - protein, 442 aa, synthetic construct
  REGION 1..442: note = Synthetic polypeptide
DVQLVESGGGVVQPGGSLRLSCAASGRTFSDYVMGWFRQAPGKEREFVAAISESGGRTHYADSVKGRFTISRDNSKNTLYLQMNSLRPEDTALYYCATTLLWWTSEYAPIKANDYDYWGQGTLVTVSSGGGGSGGGGSGGGGSGGGGSGGGGSGGGGSGGGGSEVQLVESGGGVVQPGGSLRLSCAASGRTFSDYVMGWFRQAPGKEREFVAAISESGGRTHYADSVKGRFTISRDNSKNTLYLQMNSLRPEDTALYYCATTLLWWTSEYAPIKANDYDYWGQGTLVTVSSGGGGSGGGGSGGGGSGGGGSGGGGSGGGGSGGGGSEVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 330 - protein, 524 aa, synthetic construct
  REGION 1..524: note = Synthetic polypeptide
MWEAQFLGLLFLQPLWVAPVKPLQPGAEVPWWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 331 - protein, 749 aa, synthetic construct; identical to SEQ ID NO: 184
  REGION 1..749: note = Synthetic polypeptide

SEQ ID NO: 332-400 - skipped (69 entries)

SEQ ID NO: 401 - protein, 1288 aa, synthetic construct
  REGION 1..1288: note = Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 402 - protein, 119 aa, synthetic construct
  REGION 1..119: note = Synthetic polypeptide
QVQLVESGGGLVQAGGSLRLSCAASGIIFGRNAMGWYRQAPGKERELVAGITRRGSITYYADSVKGRFTISRDNAKNTVYLQMNSLKPEDTAVYYCAADPASPAPGDYWGQGTQVTVSS

SEQ ID NO: 403 - protein, 1288 aa, synthetic construct; identical to SEQ ID NO: 401
  REGION 1..1288: note = Synthetic polypeptide

SEQ ID NO: 404 - protein, 134 aa, synthetic construct
  REGION 1..134: note = Synthetic polypeptide
MAQVQLVETGGGLVQPGGSLRLSCAASGFTFSSVYMNWVRQAPGKGPEWVSRISPNSGNIGYTDSVKGRFTISRDNAKNTLYLQMNNLKPEDTALYYCAIGLNLSSSSVRGQGTQVTVSSGGLPETGGHHHHHH

SEQ ID NO: 405-500 - skipped (96 entries)

SEQ ID NO: 501 - DNA, 822 bp, unidentified
  source 1..822: mol_type = unassigned DNA; note = PDCD1LG2 polypeptide sequence
atgatcttcctcctgctaatgttgagcctggaattgcagcttcaccagatagcagctttattcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaagtcaaagcttcctacaggaaaataaacactcacatcctaaaggttccagaaacagatgaggtagagctcacctgccaggctacaggttatcctctggcagaagtatcctggccaaacgtcagcgttcctgccaacaccagccactccaggacccctgaaggcctctaccaggtcaccagtgttctgcgcctaaagccaccccctggcagaaacttcagctgtgtgttctggaatactcacgtgagggaacttactttggccagcattgaccttcaaagtcagatggaacccaggacccatccaacttggctgcttcacattttcatccccttctgcatcattgctttcattttcatagccacagtgatagccctaagaaaacaactctgtcaaaagctgtattcttcaaaagacacaacaaaaagacctgtcaccacaacaaagagggaagtgaacagtgctatctga

SEQ ID NO: 502-510 - skipped (9 entries)

SEQ ID NO: 511 - protein, 273 aa, synthetic construct
  REGION 1..273: note = Synthetic polypeptide
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 512-520 - skipped (9 entries)

SEQ ID NO: 521 - DNA, 2097 bp, unidentified
  source 1..2097: mol_type = unassigned DNA; note = PDCD-1 nucleic acid sequence
gctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaa

SEQ ID NO: 522 - DNA, 864 bp, unidentified
  source 1..864: mol_type = unassigned DNA; note = PDCD-1 nucleic acid sequence
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctga

SEQ ID NO: 523 - DNA, 597 bp, unidentified
  source 1..597: mol_type = unassigned DNA; note = PDCD-1 nucleic acid sequence
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgaggctaa

SEQ ID NO: 524 - DNA, 867 bp, unidentified
  source 1..867: mol_type = unassigned DNA; note = PDCD-1 nucleic acid sequence
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctga

SEQ ID NO: 525 - DNA, 867 bp, unidentified
  source 1..867: mol_type = unassigned DNA; note = PDCD-1 nucleic acid sequence
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagccgacggccctcggagtgcccagccactgaggcctgaggatggacactgctcttggcccctctga

SEQ ID NO: 526 - DNA, 366 bp, unidentified
  source 1..366: mol_type = unassigned DNA; note = PDCD-1 nucleic acid sequence
caacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagggacaataggagccaggcgcaccggccagcccctgaaggaggacccctcagccgtgcctgtgttctctgtggactatggggagctggatttccagtggcg

SEQ ID NO: 527-530 - skipped (4 entries)

SEQ ID NO: 531 - protein, 287 aa, unidentified
  source 1..287: note = PDCD-1 polypeptide sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLEDPSAVPVFSVDYGELDFQWREKTPEPPVPCVPEQTEYATIVFPSGMGTSSPARRGSADGPRSAQPLRPEDGHCSWPL

SEQ ID NO: 532 - protein, 198 aa, unidentified
  source 1..198: note = PDCD-1 polypeptide sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARG

SEQ ID NO: 533 - protein, 288 aa, unidentified
  source 1..288: note = PDCD-1 polypeptide sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 534 - protein, 288 aa, unidentified; identical to SEQ ID NO: 533
  source 1..288: note = PDCD-1 polypeptide sequence

SEQ ID NO: 535 - protein, 122 aa, unidentified
  source 1..122: note = PDCD-1 polypeptide sequence
NTSESFVLNWYRMSPSNQTDKLAAFPEDRSQPGQDCRFRVTQLPNGRDFHMSVVRARRNDSGTYLCGAISLAPKAQIKESLRAELRVTGTIGARRTGQPLKEDPSAVPVFSVDYGELDFQWR

SEQ ID NO: 536-540 - skipped (5 entries)

SEQ ID NO: 541 - DNA, 867 bp, unidentified
  source 1..867: mol_type = unassigned DNA; note = PD-1 nucleic acid sequence
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggtcagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctga

SEQ ID NO: 542 - DNA, 27 bp, unidentified
  source 1..27: mol_type = unassigned DNA; note = PD-1 nucleic acid sequence
atgcagatcccacaggcgccctggcca

SEQ ID NO: 543 - DNA, 1563 bp, unidentified
  source 1..1563: mol_type = unassigned DNA; note = PD-1 nucleic acid sequence
atggggaagcccagttcaatggatactaaattcaaggatgacttatttcggaagtacgtgcagttccatgagagcaaagtggataccaccaccagcaggcagcggcctggcagcgatgagtgcctgcgggtggcagcctcaaccctgctcagcctgcacaaggtggatcccttttatcgattccggctgatccagttctatgaggtggtggagagctccttgcgctcgctcagctcctctagcctgcgggctctgcacggcgccttcagcatgctggagacggtgggcatcaacctcttcctctacccgtggaagaaggaattcagaagcatcaagacctacacgggcccttttgtttattatgtcaagtcgacattactggaagaggacatccgagccatcctgagctgcatgggctacacacctgagctgggcactgcatacaagctcagagagctcgtggagaccctccaggtgaagatggtctcctttgagctctttctggccaaagtcgattgtgagcagatgctagaaatccactcacaagtgaaggacaagggctactccgagctggacattgtgagcgagcgcaagagcagtgcagaggatgtgcgcggctgctcggacgccctgcggcggcgggcagagggccgggagcacctgacggcctccatgtcacgagtggcactccagaagtcggccagcgagcgggcggccaaggactactacaagccccgcgtgaccaagccctcgaggtcagtggatgcctatgacagctactgggagagccggaagccacccctgaaggcctcattgagtcttcggaaggagcctgtggcaacggatgtgggggacgacctcaaggatgagatcatccgcccatccccttcgctgctgaccatggccagctccccccacggcagcccggatgtgcttccacccgcctcccccagcaacggcccggccctgctgcgcggtacctacttctccactcaggatgacgtggatctgtacacagactctgaacccagggccacctaccgtcggcaggatgctctgcggccggatgtgtggctgctcagaaacgatgcccactccctctaccacaagcgctcgccccctgccaaagagtctgccctctccaagtgccaaagctgcgggctgtcctgcagctcctccctctgccagcgctgtgacagcctgctcacctgtcctccagcttccaagcccagcgccttccccagcaaggcctcgactcatgacagcctggcccacggggcatctctgcgggagaagtacccaggccagactcagggcctcgaccgcctcccgcaccttcactccaaatccaagccctccaccacgcccacttcccgctgtggcttctgtaaccgcccaggcgccaccaacacctgcacccagtgttcaaaagtctcatgtgacgcctgcctcagcgcttaccattatgacccctgctacaaaaagagtgagctgcacaagttcatgcccaacaaccagctgaactacaagtccacccagctctcccatctcgtgtacagatag

SEQ ID NO: 544 - DNA, 201 bp, unidentified
  source 1..201: mol_type = unassigned DNA; note = PD-1 nucleic acid sequence
atgcagatcccacaggcgccctggccagtcgtctgggcggtgctacaactgggctggcggccaggatggttcttagactccccagacaggccctggaacccccccaccttctccccagccctgctcgtggtgaccgaagggnacaacgccaccttcacctgcagcttctccaacacatcggagagcttcgtgctaaactga

SEQ ID NO: 545 - DNA, 50 bp, unidentified
  source 1..50: mol_type = unassigned DNA; note = PD-1 nucleic acid sequence
cagggaaggtcagaagagctcctggctgtggtgggcagggcaggaaaccc

SEQ ID NO: 546-550 - skipped (5 entries)

SEQ ID NO: 551 - protein, 288 aa, unidentified [sequence omitted]

SEQ ID NO: 552 - protein, 9 aa, unidentified
  source 1..9: note = PD-1 polypeptide sequence
MQIPQAPWP

SEQ ID NO: 553 - protein, 520 aa, unidentified [sequence omitted]

SEQ ID NO: 554 - protein, 66 aa, unidentified
  MOD_RES 48: note = Any amino acid
  source 1..66: note = PD-1 polypeptide sequence
MQIPQAPWPVVWAVLQLGWRPGWFLDSPDRPWNPPTFSPALLVVTEGXNATFTCSFSNTSESFVLN

SEQ ID NO: 555-560 - skipped (6 entries)

SEQ ID NO: 561 - DNA, 1119 bp, unidentified
  source 1..1119: mol_type = unassigned DNA; note = LAG3P nucleic acid sequence
atgaggaagctacaaaaggaacgtaaagtttttgaaaagtatactacagctgcaagaacttttccagataaaaaggaacgtgaagaaatacagactttaaaacagcaaatagcagatttacgggaagatttgaaaagaaaggaaaccaaatggtcaagtacacacagccgtctcagaagccagatacaaatgttagtcagagagaacacagacctccgggaagaaataaaagtgatggaaagattccgactggatgcctggaagagagcagaagccatagagagcagcctcgaggtggagaagaaggacaagcttgcgaacacatctgttcgatttcaaaacagtcagatttcttcaggaacccaggtagaaaaatacaagaaaaattatcttccaatgcaaggcaatccacctcgaagatccaagtctgcacctcctcgtgatttaggcaatttggataagggacaagctgcctctcccagggagccacttgaaccactgaacttcccagatcctgaatataaagaggaggaggaagaccaagacatacagggagaaatcagtcatcctgatggaaaggtggaaaaggtttataagaatgggtgccgtgttatactgtttcccaatggaactcgaaaggaagtgagtgcagatgggaagaccatcactgtcactttctttaatggtgacgtgaagcaggtcatgccagaccaaagagtgatctactactatgcagctgcccagaccactcacacgacatacccggagggactggaagtcttacatttctcaagtggacaaatagaaaaacattacccagatggaagaaaagaaatcacgtttcctgaccagactgttaaaaacttatttcctgatggacaagaagaaagcattttcccagatggtacaattgtcagagtacaacgtgatggcaacaaactcatagagtttaataatggccaaagagaactacatactgcccagttcaagagacgggaatacccagatggcactgttaaaaccgtatatgcaaacggtcatcaagaaacgaagtacagatccggtcggataagagttaaggacaaggagggtaatgtgctaatggacacggagctgtga

SEQ ID NO: 562-570 - skipped (9 entries)

SEQ ID NO: 571 - protein, 372 aa, unidentified [sequence omitted]

SEQ ID NO: 572-583 - skipped (12 entries)

SEQ ID NO: 584 - DNA, 906 bp, unidentified
  source 1..906: mol_type = unassigned DNA; note = TIM3 nucleic acid sequence
atgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatag

SEQ ID NO: 585-589 - skipped (5 entries)

SEQ ID NO: 590 - protein, 301 aa, unidentified
  source 1..301: note = TIM3 polypeptide sequence
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 591 - DNA, 733 bp, unidentified
  source 1..733: mol_type = unassigned DNA; note = TIGIT nucleic acid sequence
atgggctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatgcaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggtt

SEQ ID NO: 592 - DNA, 733 bp, unidentified
  source 1..733: mol_type = unassigned DNA; note = TIGIT nucleic acid sequence
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggtt

SEQ ID NO: 593 - DNA, 735 bp, unidentified
  source 1..735: mol_type = unassigned DNA; note = TIGIT nucleic acid sequence
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttag

SEQ ID NO: 594 - DNA, 735 bp, unidentified; identical to SEQ ID NO: 593
  source 1..735: mol_type = unassigned DNA; note = TIGIT nucleic acid sequence

SEQ ID NO: 595 - DNA, 21389 bp, unidentified
  source 1..21389: mol_type = unassigned DNA; note = TIGIT nucleic acid sequence
agtcggctactttcagtggcagaagaggccacatctgcttcctgtaggccctctgggcagaagcatgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttagcaaccagaggcatcttctggaagatacacttttgtctttgctattatagatgaatatataagcagctgtactctccatcagtgctgcgtgtgtgtgtgtgtgtgtatgtgtgtgtgtgttcagttgagtgaataaatgtcatcctcttctccatcttcatttccttggccttttcgttctattccattttgcattatggcaggcctagggtgagtaacgtggatcttgatcataaatgcaaaattaaaaaatatcttgacctggttttaaatctggcagtttgagcagatcctatgtctctgagagacacattcctcataatggccagcattttgggctacaaggttttgtggttgatgatgaggatggcatgactgcagagccatcctcatctcattttttcacgtcattttcagtaactttcactcattcaaaggcaggttataagtaagtcctggtagcagcctctatggggagatttgagagtgactaaatcttggtatctgccctcaagaacttacagttaaatggggagacaatgttgtcatgaaaaggtattatagtaaggagagaaggagacatacacaggccttcaggaagagacgacagtttggggtgaggtagttggcataggcttatctgtgatgaagtggcctgggagcaccaaggggatgttgaggctagtctgggaggagcaggagttttgtctagggaacttgtaggaaattcttggagctgaaagtcccacaaagaaggccctggcaccaagggagtcagcaaacttcagattttattctctgggcaggcatttcaagtttccttttgctgtgacatactcatccattagacagcctgatacaggcctgtagcctcttccggccgtgtgtgctggggaagccccaggaaacgcacatgcccacacagggagccaagtcgtagcatttgggccttgatctaccttttctgcatcaatacactcttgagcctttgaaaaaagaacgtttcccactaaaaagaaaatgtggatttttaaaatagggactcttcctaggggaaaaaggggggctgggagtgatagagggtttaaaaaataaacaccttcaaactaacttcttcgaacccttttattcactccctgacgactttgtgctggggttggggtaactgaaccgcttatttctgtttaattgcattcaggctggatcttagaagacttttatccttccaccatctctctcagaggaatgagcggggaggttggatttactggtgactgattttctttcatgggccaaggaactgaaagagaatgtgaagcaaggttgtgtcttgcgcatggttaaaaataaagcattgtcctgcttcctaagacttagactggggttgacaattgttttagcaacaagacaattcaactatttctcctaggatttttattattattattttttcacttttctaccaaatgggttacataggaagaatgaactgaaatctgtccagagctccaagtcctttggaagaaagattagatgaacgtaaaaatgttgttgtttgctgtggcagtttacagcatttttcttgcaaaattagtgcaaatctgttggaaatagaacacaattcacaaattggaagtgaactaaaatgtaatgacgaaaagggagtagtgttttgatttggaggaggtgtatattcggcagaggttggactgagagttgggtgttatttaacataattatggtaattgggaaacatttataaacactattgggatggtgataaaatacaaaagggcctatagatgttagaaatgggtcaggttactgaaatgggattcaatttgaaaaaaatttttttaaatagaactcactgaactagattctcctctgagaaccagagaagaccatttcatagttggattcctggagacatgcgctatccaccacgtagccactttccacatgtggccatcaaccacttaagatggggttagtttaaatcaagatgtgctgttataattggtataagcataaaatcacactagattctggagatttaatatgaataataagaatactatttcagtagttttggtatattgtgtgtcaaaaatgataatattttggatgtattgggtgaaataaaatattaacattagtttcatgcttctttttatctttttaacgtggctactggaaagtctaaaattgcatatgtggctctcatatttctatgtgactgtgctgctctagtagctaaacccaagccaaactgatacccagggagggtgtctatctatgtctattggtctccatttctttttctttcaaaatagtgtttgaggaagattttcctgcttatgatgtcttttgggaaatatgtagggaagcatcacagagaagattgaggaaggggaacactcagtgctgacaggaagcgatgccaggcttttctgggtcccctcgtggattccctaacaggaggagcgaatagacaaatctatgaatgagcattcaaggaggaatacattactgattccctgaccacattctaagcctgggcgtacaagtctgatctataatcctgagaattatactaaagaaacgtgagaaactttgagtcatggagctttgagatagatggtcttcatgagtgaacaggacctcactatgcaattttatttcctgccttttccccttttcatttctcaccactgtgagccccagaaatgagtcgtcatacactcaggttcattcagattccatcagtgctgagctggacaggcagggaggctgggtgtgaggtccaccaatgacagcattctggtgggcacacctcaccccacagagagggggcaatgggcagtgtggctgtcatttgtggggtgtgagttttgtttagtctcctgattctttcaggtgctgcttcatagctgtaactggtagtttcccacacttactccaagtcacacctgtcaagaggtacaggggagctttttgtgtggcaagagcctgagctttctggaagtcacaacgatatactcaatgtttgaggtaatttatttgcataaggtagtgtaggacaatgctatccaaagaactttctgcaattatggaaatgctctatgtctgtcccatccagtatggtagccattagctgcatgtggctgttgagcacttgaaatttggctaatgtgacaaagaaactgaattttaaattttatttcattgtaattaacaaatttaaatgtaaatagcaacatgtgactggtggctagtatattggacattccaattctagtaattgccttgcatatagagaaaacacagacagctgtgtttataggacattggatggctcgccagttgttatgcatttgaaatggctatcaccaggaagaaacttgaaggttctcctctactctcctatatccatcctctgatcgagacaaaccatgaggtcaaagcaggctgtacatttatttatttcaggttttttcaaaaaaaaaaatctgaggcagctaagtggtccagttcatgtaaatagggccaaaattgtcaagaaatttgcaactcataggggttatgcagaaatagactgtcatcacaggacttaccatagactatattcattctgacttcatgtaggctgtgaagagcaccttctctgggactaaaactctgatggacttcacagagatcaaaaaagaagtaatcatggagtgtagtggacagggaatttagaaacttggtccaagtgctgagctctgcaattagtttcacgactttggataagccacttaacctatcaaagactcataaatatttcctcatttataaaatggtaatattactatctttctaagattcttaagaagatgaggcaggaacgtggctgactgctttgtgagctgcaagatgctgggttaatattattgtgttctgccccaggaacacgtgcaccaggtaatgctaatgatgccacagaagttttcatggaggcaacaaaactggcttggagcaactgaggtacagaagaaaacttaagagtgtgtggatagtgcaaagagagatccaagactttgggtgaagctggctggcattgttaagaagatgttggttcacagaaatgatatagagacaaattatgcatcatgaaaatattttcttcctctttatacttctagactatcaatcaggttgacttagaagttcagcaattgagttgcaagaagtaaatatttaaattcacagtggtaatgttgaaatctcaggaatttattgacatcctttcagtctagaggtctcttgatgactctccccatcccaagctcatgcctggatatttttgtgctcctcacactggtggaaatgtctgtccacactcagcttctccaactcaaagcttagtgttcttgcgagtccccaaactagaccgcttattctttggaccctttcttgactcactctagccattatcaacttcctgttagtttagaaactcccttggcattgagagtctgaatggcacaatctagctcttgattttatacaaacctctcttcatgtctagttgttagattttaagccttctcatggctgaaataatttcctctcttttattcatacattgcacagtgctgactgaaatacagaagacattgtgaatttaatttgtgttgacttcaccaaggacaaacaagatataagttttcttcaccatattagcactttctgtaagaatgatgagatcagagagctctttatggccagggtcactataacaaagaatctatgggccaaataggagtcccttatgagtctccaaagtcattaaagaggggcatgttttgtgccatgaactttccttttttgcatatggagctgagaaacccaaaagttaaaaaggagactcagtgatagattcatggataacagagatgggtggaaacaagaataggtgggcatagtgttataatatgggattgattatttccacagcaggcagactgtgatttgcatgtggaaaaggaaaaaatgtgaatcaatttgtagagcaataagtgatccttgtcatttttgaggtttgaatatgtataagacaatacaccaatttgctttatatttcctcaaagtgacttcgacatcaccaatcctgtctgtagttcctacttcttgtattgagaagactaatatactcagagtttttctggccttgtaggtagagtcctcagtttggataaataatctcagattagacatagaatacaatgtgtgaaagaatgtggagaactgcctaaaacagaaactacagtataaagaagacacaataggaaaccaagagaagagtttttttttttttaattattccttcatattcaaacttcacaaacagtgtgaacttgtacaatacctcggaaagtgaaacttacaaaaaaagtgctggtaacatttaaaaaaaaaacaacaaaaaccccaaaaaaacaaacatcattcttagcaacatcaattactcttccacacaaaacagaaaccttgtaaaatttattttcgtatttttaaggcgtaatacttccgtataaagtatatgcaagagataaaacttcacagtattccaaaatgtcacaataataataataatataatagtataatgaagcgctacagttaatttttctttttttgaatgttttttttcctgtttaaataacaaatacaagtcacaggtaaatatacgtgagaaaaatacgaggctaatattaaatggcaggtaaggatactgtcactgtaagaaaaaactggcaggatgtgtgtatttagtctatattttaaagcacttgatagaaaaggcgtatgcaaggtttttccaaacaatttttctgtttgtttgtttgataactgacaacaatttaagagtataatgagaaaaaaaaaggaacaaactccctctcaaaactattgacctgctcatcccaaaccatgtatatgttgatggatggatgtgtgtttttgtggatatatggatttataccgtgcacagagtgatacaagcagtgttccatgcccacctgcaaagattggagggtataacactgcagtcattcagcccaggttcctcttaggaaattaacctttcaatttaaagctctctttccttgcctctccctctttccaaactcttcctcaattcccaggtctttgttctgaaaagacaatctggttagaaaagttgccctccactcataattcactgggaaatggaaactgcataaacagtgacacacagacactggaaacaccacttgaatgaatgaatgaatggatatgtatgtgtgtgtgcatgtgtgtatgttttaggtcacataaacatacagtgtgtgcgtgggtgtgtgtattttaggtctaaacatacagtgtgtgtgtgtgtgtgtgtgtgtgtgtgtgtgtgtgtacacagtagcaattgcaaaaaagggaccatattcctttccatactaagatacaatgtgataagaaacatttctgtgcagggaaaaaggtttcttcgaatcttatcacaacactgcggcgggaaaatcaggaaatgggttcacctttcagcagtcaccacagtaaacttcattgagaacatacatgcccagatatctcttctgtgcacatgtatatgttaacagctagccattcagccaacaggtaacagaaagacagcagatagcaaacacatcaataaggtgctcaaagaaaaaagtggcacccacttctcaaatggatggccacgttaaggtgcttcaaaaaaggttttttgtttttttgttttttttttcagtttttatttcaaacattaagagagtactgattttcacatggtagttctgagtcaggccaaaggttttgaggtttctgacatctttcactgaaaagggctttcactgtagtagttttgttcagtttgttgtgggtgacgagtttaaaaatgtttttcatagccagaaactgaaatcaaatcaacatgactaaaagaaataactgatgaaatggtatatagaacattactgcattcgcatcggatcaagatggtttcgcccatttttcctttttcactaacactgttcctttttttccttgttacaatccattctttatgaacatacagaaatgaccaaagtcactgcaagtagctgtttttattttttgtgatctgttgcaagagtccaaccttgtttcctctgctgctgtttgtgtagcatctggttttgtaatgagcatctggatttgtaatgagcatctgattttgttatgtgcatgtgtgtatatatgcacatatacacacctatacatgtataatatatacactatatatatgtggatacatataggaagtgtgtatacatttatacataatatagtgtatatcgtttcaactggagataaactgaactgggttcagacactagcacatgcttaactttcccctgctccctctgtatattttaaacagtctggagctttaatttattttttaaagtgcattttcaatgcagaaaaactgtaatccactgattccttggaaaacatttttgatgagaaaaatctgacagttctcagccccaagcaagagaaacctgggtttggtcatgctgggggctgacgtggcagtgccaggctcgtgcctgagtgattaatggacattctggactccgggcaccttagcagcagcagcagcacctttatgagaaggtagagatagaaaaggaagaaggaaggaaagctcagatgaggaagaatgaagtattgtgtttacgtatcaaaattgctaactttgactataatggacttgtgcagttcctgttggcctcggctaagagatgccatctagtcccttctccaccaactgacaccaaactccccatggcccctgcatgctcaaacatcacccatatgtccccacgcctaaggctgacttctgtgtcagcttcttgtgggtatgtgagtgccagcatcaagtaggaataaggcaccagagaaatcaaaatgttacaaaaagatgcaaaccccatttcaaagagcctctcccattgctccattaaaatattgcatgtgataccattcgtttgtgtgtacctaaagtgtgtcaaatacagtctctgaaaggaactcgaggcccttcagcaccagagatcttcaggaatgcatcttttttctaaacacacccacaccacccccaaccccccatgcccaccccacaccaaaagcaaaagcaccaaattgggagaacaaggctgcttgccattgctcacctcatttttgaaaaaatcagagaagtctatgaaatacaatgttcaggaaatgctctgaaatcaaacggatttaggctgtcatgtacatggttactcaatgctttaaatgtccaaagagtgtcatgaaggggttgaaatgtttgtgttcaatgtttagggggaatgtgttaaacaaaagttacttctcaaacgatgcccaaatcttccactaacacatcaacctcccctatcactttttaaaaaaattgtaaagaggaagaggttcatctaactataaaaggaaaagatgaaggcacgtgatattattcggtcccacaatccttaagaaaaacggtattttgcaagtttgtgtgtgtgtgtgctcgcatacatgaaacgcgagtccaaggggccattggggcagtagtccaagggaagctggaaaaggggaacaagagagagtttttagacagaagaaaggaaaaaggaagaaatagaggcctctcttcagctttctgtcattgacagtctcaagattcggctgctttagagtccagaaaggaatttgtttgtcttggtctggaaagtgccccagttaaatatctccagttcatcatggtacagcttttggtgtttcccttttcccttccctccagccaaatgattttttaaaaaaatcacagtttttaaaaattattttttcaatgttgaaaaaatatggaaaagctattgaacagccttagggcactgcctctccatcttcacagtgagcagatggatggataatccacagagtcaaagaggaggtgggcaagatcccttgtgctcagtggtctcttgccccaccttcctgagggctggggggccttcacaccacccacagccatttccctcctttgggtccctgcccccctgctcccatccatggaatgtgggctgctgagaaaggcagagccaggctagggaacgatgataacagtaacaggagggtctcagaagcagagggtgaaggagacaggaggcgtgctctcctcaggcagtgttagcagtgtgctctcttggcagcctcatctcaggatgggaaagtgtggttgcccactgagagggtgaagtcccactcacattttacttcgtgagacctcaactctcaggctcaccacctttatgcctctctttgcatcaggttggcccccagtccctaagaaggcaatgatttgagatagagaggcagagcccggctgtgtcatacaaaaaagggcaacgcacagcaggatttcagaaacaaatttaactttggttttccaactttttcaaaccataaaaattaaagcagggcttcggtggctttgataatctgacagctgttatatttttctcatttggcaaggctaaggcagggggtagggggaagagaaaaggggtttcccataattggttgaagacacagggtggtaaaatgcttccacacttaaactcacagtccagtgtttttgacatttcataaatagctttttttctctaaaaaaagaataaaacaagaacaatccctttacttactcatctgaaacaaaataaacagcagtgggtacaaacttcttcttgtaacaattacaaaaaacaacaatataatcccaacttttaacacgcatcttgaatcagtaatttttttttaacctcaaaactgcagcatatccttaagggcaaacttttcgattagtttttttctttttttttttttaaataatgtcttcaccaaaaaaacagtcttgtaccaataaaatgcattcaaaaatagaaaatattacacaacaaaaatatgtatccttcctttccaaaaaagattcttcacaaaaaaatgtagaaaaaattccaacaatttttttcctctcctaaacattaaccttcagtctagggcacaattatttattgatttaaatgtctgtttttgcataaaacatggaagatgcaaaacatttactgtattaggattgtgaagttacagccactcccacccctgctctaaaacaaaacaaaaccagaaaaaaaccagcaaacattaaacaaatgatagagtcagttggctaggaaaaataccgctgttcttccctttactgtgcatttgtatgggtaaacgggccagtttggtattggggaatgttgacgaagggagatcaggtttcaagattggattcaagagcttctttagcatatgctgataccaatgagtcactgacagtccccaggttgtacaactatccattgcaaatcctcatgagaagagaaacacatgaggtagcacatgggaagtcagaggactgaagcgtgtccatgtgtgtactgtacacatacatcttgaaaaaccactttctcaagcatatacatatatatatatatataagcatatacatatatatatacacacacacacacaaatttgttacatagccaatttgtttttaaatattttttctcaaaagggagctcaagcataagttaaaaggtgatgtggaaatccagtggtatgaggaaggctggggttatttcctccatactttcttccacatttaacctgtttgggcattaattgcaataatgacaagatctctgctctcagaggaatgctctgttttctccatccccattgaactaccaggttccttttatattttcagcattgtagtttaaactatgtatttttcttacttgatgatattaatctttcatgtgggatgtagtttgtatgtgtattcagtacaaatatatttttttcatagctgttttcccctgttgcttatttgctgtttttttccctcatggttttgctttgttttgtttctgattttttaaacagtgaaatatgtccaattttatttccagtgcattaaacaaattttaaattaaaagaagaaataaacgtttccccccatctttctaaaggaaatgactaacaaaagtacaatacaatagaatcaggcattgccaagaatagcatgatgtgcaacctcaattatatcaaagaaaacaaatagacaaacaagaattatagaacataagtatgaaggagccaagattacataattccaactatggtatgattggcaagcactggttaaaaaacaggtaaggctactcttagatttctaactcaacatagttaaccactgctcttctgtcttttccttcaacttttaatcttttactccctagttctttatatctttaaaatgaacaaatttgtttttatatttttggtctacagtagaattacatattaataatataataaaaagatatttcattaacaggttggtactgcatccatgtggggacaatagaaaaaaagtactcaaatagacatagcaaatggactctattcttataaaaataaccacaaatataaagaaattataatcagggcaacagattaaaatgaaaaggggtatagaaagatatattttagataatataaactcctgacaagctatgccctagaattgcagcctccaaaccttgcccatgatttccacctttccatagcctcacttcaagttcaaatggaaatgaaaacaaaataaagtgaacaggtggttgagccagccaaagcctccaagttatgttaggtggacagtaagaccaggtggacagcatttgagaaactggctggacacaggggagctgctactggtatttatatttaatttggaggaatttccagcccactgataaagagagaaaccaaggaaccaaatgacttttctagtaacatataatatatgagatgaaatataattctgttagagaggacatggtggtgagatgggaaagaaaagagaagagaaacaggcagacttcgggcaccatctccctttgttttctacaatgcattggtttccttctgttgcggagcattctttaggattagctacagaccccaatgtcctctgagtaaatatatgtcagctcagctcctgacatgatatccatgcttattggcctcctggagggctgggtggttctggagggtccctcccctgctatcactgcccactggcccttgccttcaataaagcagagaaggtgtgcaaagtttcaatggcaggggctctttacgttaactatacctaactgatgactaattcacacagatggttccatatggaaacaaaaaataaagagaaagaactggtaagagttgagctcggtagccaactagtgtgaagttttcttccaggaaaaaaagaaggagaaaaagaaatggtatctgtcagaaaatactgcttttggtttcttttaactttgagctgtccttggtttttctctcagtcagttgctccagtttaaaggagagtttagaatctagtgcaaatatcatcaaatcacagcattccagagcaatatcctgtcccttcatcctaagtggatactgtcccttcaaacctcctcagaagactggaggcaatggtacagaaaagcgtaactgaataaattattaatctagtactcactacgacaagaaaggcactatcacaacagatcagcctccaagcattttccttgccatgggcatgggcgtttcaaccactccagaaatcacaacaaaatccagactgtggctttttcaacacttgaattcattggccagttgtctgactctcccaccggtcactgctgttggctctggccatacagaaccctgatctgcactgtgtacatgctgccctatgtttcaccagtgacctttgtttggttctaaatgatttagctcactctcttccatggaagtttctttctctccatcagtctccaggtcatgactctattaaagatcatttacagtgtcaagtagtgttgtttatttttttctattttttaaacctctgatcaaatccaaagtcataaagatagttcagtacgtcctctgatgacaaggtctcctgtaaataccattgtgaacagaaggaaaaccatagtgtaatcccttgaccaccaaagcatatcccttgagcctgttgtagtacttttttctgggagagaggaaattgcgatctgaagattaaaatctttggtgacagaactggagtggatatgcaaatttcaaggaagaagttttccaaagcctcaagagaagtatcctaggacagttaatgtgaatatattttaaaaagtcaatcatttctttctatagctttggtttggtgcagtcaatgtcctcttagtgattacccaatacttatttctaagtacctgttaaaagctctggatttagtttcttacttaaaacagagataatgctttgagtgcttaaggaatttcttatgctagtttggatggctctaaatggagtttttctgactaacaaggctgacaactttctgaacaatatttaaacttggaatagtagacttggacggatagtaaagaagagagagaaaaagacaggttatcaatgttgtaatcttagggaaggggcctcaattgagaattttccaaattgatgccccatatgtgacagtgttcagagaaattcaacattagccatttatatgggttgtgatcctttcctactgtgccagaaccttcaaaactgtcactttgagttccagaagagtccttcagcatctttactcttgtgttattatttcccacacaatctattgcttatcctgtgatgaaagattcttttattaacagttactaaagagctgtcaaactctgattacttctaagattaaaaaagataataaaaataaatttaacaaagaaaagttaggaaaaaaccaatttcaaaactaaacttttaggcactattaaggtcttctgcttagtaaggatagtctatgatggagcagctagtatgttctaatccatcctccaatgtccaagttggcctagcttgtttcaaaataaacctttcacttcactggttttcagtgtttattgcactgttccctatctgtcatcttaaaggactttaccacatgcatagccttcctctttggttagcctgatctaggtttcacccagggatggagaagcataatcaggctgaaaagtaattccaatatggacaaggctatgtgtttttgaaaggagcacatcctactgccctctgccacactaaaatgtgcacaagcaaggggctttgtggaggaggccaaggcaaagacttctacatcatatcaattggcttcttcactgttaaaactcttactgagagctcagtcagaaacaagattgggtaaactttttcttaaatgttcttaattgctgtactaactcagcttctttacagtaagaaaacagatcctatcagcatggtggttcctaggcctgatcaagagttgacagagattccacagattctcgccaaccctgctaagatcaagcatgatgaaaataactctgcattttagtgatgctcctacttccataatttgcctttttctttcccatacgtaacacactctctcccagcacaagtaagtacatttcagtagtgcaatgcagtgcagtcatatttagtgcaaaaaaaagccctattataacccaaagctcaactctaaacagctcttacaaaggttaccagtgctacgaaggtgggaaattcagtctatctcacacttatgtgagtcatctcttttgttgctcctgtttgtttcagctcaaagtagagaaactagcatgcacctatgtattttctgtaaaacattaagccaataggcagaaaataaaaggaagtagtctagttaaaatcaagggtattatttagaagaggaaaacagctatttaatgtaaaggtcagtagtggtttttacttaatagttttcatcatgtcagatggctctaatgctaaggggaatgggttcagaaaaagataaagctttcctgtttcttgaaacacggtcctaaatttaacagtgctgggcttttatttttgaatagtgcaatcagctattcctcaagggctacaggcagcagccacctaacataacataaataggtacactaggagcactcacttttctacattgatcactttttccctcacctcattagtctcctattcttttttttttctgactacacataaatacagatatacatacacatacacagaaattctcaaggcccctctcagaatgatcaattgaaaattggtacaaatagctcagtgacacactcacacagaagtacaattattaaatacaaaagtgaattattacagcaaaattaagagtctgcattgtacctcttcttgtgttattccataagaagaaaaaacattcatagtaggatttcttaaggagcttacttctttctaaagattagtctagaattagtttgggatgtggcaatattgagtgattctggaaaaatgatttgtatttgatcatcctcaggaatccgtttcttcagtttgtctgggtcttcaccctccaaaggtaagtctgcttgtctgctcatacccacacatgcatgctgtaagtctcaagagaatcaaagtgtagtttaaaatagattgtgggaaaggaattaagcatagtggggtcagcacactcaaattaccttaaaatatcaagttaccatagattttctctatcatatcagcttctcacctttgtttgccatgatgaagattactaagacaccctgctttcatctactctgaaaaccataattctttcaacaatagtgtaggaacaattttaattagatatatgcaaattacccttaatttatatctcaataggctcctcaggaaaactgaggttcaaatcaacgggcctaaagaagaagtttagcagctgaaaatacctttgagaattttctcctgtaaatctacagctggttccaaattaaagtttgtcccaaggagagcaagagcagagtaactagcctttgttttcaaagattcccctagttgtgaacatgcataaagcactttgaactacaggaaaatcttatctcaaatttgaactattattttattttcagtgatctctctgtacactcctctcgggcccggatagcacattaggtatagaagtcaggagtttctacaatttctctagaagtgtttggcagcatgaagagaaatctttaatcacaaagaagtatagttaacttactaattctttgaattgtaataatgagagataggcaagaacaacaaggggtgagtagtgctaactttctccggtaaccttgacaactggctttaggaatccagacctgtatcaaatttggtcccatcacattgtcctgttccctctctttgtttttctctgttcacttaattgaaattgcaattattttaaagattgagccaagaatgtcaattatagcacaaaagaaaggttacaaaaacgttgtcaggctatctgttaatcgagttgagttgaaaacagtatgctttcaaatgaagtactacaatttccttttttaaaaaaatagtttctaatttgttttttccttcttttgtggtggaggggtatagatgggtgaagtactggcaatgcagtgccaatctccctctgttactgttccctctagtgcctggggttggggcaggtgcaggggtgtggggaaggagagagaagcaatggggaaacaggtgtttctctacttaagacttcatgaacacatacagtcttgaaatggctagaaaagcaacaactacaatgataaaaatcaacagatcaattgattttggcatttcggcagcaccccttccttttctttcctgggtatcttgcagtagatattaattagaaatacccaaagaatttaattaaactaatttattcccagccttattccaaaaaggatttgaggaggcaataaggatttgtagtacaaaatgcatggtcttcatctgcaatcaataaaaaaatctattctttggccccagacctgttagtttaggtaaatacggtttctttctcaaggaacaggtttttgttttaactgttctatcctcaaagtaaagagagaatttctctcttaaaagctcatgagtgctatttgaaatggaagataagattttttggaggaaaaattccttcatggtaggatggaaggcttttctttctaaggggaaaataatacagtaagggtgagcagttatttgactgtccatggacagtaacaaattaagaaataaaaatcctttactatcttgagaagaaagaaagggaaacaaataaatcaacaaaaaaaccttactaaaatacacattcctccagtttcagatttctaaaaagtaataattatgcttaaatcaaggtttatagatagcaaacaactgtttgaatgtgtgaaagtattatccctttgctctaccatgtttcactttctttgaaggaattgcatctatgcacacttatcctgaagtccagtgcagaaaataaacttgtttgctaccagcctcaatctcagaacatttcctaagctatatttgtaaattctagagctacatatctgtaaaataagatgcagactttctgaaatgcattgtatgtgtgtgtgttgcagagggagaggggagcaaagcaagcttttcctggagaaaagaagaaaaaaaaatcttcctatcctcacgaatatatactatagtgtttctgtctattattttgatgcattagttactgttagggctattgatgctcctgacaggtgtcagcttggaaagacaggaggctcataagcaataataaattcacaccctggagtctctagaatatcaaacaggatttagacattaatctgagactaacacagaggttgtggtgagaaattctgggtcttggggttggaatacttcttggggaactagaaagaacagcatcaagaaggcatcccagattgaggaccaagtaagatctatcagaaccagagcctctcctcctccatattatactttaaatgacagcattttaggatttaaaagagtctttcttcctttggaagccagtgcctaaggagaggattactttagataaagagtactttaagaaaaaaaaaataccaggagttagaatggacaaagaaaaagaaggatataggattgattgggagaacattccaagtgaagatcatagtcctaagcaatgtaaatattgcctttaagagatgttgttttcctactagatgcatgcctcataaaggctttcaaaactgctatttatttcattgttctctatggtgtgagttgatgttataaactggtttgattaagataaaagctgggtcaaatctcaagtcaaataaaaatacatttctgatattaaatgttgattctttttgtcaaagggtaaagcaagaactgaaagcataccgcctatgcactcaaaaccacatagtactctaaatgtcacaggacttgtcttcattaaagctgagaggcgatgtggtatcatggcaagaatattggaccagaagtcaggaaagactgtgttacgaagttgttgtgaatgtgatattataagcatcctgaaaaggatacacccagaatggtatcagtcagtcttttgggcaatgctctggtgaccagacagttgtaatccactgcagggagatctagcatgacaaccaaattatatggggcgtatttaggtgctggggagtgcactgcatggctctttaggtatccatttagctctgttattattttatttaagtatatattaagtttatgtttaggcaatctctggttgcaaaaacagacactttcaaaactttatggatcttttcatccattagtaagttactgctcaaagttctaaacaaatcaatgatttttttttcaaaaaggcaataaataaaaaaactcttcctaatttttcctacaatgtgttcaagtggatgtggcttttcttgtttgagaagagctcactaaggcttgaacagttatcaaaattattttgaaatgttactcttttctttagattcctataatctaaagtctgataaacaagtaatttgactagtggaaacagtcaacattaatgattctttttttctccgtcaaaagtgatttactcatatgaagaacctgaacactgtggtatttaaaaaaaaaacagaaaatatttttctcttcatttttggccaaacgtgaaggtaaggggaattaatgacctcagctagtacagttgacatttaaaactgtgactcaggtcctcccactctcaaaagaatattgataacttttaaactctgaagctaaaatgaagacatgcttcactctgcctacgagtctcaagtttatgaactcactcactttagtgttctttcattaaataaaacttaaagaaggatgttcagaatggggtggagaaaggaagagaaatgaacagttaggggtcacaattattagagttttgggggcagtaatgaagacaaataggtaatttgaagtagtcttcaagagaaacaagaaataaagggaaagggttaggaaaaggaaagagggaaataatgaagttgatataatttggatattatgtgatcattagagaagaaacagcctcactaacagtcaattcacaattatgtttgtctagtagaaatatatctgctggaacactttggagaacagattggtaattttgccattacactagacattagccctttcaaagcagactaagagtacgtagttccttagtgaatacaaggcttcacaactgaagagcattgcagggagcttggctaaggtgaaaagggacttgctggagaggtacagcagaatgggatgggattagaaaacaacaatctattaggaaagatgaaaggaaggaattccccagtgttcacgctgagaagtcttcatattggaccagtgtgagggggtcttttatactgaggttttaaaaacctgacttcattttgtcctgaatgtagaagtgaagaaggacactagtttcaggcacctgggatggtggaaaagtttattcaactgaggctttccatgcattgggttgaatagctgagggaagctgaagactcttatctgaaaatatgatctgaaaatatttattgtataggaagacagaatgcctgatttctgaagttttcttgatggcccaactaaagaagaaaggcaccaaatcctcactttaacaggtaaatggagatcctgagggctggatacccaagagccaccattttacggattcctcagatttaaaaatgtctcagatagatatttccaaattctatttctattagcctctgtggataaagagttcctgaaactctggttttactttttttggaaaaaaaaaaaaaaaaaaaaaaaaaaaacaactcccag

SEQ ID NO: 596 - DNA, 735 bp, unidentified; identical to SEQ ID NO: 593
  source 1..735: mol_type = unassigned DNA; note = TIGIT nucleic acid sequence

SEQ ID NO: 597 - DNA, 735 bp, unidentified; identical to SEQ ID NO: 593
  source 1..735: mol_type = unassigned DNA; note = TIGIT nucleic acid sequence

SEQ ID NO: 598 - DNA, 735 bp, unidentified
  source 1..735: mol_type = unassigned DNA; note = TIGIT nucleic acid sequence
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatgcaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttag

SEQ ID NO: 599 - DNA, 735 bp, unidentified; identical to SEQ ID NO: 598
  source 1..735: mol_type = unassigned DNA; note = TIGIT nucleic acid sequence

SEQ ID NO: 600 - DNA, 735 bp, unidentified; identical to SEQ ID NO: 593
  source 1..735: mol_type = unassigned DNA; note = TIGIT nucleic acid sequence

SEQ ID NO: 601 - DNA, 735 bp, unidentified; identical to SEQ ID NO: 593
  source 1..735: mol_type = unassigned DNA; note = TIGIT nucleic acid sequence

SEQ ID NO: 602-610 - skipped (9 entries)

SEQ ID NO: 611 - protein, 244 aa, unidentified
  source 1..244: note = TIGIT polypeptide sequence
MGWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDAGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 612 - protein, 244 aa, unidentified
  source 1..244: note = TIGIT polypeptide sequence
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 613 - protein, 244 aa, unidentified; identical to SEQ ID NO: 612
  source 1..244: note = TIGIT polypeptide sequence

SEQ ID NO: 614 - protein, 244 aa, unidentified; identical to SEQ ID NO: 612
  source 1..244: note = TIGIT polypeptide sequence

SEQ ID NO: 615 - protein, 244 aa, unidentified; identical to SEQ ID NO: 612
  source 1..244: note = TIGIT polypeptide sequence

SEQ ID NO: 616 - protein, 244 aa, unidentified; identical to SEQ ID NO: 612
  source 1..244: note = TIGIT polypeptide sequence

SEQ ID NO: 617 - protein, 244 aa, unidentified [sequence omitted]

SEQ ID NO: 618 - protein, 244 aa, unidentified; identical to SEQ ID NO: 612
  source 1..244: note = TIGIT polypeptide sequence

SEQ ID NO: 619 - protein, 244 aa, unidentified; identical to SEQ ID NO: 612
  source 1..244: note = TIGIT polypeptide sequence

SEQ ID NO: 620-700 - skipped (81 entries)

SEQ ID NO: 701 - DNA, 936 bp, unidentified
  source 1..936: mol_type = unassigned DNA; note = VISTA nucleic acid sequence
atgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctag

SEQ ID NO: 702 - DNA, 3887 bp, unidentified
  source 1..3887: mol_type = unassigned DNA; note = VISTA nucleic acid sequence
aaaaatagctaaaaaggcttcttagaagagcagtaataatagtaatactatatatttgaaagggttgggaaatactgggtagtaaggaagtgtttaaaaacacaccagcgctgtttctttacactccccaagaaaaagacatactccccaagaaaaaagattcatgtgtacactccccaagaaaaaaacatatattggaggatagaagacttgagttataattttcatatttgccaccaatgagcacagccatgtatttacttatttatcaaatatttattgaatgtcttgtatgttccaggcactgtattagatagatacactatagcaaagctcctattcttgtagagtttatatttggggatgggacaggcaataaataagcttcgaatgcctaataataatgtaacaatgatgatagcaacaaatataattataactgctgtctagagtaaataatcaattttaataattaataataatacatgatatacagaaatttcaagaggacaacagatacagttgagcttatagagtgataacagagatggtttcctaagacagcggtctatgacctgagtctcaaatagtgagaaggatagagtcccaaaaagatctgggggcagggctttccaggcagggaggagagcaaggcagaggtcctgaggctggagtgagtttgttttatctgaggaacaaaaaaaggtagatgtggctggaatgaactgagactgggagagacaggctggagatcagctagggccagattatcctggacctgcaggcactgatggggagcttgattttattttaaatgcagtgtgacatcctgcaagtcattctgtttctgtcaaatggcaaggatggggtgatgaggatggagcagctgaatgattctcacagtctggaatgctaagtctaaccttatctaattcaggacctgggagaggtgctttgagtatctacagtgaggaaagtaaggtagcaaaaagacctcagatcctgcctcccagaatcctgtcactagggctggggaaagaaaggagtggagagcgttcagggctgggatgctatattcctgtaaaaccagaacctgttcttcttccacttttaatttttgaggtgcataggattttttttcactttgacaatctgcttctcatattactttagctttctgtagtgagaaaaagcacaggattaggggtcaggtgcctcaggctccagaaccccatctgttagtaaatagctaacctgatccagaccattcccggatctgggcctccagaccctcatctggggaccatcattaggattccttctgtgatttgattattttcctccctgccagagccggggaggaggctaaacccttgcatgcaagtccaccaaagctgtcctaaaataataaagcaatttcctttgaaatggtcagaagaaaagtcaggaggcccttgcaaaatgtgatgaaacccagccccagctgaatattaagttagcgaggggtctgcaatgagggtggggggctggggagagaagagagatgcagggttggggagagagaaattttaattgcaattgtttggcctgctgatcctaggcaaatgacaagaagcccccaagcagcctcaaacacagatatttgaaaggcagagacggctgggggatggtttggtttctccctcccctctgaggctgtttgaggaacggtctacagtgctgacaagcacgggttttggaggccattgaaaagttttgggcctaaatagggtaggcagcttggcttaagtgttgctaaggaatacctggcaaccagcaacaataggccttttagagaatccagggcaaacagtgaccgtggtaatggggtgggcttaagggtctccttgcccggctgggcccctcctcttctcacacagaggcctgggcactgggctatgttccggaaattccctgcacaaagccccattcagtgggaggagggccaatgcgtgtgctttgggggcagtgggggtggagacaggggatagggagagggcgggggagggggtaccggagaaggagctttactgccaaaaaaaaaaaaaaaaacctctcaagtctctttcttctcgcaaagactaaaagaggcacagaaagtgctaagtttgtaaaagagtctgaaaaggttcagaaaagtagcataaaaggaactggggaacgccccgtgaattttgaacagagagcagaaaagggaccctaaaaagcaattagtgatgctgcttgtaaatgaagaggcacctagtttggggtggggagctgagtctggaggtaggtgtgcaggggtgcagcaggtggtgggggttgaggggcaggtgaggagaatgaagtggggcgggaagcgggtgaggggaaggggaggccagtgaagaatggatggatggaaactggggaactgtggcctcagtccccactctaaccacatgccttcaggctgctctctccttgggctgtgcagatgccccacccactcagtttctgtcccctggtaaggcagccccccgaaagtcatgggtgcctgggacagtgaccagcacccagagtctgagcagtctccccagtcccttcagtctctgataaccatgaaagaagccctgacgttctgtgtgagtgtagactgcacactcctcacaagtaggcaaatggcaggatgctccagggagctgggtgtctgcctgcgttcctgggggaggctcacatgacagtcagtgaaggaaggccagggagccaacaaattagcgctctacaaattccacgtgcctgagctgcataacattcatgctgaaataccctactcaaaggctctaaagataataattttacatttccagcagaatgtgcacggtgacaattatctgtattcctactgtgtcgcagcatcaaccgataatcaagagaattatgttccagttgaaagcagatttaaataaacagacactaaatggaagccaataatagatgtaccaaaatagtccaggcaaggaagcagccattcagataaagagctgtttagtcgaggagacatgaggcatccagagcgcagagtttacagctctctgtctgtggctcagcctgtctgattctttctagctgtcctctgcagacccgaccagaggactatcctaaactgatccttttcccctcctttccttcctccctcccttctccctcttcctccctcttctttgtttcttcaacaaaaattcactatttattatcaacgaggtacatttgggcaccatcattaaaattaataaaccctacccttctttgatttttgtgtccttctgttcaagatttaaaccggatcatgttggccctgcatggggctagggaggagggtattagtaagttctttcaaggaaaaatctaactgctgtttctttaagaagggagaatgggatactaaagagacaaactattggagttctactacatattctttcaatgattgcacatttgttggaggtcactgtgtgttccaggcattgtcctaggtactgtgaattcaaagataaaagcacttgttcctgccctcacaaagcttccaaccttgcagggaaggaataagcaggcctggtgacagctggatgggcatgggtacaatgcatgatggaagcaccagagaaatgctctgccggggtaaggtgaaggagacatcaagaggaggcagctgtgccttgaaggaaaataacttaaatgaatttacaaattcatctggaaataattgttgaagcacaggaacgagataaactgctgagagaacatgtagaatgaggcaaagagaagtgtgtacacgcaaaccaacattaaagtcaagttcaacaaaaatattgagtctcctctga

SEQ ID NO: 703 - DNA, 4492 bp, unidentified
  source 1..4492: mol_type = unassigned DNA; note = VISTA nucleic acid sequence
cctcctcccttgtctttgtatgagggagctggtctcttttttcttccttctttcttgcctattaaactttttgctccttaaaaccaaaaaaagaaaaaatactttttggctagttcagcacacgcaccaaccagtcggagtggacacccagccaatcggaacaaatgccagctgtcaacaacaggaaacactgtactgtacctgacttagtgcaaaggggcctgggaaatggagttgctggctgagcaattgttgtgcagtgatgactcaccctgtggcaccagatgcacccatcctggtggacggctagctggccctgcctcaaatcacataaggtaccacaagcttccatctcaggtgtctgctttcaacccctgcatgtcccctccaccgcaccctatcatcaaggccagctagtccacccaaaccatttcaatggcagcgaatgacacacctccaatgtagttaaggaggggcaggtgagggaaactctcatctacagtggattctcaagtcagcacacccaccccagccttcccgctctttgctattagacaaacgatgaaactaagagaggaaaaaggccctccttgggattacccagcaagtcaggagtagcccaggactctggctgcctgcacaaggctggcagcagcgggacatgtcagcccacctttcatggaagcagcccagggaattagtcctgatgccaacgcccagctctggagttcctgacaccttttcttggatctaggccctttcaaaaggagcatttcaaataagagccatcaacacttacagagggtgggtcttaatccatagaggatcattagcttcacccaagtgatgggccatccatcagcacctcccctttttccaccaattatctatctgctaattatccgggattaagaattgacacggagcgacatggtcattaaccacatatttgcacagcttattcatgttagttgggctgctccatctcactgagtctaattggacacgcccacaatgctgcacaacacatttacatcgacctggctgtcagcagccaataaaggactacaggatgtgtttggttccaaagggacttggctctcggcagaagcttgtttgcaggcacactgggtgtcttggctttctggcactgtggactacagcttaactcagagcatacattcaatcatttctcaaacatttgttagctcaagcactgtaccaggcacagcactggccctagcagggtctacaaagatgaaccaggcaccatttctgccacctcgaggaggtttaccttctgccctccatgatgtgaccctgtctagcaggccccctcctgcccacctggggcatgtgtgccatctaccagcccactccacattctctgcgcaggtctctgtgctcttccttcagcctggaattcttctgtccacctggctctgtctgaagacgccacctcctctgtgaggcctctcctgatttctgagaccacacaaatctctccctgggctgcgttttcatggcatgctgccttcagtcaagggaacacatgcaagcagaaagagctcagagctgtgtgagaaagaaaagcagcatgtgtggtctcagaaatcagaagaggcctcacggaggaggtggcatcttcagacagagccaggtggacagagggattcaggctgaaggagtagcacagagatcagcaaggagaatggtctagtgcgtggcacacatagtactgatgggaagggtgtggcctggcaggcagggtcacatcatggagatctatgagtgttctgttagggtctggaagtttctcctcagggcatgaggaaccatggaagagattttgttttttcagtgtgtatgcataggtggatgtgttttagtaaactttttattgaaatgtaacatatgtatagaaaagtacaaaatttttcacaaactgaccgcatctacgtaatagcactcagattaaaacaataacaaattattactaaaactccagagatccactctggcctctttcaggaactacttacatacaagaaaaataaccatcctaacatcataaactagttttcactattttcttgtgtgtggcttttttcactccacatatgtttgcaagactcccccatgttgttagacataaatgcactttgttcttgctcattgctggttgatatcccattgtatggatgcactgcaattgttgatgagctttgtgcagtttccagtttggggctgtaaaccttcctgcacatgcctttacgtgctcctctgggggtaaatctgttgcatgcatatctaggagtggaattgctgggttgtgggctatgcatatatctacttgagtagatacttctagtttcccaaagttgttctacccatgtacacgcccaccagggatgcacaggagttctcattgctctatattcttgtcaacacttggtattgtcagtctttttgtgttttatgcttgtttttttttttttaggggaatgaccacctggtggaggtggagtcccacttgggttgaagtatactcactctggcaggaatgaaatcagtggtaatagagctagaatgaaaaaggccaataagaagagacgccatagaagtggaatacacaggacttgcacacaaggtagaaatgaacttaagagggagatggaggaaggtttctgtatggggtggctggctggatgagggacttcagatttgaagccaggagaaaacatcccaattttataacatcatgtttgaaatgtaggacaatgagatgaagatgtccaataaactgttagctatatgagtctgttgcttagggatgaggtcaaggctgtagacagagttaggagttatcacagagatgggggttgaggctggggggtggctgagatttctcaggtggttgtgtgcaatgaaagagacaagggctgaagaccagacgcctggcttaggggatgagcggaggaagaacaagagacaacatcgcttccctctgaaatgctggcacgggctgcgaagcatgagtcagacccttctagccccagcagcagcctcaacagcctgggtctatctcaccagccacacagttttctcctgaatgaacttgcatcactaacctcagaagctgtgttgcgaaatccgcatcagattgcgtacctctggtggcgtcctagggacaatgggcactaaagactcatagcatcctggggttggagtcctcctctccgcctccacaacatccctgccaatcagttatcgagcaccccaggcagggaactcataacttcccacggcatcccatttccttgttgagcaagtgaagatctaagaaaaccttttcttgcaatctcacgatactgaaaagtcaaagaaaaagataacgctataatttaatggtaatgatccaggacagagtgaaatgcaaggactctgcatcctggaaaatccatgtctattaatgtacccatcagcatcttcttgctggggttagggtcatggtgtttgactccccacaggaagctgcagtgatttcattactatttgcacactggttgggaatgtgctcatccgctgcaggcattcctctggcttcatcaagcagtgagtacaagggccctggaatctggccagtctctcaggactgaagcagtgcatgctacagcacaaatctaccaggctggacacgtgggaagaatgagcggctgttagatgagagcgagcccactgcaagcaaagagggcagaatgccattctgttcaacatttttttctgcgtgtttagcagcatgtggtcctgtgtgggtctctggagaagaagaaagaagatggatctaagaaatggttctcacccagctgggcacagtggctcacgcctgtaatcccagcactttgggagggagaggcaagtggctcacctgaggtcaggagttcgagactagcctgaccaatatggtaaaaccctggctcgaataaaaatacaaaagaatggccaggctctgtggctcatgcctgtaatcccagcactttgggaggccgaggcaggcggatcacaaggtcagaagttcgagaccagcctggctagcatagtgaagcctcatctctactaaaaaatacaaaaattagccaggtgtcggggcaggcgcctatagtcccacctactcaagaggctgaagcaggagaatcgcttgaaccaggaaggtggttgcactgagccgagattgcaccactgcactccagcctgggcaacagagcgggactccatctcaaaaaaaaaaaaaagaaaagaaaagaaaagaaagaaagaaaaagaaaagaaaaaaagaaagggttcccacccttggggagattggtggcagaggcagagacaggcatgcaaaaggaagatgtagctgaaacttttttttttcttttgagatggagtcttgccctgtcacccaggctggaatgcaatggcacaatctcagctcactgcaaactct

SEQ ID NO: 704 - DNA, 2459 bp, unidentified
  source 1..2459: mol_type = unassigned DNA; note = VISTA nucleic acid sequence
atctcagtaatttgagtgtggtccttccccaactaggctcaagaaacccaggctcctcacccacaaaagggccagattgatggattctcaggcattttaaagaaagatgagggtggagaagggagaagattaggaaaacttaagtccttcagaaaagagatgggagaaaagtttttcaaccagactttgcccattgtatgccctcctgagggagtttagtttgaacttcaggttaaaatgcttacttctcttaacctctggttattaaaaaaaaactggacatatagttacacattgaggactactctcccaaaatattggggaaaacatgtagcagaatcttgcaacttctaaactttctgacttcaaaccccaaagtacaagtcttacttttccttcctttctttcctcctctccctggccccaatctgctgaaggtgatgggccaagcccaagggcctcagcagccttataaggcaagtgttctgaaaaaccttcaactcttaattttaacattaaagaaaaagttgtaatcagacttggaggaaacttagcttttttccctccctccttcccccttctgggggtacgaggttgtttttgcatgcttcatttgcttcttatcctgataggctgcattaatcagttttatttccattgatagagaaacctcgtctgttctgttcgagctacaaagcgtcctgcgagcttttgtgaaagtgcaaatcagtttaagcaattatcataccaggaatatgaaggggaaagaggaggccttgcccagtggtctcctttaatctcttaatccacggatccaggggggctgcctggacataatttaggacaatctccccaccctttacactgtgataaggccaagttacaatgcagggcaaaaatacaagctttgttaacatcttgccttgaaaagttaagattagacattccgtgcacgtgtggggactatagggcactatggaaattttttcttttttttctcctcttctctaaagcagaattcattctccgtctctttctcctttcccccatctctgcctgtcttccacctcccctctttccccccatttctgtccctctgtagaaacctggctgctcatagtgcattttcacagactcttcttggaaggtgtgtgtgccgtgaggggatacaccgccgccgggagttgccgggggaggagggagacggggtggcctcaggagacccgggcttgcgtctcacagggacaggtgcgcctcggggtatcctggaaaccctcgtggggcttccaggcgctttgacagcacgaccctcagtgaccaccagctgtctcaagcgctttgcagtcagtgctcccggaaagcatccctttctcttcaacaagtacaaattaccttttccttccactcaccttccaaaacccggaatacctcaaacaccgagctcattcttaaacattcttaaaattcagatggtctgtccagaaaaaaaaaaaaattccgtcttaacagaaggtttacttctgccgaaattatatactttatactttttaagtacaagaaaacggggctggtggtgaagatttttctttttttgggtaaatgtcctatgtagtgtaacagcaataaaatcatcacagaatactattagctttgaaaaaaaaactaaaagctttattacaaaccaagctttgaaaataggggggattaggcggctgaaagggtcccacaatggtaagaagaaaaggtttcatgctaatgaggttaatgccctttgtatctcaggcctccacatcttcattacgcgctatctccggctgcacggagcggctcagagagccgcaatccactccaacgccccccttcccggcccaaagaaggatttgatagcagctctgttcaaactagataattatatcttttcaagtcggaattaagataaagaaagtgaagcagaggcggctcgccttgatccactgcaaacaaatttggcgcactttcgagtccttcctccgcctcaaaaaggcaggacagtcagcttattagccgctcgtttgctttattaattcatctatttaaagtggcaggattagagtgtctaatgtggacgcgggctgccgtggcgctgaggaatataaatatttgcgagatgccatcccacgatgactatctgggagtggggcgcggggcagggtgcgcgtgtgccctgcctctgcgttcccttcgcagtgcgcgcgtgctgagctgagcagggggttgtaggagccatgagccgcaggtctatgtgccccttcgtgctatatgggtgccagccctggatgctcccgggcagggctcaatgggcatcgtgggtcagattctgcgctccttctgggacacggagaactgtatgcacgttgccattgggtcctgcctgggacccctaatggggatgtaggatacaagtttgatccatgttccgt

SEQ ID NO: 705 - DNA, 4619 bp, unidentified
  source 1..4619: mol_type = unassigned DNA; note = VISTA nucleic acid sequence
ttatccctcgataataaagtaaatgttaaaaaggatatcccccacaatctcttcttcttcaggcctttcctgatttaatcttagatgttcttgctatatcatgagtggctgagctccaatcagagaaaattcagcgatggaaactctttacttgggcttcccactatttatgtgtggtattcccaatttgtagctgtatttgtgctggagcagacagaaataaccaatgaaaaaaaataggagcaatacagttttatatttaaagtagtgtcatgaggcatcaaggcagttagtaatccaagaaggctcaaattatgttttttaactataaagtataaatttagtaaaaaagactaatttctaataaataatagacaaatattggaatgaaaaacatcactttctatataaatttactaagggagaactttcggtaaggttaccataattttattttacagccataaaacttttcaggaaataggttaccaatttgatattgtagattaattaacctcagttatatacatattttttaaatgctaatttttggcaatacagttgagaagaattattttgtaattgcatatctttctgtgtctggaattcatcccctataaagtcctctggccatgggtggcagtggcctgcggtcaaaatctaaacataagttcatcatcatcttttaattttgtggtcttgagcttttgacccaaagaggagttgggggttcactagggcaggactaaatggagcaaacacttgtctttggagcccaagcgctgaccttaaagatgtaaaatgaattgcctgctcccaaggttattgttataatcaggtatgtgaaaatgagcagtaagaatgtgaaggagagagcggaagagagagagagagaaagagagaaaacattccttggtttgctagaagtatttagtgcatattttatattttttaattttattttttaaccatgttctgctttaaaaactgtacatagaagagtcaggcttctgacttttaatgaattgctgtgaaatttggttacatacattactccaaccctgtgagggggggacaattatagttcattatatgcatctcaagttgtttattcattatgatgcacaaggatagtttaaggacttgtttctgccaagagctccccccaccaaataatttcacaacagagtaaaaacacatggactttatataaagtaaaaatagaagtgatataagggttgtatttttatagctgtatttgagtacatttcaaatgaatgtttgttaaacagggggaaaaatcagactggcattgtcgtgattctaagcgaattaggtgaggcaggaacaggaagaatctgagtagcttgcatttaattagcacttactggtacaaaataaattagacagatttgtcttgttttatgagcaagatcctgtttttctatttcattttctttgtgcttttggtaaggacggatctggcgctttggatactaaataggcaactaattcaattggtgctttctccgagggtctccttgcaaaccggtgccagactcactggtcctttgtgtactgcccgccctgcatgtgtctgggctcccctggccagtccgtttttttgtctctgatggttaataagctttgagcctagtagtcaaatgtggtgggaacggggacaagacgagggtccttagtgaaggtgtttgttgttaaatagaacaattcagctgtctctttagaaaacttgacaaaaggaaatgctccctgctgaaaattttcttagcctgttgcacaaaagatgaagtttgtttaagaaattgctgctaaagaaagcgtcaacccattaaggtaattatatggaacctgtgccgtataacagcttaattgatgttaatgtttgtctaattggtgattgggagagtgcaaaggactaaaggtcttccgagcactttcagtcacggctctgaaaggggctcttctggttttcacagttataacaatgcctgcgcgtctttctcagattacgccttgttttcagccccttacagcattctgctccactacttaaagcgtaaaataagggtgaagagccattgtctaatgcagcctttttcaaagagtgtttctgacagggattgttctaggaatcaggcaggagagaagggagtgtgagaggtgaaagccactatcacccacttgtgtcacctccccaatgagcagcaagagccgcctaattgctgcatctgttttttttttcttttcctccaagcctccattctcttaaacaagatccttacaccgatgccacatgttcctgtggaatctatcaaacaaaacaaaacaaaaaaacaccccaacctggctgacattttttaaattggcataaaaagaaccgttaggaaaacatatataacaattaaatgcatgcacaaaaatatatgaacttttctctttcctaagatttcacacattgtatggacacatttacaaatagaattagatgaaagaatacactgtgggtacctaaacacatatatgtgacttttctgtgtaaagatacatgcatgattattacatggatacttacatatgcagacacaaatgtatagatttggaaaccttgcttttgtttccatatgagaaagccatttaaatgtgtcatcaatcttggtgttttttttttaagtcatagtaaacaccgttgatccaggttataaaactcaaatgcttaaatattttacattcctagataaactgattgaagagccaagacttgtcttaaaaaaaaattctcagcttacacactcattcattcgcgcgtgcagacacacacacacaatgcatttaacatacccaccccccccccaccatcctgcaaagttatagttctcaggcttttcaatttttatgttttacattcctatatttaacagattgatattttcctgtttgttctctttaagatttcttttctacatacagagtgtattcatgtgctatcctaatgaggaaatgacgagtttttatcaaccttattaaacatacacccagaagatatccatttgaataaggggccaattactaagtgtgtcttcagttagaaagaacttagcttttaccttgtactttggctctccatgtctctaattaattgaatcttgaaatcgttggactttcagcttaagagaagactgaagaagggaacttaacaacactgcctattcctttatgtctagaacgggctaaaaaaaaaaaaaaacacctgtaatgctaaaagactcaggcttttgtctgtttctaatattacttttctgcctaattgccatcactgccacgtacattaggccctttttcagcagggttaaaacagggccagtgatgaatggaaaactgattgtactgctgtcaaacttgaatgtgaggcttattctagagatagaagtatgaggaaaaggacagagaattgcacttcattaaagctctaccaagctgattggagtggaggaggtaattgagatttaaagtggcattcccatcacactgtccctttttcaacttaacaactgtcattcaagccacatagggcaacaattggccagatttcccagctcttctgagtaataaaacagacactcaccttctggctattcgttttacttactttgttctaatttccaattcatgtgtcattagtgaacgaagattgacaagaaatagctttgacctcacagatttgaccttcaaccccaaagacccaactctagtctgaacataggttggaatatgctaagaaacacctttcctttgcacaagaatgaatggcagttttttcatcattgtaccaccccctgcttgccactacatttagactatgagacatgtcaactgcctgtcacaattacaatagacacagcaaaccagaattttccccaaatccaaggggaacttcgcgaacaaagagtaaattttattgcacagattttggagacctccaatttgtagagccataatgaatgcatcccacagacaaataggtagttgttaattatcccagcaatttctttttttaatgtgctaacaacacagcacgctaagccttcatatcctactctgttacctgatgatctcatcacatttgcaacgatggtagtctgggaagaaggcattattttagcaagtatgcatgacaataagttacatgattataatcccttattttcatgatttaataaaaagaaagtgtgatattttggaaacaaatgggtttagctacacctttactaaatcagttgaagtagtttacctatagaatcactttatgataaacgtaatctttgtattttccttgactgagtatcctaaagcttagatataccaattttgtgtcatcaaaacatgctgtggataaaaaatgttgaagggcaagacctatgacctgaataagaaccctcaatgaatttaaaagtattttatgcctttcacacagttttgaatagcaattgggtcctggaaatctattatggcaaggaaattgttacggttttttttacattaagtgacaaacatttggatgattcttgtgttacatccccctcgccccctaaacagaataa

SEQ ID NO: 706-710 - skipped (5 entries)

SEQ ID NO: 711 - protein, 311 aa, unidentified
  source 1..311: note = VISTA polypeptide sequence
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 712-800 - skipped (89 entries)

SEQ ID NO: 801 - DNA, 465 bp, synthetic construct
  misc_feature 1..465: note = Synthetic polynucleotide
  source 1..465: mol_type = other DNA
atgaccgtgctggcgccggcgtggagcccgaccacctatctgctactgctcctactcctgagcagcggcctgagcggcgctcaggttcaactggtcgaatcaggaggtggcttggctcaacctggtggatccttgagactcagttgcgccgctagtggaagcaccatttcttccgtagctgtagggtggtaccgccaaacacccgggaatcagcgggaatgggtggccacttcatccacttccagcacaacagcaacctacgccgacagtgttaagggacggtttactatttctcgggataatgccaagaacacaatatacctgcagatgaattccctgaagcctgaggatacggccgtctattactgcaagacagggctcaccaattgggggaggggaactcaagtaactgtgtcctctggcggtctccctgagacaggcggacatcaccatcaccatcactaa

SEQ ID NO: 802-850 - skipped (49 entries)

SEQ ID NO: 851 - protein, 154 aa, synthetic construct
  REGION 1..154: note = Synthetic polypeptide
MTVLAPAWSPTTYLLLLLLLSSGLSGAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGHHHHHH

SEQ ID NO: 852-900 - skipped (49 entries)

SEQ ID NO: 901 - DNA, 474 bp, synthetic construct
  misc_feature 1..474: note = Synthetic polynucleotide
  source 1..474: mol_type = other DNA
atgaccgtgctggcgccggcgtggagcccgaccacctatctgctactgctcctactcctgagcagcggcctgagcggcgctcaggttcagctggttgaaactggtggtggtctggttcagcctggtggctctctgcgtctgtcttgcaccgcaagcggcttcaccttctctatgcatgcgatgacctggtatcgtcaggccccgggtaaacagcgtgaactggttgcggttattacctctcacggtgatcgtgcaaactacactgactctgtacgtggtcgtttcactatctcccgtgacaacaccaagaacatggtatacctgcaaatgaacagcctgaaaccggaagacaccgcggtctattattgcaacgttcctcgttacgattcttggggccagggcactcaggtgactgtgtcttctggcggtctgccggaaaccggtggcgactacaaagacgatgacgacaagtaa

SEQ ID NO: 902-950 - skipped (49 entries)

SEQ ID NO: 951 - protein, 157 aa, synthetic construct
  REGION 1..157: note = Synthetic polypeptide
MTVLAPAWSPTTYLLLLLLLSSGLSGAQVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGGDYKDDDDK

SEQ ID NO: 952-1000 - skipped (49 entries)

SEQ ID NO: 1001 - DNA, 1617 bp, synthetic construct
  misc_feature 1..1617: note = Synthetic polynucleotide
  source 1..1617: mol_type = other DNA
atggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacaattatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaaagagggggaacctgtttatttttaggggaagaatgctgttattatgttaatcaatccggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttag

SEQ ID NO: 1002 - DNA, 1617 bp, synthetic construct
  misc_feature 1..1617: note = Synthetic polynucleotide
  source 1..1617: mol_type = other DNA
atggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacagttatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaaagagggggaacctgtttatttttaggggaagaatgctgttattatgttaatcaatccggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttag

SEQ ID NO: 1003 - DNA, 1617 bp, synthetic construct; identical to SEQ ID NO: 1002
  misc_feature 1..1617: note = Synthetic polynucleotide
  source 1..1617: mol_type = other DNA

SEQ ID NO: 1004 - DNA, 1617 bp, synthetic construct; identical to SEQ ID NO: 1002
  misc_feature 1..1617: note = Synthetic polynucleotide
  source 1..1617: mol_type = other DNA

SEQ ID NO: 1005 - DNA, 1617 bp, synthetic construct; identical to SEQ ID NO: 1002
  misc_feature 1..1617: note = Synthetic polynucleotide
  source 1..1617: mol_type = other DNA

SEQ ID NO: 1006 - DNA, 770 bp, synthetic construct
  misc_feature 1..770: note = Synthetic polynucleotide
  source 1..770: mol_type = other DNA
cacgaggttgcctatagccttaaaaaaaatctgcaactttccccaaaaagcagacttagcccatacgaaatgctgtatggaaggcccttcataaccaatgaccttgtgcttgacccaagacagccaacttagttgcagacatcacctccttagccaaatatcaacaagttcttaaaacattacaaggaacctatccctgagaagagggaaaagaactattccacccttgtgacatggtattagtcaagtcccttccctctaattccccatccctagatacatcctgggaaggaccctacccagtcattttatctaccccaactgcggttaaagtggctggagtggagtcttggatacatcacacttgagtcaaatcctggatactgccaaaggaacctgaaaatccaggagacaacgctagctattcctgtgaacctctagaggatttgcgcctgctcttcaaacaacaaccaggaggaaagtaactaaaatcataaatccccatggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgncaatactcattattggacagga

SEQ ID NO: 1007 - DNA, 1617 bp, synthetic construct
  misc_feature 1..1617: note = Synthetic polynucleotide
  source 1..1617: mol_type = other DNA
atgggcctgctcctgctggttctcattctcacgccttcactagcagcctaccgccatcctgatttcccgttattggaaaaagctcagcaactgctccaaagtacaggatccccttactccaccaattgctggttatgtactagctcttccactgaaacaccagggacagcttatccagcctcgcccagagaatggacaagcatagaggcggaattacatatttcctatcgatgggaccctaatctgaaaggactgatgaggcctgcaaatagtcttctttcaacagtaaagcaagatttccctgatatccgccagaaacctcccattttcggacccatctttactaatatcaacctaatgggaatagcccctatttgtgttatggccaaaaggaaaaatggaacaaatgtaggcactcttccaagtacagtctgtaatgttactttcactgtagattctaaccaacagacttaccaaacatacacccacaaccaattccgccatcaaccaagattccccaaacctccaaatattacttttcctcagggaactttgctagataaatccagccggttttgccagggacgcccaagctcatgcagtactcgaaacttctggttccggcctgctgattataaccaatgtctgcaaatttccaacctcagctctacagcggaatgggttctattggaccaaactcgaaattctcttttttgggaaaataaaaccaagggagctaaccagagccaaacaccctgcgtccaagtcttagcaggcatgactatagccaccagctacctgggcatatcagcagtctcagaattttttggaacctccctcacccccttatttcatttccatatctctacatgccttaaaactcaaggagccttttatatttgtggccagtcgattcaccaatgcctccccagtaactggactggaacttgtaccataggctatgtaaccccagacatcttcatagcccctggcaatctctctcttccaataccaatctatgggaattccccgttgcccagggtgaggagggcaatccatttcattccccttctcgcgggactcggcattctagctggtacgggaaccggaattgctggaatcacaaaagcttccctcacctatagccagctctcaaaggaaatagccaacaacattgacaccatggctaaagccttaacgaccatgcaagaacaaatcgactctttagcagccgtagtccttcaaaatcgtcgaggactagacatgttaacggcagcacagggaggaatttgtttggccttagatgaaaaatgttgcttttgggtaaatcaatcaggaaaagtacaagacaacatcagacaactcctaaatcaagcctccagtttacgggaacgagccactcagggttggttaaattgggaaggaacttggaaatggttctcttgggttcttccccttacaggcccacttgttagtctcctacttttgctcctttttggtccatgtctcctaaatctaataacccaatttgtctcctctcgccttcaggccataaagctccagacgaatctcagtgcaggacgccatcctcgcaatattcaagagtcacccttctaa

SEQ ID NO: 1008 - DNA, 1617 bp, synthetic construct
  misc_feature 1..1617: note = Synthetic polynucleotide
  source 1..1617: mol_type = other DNA
tgtcactgcactccagcctgggtgacagagtgaaaccctgtctcaaaaaataaaaataaaagtaaatacaaaattcctcctatagttagcctggaatgagcaaagacagcttgtgaggttagaaggaagatggagtcagttatgttagatttccctcactcataatttttgcaaaagtagttagtctttcttgattaagtcctcctggttgctgtaagcttttgcttaggttctagaattctaaaaaagttgattctggcagtttttgccagtttatttattgcttttttggagggacttttgatgttccttattcttccctgttcatggatgtcacccctacatgcttttcttacaatgtgacttttgcagtcctctcaccaagaggtggggcctatgttccattctatgagactgggtgggacttacgactgctccaaccaaacaaggacaacagaggccatggtttgtaactttctaagctaggttatgatacagcttctagctgactcacttttttcaagagctggggttagacaattgctctggggaagtcagctaccacactgtgagaagcccagattcacccatatggagaaaaactaaggcctcaagctgacagccagccttgacctccagacacaagaataggctggtcttcagatgcttccagcgtccagccttcaagctctctagtagagaccccagacttcagagaaaagctgtccctgctgtgccctgtctgaattcctgacccacagaatcttttgtctcttccagaattgaagctgtaaagctacaaatgattcttcaaatggagccccagaggcagtccatgactaaaatctactgcggacccttagaccggcctgccagcccatgctccaatgttgatgacatccaaggtctgtgtttgttccggttcgaactgagctttcgctcgccatccaccactgctgattgctgtcgtcgcagatgccgccgctgacttccacccctccggatccggcaggatgtccactgcacttctgatccagtgaggcagcgcccactgctgctcccaatcaggctagaggctcaccattgttcctgcgtcagctaagtgcctggggttcatcctaatcaaactgaagagagctataacactcattgcatggcccaagattccattccttggaatccgtgaggccaagaaccccaggtcagagaacaagaggcttgctgccatcttggaagtggcctgccaccatcttgggagctttaagaacaaggaccccccagtaacaccatgattcaattacctcccactgggtccctcccatgacacatgggagttatgggaactacagttcaagatgagatttgggtggaaacacagccagaccatataaggcctgtggccacagcatgtctgactgttggaagaacacaaggaggcagagggacgaacagagagaaggaaaggtgtaaaggcagaccttgttaggccttgtaaagcattttaatgattggggtttttagtctgagtacaaggagttctgagtgctgagtgatgagtgctgaagggttttaagcagaagagagatgacgatgtaagcttttttttttttttttttt

SEQ ID NO: 1009 - DNA, 480 bp, synthetic construct
  misc_feature 1..480: note = Synthetic polynucleotide
  source 1..480: mol_type = other DNA
atgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctag

SEQ ID NO: 1010 - DNA, 617 bp, synthetic construct
  misc_feature 1..617: note = Synthetic polynucleotide
  source 1..617: mol_type = other DNA
ccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctag

SEQ ID NO: 1011 - DNA, 1125 bp, synthetic construct
  misc_feature 1..1125: note = Synthetic polynucleotide
  source 1..1125: mol_type = other DNA
atggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctag

SEQ ID NO: 1012 - DNA, 1932 bp, synthetic construct
  misc_feature 1..1932: note = Synthetic polynucleotide
  source 1..1932: mol_type = other DNA
agagtgcgttcctcgtctgccagccggcttggctagcgcgcggcggccgtggctaaggctgctacgaagcgagcttgggaggagcagcggcctgcggggcagaggagcatcccgtctaccaggtcccaagcggcgtggcccgcgggtcatggccaaaggagaaggcgccgagagcggctccgcggcggggctgctacccaccagcatcctccaaagcactgaacgcccggcccaggtgaagaaagaaccgaaaaagaagaaacaacagttgtctgtttgcaacaagctttgctatgcacttgggggagccccctaccaggtgacgggctgtgccctgggtttcttccttcagatctacctattggatgtggctcaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaacctttttttttacagagcctaattaataacttaatgactgtgtacatagcaatgtgtgtgtatgtatatgtctgtgagctattaatgttattaattttcataaaagctggaaagca

SEQ ID NO: 1013 - DNA, 1587 bp, synthetic construct
  misc_feature 1..1587: note = Synthetic polynucleotide
  source 1..1587: mol_type = other DNA
atggccaaaggagaaggcgccgagagcggctccgcggcggggctgctacccaccagcatcctccaaagcactgaacgcccggcccagaaagaaccgaaaaagaagaaacaacagttgtctgtttgcaacaagctttgctatgcacttgggggagccccctaccaggtgacgggctgtgccctgggtttcttccttcagatctacctattggatgtggctcaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctag

SEQ ID NO: 1014 - DNA, 1593 bp, synthetic construct
  misc_feature 1..1593: note = Synthetic polynucleotide
  source 1..1593: mol_type = other DNA
atggccaaaggagaaggcgccgagagcggctccgcggcggggctgctacccaccagcatcctccaaagcactgaacgcccggcccaggtgaagaaagaaccgaaaaagaagaaacaacagttgtctgtttgcaacaagctttgctatgcacttgggggagccccctaccaggtgacgggctgtgccctgggtttcttccttcagatctacctattggatgtggctcaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctag

SEQ ID NO: 1015 - DNA, 1248 bp, synthetic construct
  misc_feature 1..1248: note = Synthetic polynucleotide
  source 1..1248: mol_type = other DNA
atgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctag

SEQ ID NO: 1016 - DNA, 1632 bp, synthetic construct
  misc_feature 1..1632: note = Synthetic polynucleotide
  source 1..1632: mol_type = other DNA
atggccaaaggagaaggcgccgagagcggctccgcggcggggctgctacccaccagcatcctccaaagcactgaacgcccggcccaggtgaagaaagaaccgaaaaagaagaaacaacagttgtctgtttgcaacaagctttgctatgcacttgggggagccccctaccaggtgacgggctgtgccctgggtttcttccttcagatctacctattggatgtggctcagaaggatgaggaagttgtcttttgcttctcctcattccaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctag

SEQ ID NO: 1017 - DNA, 1509 bp, synthetic construct
  misc_feature 1..1509: note = Synthetic polynucleotide
  source 1..1509: mol_type = other DNA
atggccaaaggagaaggcgccgagagcggctccgcggcggggctgctacccaccagcatcctccaaagcactgaacgcccggcccaggtgaagaaagaaccgaaaaagaagaaacaacagttgtctgtttgcaacaagctttgctatgcacttgggggagccccctaccaggtgacgggctgtgccctgggtttcttccttcagatctacctattggatgtggctcaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgtcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctag

SEQ ID NO: 1018 - DNA, 348 bp, synthetic construct
  misc_feature 1..348: note = Synthetic polynucleotide
  source 1..348: mol_type = other DNA
atgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgccaagctaaagaggaagtattggtggaaaaactgcaagatgatgatcatgctgggagccatctgtgccatcatcgtggtagttattgtaaggcgggac

SEQ ID NO: 1019 - DNA, 351 bp, synthetic construct
  misc_feature 1..351: note = Synthetic polynucleotide
  source 1..351: mol_type = other DNA
atgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgccaagctaaagaggaagtattggtggaaaaactgcaagatgatgatcatgctgggagccatctgtgccatcatcgtggtagttattgtaaggcgggactga

SEQ ID NO: 1020 - DNA, 354 bp, synthetic construct
  misc_feature 1..354: note = Synthetic polynucleotide
  source 1..354: mol_type = other DNA
atgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgcaaagctaaagaggaagtattggtggaaaaactgcaagatgatgatcatgctgggaaccatctgtgccatcatcgtggtagttattgtaatctacttttttact

SEQ ID NO: 1021 - DNA, 352 bp, synthetic construct
  misc_feature 1..352: note = Synthetic polynucleotide
  source 1..352: mol_type = other DNA
atgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgcaaagctaaagaggaagtattggtggaaaaactgcaagatgatgatcatgctgggagccatctgtgccatcatcgtggtagttattgtaagtaagtatcgct

SEQ ID NO: 1022 - DNA, 357 bp, synthetic construct
  misc_feature 1..357: note = Synthetic polynucleotide
  source 1..357: mol_type = other DNA
atgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgccaagctaaagaggaagtattggtggaaaaactgcaagatgatgatcatgctgggagccatctgtgccatcatcgtggtagttattgtaatctacttttttacttga

SEQ ID NO: 1023 - DNA, 354 bp, synthetic construct
  misc_feature 1..354: note = Synthetic polynucleotide
  source 1..354: mol_type = other DNA
atgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgccaagctaaagaggaagtattggtggaaaaactgcaagatgatgatcatgctgggagccatctgtgccatcatcgtggtagttattgtaagtaagtatcgctga

SEQ ID NO: 1024 - DNA, 351 bp, synthetic construct; identical to SEQ ID NO: 1019
  misc_feature 1..351: note = Synthetic polynucleotide
  source 1..351: mol_type = other DNA

SEQ ID NO: 1025 - DNA, 351 bp, synthetic construct
  misc_feature 1..351: note = Synthetic polynucleotide
  source 1..351: mol_type = other DNA
atgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgccaagctaaagaggaagtattggtggaaaaactgcaagatgatgatcatgctgggagccatctgtgccatcatcgtggtagttattgtaagaagaggctga

SEQ ID NO: 1026 - DNA, 965 bp, synthetic construct
  misc_feature 1..965: note = Synthetic polynucleotide
  source 1..965: mol_type = other DNA
agtaagttccagcgcagctagaccgcggggtagtcggcgcgaggcggagcttggcagttccgtccacttcagccgcagcgtccctcaccgggtgtctcgccgcagcctccggagaggaacagaccctcactctctctgtcagaaaaatgtggtggacatcatacgtgtgaacgtggacaaggtcctggagagggaccagaagctgtcagagctggatgaccgagctgatgccttgcaggcaggagcatcacaatttgagagcagtgctgccaagctaaagaggaagtattggtggaaaaactgcaagatgatgatcatgctgggagccatctgtgccatcatcgtggtagttattgtaaggcgggactgaagagctggaagaaagcagtcagtaccctcccaacggcccccctcgaaggtctccactctcctctgggctcctccttgcctaatgcagggggtcaccgctggagaagaaccaccactgtcctcgatgtgtcccaagcctggagcgaatccgtcctcttggctctcccagccctatcacaggaatcattcctgggtttctgtccctctgaggctcaccaggtgtagttggccttgttctttgggggtattagcccttctactttctttctcacccaccctgtaccctcttctgtgtgtctctgctatccccctttcctcccaccacccatgtgcatgagcaaatgtgcaacaaaaccctgggactttgcagtcaaatgaagctgagcttcccacatcccgttcttcggtttgccatgagacgatgtggggtttccactgtgtgtctgtcatcacctctttgtctttttttcctaaccctgatctcatacttgtatagagtaataacaacagttgtacttccaccaaaggcatgtggcatatttaccaatgtcatgtattctgaacaaaggcaaaaaatacaaattcctaccattaaa

SEQ ID NO: 1027 - DNA, 357 bp, synthetic construct; identical to SEQ ID NO: 1022
  misc_feature 1..357: note = Synthetic polynucleotide
  source 1..357: mol_type = other DNA

SEQ ID NO: 1028 - DNA, 200 bp, synthetic construct
  misc_feature 1..200: note = Synthetic polynucleotide
  source 1..200: mol_type = other DNA
agcggagaacagccttgggagtcatctttgccagcagtctcttccttcttgcactgatgttcctggggcttcagagacggcaagcacctacaggacttgggctgcttcaggctgaacgctgggagaccacttcctgtgctgacacacagagctcccatctccatgaagaccgcacagcgcgtgtaagccagcccagctga

SEQ ID NO: 1029 - DNA, 200 bp, synthetic construct; identical to SEQ ID NO: 1028
  misc_feature 1..200: note = Synthetic polynucleotide
  source 1..200: mol_type = other DNA

SEQ ID NO: 1030 - DNA, 286 bp, synthetic construct
  misc_feature 1..286: note = Synthetic polynucleotide
  source 1..286: mol_type = other DNA
gtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgccaagctaaagaggaagtattggtggaaaaactgcaag

SEQ ID NO: 1031 - DNA, 354 bp, synthetic construct
  misc_feature 1..354: note = Synthetic polynucleotide
  source 1..354: mol_type = other DNA
atgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgcaaagctaaagaggaagtattggtggaaaaactgcaagatgatgatcatgctgggagccatctgtgccatcatcgtggtagttattgtaatctacttttttact

SEQ ID NO: 1032 - DNA, 354 bp, synthetic construct
  misc_feature 1..354: note = Synthetic polynucleotide
  source 1..354: mol_type = other DNA
atgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgcaaagctaaagaggaagtattggtggaaaaactgcaagatgatgatcatgctgggagccatctgtgccatcatcgtggtagttattgtaagtaagtatcgctga

SEQ ID NO: 1033 - DNA, 357 bp, synthetic construct
  misc_feature 1..357: note = Synthetic polynucleotide
  source 1..357: mol_type = other DNA
atgtctgctccagctctgccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgccaagctaaagaggaagtattggtggaaaaactgcaaaatgatgatcatgctgggagccatctgtgccatcatcgtggtagttattgtaatctacttttttacttga

SEQ ID NO: 1034 - DNA, 501 bp, synthetic construct
  misc_feature 1..501: note = Synthetic polynucleotide
  source 1..501: mol_type = other DNA
gcacgaggccgcggggtggtcggcgcgaggcggagcttggcagttccgtccacttcagccgcagcgtccctcgccgggtgtctcgccgcagcctccggagaggaacagaccctcactctctctgtcagaaaaatgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgccaagctaaagaggaagtattggtggaaaaactgcaagatgatgatcatgctgngagccatctgtgccatcatcgtggtagttattgtaagtaagtatcgctgaagttgctggtgggtg

SEQ ID NO: 1035 - DNA, 706 bp, synthetic construct
  misc_feature 1..706: note = Synthetic polynucleotide
  source 1..706: mol_type = other DNA
cacgaggtccgtccacttcagccgcagcgtccctcgccgggtgtctcgccgcagcctccggagaggaacagaccctcactctctctgtcagaaaaatgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgcaaagctaaagaggaagtattggtggaaaaactgcaagatgatgatcatgctgggagccatctgtgccatcatcgtggtagttattgtaagtaagtatcgctgaagttgctggtggggtgaagaggtgggaaggtcctggagtcttctccctgcctgcctgcctgcctggggagtggggctgctctgactgaggtatggagaccgctgctatgggatcgtcancctggtccgggacgggnggaagggcagagacaagggatttctttgatggacaagccttccccctgcaaagcattgcnaggagatggcgcctcctgttctgaggaagtggggcntatgctgtgtgtcactgggcttggg

SEQ ID NO: 1036 - DNA, 498 bp, synthetic construct
  misc_feature 1..498: note = Synthetic polynucleotide
  source 1..498: mol_type = other DNA
gcacgaggctgattcctgtgtccagtctacttttttacttgagaatgtaccaccccttccctgttgtccattgccatccacattcatgtcctctgccctctgtttgctctctcaacacacttccccacccaccgtcctccattccagcccaggcttctccatcacccattcctcctttttcgttgcgttcatttgcactctgtccctcaacactagaaatgctgctcgtggcacaatctaagtcattacccgaagagcaacagctggcgcctcctccctgcctgctttttctgtactctcaagttcccccaaagccccaaagagttggaggccaagggaaggggcagggaggggagtggctgaggcgaagtacccatgaagctgcccagacttgggaggagaagagtatcggtgcccatggtgacttctagaaaacctggccactggtaggagctagggaggggctgggaagatggcagctcactcagtactgcctgg

SEQ ID NO: 1037 - DNA, 351 bp, synthetic construct
  misc_feature 1..351: note = Synthetic polynucleotide
  source 1..351: mol_type = other DNA
atgtctgctccagctcagccacctgctgaagggacagaagggactgccccaggtgggggtccccctggccctcctcctaacatgaccagtaacagacgactacagcaaacccaggcacaagtggaggaggtggtggacatcatacgtgtgaacgtggacaaggtcctggagagggaccagaagctgtcagagctggatgaccgagctgatgccttgcaggcaggagcatcacaatttgagagcagtgctgccaagctaaagaggaagtattggtggaaaaactgcaagatgatgatcatgctgggagccatctgtgccatcatcgtggtagttattgtaaggcgggacgac

SEQ ID NO: 1038 - DNA, 351 bp, synthetic construct; identical to SEQ ID NO: 1019
  misc_feature 1..351: note = Synthetic polynucleotide
  source 1..351: mol_type = other DNA

SEQ ID NO: 1039 - DNA, 351 bp, synthetic construct; identical to SEQ ID NO: 1019
  misc_feature 1..351: note = Synthetic polynucleotide
  source 1..351: mol_type = other DNA

SEQ ID NO: 1040 - DNA, 351 bp, synthetic construct
  misc_feature 1..351: note = Synthetic polynucleotide
  source 1..351: mol_type = other DNA
atgtctgctaccgctgccacggccccccctgctgccccggctggggagggtggtccccctgcaccccctccaaacctcaccagtaacaggagactgcagcagacccaggcccaggtggatgaggtggtggacatcatgagggtgaacgtggacaaggtcctggagcgagaccagaagctgtcggagctggacgaccgtgcagatgcactccaggcgggggcctcccagtttgaaacaagcgcagccaagctcaagcgcaaatactggtggaaaaacctcaagatgatgatcatcttgggagtgatttgcgccatcatcctcatcatcatcatagtttacttcagctcttaa

SEQ ID NO: 1041 - DNA, 351 bp, synthetic construct
  misc_feature 1..351: note = Synthetic polynucleotide
  source 1..351: mol_type = other DNA
atgtctgctaccgctgccacggccccccctgctgccccggctggggagggtggtccccctgcaccccctccaaacctcaccagtaacaggagactgcagcagacccaggcccaggtggatgaggtggtggacatcatgagggtgaacgtggacaaggtcctggagcgagaccagaagctgtcggagctggacgaccgtgcagatgcactccaggcgggggcctcccagtttgaaacaagcgcagccaagctcaagcgcaaatactggtggaaaaacctcaagatgatgatcatcttgggagtgatttgcgccatcatcctcatcatcatcatagtttacttcagcacttaa

SEQ ID NO: 1042 - DNA, 357 bp, synthetic construct
  misc_feature 1..357: note = Synthetic polynucleotide
  source 1..357: mol_type = other DNA
atggacaggtctgctaccgctgccacggccccccctgctgccccggctggggagggtggtccccctgcaccccctccaaacctcaccagtaacaggagactgcagcagacccaggcccaggtggatgaggtggtggacatcatgagggtgaacgtggacaaggtcctggagcgagaccagaagctgtcggagctggacgaccgtgcagatgcactccaggcgggggcctcccagtttgaaacaagcgcagccaagctcaagcgcaaatactggtggaaaaacctcaagatgatgatcatcttgggagtgatttgcgccatcatcctcatcatcatcatagtttacttcagcacttaa

SEQ ID NO: 1043 - DNA, 351 bp, synthetic construct; identical to SEQ ID NO: 1041
  misc_feature 1..351: note = Synthetic polynucleotide
  source 1..351: mol_type = other DNA

SEQ ID NO: 1044 - DNA, 351 bp, synthetic construct; identical to SEQ ID NO: 1041
  misc_feature 1..351: note = Synthetic polynucleotide
  source 1..351: mol_type = other DNA

SEQ ID NO: 1045 - DNA, 351 bp, synthetic construct; identical to SEQ ID NO: 1041
  misc_feature 1..351: note = Synthetic polynucleotide
  source 1..351: mol_type = other DNA

SEQ ID NO: 1046 - DNA, 351 bp, synthetic construct
  misc_feature 1..351: note = Synthetic polynucleotide
  source 1..351: mol_type = other DNA
atgtcggctaccgctgccacggccccccctgctgccccggctggggagggtggtccccctgcacctcctccaaatcttaccagtaacaggagactgcagcagacccaggcccaggtggatgaggtggtggacatcatgagggtgaacgtggacaaggtcctggagcgagaccagaagctgtcggagctggacgaccgtgcagatgcactccaggcgggggcctcccagtttgaaacaagcgcagccaagctcaagcgcaaatactggtggaaaaacctcaagatgatgatcatcttgggagtgatttgcgccatcatcctcatcatcatcatagtttacttcagctcttaa

SEQ ID NO: 1047 - DNA, 351 bp, synthetic construct
  misc_feature 1..351: note = Synthetic polynucleotide
  source 1..351: mol_type = other DNA
atggcaggaatagagttggagcggtgccagcagcaggcgaacgaggtgacggaaattatgcgtaacaacttcggcaaggtcctggagcgtggtgtgaagctggccgaactgcagcagcgttcagaccaactcctggatatgagctcaaccttcaacaagactacacagaacctggcccagaagaagtgctgggagaacatccgttaccggatctgcgtggggctggtggtggttggtgtcctgctcatcatcctgattgtgctgctggtcgtctttctccctcagagcagtgacagcagtagtgccccacggacccaggatgcaggcattgcctcagggcctgggaactga

SEQ ID NO: 1048 - DNA, 632 bp, synthetic construct
  misc_feature 1..632: note = Synthetic polynucleotide
  source 1..632: mol_type = other DNA
gcggtgcggcggctgcgtttnnatgagnnnctccangactttgtgctgcctgccgtgaggtggggagggggtacctccgtgccgacgtcctactccgtgatgtgaggcacaggcatgctcagtagctgatgcctacccctcctctctcggcaacggtgcctacaacactgagagaccgctggcagccggagcggggccggaaagccatctttgcgggagcggaggcagcggcggtcgggagccatgtctgctccagctcctacccaaggacccaccagtactggggctgcagggccacctcctgctaccaatgtgtcaagtaacaaacggctgcagcagacgcaagcccaagtggatgaagtggtagacatcatgagaatgaacgtggacaaggtgttagaaagagaccagaagctgtcagagcttgacaaccgggcagatgcattgcaagcaggtgcctcacagtttgaaaccagtgcagccaaactgaagagaaaatattggtggaaaaattgcaagatgatgatcatccttggtgtagtatgcacagtcattctcattataattataatttatttctccactnnnnnngatgannnnngangncntggcacaactttgttcgtatcaac

SEQ ID NO: 1049 - DNA, 349 bp, synthetic construct
  misc_feature 1..349: note = Synthetic polynucleotide
  source 1..349: mol_type = other DNA
atgtctgctaccgctgccacggccccccctgctgccccggctggggagggtggtccccctgcaccccctccaaacctcaccagtaacaggagactgcagcagacccaggcccaggtggatgaggtggtggacatcatgagggtgaacgtggacaaggtcctggagcgagaccagaagctgtcggagctggacgaccgtgcagatgcactccaggcgggggcctcccagtttgaaacaagcgcagccaagctcaagcgcaaatactggtggaaaaacctcaagatgatgatcatcttgggagtgatttgcgccatcatcctcatcatcatcatagtttacttcagcactt

SEQ ID NO: 1050 - DNA, 439 bp, synthetic construct
  misc_feature 1..439: note = Synthetic polynucleotide
  source 1..439: mol_type = other DNA
gcacgaggccagtcggagccagcgcgagccgccgccgccatcactgccgctgccaagtcctccacccgctgcccccgccatgtctgctaccgctgccacggccccccctgctgccccggctggggagggtggtccccctgcaccccctccaaacctcaccagtaacaggagactgcagcacacccaggcccaggtggatgaggtggtggacatcatgagggtgaacgtggacaaggtcctggagcgagaccagaagctgtcggagctggacgaccgtgcagatgcactccaggcgggggcctcccagtttgaaacaagcgcagccaagctcaagcgcaaatactggtggaaaaacctcaagatgatgatcatcttgggagtgatttgcgccatcatcctcatcatcatcatagtttacttcagcacttaaatccccgag

SEQ ID NO: 1051 - DNA, 300 bp, synthetic construct
  misc_feature 1..300: note = Synthetic polynucleotide
  source 1..300: mol_type = other DNA
atgtctacaggtccaactgctgccactggcagtaatcgaagacttcagcagacacaaaatcaagtagatgaggtggtggacataatgcgagttaacgtggacaaggttctggaaagagaccagaagctctctgagttagacgaccgtgcagacgcactgcaggcaggcgcttctcaatttgaaacgagcgcagccaagttgaagaggaaatattggtggaagaattgcaagatgtgggcaatcgggattactgttctggttatcttcatcatcatcatcatcgtgtgggttgtctcttca

SEQ ID NO: 1052 - DNA, 303 bp, synthetic construct
  misc_feature 1..303: note = Synthetic polynucleotide
  source 1..303: mol_type = other DNA
atgtctacaggtccaactgctgccactggcagtaatcgaagacttcagcagacacaaaatcaagtagatgaggtggtggacataatgcgagttaacgtggacaaggttctggaaagagaccagaagctctctgagttagacgaccgtgcagacgcactgcaggcaggcgcttctcaatttgaaacgagcgcagccaagttgaagaggaaatattggtggaagaattgcaagatgtgggcaatcgggattactgttctggttatcttcatcatcatcatcatcgtgtgggttgtctcttcatga

SEQ ID NO: 1053 - DNA, 301 bp, synthetic construct
  misc_feature 1..301: note = Synthetic polynucleotide
  source 1..301: mol_type = other DNA
atgtctacaggtccaactgctgccactggcagtaatcgaagacttcagcagacacaaaatcaagtagatgaggtggtggacataatgcgagttaacgtggacaaggttctggaaagagaccagaagctctctgagttagacgaccgtgcagacgcactgcaggcaggcgcttctcaatttgaaacgagcgcagccaagttgaagaggaaatattggtggaagaattgcgagatgtgggcaatcgggattactgttctggttatcttcatcatcatcatcatcgtgtgggttgtctcttcat

SEQ ID NO: 1054 - DNA, 301 bp, synthetic construct
  misc_feature 1..301: note = Synthetic polynucleotide
  source 1..301: mol_type = other DNA
atgtctacaggtccaactgctgccactggcagtaatcgaagacttcagcagacacaaaatcaagtagatgaggtggtggacataatgcgagttaacgtggacaaggttctggaaagagaccagaagctctctgagttagacgaccgtgcagacgcactgcaggcaggcgcttctcaatttgaaacgagcgcagccaagttgaagaggaaatattggtggaagaattgcaagatgtgggcaatcgggattactgttctggttatcttcatcatcatcatcatcgtgtgggttgtctcttcat

SEQ ID NO: 1055 - DNA, 303 bp, synthetic construct; identical to SEQ ID NO: 1052
  misc_feature 1..303: note = Synthetic polynucleotide
  source 1..303: mol_type = other DNA

SEQ ID NO: 1056 - DNA, 369 bp, synthetic construct
  misc_feature 1..369: note = Synthetic polynucleotide
  source 1..369: mol_type = other DNA
atgtctacaggtccaactgctgccactggcagtaatcgaagacttcagcagacacaaaatcaagtagatgaggtggtggacataatgcgagttaacgtggacaaggttctggaaagagaccagaagctctctgagttagacgaccgtgcagacgcactgcaggcaggcgcttctcaatttgaaacgagcgcagccaagttgaagaggaaatattggtggaagaattgcaagatgtgggcaatcgggattactgttctggttatcttcatcatcatcatcatcggtgagttacccttttctaaactgattggaaaagtcttctccatgtgttcacagaccattgattttgtgtacaagaactcagattaa

SEQ ID NO: 1057 - DNA, 300 bp, synthetic construct; identical to SEQ ID NO: 1051
  misc_feature 1..300: note = Synthetic polynucleotide
  source 1..300: mol_type = other DNA

SEQ ID NO: 1058 - DNA, 1115 bp, synthetic construct
  misc_feature 1..1115: note = Synthetic polynucleotide
  source 1..1115: mol_type = other DNA
gtacaagaaagttgggcatgaagagacaacccacacgatgatgatgatgatgaagataaccagaacagtaatcccgattgcccacatcttgcaattcttccaccaatatttcctcttcaacttggctgcgctcgtttcaaattgagaagctccagcctgcagtgcctctgctcggtcgtctaactcacagaggttatggtctctttcaagaacctagtctacagttaacttgtattatgacaacctccttcactacatgaattcttaattcagatcaactcatcaatctccatcacaccgttttcagtagcgtaattatgtcattcatacttctcatatactattgcagatatcaattggtttcgacatttcatgttctattactgtctacacgtatatgatttaccggtaatcatattatagttcgttctcactgggtcggcttatgataatccatcatcagtacctatttattatccaatcttttttggttgctcattacctctttagcctattattatacctttattctatggtcgtcgtgaacatttgtttattctttagttcactgtttttccttaatacatcgtaatctgtctcagatcaacttgtttttacatttattagtgtcggcctatatttatatcatcattctattctgtttgtcctgcccccggctggatgtattgactctgtatcttgtttgctcatatcatgtgtacctttaggtgtctccctaagctatatatctcaccaaactacgcttactttgcagcttgtttacctctccaccattgctaattttgtgtgctcgatcgcttggtacattcgcttacagtaattgactttactacgtcgacaatcaattttacggctcacttttttaaaagtttggactaggataaatcaatttcccctatcttggctttattaccacttactagatctgacagaattattatttatatattatatcctttcatgtttttctcaccctctcttcgccttatcagttggtacctgtctatacctcacatgcgcttcttcgaatccgatgcgtcccaacactattttcgctatactcctctacccagtacacactaagaatgactaga

SEQ ID NO: 1059 - DNA, 351 bp, synthetic construct; identical to SEQ ID NO: 1047
  misc_feature 1..351: note = Synthetic polynucleotide
  source 1..351: mol_type = other DNA

SEQ ID NO: 1060 - DNA, 303 bp, synthetic construct
  misc_feature 1..303: note = Synthetic polynucleotide
  source 1..303: mol_type = other DNA
atggaggaagccagtgaaggtggaggaaatgatcgtgtgcggaacctgcaaagtgaggtggagggagttaagaatattatgacccagaatgtggagcggatcctggcccggggggaaaacttggaacatctccgcaacaagacagaggatctggaagccacatctgagcacttcaagacgacatcgcagaaggtggctcgaaaattctggtggaagaacgtgaagatgattgtccttatccgcgtgattgtttttatcatcatcctcttcattgtgctctttgccactggtgccttctcttaa

SEQ ID NO: 1061 - DNA, 351 bp, synthetic construct; identical to SEQ ID NO: 1047
  misc_feature 1..351: note = Synthetic polynucleotide
  source 1..351: mol_type = other DNA

SEQ ID NO: 1062 - DNA, 351 bp, synthetic construct; identical to SEQ ID NO: 1047
  misc_feature 1..351: note = Synthetic polynucleotide
  source 1..351: mol_type = other DNA

SEQ ID NO: 1063 - DNA, 783 bp, synthetic construct
  misc_feature 1..783: note = Synthetic polynucleotide
  source 1..783: mol_type = other DNA
atggcgattctttttgctgttgttgccagggggaccactatccttgccaaacatgcttggtgtggaggaaacttcctggaggtgacagagcagattctggctaagataccttctgaaaataacaaactaacgtactcacatggcaattatttgtttcattacatctgccaagacaggattgtatatctttgtatcactgatgatgattttgaacgttcccgagcctttaattttctgaatgagataaagaagaggttccagactacttacggttcaagagcacagacagcacttccatatgccatgaatagcgagttctcaagtgtcttagctgcacagctgaagcatcactctgagaataagggcctagacaaagtgatggagactcaagcccaagtggatgaactgaaaggaatcatggtcagaaacatagtctgtcaccttcaaaactaccagcagaaatcttgctcgagccatgtgtatgaagaacctcaagctcactattatcatcatcatcgtatcaattgtgttcatctatatcattgtttcacctctctgtggtggatttacatggccaagctgtgtgaagaaataggaaagaagaagttaccattaaccaaggatatgagagaacaaggagttaaaagcaatccatgtgactcaagcctttcacatactgacagatggtatctgccagtctcttcaaccctcttctcactttttaaaatcttgttccatgcctccaggtttatctttgtcttatctaccagtttattcctgtga

SEQ ID NO: 1064 - DNA, 540 bp, synthetic construct
  misc_feature 1..540: note = Synthetic polynucleotide
  source 1..540: mol_type = other DNA
atggcgattctttttgctgttgttgccagggggaccactatccttgccaaacatgcttggtgtggaggaaacttcctggaggattttgaacgttcccgagcctttaattttctgaatgagataaagaagaggttccagactacttacggttcaagagcacagacagcacttccatatgccatgaatagcgagttctcaagtgtcttagctgcacagctgaagcatcactctgagaataagggcctagacaaagtgatggagactcaagcccaagtggatgaactgaaaggaatcatggtcagaaacatagatctggtagctcagcgaggagaaagattggaattattgattgacaaaacagaaaatcttgtggattcttctgtcaccttcaaaactaccagcagaaatcttgctcgagccatgtgtatgaagaacctcaagctcactattatcatcatcatcgtatcaattgtgttcatctatatcattgtttcacctctctgtggtggatttacatggccaagctgtgtgaagaaatag

SEQ ID NO: 1065 - DNA, 663 bp, synthetic construct
  misc_feature 1..663: note = Synthetic polynucleotide
  source 1..663: mol_type = other DNA
atggcgattctttttgctgttgttgccagggggaccactatccttgccaaacatgcttggtgtggaggaaacttcctggaggtgacagagcagattctggctaagataccttctgaaaataacaaactaacgtactcacatggcaattatttgtttcattacatctgccaagacaggattgtatatctttgtatcactgatgatgattttgaacgttcccgagcctttaattttctgaatgagataaagaagaggttccagactacttacggttcaagagcacagacagcacttccatatgccatgaatagcgagttctcaagtgtcttagctgcacagctgaagcatcactctgagaataagggcctagacaaagtgatggagactcaagcccaagtggatgaactgaaaggaatcatggtcagaaacatagatctggtagctcagcgaggagaaagattggaattattgattgacaaaacagaaaatcttgtggattcttctgtcaccttcaaaactaccagcagaaatcttgctcgagccatgtgtatgaagaacctcaagctcactattatcatcatcatcgtatcaattgtgttcatctatatcattgtttcacctctctgtggtggatttacatggccaagctgtgtgaagaaatag

SEQ ID NO: 1066 - DNA, 663 bp, synthetic construct; identical to SEQ ID NO: 1065
  misc_feature 1..663: note = Synthetic polynucleotide
  source 1..663: mol_type = other DNA

SEQ ID NO: 1067 - DNA, 663 bp, synthetic construct; identical to SEQ ID NO: 1065
  misc_feature 1..663: note = Synthetic polynucleotide
  source 1..663: mol_type = other DNA

SEQ ID NO: 1068 - DNA, 303 bp, synthetic construct
  misc_feature 1..303: note = Synthetic polynucleotide
  source 1..303: mol_type = other DNA
atggaggaagccagtgaaggtggaggaaatgatcgtgtgcggaacctgcaaagtgaggtggagggagttaagaatattatgacccagaatgtggagcggatcctggcccggggggaaaacttggaacatctccgcaacaagacagaggatctggaagccacatctgagcacttcaagacgacatcgcagaaggtggctcgaaaattctggtggaagaacgtgaagatgattgtccttatctgcgtgattgtttttatcatcatcctcttcattgtgctctttgccactggtgccttctcttaa

SEQ ID NO: 1069 - DNA, 300 bp, synthetic construct
  misc_feature 1..300: note = Synthetic polynucleotide
  source 1..300: mol_type = other DNA
atggaggaagccagtgaaggtggaggaaatgatcgtgtgcggaacctgcaaagtgaggtggagggagttaagaatattatgacccagaatgtggagcggatcctggcccggggggaaaacttggaacatctccgcaacaagacagaggatctggaagccacaccaggggctgaggccagctgtttcagggaggaagtaactccaaagttgagcaaagtctggagcctcaagttctgtgttcagcggggaggctggctctggctttctgagtcctgtgtgaactcttggctcttggtctga

SEQ ID NO: 1070 - DNA, 303 bp, synthetic construct; identical to SEQ ID NO: 1068
  misc_feature 1..303: note = Synthetic polynucleotide
  source 1..303: mol_type = other DNA

SEQ ID NO: 1071 - DNA, 1701 bp, synthetic construct
  misc_feature 1..1701: note = Synthetic polynucleotide
  source 1..1701: mol_type = other DNA
atgaaggcaatactagtagttctgctatatacatttgcaaccgcaaatgcagacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccaccgcaagttcatggtcctacattgtggaaacatctagttcagacaatggaacgtgttacccaggagatttcatcaattatgaggagctaagagagcaattgagctcagtgtcatcatttaaaaggtttgagatattccccaagacaagttcatggcccaatcatgactcgaacaaaggtgtaacggcagcatgtcctcacgctggagcaaaaagcttctacaaaaatttaatatggctagttaaaaaaggaaattcatacccaaagctcagccaatcctacattaatgataaagggaaagaagtcctcgtgctgtggggcatccaccatccatctactactgctgaccaacaaagtctctatcagaatgcagatgcatatgtttttgtggggacatcaagatacagcaaaaagttcaagccggaaatagcaataagacccaaagtgagggatcaagaagggagaatggactattactggacactagtagagccgggagacaaaataacattcgaagcaactggaaatctagtggtaccgagatatgcattcacaatggaaagaaatgctggatctggtattatcatttcagatacaccagtccacgattgcaatacaacttgtcagacacccgagggtgctataaacaccagcctcccatttcagaatatacatccgatcacaattggaaaatgtccaaagtatgtaaaaagcacaaaattgagactggccacaggattgaggaatgtcccgtctattcaatctagaggcctattcggggccattgccggcttcattgaaggggggtggacagggatggtagatggatggtacggttatcaccatcaaaatgagcaggggtcaggatatgcagccgacctgaagagcacacaaaatgccattgacaaaattactaacaaagtaaattctgttattgaaaagatgaatacacagttcacagcagtggataaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttgtatgaaaaggtaagaaaccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagagaaaatagatggggtaaagctggaatcaacaaggatttaccagattttggcgatctattcaactgtcgccagttcattggtactggtagtctccctgggggcaatcagcttctggatgtgctctaatgggtctctacaatgtagaatatgtatttaa

SEQ ID NO: 1072 - DNA, 1716 bp, synthetic construct
  misc_feature 1..1716: note = Synthetic polynucleotide
  source 1..1716: mol_type = other DNA
atggaccgcgcggttaacagagtcgtgctagagaatgaggaaagagaagcaaagaacacatggcgcctggtcttccggatcgcagtcttacttttaatggtaatgactctagctatctccgcagctgccctggcatacagtatggaggccagcacgccgcacgactttgcaggcatatcgactgtgatctctaagacagaagataaggttacgtctttactcagttcgagtcaagatgtgatagataggatatacaagcaggtggctcttgaatccccactggcgctactaaacactgaatctacaattatgaatgcaataacctctctttcttatcaaattaacggggctgcgaacaatagcggatgtggggcgcctgttcatgacccagattatatcggggggataggcaaagaactcatagtggacgacatcagtgatgtcacatcattttatccttctgcatatcaagaacacctgaatttcatcccggcgcctactacaggattcggttgcactcggataccctcatttgacatgagcaccacccattattgttatactcacaatgtaatactatccggttgcagagatcactcacactcacatcaatacttagcgcttggtgtgcttcggacatctgcaacagggagggtattcttctctactctgcactccatcaatttagatgatgcccaaaatcggaagtcctgcagtgtgagtgcaacccctttaggttgtgatatgctgtgctctaaggtcacagggactgaagaggaggattacaagtcagctgccccaacatcaatggtgcacggaaggctagggtttgacggtcaataccatgagaaggacttagacaccacggtcttatttaaggattgggtggcaaattacccaggagtgggaggagggtcttttattgacgaccgtgtatggttcccagtttacggagggctcaaacccaattcacccagtgacactgcacaagaagggaaatatgtaatatacaagcgccataacaacacatgccccgatggacaagattaccaaattcggatggctaagtcttcatataaacccgggcgatttggtggaaagcgcgtacagcaagccatactatccatcaaagtgtcaacatccttgggtaaggacccggtgctgactattccacctaatacaatcacactcatgggagccgaaggcagaatcctcacagtagggacatctcacttcttgtaccaacgagggtcttcatatttctcccctgccttattatatcccatgacagtaaataacaaaacggctacactccatagtccttatacgtttaatgctttcactcggccaggtagtgtcccttgccaggcatcagcaagatgccccaactcatgcatcactggggtctatactgatccatatcccttaatcttctataggaatcatactctacgaggggtctttgggacgatgcttgatgctgaacaagcgagacttaaccccgtatctgcagtattcgacaacatatcccgcagtcgtgtcacccgggtgagttcaagcagcaccaaggcagcatacacgacatcgacatgttttaaagttgtcaagaccaataaagcttattgtcttagtattgcagagatatccaataccctattcggggaatttaggatcgttcccttattagttgagatcctcaaggatgatagagtttaa

SEQ ID NO: 1073 - DNA, 2550 bp, synthetic construct
  misc_feature 1..2550: note = Synthetic polynucleotide
  source 1..2550: mol_type = other DNA
atgagagtgatggggatacagaagaattatccactcttatggagatggggtatgacaatatggggtatggcaatattttggataatgataatttgcaatgctgaagatttgtgggtcacggtctactatggggtacctgtgtggaaagaggcagagaccaccctattttgtgcatcagatgctaaggcatacgatacagaagtacataatgtttgggctacacatgcctgcgtacccacagaccctaacccacaagaaatacatttggaaaatgtaacagaaaattttaacatgtggaaaaataacatggtagagcagatgcatgaggatgtaattagtctatgggatcaaagcctaaagccatgtgtaaagttaacccctctctgcgttactttaaattgtagtaacctcaacaacatcactagcaacagcagcaacagcactagcaacaacagcacattgagtgacattgacatgaaaaactgctctttcattgtaaccacagaactaagagataagaaaaagagagagtatgcacttttttataaacaagatgtagtccaaataaatgaaaatgaaagtaacagtagtcagtatatgttaataaattgtaatacctcaaccattacacaggcttgtccaaaggtaacctttgagccaattcccatacattattgtgctccagctggttttgcaattctaaaatgtaatgatagagagttcaatggaacagggccatgcgaaaatgtcagcacagtacaatgtacacatggaatcaagccagtagtaacaactcaactgctgttaaatggcagtacagcagaagaagagataataattagatctgaagatatcataaacaatgctaaaaccataatagtacagttagttaagcctgtaaacattacttgtgtcagacctggcaacaatacaagaaaaagtgtacgtataggaccagggcaagcattctatacaacaggtgaagtaataggaaatataagaaaggcatattgtattgtcaacggtacagaatggaatgacactttctaccgggtggctgaaaaattagcgaagtactttaacagcaaaacaataatcttcaatcaaccctcaggaggggatgtagaaattacaacacatagttttaattgtggaggagaatttttctattgcaatacatcagacctgtttaatgcatcaagcacttggggtaacaatataactctcccatgcagaataaagcaaattgtaaatatgtggcagaaagtaggacgagcaatgtatgcccctcccattccaggagaaataaggtgtgaatcaaacataacaggactactattaacaagagatggagggaataataataatacaaatgagaccttcagacctggaggaggagatatgagggacaattggagaagtgaattatataagtataaagtagtaaaaattgaaccactaggtgtagcacccacccatgcaaaaagaagagtggtgcagagagaaaaaagagcagttggactgggagctgtcttccttgggttcttaggagcagcaggaagcactatgggcgcggcgtcaataacgctgacggtacaggccagacaattgctatctggtatagtgcaacagcagagcaatttgctgaaggctatagaggctcaacaacatctgttgagactcacggtctggggcattaaacagctccaggcaagagtcctggctctggaaagatacctaaaggatcaacagctcctaggaatttggggctgctctggaaaactcatctgccacactactgtaccctggaactccagttggagtaataaaactttccatgacatatgggacaacatgacctggctgcaatgggataaagaaattagtaattacacaagcataatatatggcctaattgaagaatcgcagaaccagcaggaaaaaaatgaacaagacctattggcattagacaagtgggcaaatctgtggaattggtttgacataacaaattggctatggtatataaaaatatttataataatagtaggaggtttgataggtttacgaataatttttgctgtgcttaatataataaatagagttaggcagggatactcacctttgtcattccagacccttacccaccaccagagggaacccgacagacccgaaagaatcgaagaaggaggtggcgagcaagacagagacagatccgtgagattagtcaacgggttcttagcgcttgcctgggacgatctgcggagcctgtgcctcttcagctaccaccaattgagagactttgtcttgattgtagtgaggattgtggaacttctgggacacagagggtgggaagccctcaaatatctgtggaatcttctatcatactggggtcaggaactaaagaatagtgctattaatctgattgatacaacagcaatagcagtagctaactggacagacagagttatagaaataggacaaagagctggtagagctatccgtaacatacctacaagaattagacagggcctagaaagagctttgctataa

SEQ ID NO: 1074 - DNA, 813 bp, synthetic construct
  misc_feature 1..813: note = Synthetic polynucleotide
  source 1..813: mol_type = other DNA
ccagtagtatcaactcaactactgttaaatggcagtctagcagaagaagaggtagtaattaggtctgacaatttcacaaacaatgctaaaatcataatagtacagctgaatgaatctgtaaatattacttgtatgagacccaacaacaatacaagaagagatatacatataggaccaggcagagcattttatgcaacagacataataggagatataagacaagcacattgtaaccttagtgaagcaaaatggaataacactttaaaacagatagttacaagattaagagaccaatttgggaataaaacaatagtctttaatcaatcctcaggaggggatccagaaattgtaatgcacagttttaattgtggaggggaatttttcttctgtaatacaaacaaactgtttaatagtatttggcatgttaatgatacttggaatgttactgaaaagtcaaatagcactggaggaaatgaaacaatcatactcccatgcagaataaaacaaattataaacatgtggcaggaggtaggaaaagcaatgtatgcccctcccatcagaggagtaattaaatgctcatcaaatattacagggctgatattaacaagagatgggggtagcggtaacgggacaacaaacaataccgagaccttcaggcctggaggaggagatatgagggacaattggagaagtgaattatataaatataaagtagtacaaattgaacccttaggaatagcacccaccagggcaaagagaagagtggtgcagagagaaaaaagagcagtgggaataggagctttgttccttggg

SEQ ID NO: 1075 - DNA, 414 bp, synthetic construct
  misc_feature 1..414: note = Synthetic polynucleotide
  source 1..414: mol_type = other DNA
tctggcatagtgcaacagcaaagcaatttgctcctgtatatagaggctcaacagcatctgttgaaactcacggtctggggcattaaacagctccaggcacgagtcctggctgtggaaagatacctaaaggatcaacagctcctagggatttggggctgctctggaaaactcatctgccccactactgtgccctggaactctagttggagtaataaatcctacagtgagatatgggacaacatgacctggctgcaatgggataaagaagttaacaattacacacaggaaatatataggttaattgaacaatcgcagaaccagcaggaaaagaatgaacaagacttattggcattggacaagtgggcaaatctgtggaattggtttgacatatcaagctggctgtggtatataaaa

SEQ ID NO: 1076 - DNA, 10176 bp, synthetic construct
  misc_feature 1..10176: note = Synthetic polynucleotide
  source 1..10176: mol_type = other DNA
atgaatgaccaacggaaaaaggcgaaaaacacgccattcaatatgctgaaacgcgagagaaaccgcgtgtcgactgtgcaacagctgacaaagagattctcacttggaatgctacagggacgaggaccactaaaactgtacatggccctggtggcgttccttcgtttcctaacaatcccaccaacagcagggatattgaagagatggggaacaattaaaaaatccaaagctattaatgttttgagagggttcaggaaagagattggaaggatgctgaacatcttgaataggagacgcagatctgcaggcatgatcattatgctgattccaacagtgatggcgttccatttaaccacacgtaacggagaaccacacatgatcgtcagcagacaagagaaagggaaaagtcttctgtttaaaacagaggatggcgtgaacatgtgtaccctcatggccatggaccttggtgaattgtgtgaagacacaatcacgtacaagtgtccccttctcaggcagaatgagccagaagacatagactgttggtgcaactctacctccacgtgggtaacttatgggacgtgtaccaccatgggagaacatagaagacaaaaaagatcagtggcactcgttccacatgtgggaatgggactggagacacgaactgaaacatggatgtcatcagaaggggcctggaaacatgtccagagaattgaaacttggatcttgagacatccaggcttcaccatgatggcagcaatcctggcatacaccataggaacgacacatttccaaagagccctgattttcatcttactgacagctgtcactccttcaatgacaatgcgttgcataggaatgtcaaatagagactttgtggaaggggtttcaggaggaagctgggttgacatagtcttagaacatggaagctgtgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggactatttggaaagggaggcattgtgacctgtgctatgttcagatgcaaaaagaacatggaaggaaaagttgtgcaaccagaaaacttggaatacaccattgtgataacacctcactcaggggaagagcatgcagtcggaaatgacacaggaaaacatggcaaggaaatcaaaataacaccacagagttccaccacagaagcagaattgacaggttatggcactgtcacaatggagtgctctccaagaacgggcctcgacttcaatgagatggtgttgctgcagatggaaaataaagcttggctggtgcacaggcaatggttcctagacctgccgttaccatggttgcccggagcggacacacaagggtcaaattggatacagaaagagacattggtcactttcaaaaatccccatgcgaagaaacaggatgttgttgttttaggatcccaagaaggggccatgcacacagcacttacaggggccacagaaatccaaatgtcatcaggaaacttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgcaagatcccttttgagataatggatttggaaaaaagacatgtcttaggtcgcctgattacagtcaacccaattgtgacagaaaaagatagcccagtcaacatagaagcagaacctccattcggagacagctacatcatcataggagtagagccgggacaactgaagctcaactggtttaagaaaggaagttctatcggccaaatgtttgagacaacaatgaggggggcgaagagaatggccattttaggtgacacagcctgggattttggatccttgggaggagtgtttacatctataggaaaggctctccaccaagtctttggagcaatctatggagctgccttcagtggggtttcatggactatgaaaatcctcataggagtcattatcacatggataggaatgaattcacgcagcacctcactgtctgtgacactagtattggtgggaattgtgacactgtatttgggagtcatggtgcaggccgatagtggttgcgttgtgagctggaaaaacaaagaactgaaatgtggcagtgggattttcatcacagacaacgtgcacacatggacagaacaatacaagttccaaccagaatccccttcaaaactagcttcagctatccagaaagcccatgaagagggcatttgtggaatccgctcagtaacaagactggagaatctgatgtggaaacaaataacaccagaattgaatcacattctatcagaaaatgaggtgaagttaactattatgacaggagacatcaaaggaatcatgcaggcaggaaaacgatctctgcggcctcagcccactgagctgaagtattcatggaaaacatggggcaaagcaaaaatgctctctacagagtctcataaccagacctttctcattgatggccccgaaacagcagaatgccccaacacaaatagagcttggaattcgttggaagttgaagactatggctttggagtattcaccaccaatatatggctaaaattgaaagaaaaacaggatgtattctgcgactcaaaactcatgtcagcggccataaaagacaacagagccgtccatgccgatatgggttattggatagaaagtgcactcaatgacacatggaagatagagaaagcctctttcattgaagtcaaaaactgccactggccaaaatcacacaccctctggagcaatggagtgctagaaagtgagatgataattccaaagaatctcgctggaccagtgtctcaacacaactatagaccaggctaccatacacaaataacaggaccatggcatctaggtaagcttgagatggactttgatttctgtgatggaacaacagtggtagtgactgaggactgcggaaatagaggaccctctttgagaacaaccactgcctctggaaaactcataacagaatggtgctgccgatcttgcacattaccaccgctaagatacagaggtgaggatgggtgctggtacgggatggaaatcagaccattgaaggagaaagaagagaatttggtcaactccttggtcacagctggacatgggcaggtcgacaacttttcactaggagtcttgggaatggcattgttcctggaggaaatgcttaggacccgagtaggaacgaaacatgcaatactactagttgcagtttcttttgtgacattgatcatagggaacatgtcctttagagacctgggaagagtaatggttatggtaggcgccactatgacggatgacataggtatgggcgtgacttatcttgccctactagcagccttcaaagtcagaccaacttttgcagctggactactcttgagaaagctgacctccaaggcattgatgatgactactataggaattgtactctcctcccagagcactacaccagagaccattcttgagttgactgatgcgttagccttaggcatgatggtcctcaaaatggtgagaaatatggaaaagtatcaattggcagtgactatcatggctatcttgtgcgtcccaaacgcagtgatattacaaaacgcatggaaagtgagttgcacgatattggcagtggtgtccgtttccccactgttcttaacatcctcacagcaaaaaacggattggataccattagcgttgacgatcaaaggtctcaatccaacagctatttttctaacaaccctctcaagaaccagcaagaaaaggagctggccattaaatgaggctatcatggcagtcgggatggtgagcattttagccagttctctcctaaaaaatgatattcccatgacaggaccattagtggctggagggcccctcactgtgtgctacgtgctcactggacgatcggccgatttggaactggagagagcagccgatgtcaaatgggaagaccaggcagagatatcaggaagcagcccaatcctgtcaataacaatatcagaagatggtagcatgtcgataaaaaatgaagaggaagaacaaacactgaccatactcattagaacaggattgctggtgatctcaggactttttcctgtatcaataccaatcacggcagcagcatggtacctgtgggaagtgaagaaacaacgggccggagtattgtgggatgttccttcacccccacccatgggaaaggctgaactggaagatggagcctatagaattaagcaaaaagggattcttggatattcccagatcggagccggagtttacaaagaaggaacattccatacaatgtggcatgtcacacgtggcgctgttctaatgcataaaggaaagaggattgaaccatcatgggcggacgtcaagaaagacctaatatcatatggaggaggctggaagttagaaggagaatggaaggaaggagaagaagtccaggtattggcactggagcctggaaaaaatccaagagccgtccaaacgaaacctggtcttttcaaaaccaacgccggaacaataggtgctgtatctctggacttttctcctggaacgtcaggatctccaattatcgacaaaaaaggaaaagttgtgggtctttatggtaatggtgttgttacaaggagtggagcatatgtgagtgctatagcccagactgaaaaaagtattgaagacaacccagagatcgaagatgacattttccgaaaaagaagactgaccatcatggacctccacccaggagcgggaaagacgaagagataccttccggccatagtcagagaagctataaaacggggtttgagaacattaatcttggcccccactagagttgtggcagctgaaatggaggaagcccttagaggacttccaataagataccagaccccagccatcagagctgagcacaccgggcgggagattgtggacctaatgtgtcatgccacatttaccatgaggctgctatcaccagttagagtgccaaactacaacctgattatcatggacgaagcccatttcacagacccagcaagtatagcagctagaggatacatctcaactcgagtggagatgggtgaggcagctgggatttttatgacagccactcccccgggaagcagagacccatttcctcagagcaatgcaccaatcatagatgaagaaagagaaatccctgaacgttcgtggaattccggacatgaatgggtcacggattttaaagggaagactgtttggttcgttccaagtataaaagcaggaaatgatatagcagcttgcctgagcaaaaatggaaagaaagtgatacaactcagtaggaagacctttgattctgagtatgccaagactagaaccaatgattgggacttcgtggttacaactgacatttcagaaatgggtgccaatttcaaggctgagagggttatagaccccagacgctgcatgaaaccagtcatactaacagatggtgaagagcgggtgattctggcaggacctatgccagtgacccactctagtgcagcacaaagaagagggagaataggaagaaatccaaaaaatgagaatgaccagtacatatacatgggggaacccctggaaaatgatgaagactgtgcacactggaaagaagctaaaatgctcctagataacatcaacacgccagaaggaatcattcctagcatgttcgaaccagagcgtgaaaaggtggatgccattgatggcgaataccgcttgagaggagaagcaaggaccacctttgtagacttaatgagaagaggagacctaccagtctggttggcctacagagtggcagctgaaggcatcaactacgcagacagaaggtggtgttttgatggagtcaagaacaaccaaatcctagaagaaaacgtggaagttgaaatctggacaaaagaaggggaaaggaagaaattgaaacccagatggttggatgctaggatctattctgacccactggcgctaaaagaatttaaggaatttgcagccggaagaaagtctctgaccctgaacctaatcacagaaatgggtaggctcccaaccttcatgactcagaaggcaagagacgcactggacaacttagcagtgctgcacacggctgaggcaggtggaagggcgtacaaccatgctctcagtgaactgccggagaccctggagacattgcttttactgacacttctggctacagtcacgggagggatccttttattcttgatgagcggaaggggcatagggaagatgaccctgggaatgtgctgcataatcacggctagcatcctcctatggtacgcacaaatacagccacactggatagcagcttcaataatactggagttttttctcatagttttgcttattccagaacctgaaaaacagagaacaccccaagacaaccaactgacctacgttgtcatagccatcctcacagtggtggccgcaaccatggcaaacgagatgggtttcctagaaaaaacgaagaaagatctaggattaggaagcattgcaacccagcaacccgagagcaacatcctggacatagatctacgtcctgcatcagcatggacgctgtatgccgtggccacaacatttgttacaccaatgttgagacatagcattgaaaattcctcagtgaatgtgtccctaacagctatagccaaccaagccacagtgttaatgggtctcgggaaaggatggccattgtcaaagatggacatcggagttccccttctcgccattggatgctactcacaagtcaaccccacaactctcacagcagctcttttcttattggtagcacattatgccatcatagggccagcactccaagcaaaagcatccagagaagctcagaaaagagcagcggcgggcatcatgaaaaacccaactgtcgatggaataacagtgattgacctagatccaataccttatgatccaaagtttgaaaagcagttgggacaagtaatgctcctagtcctctgcgtgactcaagtattgatgatgaggactacatgggctctgtgtgaggttttaaccttagctaccgggcccatctccacattgtgggaaggaaatccagggaggttttggaacactaccattgcggtgtcaatggctaacatttttagagggagttacttggccggagctggacttctcttttctattatgaagaacacaaccaacgcaagaaggggaactggcaacataggagagacgcttggagagaaatggaaaagccgattgaacgcattgggaaaaagtgaattccagatctacaagaaaagtggaatccaggaagtggatagaaccttagcaaaagaaggcattaaaagaggagaaacggaccatcacgctgtgtcgcgaggctcagcaaaactgagatggttcgttgagagaaacatggtcacaccagaagggaaagtagtggacctcggttgtggcagaggaggctggtcatactattgtggaggactaaagaatgtaagagaagtcaaaggcctaacaaaaggaggaccaggacacgaagaacccatccccatgtcaacatatgggtggaatctagtgcgtcttcaaagtggagttgacgttttcttcatcccgccagaaaagtgtgacacattattgtgtgacataggggagtcatcaccaaatcccacagtggaagcaggacgaacactcagagtccttaacttagtagaaaattggttgaacaacaacactcaattttgcataaaggttctcaacccatatatgccctcagtcatagaaaaaatggaagcactacaaaggaaatatggaggagccttagtgaggaatccactctcacgaaactccacacatgagatgtactgggtatccaatgcttccgggaacatagtgtcatcagtgaacatgatttcaaggatgttgatcaacagatttacaatgagatacaagaaagccacttacgagccggatgttgacctcggaagcggaacccgtaacatcgggattgaaagtgagataccaaacctagatataattgggaaaagaatagaaaaaataaagcaagagcatgaaacatcatggcactatgaccaagaccacccatacaaaacgtgggcataccatggtagctatgaaacaaaacagactggatcagcatcatccatggtcaacggagtgttcaggctgctgacaaaaccttgggacgtcgtccccatggtgacacagatggcaatgacagacacgactccatttggacaacagcgcgtttttaaagagaaagtggacacgagaacccaagaaccgaaagaaggcacgaagaaactaatgaaaataacagcagagtggctttggaaagaattagggaagaaaaagacacccaggatgtgcaccagagaagaattcacaagaaaggtgagaagcaatgcagccttgggggccatattcactgatgagaacaagtggaagtcggcacgtgaggctgttgaagatagtaggttttgggagctggttgacaaggaaaggaatctccatcttgaaggaaagtgtgaaacatgtgtgtacaacatcatgggaaaaagagagaagaagctaggggaattcggcaaggcaaaaggcagcagagccatatggtacatgtggcttggagcacgcttcttagagtttgaagccctaggattcttaaatgaagatcactggttctccagagagaactccctgagtggagtggaaggagaagggctgcacaagctaggttacattctaagagacgtgagcaagaaagagggaggagcaatgtatgccgatgacaccgcaggatgggatacaagaatcacactagaagatctaaaaaatgaagcaatggtaacaaaccacatggaaggagaacacaagaaactagccgaggccattttcaaactaacgtaccaaaacaaggtggtccgtgtgcaaagaccaacaccaagaggcacagtaatggacatcatatcgagaagagaccaaagaggtagtggacaagttggcacctatggactcaatactttcaccaatatggaagcccaactaatcagacagatggagggagaaggagtctttaaaagcattcagcacctaacaatcacagaagaaatcgctgtgcaaaactggttagcaagagtggggcgcgaaaggttatcaagaatggccatcagtggagatgattgtgttgtgaaacctttagatgacaggttgccaagcgctttaacagctctaaatgacacaggaaagattaggaaagacatacaacaatgggaaccttcaagaggatggaatgattggacacaagtgcccttctgttcacaccatttccatgagttaatcatgaaagacggccgcgtactcgttgttccatgtagaaaccaagatgaactgattggcagagcccgaatctcccaaggagcagggtggtctttgcgggagacggcctgtttggggaagtcttacgaccaaatgtggagcttgatgtacttccacagacgcgacctcaggctggcggcaaatgctatttgctcggcagtaccatcacattgggttccaacaagtcgaacaacctggtccatacatgctaaacatgaatggatgacaacggaagacatgctgacagtctggaacagggtgtggattcaagaaaacccatggatggaagacaaaactccagtggaatcatgggaggaaatcccatacttggggaaaagagaagaccaatggtgcggctcattgattgggttaacaagcagggccacctgggcaaagaacatccaagcagcaataaatcaagttagatcccttataggcaatgaagaatacacagattacatgccatccatgaaaagattcagaagagaagaggaagaagcaggagttctgtggtag

SEQ ID NO: 1077 - DNA, 1485 bp, synthetic construct
  misc_feature 1..1485: note = Synthetic polynucleotide
  source 1..1485: mol_type = other DNA
atgcgatgcgtaggggtagggaacagagactttgtggaaggagtctcgggtggagcatgggtcgacttggtgctagaacatggaggatgcgtcacaaccatggcccagggaaaaccaaccttggattttgaactgaccaagacaacagctaaggaagtggctctgttaagaacctattgcattgaagcctcgatatcaaacataaccacggcaacaagatgcccaacgcaaggagagccttatctcaaagaggaacaagaccaacagtacatttgccggagagatgtggtagacagagggtggggcaatggctgtggcttatttggaaaaggaggagttgtgacatgtgcaaaatttttgtgctcggggaagataacaggcaatctggtccaaattgaaaaccttgaatatacagtagttgtgacagtccacaatggagacacccatgcagtaggaaatgacacatccaatcatggagtgacggccacgataactcccaggtcaccatcggtagaagttaaattgccggactatggagaactaatactcgattgtgaacccaggtccggaattgatttcaatgagatgatcctgatgaaaatgaaaaagaagacgtggcttgtgcataagcaatggtttttggacctacctctaccatggacagcaggagcagacacatcagaacttcattggaattataaagagagaatggtgacattcaaggttcctcatgccaagagacaggatgtgacagtgctagggtctcaggagggagctatgcattctgccctcgccggagccacagaagtagattctggtgatggaaatcacatgtttgcaggacatctcaagtgcaaagtccgcatggagaaattgagaattaagggaatgtcatacacgatgtgttcaggaaagttctcaattgacaaagagatggcagaaacacagcatgggacaacagtggtgaaagtcaagtatgaaggcgctggagctccgtgtaaagtccccatagagataagagatgtgaacaaggaaaaagtggttgggcgcatcatttcatccaccccttttgctgagaataccaacagcgtaaccaacatagaattagaacccccttttggggacagctatatagtgataggtgttggagatagcgcattaacactccattggttcagaaaagggagctccattggcaagatgtttgagtccacatacagaggtgcaaaacgaatggccattctaggtgaaacagcttgggattttggttctgttggtggactgttcacatcactgggaaaggctgtgcaccaggtttttggaagtgtgtatacaaccatgtttggaggggtctcatggatggtcagaatcctaattgggttcttagtgttatggattggcacgaattcaagaaacacttcaatggcaatgacgtgcatagctgttggaggaatcactctgtttctaggcttcacagttcaagca

SEQ ID NO: 1078 - DNA, 1503 bp, synthetic construct
  misc_feature 1..1503: note = Synthetic polynucleotide
  source 1..1503: mol_type = other DNA
ttcaactgcttaggaatgagcaacagagacttcctggagggagtgtctggagctacatgggttgatctggtactggaaggcgacagctgtgtgaccataatgtcaaaagacaagccaaccattgatgtcaaaatgatgaacatggaagcagccaatcttgcagatgtgcgcagttactgttacctagcttcagtcagtgacttgtcaacaagagccgcgtgtccaaccatgggtgaagcccacaacgagaaaagagctgatcccgccttcgtttgcaagcaaggcgttgtggatagaggatggggaaacggatgcggactgtttggaaaggggagcattgacacatgtgcgaagtttgcctgtacaaccaaagcaactggttggatcatccagaaggaaaacatcaagtatgaggttgccatatttgtgcatggcccgacgaccgttgaatctcatggcaattattcaacacagataggggccacccaggctggaagattcagcataactccatcggcgccatcttacacgctaaagttgggtgagtatggtgaggttacggttgattgtgagccacggtcaggaatagacactagcgcctattacgttatgtcagttggtgcgaagtccttcctggttcaccgagaatggtttatggatctgaacctgccatggagcagtgctggaagcaccacgtggaggaaccgggaaacactgatggagtttgaagaacctcatgccaccaaacagtctgttgtggctctagggtcgcaggaaggtgctttgcaccaagctctggccggagcgattcctgttgaattctcaagcaacactgtgaagttgacatcaggacatctgaagtgtagggtgaagatggagaagttgcagctgaagggaacaacatatggagtatgttcaaaagcgttcaaattcgctgggactcccgctgacactggccatggaacggtggtgttggaactgcagtacaccggaaaggacggtccctgcaaagtgcccatttcttccgtagcttccctgaatgacctcacacctgttggaagactggtgacagtgaatccatttgtgtctgtggccacggccaactcgaaggttttgattgaactcgaacccccgtttggtgactcttacatcgtggtgggaagaggagaacagcagataaaccatcactggcacaaatctgggagcagcattggaaaggcctttactaccacactcagaggagctcaacgactcgcagctcttggagatactgcttgggattttggatcagttggaggggttttcacctcggtggggaaagctatacaccaagtctttggaggagcttttagatcactttttggagggatgtcctggatcacacagggacttctgggagctcttctgttgtggatgggaatcaatgcccgtgacaggtcaattgctatgacgttccttgcggttggaggagttttgctcttcctttcggtcaacgtccatgct

SEQ ID NO: 1079 - DNA, 1536 bp, synthetic construct
  misc_feature 1..1536: note = Synthetic polynucleotide
  source 1..1536: mol_type = other DNA
atgaagtgccttttgtacttagcttttttattcattggggtgaattgcaagttcaccatagtttttccacacaaccagaaaggaacctggaaaaatgttccttccaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctccccaaagttgtggatatgcaactgtgacggatgctgaagcagtgattgtccaggtgactcctcaccatgtgcttgttgatgaatacacaggagaatgggttgattcacagttcatcaacggaaaatgcagcaatgacatatgcccaactgtccataactccacaacctggcattctgactataaggtcaaagggctatgtgattctaacctcatttccacggacatcaccttcttctcagaggacggagagttatcatctctaggaaaggggggtacagggttcagaagtaaccacttcgcttatgaaactggagacaaggcctgcaaaatgcagtactgcaggcattggggagtcagactcccatcaggtgtttggttcgagatggctgataaggatctctttgctgcagccagattccctgaatgcccagaagggtcaagtatctctgctccatctcagacctcagtggatgtaagtctcattcaggacgttgagaggatcttggattattccctctgccaagaaacctggagcaaaatcagggcaggtcttcccatctctccagtggatctcagctatcttgctcctaaaaacccaggaaccggccctgccttcactataattaatggtaccctaaaatactttgagaccagatacatcagggttgatattgttgctccaatcctctcaagaatggtcggaatgatcagtggaactaccacagaaagggaactgtgggatgactgggcaccatatgaagacgtggaaattggacccaatggagttctgaggaccagttcaggatataagtttcctttatatatggttggacatggtatgttggactccgatcttcatcttagctcaaaggctcaggtgttcgaacatcctcacattcaggatgctgcttcgcagcttcctgatgatgagactttgttttttggtgacactgggttatccaaaaatccaatcgagcttgtagagggttggttcagtggttggaagagctctattgcctcttttttctttatcatagggttaatcattggactattcttggttctccgagttggtatttatctttgcattaaattaaagcacaccaagaaaagacagatttatacagatatagagatgaatcgacttggaaagtaa

SEQ ID NO: 1080 - DNA, 70 bp, synthetic construct
  misc_feature 1..70: note = Synthetic oligonucleotide
  source 1..70: mol_type = other DNA
ctcacatgggactcagacaggaattcctgttgagctaacaatgcccaagggactaacaacacatcaggtt

SEQ ID NO: 1081 - DNA, 514 bp, synthetic construct
  misc_feature 1..514: note = Synthetic polynucleotide
  source 1..514: mol_type = other DNA
cgagatggctgataaggctctctttgctgcagccagattccctgaatgcccagaagggtcaagtatctctgctccatctcagacctcagtggatgtaagtctcattcaggacattgagaggatcttggattattccctctgccaagaaacctggagcaaaatcagagcgggtcttcccatctctccagtggatctcagctatcttgctcctaaaaacccaggaaccggtcctgtctttaccataatcaatggtaccctaaaatactttgagaccagatacatcagagtcgatattgctgctccaatcctctcaagaatggtcggaatgatcagtggaactaccacagaaagggtactgtgggatgactgggctccatatgaagacgtggaaattggacccaatggagttctgaggaccagttcaggatataagtttcctttatatatgattggacatggtatgttggactccgatcttcatcttagctcaaaggctcaggtgtttgaacatcct

SEQ ID NO: 1082 - DNA, 514 bp, synthetic construct; identical to SEQ ID NO: 1081
  misc_feature 1..514: note = Synthetic polynucleotide
  source 1..514: mol_type = other DNA

SEQ ID NO: 1083 - DNA, 444 bp, synthetic construct
  misc_feature 1..444: note = Synthetic polynucleotide
  source 1..444: mol_type = other DNA
gctccatctcagacctcagtggatgtaagtctcattcaggacattgagaggatcttggattattccctctgccaagaaacctggagcaaaatcagagcgcgtcttcccatctctccagtggatctcagctatcttgctcctaaaaacccaggaaccggtcctgtctttaccataatcaatggtaccctaaaatactttgagaccagatacatcagagtcgatattgctgctccaatcctctcaagaatggtcggaatgatcagtggaactaccacagaaagggtactgtgggatgactgggctccatatgaagacgtggaaattggacccaatggagttctgaggaccagttcaggatataagtttcctttatatatgattggacatggtatgttggactccgatcttcatcttagctcaaaggctcaggtgtttgaacatcct

SEQ ID NO: 1084 - DNA, 514 bp, synthetic construct
  misc_feature 1..514: note = Synthetic polynucleotide
  source 1..514: mol_type = other DNA
cgagatggctgataaggctctctttgctgcagccagattccctgaatgcccagaagggtcaagtatctctgctccatctcagacctcagtggatgtaagtctcattcaggacgttgagaggatcttggattattccctctgccaagaaacctggagcaaaatcagagcgggtcttcccatctctccagtggatctcagctatcttgctcctaaaaacccaggaaccggtcctgtctttaccataatcaatggtaccctaaaatactttgagaccagatacatcagagtcgatattgctgctccaatcctctcaagaatggtcggaatgatcagtggaactaccacagaaagggtactgtgggatgactgggctccatatgaagacgtggaaattggacccaatggagttctgaggaccagttcaggatataagtttcctttatatatgattggacatggtatgttggactccgatcttcatcttagctcaaaggctcaggtgtttgaacatcct

SEQ ID NO: 1085 - DNA, 514 bp, synthetic construct; identical to SEQ ID NO: 1081
  misc_feature 1..514: note = Synthetic polynucleotide
  source 1..514: mol_type = other DNA

SEQ ID NO: 1086 - DNA, 1575 bp, synthetic construct
  misc_feature 1..1575: note = Synthetic polynucleotide
  source 1..1575: mol_type = other DNA
atggttcctcaggctctcctgtttgtaccccttctggtttttccattgtgttttgggaaattccctatttacacgataccagacaagcttggtccctggagcccgattgacatacatcacctcagatgcccaaacaatttggtagtggaggacgaaggatgcaccaacctgtcagggttctcctacatggaacttaaagttggatacatcttagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccgctggcttcgaactgtaaaaaccaccaaggagtctctcgttatcatatctccaagtgtggcagatttggacccatatgacagatcccttcactcgagggtcttccctagcgggaagtgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccgatcagttggtgaacctgcacgactttcgctcagacgaaattgagcaccttgttgtagaggagttggtcaggaagagagaggagtgtctggatgtactagagtccatcatgacaaccaagtcagtgagtttcagacgtctcagtcatttaagaaaacttgtccctgggtttggaaaagcatataccatattcaacaagaccttgatggaagccgatgctcactacaagtcagtcagaacttggaatgagatcctcccttcaaaagggtgtttaagagttggggggaggtgtcatcctcatgtgaacggggtgtttttcaatggtataatattaggacctgacggcaatgtcttaatcccagagatgcaatcatccctcctccagcaacatatggagttgttggaatcctcggttatcccccttgtgcaccccctggcagacccgtctaccgttttcaaggacggtgacgaggctgaggattttgttgaagttcaccttcccgatgtgcacaatcaggtctcaggagttgacttgggtctcccgaactgggggaagtatgtattactgagtgcaggggccctgactgccttgatgttgataattttcctgatgacatgttgtagaagagtcaatcgatcagaacctacgcaacacaatctcagagggacagggagggaggtgtcagtcactccccaaagcgggaagatcatatcttcatgggaatcacacaagagtgggggtgagaccagactgtga

SEQ ID NO: 1087 - DNA, 1575 bp, synthetic construct
  misc_feature 1..1575: note = Synthetic polynucleotide
  source 1..1575: mol_type = other DNA
atgattcttcaggcccttctgtttgtgcctctcctaatctcttcattgtgtctcgggaaattccccatctatacaataccagataagcttggtccttggagccccatcgatatacatcacctcagctgtccaaataatttagttgtggaggacgaggggtgcaccaatctatcagggttctcttacatggaactaaaggtgggatacatctctgccataaaggtgaacgggttcacttgtaccggtgtggtgacagaggctgagacctataccaactttgttggttatgtcaccaccacattcaagaggaaacattttcgccctataccggatgcatgcagggctgcatacaactggaagatggctggtgaccctagatatgaggagtctcttcaaaatccctatcctgattaccactggctacggactgtaaagaccactaaggagtctcttatcatcatatctcctagtgtggctgatctagacccatacgacaaatccctccattctagggtgttccctggtgggaagtgtttgggaataacggtttcttccacctactgctcaaccaaccatgattacaccatctggatgcccgaggaaccaagactcgggacatcttgcgacatttttaccagcagtaaagggaagaaggcatctaagggaggcaagacttgcggatttgtggatgaaaggggcttgtacaagtctctaaaaggagcatgtaaactcaagctgtgcggagttctcggacttagacttatggacggaacctgggtttcgattcaaacatcagacgataccaaatggtgcccttcggatcaattggtgaatctacatgactttcactcagacgaaatagagcatctcgtcgtggaggagttggtcaagaagagggaagagtgtttggacgcattggagtccatcatgaccaccaaatctgtaagttttagacgtctcagctatttgagaaaacttgtccctgggtttggaaaggcatacactatattcaacaagaccttgatggaggctgacgcccactacaagtcagttcggacttggaacgagatcatcccctcaaaagggtgtttaaaagtcagagaaaggtgtcatcctcctgtggacggagtgttcttcaatggcataattctgggtccggacgggaatgtcctgatacctgaaatgcaatcgtctctacttcaacaacatatggagctgttggagtcttctgtaatccccttaatgcatcccttggcggacccgtcaacagtcttcaaggaaggggatgaagcggaggactttgttgaagttcacctcccagatgttcacaaacagatctctggggttgaccttggtctcccgagttgggggaaatatctcctgatgattgcaggtggtctggcggcactagttctgataatctgctcgatggcatgctgtagaagaaccaagcgaacagagtcaagaagacgaggctctggagagtcagaaaagaaggtaacggcaaccccccaaactaggaaagtcgtatcttcatgggagttgtacaaaagtgaaggcgatgccaggttgtga

SEQ ID NO: 1088 - DNA, 1575 bp, synthetic construct
  misc_feature 1..1575: note = Synthetic polynucleotide
  source 1..1575: mol_type = other DNA
atgatctttcaggcccttctgtttgtgcctctcctaatctcttcgttgtgtctcgggaaattccccatctacacaataccagacaagcttggtccttggagccccatcgatatacatcacctcagctgtccaaataatttagttgtggaggacgaagggtgcaccaatctatcaggattctcttacatggaactaaaggtgggatacatctctgccataaaggtgaacgggttcacttgtaccggtgttgtgacagaggctgaaacctataccaactttgttggttatgtcaccaccacattcaagaggaaacatttccgccctataccggatgcatgcagggctgcatacaactggaagatggctggtgatcctagatatgaggagtctcttcaaaatccttaccctgattaccactggctacggaccgtaaaaaccactaaggagtctcttatcatcatatctccgagtgtggctgacttagacccatacgacaaatccctgcattctagggtgttccctagtgggaaatgtttgggaataacggtttcttccacctactgctcaaccaaccatgattacaccatctggatgcccgaggaaccaagactcggaacatcttgcgacatttttaccagcagcaaagggaaaaaggcatctaaaggaggcaagacttgcggatttgtggatgaaaggggcttgtacaagtctctaaaaggagcatgtaaactcaagctgtgcggagttctcggacttagacttatggatggaacctgggtttcaattcaaacatcagacgataccaaatggtgccctccggatcaattggtgaatctacatgactttcactcagacgaaatagagcatctcgtcgtggaggagctggtcaagaagagggaagagtgtttggacgcattggagtctatcatgaccaccaaatctgtaagttttagacgtctcagctatttgagaaaacttgtccctgggtttggaaaggcatacactatattcaacaagactttgatggaggctgacgcccactacaagtcagttcggacttggaacgagatcatcccctcaaaagggtgtttgaaagtcagagagaggtgtcatcctcctgtggacagagtgttcttcaatggcataattctgggtccggacgggaatgtcctgataccagagatgcaatcgtctctacttcaacaacatatggagctgttggagtcttctgtaatccccttaatgcatcccttggcggacccgtcaacagtcttcaaggagggggatgaagcggaggattttgttgaagtccacctcccagatgttcacaaacagatctcaggggttgaccttggtctcccgagttgggggaaatatcttctgatgattgcaggtggtctggcgacactggttctgataatctgctcaatggcatgctgtagaagaaccaagcgaacagagtcaagaagacgaggctctcgagagtcagagaaaaaggtaacggcagccccccagactaggaaagtcgtatcttcatgggagttgtacaagagtaaaggcgatgccaggctgtga

SEQ ID NO: 1089 - DNA, 1575 bp, synthetic construct
  misc_feature 1..1575: note = Synthetic polynucleotide
  source 1..1575: mol_type = other DNA
atgattcttcaggcccttctgtttgtgcctctcctaatctcttcattgtgtctcgggaaattccccatctatacaataccagataagcttggtccttggagccccatcgatatacatcacctcagctgtccaaataatttagttgtggaggacgaggggtgcaccaatctatcagggttctcttacatggaactaaaggtgggatacatctctgccataaaggtgaacgggttcacttgtaccggtgtggtgacagaggctgagacctataccaactttgttggttatgtcaccaccacattcaagaggaaacattttcgccctataccggatgcatgcagggctgcatacaactggaagatggctggtgaccctagatatgaggagtctcttcaaaatccctatcctgattaccactggctacggactgtaaagaccactaaggagtctcttatcatcatatctcctagtgtggctgatctagacccatatgacaaatccctccattctagggtgttccctggtgggaagtgtttgggaataacggtttcttccacctactgctcaaccaaccatgattacaccatctggatgcccgaggaaccaagactcgggacatcttgcgacatttttaccagcagtaaagggaagaaggcatctaagggaggcaagacttgcggatttgtggatgaaaggggcttgtacaagtctctaaaaggagcatgtaaactcaagctgtgcggagttctcggacttagacttatggacggaacctgggtttcgattcaaacatcagacgataccaaatggtgccctccggatcaattggtgaatctacatgactttcactcagacgaaatagagcatctcgtcgtggaggagttggtcaagaagagggaagagtgtttggacgcattggagtccatcatgaccaccaaatctgtaagttttagacgtctcagctatttgagaaaacttgtccctgggtttggaaaggcatacactatattcaacaagaccttgatggaggctgacgcccactacaagtcagttcggacttggaacgagatcatcccctcaaaagggtgtttaaaagtcagagagaggtgtcatcctcctgtggacggagtgttcttcaatggcataattctggttccggacgggaatgtcctgatacctgaaatgcaatcgtctctacttcaacaacatatggagctgttggagtcttctgtaatcccgttaatgcatcccttggcggacccgtcaacagtcttcaaggaaggggatgaagcggaggactttgttgaagttcacctcccagatgttcacaaacagatctctggggttgaccttggtctcccgagttgggggaaatatctcctgatgattgcaggtggtctggcggcactagttctgataatctgctcgatggcatgctgtagaagaaccaagcgaacagagtcaagaagacgaggctctggagagtcagaaaagaaggtaacggcaaccccccaaactaggaaagtcgtatcttcatgggagttgtacaaaagtgaaggcgatgccaggttgtga

SEQ ID NO: 1090 - DNA, 2715 bp, synthetic construct
  misc_feature 1..2715: note = Synthetic polynucleotide
  source 1..2715: mol_type = other DNA
atgcgccagggcgcccccgcgcgggggtgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccttggcgccgccccaacgggggacccgaaaccgaagaagaacaaaaaaccgaaaaacccaacgccgccacgccccgccggcgacaacgcgaccgtcgccgcgggccacgccaccctgcgcgagcacctgcgggacatcaaggcggagagcaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaatgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgctggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcctccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacattaaggtgggccagccgcagtactacctggccaatgggggctttctgatcgcgtaccagccccttctcagcaacacgctcgcggagctgtacgtgcgggaacacctccgagagcagagccgcaagcccccaaaccccacgcccccgccgcccggggccagcgccaacgcgtccgtggagcgcatcaagaccacctcctccatcgagttcgcccggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccattgcatcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgctttgccgacatcgacacggtcatccacgccgacgccaacgccgccatgttcgcgggcctgggtgcgtttttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtcggccatggagcacacggaacacaaggccaagaagaagggcacgagcgcgctgcttagcgccaaggtcaccgacatggtcatgcgcaagcgccgcaacaccaactacacccaagttcccaacaaagacggtgacgccgacgaggacgacctgtga

SEQ ID NO: 1091 - DNA, 2715 bp, synthetic construct
  misc_feature 1..2715: note = Synthetic polynucleotide
  source 1..2715: mol_type = other DNA
atgcgccagggcgcccccgcgcgggggcgccggtggttcgtcgtatgggcgctcttggggttgacgctgggggtcctggtggtgtcggcggctccgagttcccccggcacgcctggggtcgcggccgcgacccaggcggcgaacgggggccctgccactccggcgccgcccgcccctggcgcccccccaacgggggacccgaaaccgaagaagaacaaaaaaccgaaacccccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgctggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcctccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacatcaaggtgggccagccgcagtactacctggccaatgggggctttctgatcgcgtaccagccccttctcagcaacacgctcgcggagctgtacgtgcgggaacacctccgagagcagagccgcaagcccccaaaccccacgcccccgccgcccggggccagcgccaacgcgtccgtggagcgcatcaagaccacctcctccatcgagttcgcccggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggcgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgttcgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggctttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgccatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 1092 - DNA, 2715 bp, synthetic construct
  misc_feature 1..2715: note = Synthetic polynucleotide
  source 1..2715: mol_type = other DNA
atgcgccagggcgcccccgcgcgggggcgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccctggcgcccccccaacgggggacccgaaaccgaagaagaacaaaaaaccgaaacccccaaagccgccgcgccccgccggcgacaacgcgaccgtcgccgcgggccacgccaccctgcgcgagcacctgcgggacatcaaggcggagaacaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaacgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacaacgagtttgtgttggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcttccgacgccatatccaccaccttcaccaccaacctgaccgagtacccgctctcgcgcgtggacctgggggactgcatcggcaaggacgcccgcgacgccatggaccgcatttttgcccgcaggtacaacgcgacgcacatcaaggtgggccagccgcagtactacctggccaatgggggctttctgatcgcgtaccagccccttctcagcaacacgctcgcggagctgtacgtgcgggaacacctccgcgagcagagccgcaagcccccaaaccccacgcccccgccgcccggggccagcgccaacgcgtccgtggagcgcatcaagaccacctcctccatcgagttcgcccggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccatcgcctcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggcgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgcttcgccgacatcgacacggtcatccacgccgacgccaacgccgccatgtttgcgggcctgggcgcgttcttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtctgtcatggagcgcacggaacacaaggccaagaagaagggcacgagcgcgctgctcagcgccaaggtcaccgacatggtcatgcgcaagcgccgcaacaccaactacacccaagttcccaacaaagacggtgacgccgacgaggacgacctgtga

SEQ ID NO: 1093 - DNA, 2031 bp, synthetic construct
  misc_feature 1..2031: note = Synthetic polynucleotide
  source 1..2031: mol_type = other DNA
atgggcgttacaggaatattgcagttacctcgtgatcgattcaagaggacatcattctttctttgggtaattatccttttccaaagaacattttccatcccacttggagtcatccacaatagcacattacaggttagtgatgtcgacaaactagtttgtcgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccataaagagggtgctttcttcctgtatgatcgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaggctaccggttttggaaccaatgagacagagtacttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaaacctcactagaaaaattcgcagtgaagagttgtctttcacagttgtatcaaacggagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctaacaacccttgccacaatctccacgagtccccaatccctcacaaccaaaccaggtccggacaacagcacccataatacacccgtgtataaacttgacatctctgaggcaactcaagttgaacaacatcaccgcagaacagacaacgacagcacagcctccgacactccctctgccacgaccgcagccggacccccaaaagcagagaacaccaacacgagcaagagcactgacttcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggagaagaactcgaagagaagcaattgtcaatgctcaacccaaatgcaaccctaatttacattactggactactcaggatgaaggtgctgcaatcggactggcctggataccatatttcgggccagcagccgagggaatttacatagaggggctaatgcacaatcaagatggtttaatctgtgggttgagacagctggccaacgagacgactcaagctcttcaactgttcctgagagccacaactgagctacgcaccttttcaatcctcaaccgtaaggcaattgatttcttgctgcagcgatggggcggcacatgccacattctgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

SEQ ID NO: 1094 - DNA, 2031 bp, synthetic construct
  misc_feature 1..2031: note = Synthetic polynucleotide
  source 1..2031: mol_type = other DNA
atggggggtcttagcctactccaattgcccagggacaagtttcggaaaagctctttctttgtttgggtcatcatcttattccaaaaggccttttccatgcctttgggtgttgtgactaacagcactttagaagtaacagagattgaccagctagtctgcaaggatcatcttgcatctactgaccagctgaaatcagttggtctcaacctcgaggggagcggagtatctactgatatcccatctgcaacaaagcgttggggcttcagatctggtgttcctcccaaggtggtcagctatgaagcgggagaatgggctgaaaattgctacaatcttgaaataaagaagccggacgggagcgaatgcttacccccaccgccagatggtgtcagaggctttccaaggtgccgctatgtgcacaaagcccaaggaaccgggccctgcccaggtgactacgcctttcacaaggatggagctttctttctctatgacaggctggcttcaactgtaatttacagaggagtcaattttgctgagggggtgattgcattcttgatattggctaaaccaaaagaaacgttccttcagtcaccccccattcgagaggcagtaaactacactgaaaatacatcaagttattatgccacatcctacttggagtatgaaatcgaaaattttggtgctcaacactccacgacccttttcaaaattgacaataatacttttgttcgtctggacaggccccacacgcctcagttccttttccagctgaacgacaccattcaccttcaccaacagttgagtaatacaactgggagactaatttggacactagatgctaatatcaatgctgatattggtgaatgggctttttgggaaaataaaaaaaatctctccgaacaactacgtggagaagagctgtctttcgaagctttatcgctcaacgagacagaagacgatgatgcggcatcgtcgagaattacaaagggaagaatctccgaccgggccaccaggcagtattcggacctggttccaaagaatccccctgggatggttccattgcacataccagaaggggaaacaacattgccgtctcagaattcgacagaaggtcgaagagtaagtgtgaacactcaggagaccatcacagagacagctgcaacaattataggcactaacggcaaccatatgcagatctccaccatcgggataagaccgagctccagccaaatcccgagttcctcaccgaccacggcaccaagccctgaggctcagacccccacaacccacacatcaggtccatcagtgatggccaccgaggaaccaacaacaccaccgggaagctcccccggcccaacaacagaagcacccactctcaccaccccagaaaatataacaacagcggttaaaactgtcctgccacaggagtccacaagcaacggtctaataacttcaacagtaacagggattcttgggagtcttgggcttcgaaaacgcagcagaagacaaactaacaccaaagccacgggtaagtgcaatcccaacttacactactggactgcacaagaacaacataatgctgctgggattgcctggatcccgtactttggaccgggtgcggaaggcatatacactgaaggcctgatgcataaccaaaatgccttagtctgtggacttaggcaacttgcaaatgaaacaactcaagctctgcagcttttcttaagagccacaacggagctgcggacatataccatactcaacaggaaggccatagatttccttctgcgacgatggggcgggacatgcaggatcctgggaccagattgttgcattgagccacatgattggacaaaaaacatcactgataaaatcaaccaaatcatccatgatttcatcgacaaccccttacctaatcaggataatgatgataattggtggacaggctggagacagtggatccctgcaggaataggcattactggaattattattgcaattattgctcttctttgcgtttgcaagctgctttgctga

SEQ ID NO: 1095 - DNA, 250 bp, synthetic construct
  misc_feature 1..250: note = Synthetic polynucleotide
  source 1..250: mol_type = other DNA
tttcaggacaggtgtacctcctaagaatgttgagtatacggaaggggaggaagccaaaacatgctacaatataagtgtaacagatccctctggaaaatccttgctgttagatcctcccaccaacgtccgtgactatcctaaatgcaaaactatccatcacattcaaggtcaaaaccctcatgcgcaggggatcgccctccatttgtggggagcatttttcctatatgatcgcattgcctccacaacaatg

SEQ ID NO: 1096 - DNA, 247 bp, synthetic construct
  misc_feature 1..247: note = Synthetic polynucleotide
  source 1..247: mol_type = other DNA
tcaggacaggtgtacctcctaagaatgttgagtatacagaaggggaggaagccaaaacatgctacaatataagtgtaacggatccctctggaaaatccttgctgttagatcctcccaccaacgtccgtgactatcctaaatgcaaaactatccatcacattcaaggtcaaaaccctcatgcgcagggaatcgccctccatttgtggggagcatttttcctatatgatcgcattgcctccacaacaag

SEQ ID NO: 1097 - DNA, 449 bp, synthetic construct
  misc_feature 1..449: note = Synthetic polynucleotide
  source 1..449: mol_type = other DNA
cgggcagaaagttgctgattcccctttggaggcatccaaacgatgggctttcaggacaggtgtacctcctaagaatgttgagtatacagaaggggaggaagccaaaacatgctacaatataagtgtaacggatccctctggaaaatccttgctgttagatcctcccaccaacgtccgtgactatcctaaatgcaaaactatccatcacattcaaggtcaaaaccctcatgcgcagggaatcgccctccatttgtggggagcatttttcctatatgatcgcattgcctccacaacaatgtaccgaggcaaagtcttcactgaagggaacatagcagccatgattgtcaataagacagtgcacaaaatgattttctcgaggcaaggacaagggtaccgtcacatgaatctgacttctactaataaatattggacaagtagcaacggaacgc

SEQ ID NO: 1098 - DNA, 2046 bp, synthetic construct
  misc_feature 1..2046: note = Synthetic polynucleotide
  source 1..2046: mol_type = other DNA
atgaagaccacatgtttccttatcagtcttatcttaattcaagggacaaaaaatctccccattttagagatagctagtaataatcaaccccaaaatgtggattcggtatgctccggaactctccagaagacagaagacgtccatctgatgggattcacactgagtgggcaaaaagttgctgattcccctttggaggcatccaagcgatgggctttcaggacaggtgtacctcccaagaatgttgagtacacagagggggaggaagccaaaacatgctacaatataagtgtaacggatccctctggaaaatccttgctgttagatcctcctaccaacatccgtgactatcctaaatgcaaaactatccatcatattcaaggtcaaaaccctcatgcacaggggatcgcccttcatttatggggagcattttttctgtatgatcgcattgcctccacaacaatgtaccgaggcaaagtcttcactgaagggaacatagcagctatgattgtcaataagacagtgcacaaaatgattttctcgcggcaaggacaagggtaccgtcatatgaatctgacttctactaataaatattggacaagtagtaacggaacgcaaacgaatgacactggatgtttcggcgctcttcaagaatacaattctacaaagaaccaaacatgtgctccgtccaaaatacctccaccactgcccacagcccgtccggagatcaaactcacaagcaccccaactgatgccaccaaactcaataccacggacccaagcagtgatgatgaggacctcgcaacatccggctcagggtccggagaacgagaaccccacacaacttctgatgcggtcaccaagcaagggctttcatcaacaatgccacccactccctcaccacaaccaagcacgccacagcaaggaggaaacaacacaaaccattcccaagatgctgtgactgaactagacaaaaataacacaactgcacaaccgtccatgccccctcataacactaccacaatctctactaacaacacctccaaacacaacttcagcactctctctgcaccattacaaaacaccaccaatgacaacacacagagcacaatcactgaaaatgagcaaaccagtgccccctcgataacaaccctgcctccaacgggaaatcccaccacagcaaagagcaccagcagcaaaaaaggccccgccacaacggcaccaaacacgacaaatgagcatttcaccagtcctccccccacccccagctcgactgcacaacatcttgtatatttcagaagaaagcgaagtatcctctggagggaaggcgacatgttcccttttctggatgggttaataaatgctccaattgattttgacccagttccaaatacaaaaacaatctttgatgaatcctctagttctggtgcctcggctgaggaagatcaacatgcctcccccaatattagtttaactttatcttattttcctaatataaatgagaacactgcctactctggagaaaatgagaatgattgtgatgcagagttaagaatttggagcgttcaggaggatgacctggccgcagggctcagttggataccgttttttggccctggaattgaaggactttacactgctgttttaattaaaaatcaaaacaatttggtctgcaggttgaggcgtctagccaatcaaactgccaaatccttggaactcttattgagagtcacaactgaggaaagaacattctccttaatcaatagacatgctattgactttctactcacaagatggggaggaacatgcaaagtgcttggacctgattgttgcatcgggatagaagacttgtccaaaaatatttcagagcaaattgaccaaattaaaaaggacgaacaaaaagaggggactggttggggtctgggtggtaaatggtggacatccgactggggtgttcttactaacttgggcattttgctactattatccatagctgtcttgattgctctatcctgtatttgtcgtatctttactaaatatatcggataa

SEQ ID NO: 1099 - DNA, 2046 bp, synthetic construct
  misc_feature 1..2046: note = Synthetic polynucleotide
  source 1..2046: mol_type = other DNA
atgaggactacatgcttctttatcagtctcatcttaatccaagggataaaaactctccctattttggagatagccagtaacgatcaaccccaaaatgtggattcggtatgctccggaactctccagaaaacagaagacgtccatctgatgggatttacactgagcgggcagaaagttgctgattcccctttggaggcatccaagcgatgggctttcaggacaggtgtacctcctaagaatgttgagtatacggaaggggaggaagccaaaacatgctacaatataagtgtaacggatccctctggaaaatccttgctgttagatcctcccaccaacgtccgtgactatcctaaatgcaaaactatccatcacattcaaggtcaaaaccctcatgcgcaggggatcgccctccatttgtggggagcatttttcctatatgatcgcattgcctccacaacaatgtaccgaggcaaagtcttcactgaagggaacatagcagccatgattgtcaataagacagtgcacaaaatgattttctcgaggcaaggacaagggtaccgtcacatgaatctgacttctactaataaatattggacaagtagcaacggaacgcaaacaaatgacactggatgctttggtactcttcaagaatacaattctacgaagaaccaaacatgtactccgtctaaaacacccccaccaccgcccacagcctatccggagatcaaacccacaagcacctcaaccgatgccactagactcaacaccacaaacccaaacagtgatgatgaggatctcacaacatccggctcggggtctggggaacaggaacccgacacgacttctgatgcggtcaccaagcaagggctttcatcaacaatgccacccactccctcaccgcaaccaggcacgccacagcaaggaggaaacaacacaaaccactcccaagacgctgcaactgaacttgacaacaccaatacaactgcacaaccgcccacgccctcccacaacaccaccacaatctccaccaacaacacctccaaacacaacctcagcaccctctccgaactaccacaaaacaccaccaatcccaacacacaaagcatggccactgaaaatgagaaaaccagtgcctccccgaaaacaactctgcctccaacagaaagtcctaccacagaaaagagcaccaacaatacaaaaagccccaccacaatggaaccaaatacaacaaatggacatttcactagtccctcctccacccccaactcgactactcaacatcttatatatttcaggaggaaacgaagtatcctctggagggaaggcgacatgttcccttttctagatgggttaataaatgctccaattgattttgatccagttccaaatacaaagacaatctttgatgaatcttctagttctggtgcttcagctaaggaagatcaacatgcatcctccaatatcagtttaactttatcttatcttcctcatacaagtgaaaacactgcctactctggagaaaatgaaaatgattgtgatgcagagctaagaatttggagtgttcaggaggacgacctggcagcagggcttagttggataccattttttggccctggaatcgaaggactttataccgctggtttaattaaaaatcaaaacaatttggtctgcaggttgaggcgtctagccaatcaaactgcaaaatctttggaactcttactaagggtcacaaccgaggaaagaacattttccttaatcaatagacatgctattgactttctactcacaaggtggggaggaacatgcaaagtgcttggacctgattgttgcataggaatagaggacttgtccagaaatatttcagaacagattgaccaaatcaagaaggacgaacaaaaagaagggactggttggggtctgggtggtaaatggtggacatccgactggggtgttcttactaacttgggcatcttactactattgtccatagctgtcttgattgctctatcctgtatttgtcgtatctttactaaatatattggatag

SEQ ID NO: 1100 - DNA, 2046 bp, synthetic construct
  misc_feature 1..2046: note = Synthetic polynucleotide
  source 1..2046: mol_type = other DNA
atgaagaccatatattttctgattagtctcattttaatccaaagtataaaaactctccctgttttagaaattgctagtaacagccaacctcaagatgtagattcagtgtgctccggaaccctccaaaagacagaagatgttcatctgatgggatttacactgagtgggcaaaaagttgctgattcccctttggaagcatctaaacgatgggctttcaggacaggtgttcctcccaagaacgttgagtatacggaaggagaagaagccaaaacatgttacaatataagtgtaacagacccttctggaaaatccttgctgctggatcctcccagtaatatccgcgattaccctaaatgtaaaactgttcatcatattcaaggtcaaaaccctcatgcacaggggattgccctccatttgtggggggcatttttcttgtatgatcgcgttgcctctacaacaatgtaccgaggcaaggtcttcactgaaggaaatatagcagctatgattgttaataagacagttcacagaatgattttttctaggcaaggacaaggttatcgtcacatgaacttgacctccaccaataaatattggacaagcagcaatgaaacgcagagaaatgatacgggatgttttggcatcctccaagaatacaactccacaaacaatcaaacatgccctccatctcttaaacctccatccctgcccacagtaactccgagcattcactctacaaatactcaaattaatactgctaaatctggaactatgaacccaagtagcgacgatgaggaccttatgatttccggctcaggatctggagaacaggggccccacacaactcttaatgtagtcactgaacagaaacaatcgtcaacaatattgtccactccttcactacatccaagcacctcacaacatgagcaaaacagtacgaatccttcccgacatgctgtaactgagcacaatggaaccgacccaacaacacaaccagcaacgctcctcaacaatactaatacaactcccacctataacactctcaagtacaacctcagtactccttcccctccaacccgcaacatcaccaataatgatacacaacgtgaactagcagaaagcgaacaaaccaatgctcagttgaacacaactctagatccaacagaaaatcccaccacaggacaagacaccaacagcacaaccaacatcatcatgacgacatcagatataacaagcaaacaccccacaaattcttctccggattctagtccgacaacccgccctcctatatactttagaaagaaacgaagcattttctggaaagaaggtgatatattcccgtttttagatgggttaataaatactgaaattgattttgatccaatcccaaacacagaaacaatctttgatgaatctcccagctttaatacttcaactaatgaggaacaacacactcccccgaatatcagtttaactttctcttattttcctgataaaaatggagatactgcctactctggggaaaacgagaatgattgtgatgcagagttgaggatttggagtgtgcaggaggacgatttggcggcagggcttagctggataccattttttggccctggaatcgaaggactctatactgccggtttaatcaaaaatcagaacaatttagtttgtaggttgaggcgcttagctaatcaaactgctaaatccttggagctcttgttaagggtcacaaccgaggaaaggacattttccttaatcaataggcatgcaattgactttttgcttacgaggtggggcggaacatgcaaggtgctaggacctgattgttgcataggaatagaagatctatctaaaaatatctcagaacaaatcgacaaaatcagaaaggatgaacaaaaggaggaaactggctggggtctaggtggcaaatggtggacatctgactggggtgttctcaccaatttgggcatcctgctactattatctatagctgttctgattgctctgtcctgtatctgtcgtatcttcactaaatacattggatga

SEQ ID NO: 1101 - DNA, 666 bp, synthetic construct
  misc_feature 1..666: note = Synthetic polynucleotide
  source 1..666: mol_type = other DNA
atggggacgctggtggccaagctgctcctgcccaccctcagcagcctggccttcctccccactgtcagcatcgcggccaagaggcggttccacatggaggccatggtctacctcttcaccctgttcttcgtggcgctccaccatgcctgcaatggacccggcttgtctgtgctgtgcttcatgcgtcacgacatcctggagtatttcagtgtctacgggacagccctgagcatgtgggtctcgctgatggcactggccgacttcgacgaacccaagaggtcaacatttgtgatgttcggcgtcctgaccattgctgtgcggatctaccatgaccgatggggctacggggtgtactcgggccccatcggcacagccatcctcatcatcgcggcaaagtggctacagaagatgaaggagaagaagggcctgtacccagacaagagcgtctacacccagcagataggccccggcctctgcttcggggcgctggccctgatgctacgcttcttctttgaggactgggactacacttatgtccacagcttctaccactgtgccctggctatgtcctttgttctgctgctgcccaaggtcaacaagaaggctggatccccggggaccccggccaagctggactgctccaccctgtgctgtgcttgtgtctga

SEQ ID NO: 1102 - DNA, 255 bp, synthetic construct
  misc_feature 1..255: note = Synthetic polynucleotide
  source 1..255: mol_type = other DNA
atgcccacgccactgctcccgctgctgcttcgattgctgctgtcctgcctgctgctgcctgctgcccgcctggcccgccaatacctcctgcccctgctgcgccgattggcccgccgcctgggctcccaggacatgcgagaggctttgctgggctgtctgctgttcattctcagccagcgacactcgccagacgctggggaggcctcaagagtggaccgcctggagaggagggagaggttaggcccccaaaagtga

SEQ ID NO: 1103 - DNA, 255 bp, synthetic construct; identical to SEQ ID NO: 1102
  misc_feature 1..255: note = Synthetic polynucleotide
  source 1..255: mol_type = other DNA

SEQ ID NO: 1104 - DNA, 2352 bp, synthetic construct
  misc_feature 1..2352: note = Synthetic polynucleotide
  source 1..2352: mol_type = other DNA
atgtctaccttcataaggcgctatagtagatatttgaatgaaaaggctttttcttacagacagatggcctttgattttgccagggtgaagaaaggggccgatggtgtaatgaggacaatggctcccgaaaagctgctaaagagtatgccaatactacagggacaaattgatgcactgcttgaatttgatgtgcatccaaatgaactaacaaatggtgtcataaatgcagcatttatgcttcttttcaaagatcttatcaaactttttgcttgctacaatgatggtgttattaacttactcgaaaagttttttgaaatgaagaaaggacaatgtaaagatgctctagaaatttacaaacgatttctaactagaatgacacgagtgtctgaatttctcaaggttgcagagcaagttggtattgataaaggtgacattcctgacctcacacaggctcccagcagtcttatggagacgcttgaacagcatctaaatacattagaaggaaagaaacctggaaacaatgaaggatctggtgctccctctccattaagtaagtcttctccagccacaactgttacgtctcctaattctacaccagctaaaactattgacacatccccaccggttgatttatttgcaactgcatctgcggctgtcccagtcagcacttctaaaccatctagtgatctcctggacctccagccagacttttcctctggaggggcagcagcagccgcagcaccagcaccaccaccacctgctggaggagccactgcatggggagaccttttgggagaggattctttggctgcactttcctctgttccctctgaagcacagatttcagatccatttgcaccagaacctacccctcctactacaactgctgaaattgcaactgcctcagcttctgcctccactactacaactgttactgctgtcactgctgaagtggatctctttggagatgcctttgcagcttctcctggggaggcccctgcagcatccgaaggggccgccgcaccagctaccccaacccctgtagcagcagcacttgatgcatgttcaggaaatgacccctttgccccgtctgaaggtagtgcagaggctgcacctgagctggacctctttgcaatgaagccacctgagaccagtgttcctgtagttacccctacagctagcacagcccctccagttcccgcaactgctccttctcctgctcctgccgttgcagctgctgctgctgccactactgctgccaccgccgctgccaccaccactaccaccacctccgctgccaccgccaccactgctcctcctgctctagatatctttggtgatttatttgagtccactcctgaagttgctgcagcgcctaagccagatgctgctcctagcatagacctgtttagtacagatgctttctcctctccaccacaaggggcctctcctgtgcctgagagttctctcactgctgacctcttatctgtggatgcatttgcagcaccatctcctgcaaccactgcctcgccagcaaaggtggattcttcaggtgtcatagacctttttggggatgcatttggaagtagtgcttctgaaccccaacctgcatctcaggctgcttctagttcatcagcatcggcagacctactagctggatttgggggttctttcatggcgccttccccatctccagtgactccagctcagaataacctgctacagcccaattttgaggcagcttttgggacaacgccttcaacttccagcagcagctcctttgatccatcaggtgatcttttgatgccaaccatggcaccagctgggcagcctgcacctgtctcaatggtaccacccagtcctgcaatggcagccagcaaagcccttggaagtgatcttgattcatctcttgccagcttagtaggcaatcttggaatttctggtaccacaacaaaaaagggagatcttcagtggaatgctggagagaaaaagttgactggtggagccaactggcagcctaaagtagctccagcaacctggtcagcaggcgttccaccaagtgcacctttgcaaggagctgtacctccaaccagttcagttcctcctgttgccggggccccatcggttggacaacctggagcaggatttggaatgcctcctgctgggacaggcatgcccatgatgcctcagcagccggtcatgtttgcacagcccatgatgaggcccccctttggagctgccgctgtacctggcacgcagctttctccaagccctacacctgccagtcagagtcccaagaaacctccagcaaaggacccattagcggatcttaacatcaaggatttcttgtaa

SEQ ID NO: 1105 - DNA, 2724 bp, synthetic construct
  misc_feature 1..2724: note = Synthetic polynucleotide
  source 1..2724: mol_type = other DNA
atgtcgggccaaacgctcacggatcggatcgccgccgctcagtacagcgttacaggctctgctgtagcaagagcggtctgcaaagccactactcatgaagtaatgggccccaagaaaaagcacctggactatttgatccaggctaccaacgagaccaatgttaatattcctcagatggccgacactctctttgagcgggcaacaaacagtagctgggtggttgtgtttaaggctttagtgacaacacatcatctcatggtgcatggaaatgagagatttattcaatatttggcttctagaaatacactattcaatctcagcaattttttggacaaaagtggatcccatggttatgatatgtctaccttcataaggcgctatagtagatatttgaatgaaaaggctttttcttacagacagatggcctttgattttgccagggtgaagaaaggggccgatggtgtaatgaggacaatggctcccgaaaagctgctaaagagtatgccaatactacagggacaaattgatgcactgcttgaatttgatgtgcatccaaatgaactaacaaatggtgtcataaatgcagcatttatgcttcttttcaaagatcttatcaaactttttgcttgctacaatgatggtgttattaacttactcgaaaagttttttgaaatgaagaaaggacaatgtaaagatgctctagaaatttacaaacgatttctaactagaatgacacgagtgtctgaatttctcaaggttgcagagcaagttggtattgataaaggtgacattcctgacctcacacaggctcccagcagtcttatggagacgcttgaacagcatctaaatacattagaaggaaagaaacctggaaacaatgaaggatctggtgctccctctccattaagtaagtcttctccagccacaactgttacgtctcctaattctacaccagctaaaactattgacacatccccaccggttgatttatttgcaactgcatctgcggctgtcccagtcagcacttctaaaccatctagtgatctcctggacctccagccagacttttcctctggaggggcagcagcagccgcagcaccagcaccaccaccacctgctggaggagccactgcatggggagaccttttgggagaggattctttggctgcactttcctctgttccctctgaagcacagatttcagatccatttgcaccagaacctacccctcctactacaactgctgaaattgcaactgcctcagcttctgcctccactactacaactgttactgctgtcactgctgaagtggatctctttggagatgcctttgcagcttctcctggggaggcccctgcagcatccgaaggggccgccgcaccagctaccccaacccctgtagcagcagcacttgatgcatgttcaggaaatgacccctttgccccgtctgaaggtagtgcagaggctgcacctgagctggacctctttgcaatgaagccacctgagaccagtgttcctgtagttacccctacagctagcacagcccctccagttcccgcaactgctccttctcctgctcctgccgttgcagctgctgctgctgccactactgctgccaccgccgctgccaccaccactaccaccacctccgctgccaccgccaccactgctcctcctgctctagatatctttggtgatttatttgagtccactcctgaagttgctgcagcgcctaagccagatgctgctcctagcatagacctgtttagtacagatgctttctcctctccaccacaaggggcctctcctgtgcctgagagttctctcactgctgacctcttatctgtggatgcatttgcagcaccatctcctgcaaccactgcctcgccagcaaaggtggattcttcaggtgtcatagacctttttggggatgcatttggaagtagtgcttctgaaccccaacctgcatctcaggctgcttctagttcatcagcatcggcagacctactagctggatttgggggttctttcatggcgccttccccatctccagtgactccagctcagaataacctgctacagcccaattttgaggcagcttttgggacaacgccttcaacttccagcagcagctcctttgatccatcagtgtttgatggtctaggtgatcttttgatgccaaccatggcaccagctgggcagcctgcacctgtctcaatggtaccacccagtcctgcaatggcagccagcaaagcccttggaagtgatcttgattcatctcttgccagcttagtaggcaatcttggaatttctggtaccacaacaaaaaagggagatcttcagtggaatgctggagagaaaaagttgactggtggagccaactggcagcctaaagtagctccagcaacctggtcagcaggcgttccaccaagtgcacctttgcaaggagctgtacctccaaccagttcagttcctcctgttgccggggccccatcggttggacaacctggagcaggatttggaatgcctcctgctgggacaggcatgcccatgatgcctcagcagccggtcatgtttgcacagcccatgatgaggcccccctttggagctgccgctgtacctggcacgcagctttctccaagccctacacctgccagtcagagtcccaagaaacctccagcaaaggacccattagcggatcttaacatcaaggatttcttgtaa

SEQ ID NO: 1106 - DNA, 4152 bp, synthetic construct
  misc_feature 1..4152: note = Synthetic polynucleotide
  source 1..4152: mol_type = other DNA
gcagctcggagcgctagagagacgcggcggcgctggcagaagaggcggcggcgggcgggtactggcttctggggccaggggccaggggcggtgggcgccgggaccgcggagctgaggagcggggcccggccagggctggagactttgcgcccgggggcaccggggctacgcgcggtcgcacacatccaccggcgcggcttccctcggcggcccgggctccgctcatcctgcggcgggcggcgccgctcaggggcgggaagaggaggcggtagacgcgaccacagaagatgtcgggccaaacgctcacggatcggatcgccgccgctcagtacagcgttacaggctctgctgtagcaagagcggtctgcaaagccactactcatgaagtaatgggccccaagaaaaagcacctggactagatttattcaatatttggcttctagaaatacactattcaatctcagcaattttttggacaaaagtggatcccatggttatgatatgtctaccttcataaggcgctatagtagatatttgaatgaaaaggctttttcttacagacagatggcctttgattttgccagggtgaagaaaggggccgatggtgtaatgaggacaatggctcccgaaaagctgctaaagagtatgccaatactacagggacaaattgatgcactgcttgaatttgatgtgcatccaaatgaactaacaaatggtgtcataaatgcagcatttatgcttcttttcaaagatcttatcaaactttttgcttgctacaatgatggtgttattaacttactcgaaaagttttttgaaatgaagaaaggacaatgtaaagatgctctagaaatttacaaacgatttctaactagaatgacacgagtgtctgaatttctcaaggttgcagagcaagttggtattgataaaggtgacattcctgacctcacacaggctcccagcagtcttatggagacgcttgaacagcatctaaatacattagaaggaaagaaacctggaaacaaatctggtgctccctctccattaagtaagctaaaactattgacacatccccaccggttgatttatttgcaactgcatctgcggctgtcccagtcagcacttctaaaccatctagtgatctcctggacctccagccagacttttcctctggaggggcagcagcagccgcagcaccagcaccaccaccacctgctggaggagccactgcatggggagaccttttgggagaggattctttggctgcactttcctctgttccctctgaagcacagatttcagatccatttgcaccagaacctacccctcctactacaactgctgaaattgcaactgcctcagcttctgcctccactactacaactgttactgctgtcactgctgaagtggatctctttggagatgcctttgcagcttctcctggggaggcccctgcagcatccgaaggggccgccgcaccagctaccccaacccctgtagcagcagcacttgatgcatgttcaggaaatgacccctttgccccgtctgaaggtagtgcagaggctgcacctgagctggacctctttgcaatgaagccacctgagaccagtgttcctgtagttacccctacagctagcacagcccctccagttcccgcaactgctccttctcctgctcctgccgttgcagctgctgctgctgccactactgctgccaccgccgctgccaccaccactaccaccacctccgctgccaccgccaccactgctcctcctgctctagatatctttggtgatgctttctcctctccaccacaaggggcctctcctgtgcctgagagttctctcactgctgacctcttatctgtggatgcatttgcagcaccatctcctgcaaccactgcctcgccagcaaaggtggattcttcaggtgtcatagacctttttggggatgcatttggaagtagtgcttctgaaccccaacctgcatctcaggctgcttctagttcatcagcatcggcagacctactagctggatttgggggttctttcatggcgccttccccatctccagtgactccagctcagaataacctgctacagcccaattttgaggcagcttttgggacaacgccttcaacttccagcagcagctcctttgatccatcagtgtttgatggtctaggtgatcttttgatgccaaccatggcaccagctgggcagcctgcacctgtctcaatggtaccacccagtcctgcaatggcagccagcaaagcccttggaagtgatcttgattcatctcttgccagcttagtaggcaatcttggaatttctggtaccacaacaaaaaagggagatcttcagtggaatgctggagagaaaaagttgactggtggagccaactggcagcctaaagtagctccagcaacctggtcagcaggcgttccaccaagtgcacctttgcaaggagctgtacctccaaccagttcagttcctcctgttgccggggccccatcggttggacaacctggagcaggatttggaatgcctcctgctgggacaggcatgcccatgatgcctcagcagccggtcatgtttgcacagcccatgatgaggcccccctttggagctgccgctgtacctggcacgcagctttctccaagccctacacctgccagtcagagtcccaagaaacctccagcaaaggacccattagcggatcttaacatcaaggatttcttgtaaacaatttaagctgcaatatttgtgactgaataggaaaataaatgagtttggagacttcaaataagattgatgctgagtttcaaagggagccaccagtaccaaacccaatacttactcataacttctcttccaaaatgtgtaacacagccgtgaaagtgaacattaggaatatgtactaccttagctgttatccctactcttgaaattgtagtgtatttggattatttgtgtattgtacgatgtaaacaatgaatggatgttactgatgccgttagtgcttttttggacttcacctgaggacagatgatgcagctgttgtgtggcgagctatttggaaagacgtctgtgtttttgaaggtttcaatgtacatataacttttgaacaaaccccaaactcttcccataaattatcttttcttctgtatctctgttacaagcgtagtgtgataataccagataataaggaaaacactcataaatatacaaaactttttcagtgtggagtacatttttccaatcacaggaacttcaactgttgtgagaaatgtttatttttgtggcactgtatatgttaagaaattttattttaaaaaatataaaggttaacgtccataataaatacttctctttgaagctaccttatcaagaacgaaaaatcgtatgggaagaatcccctatttatcactgctatattaaaatatatatattttaattatatttgacaggttttgcatctaaattgacctatttattcattcttgattaaatgcactgaaaagtaaagggtctgtttgtgtcatgttcatgaaaatgcggttagagaggtgctattcaagtgattctgaaggcaccccaaggtatatctgtaatttaaagattactgcaaatatctttactttactgtgggtttttagtacatctgttaatttagtgtttctttgtgtgttttgtagactagtgttcttccatccttcaactgagctcaaagtaggttttgttgtaacattgtgattaggatttaaactaattcagagaattgtatcttttactgtacatactgtattctttaagttttaatttgttgtcatactgtctgtgctgatggcttggcttaagattttgatgcataaatgaggtcactgttgatcagtgttgctagtagcttggcagctcttcataaaagcatattgggttggaaaggtgtttgcctatttttcaaattatttaatagatgtatggtaccatttaaaagtggttgtatctgaatttactgtggggataacatacactgtaatggggaaaaattacctaaaaccaatttcaaaatggctttctttgtatttcagtttaaaaacccagtgcatgtacgccctctgagatgcaataaacaccttgaacaaagaaatgcaaa

SEQ ID NO: 1107-1200 - skipped (94 entries)

SEQ ID NO: 1201 - protein, 538 aa, synthetic construct [sequence omitted]

SEQ ID NO: 1202 - protein, 538 aa, synthetic construct
  REGION 1..538: note = Synthetic polypeptide
MALPYHIFLFTVLLPSFTLTAPPPCRCMTSSSPYQEFLWRMQRPGNIDAPSYRSLSKGTPTFTAHTHMPRNCYHSATLCMHANTHYWTGKMINPSCPGGLGVTVCWTYFTQTGMSDGGGVQDQAREKHVKEVISQLTRVHGTSSPYKGLDLSKLHETLRTHTRLVSLFNTTLTGLHEVSAQNPTNCWICLPLNFRPYVSIPVPEQWNNFSTEINTTSVLVGPLVSNLEITHTSNLTCVKFSNTTYTTNSQCIRWVTPPTQIVCLPSGIFFVCGTSAYRCLNGSSESMCFLSFLVPPMTIYTEQDLYSYVISKPRNKRVPILPFVIGAGVLGALGTGIGGITTSTQFYYKLSQELNGDMERVADSLVTLQDQLNSLAAVVLQNRRALDLLTAERGGTCLFLGEECCYYVNQSGIVTEKVKEIRDRIQRRAEELRNTGPWGLLSQWMPWILPFLGPLAAIILLLLFGPCIFNLLVNFVSSRIEAVKLQMEPKMQSKTKIYRRPLDRPASPRSDVNDIKGTPPEEISAAQPLLRPNSAGSS

SEQ ID NO: 1203 - protein, 538 aa, synthetic construct; identical to SEQ ID NO: 1202
  REGION 1..538: note = Synthetic polypeptide

SEQ ID NO: 1204 - protein, 538 aa, synthetic construct; identical to SEQ ID NO: 1202
  REGION 1..538: note = Synthetic polypeptide

SEQ ID NO: 1205 - protein, 538 aa, synthetic construct; identical to SEQ ID NO: 1202
  REGION 1..538: note = Synthetic polypeptide

SEQ ID NO: 1206 - skipped

SEQ ID NO: 1207 - protein, 538 aa, synthetic construct
  REGION 1..538: note = Synthetic polypeptide
MGLLLLVLILTPSLAAYRHPDFPLLEKAQQLLQSTGSPYSTNCWLCTSSSTETPGTAYPASPREWTSIEAELHISYRWDPNLKGLMRPANSLLSTVKQDFPDIRQKPPIFGPIFTNINLMGIAPICVMAKRKNGTNVGTLPSTVCNVTFTVDSNQQTYQTYTHNQFRHQPRFPKPPNITFPQGTLLDKSSRFCQGRPSSCSTRNFWFRPADYNQCLQISNLSSTAEWVLLDQTRNSLFWENKTKGANQSQTPCVQVLAGMTIATSYLGISAVSEFFGTSLTPLFHFHISTCLKTQGAFYICGQSIHQCLPSNWTGTCTIGYVTPDIFIAPGNLSLPIPIYGNSPLPRVRRAIHFIPLLAGLGILAGTGTGIAGITKASLTYSQLSKEIANNIDTMAKALTTMQEQIDSLAAVVLQNRRGLDMLTAAQGGICLALDEKCCFWVNQSGKVQDNIRQLLNQASSLRERATQGWLNWEGTWKWFSWVLPLTGPLVSLLLLLLFGPCLLNLITQFVSSRLQAIKLQTNLSAGRHPRNIQESPF

SEQ ID NO: 1208 - skipped

SEQ ID NO: 1209 - protein, 159 aa, synthetic construct
  REGION 1..159: note = Synthetic polypeptide
MMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 1210 - protein, 228 aa, synthetic construct
  REGION 1..228: note = Synthetic polypeptide
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 1211 - protein, 374 aa, synthetic construct
  REGION 1..374: note = Synthetic polypeptide
MVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 1212 - skipped

SEQ ID NO: 1213 - protein, 528 aa, synthetic construct
  REGION 1..528: note = Synthetic polypeptide
MAKGEGAESGSAAGLLPTSILQSTERPAQKEPKKKKQQLSVCNKLCYALGGAPYQVTGCALGFFLQIYLLDVAQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 1214 - protein, 530 aa, synthetic construct
  REGION 1..530: note = Synthetic polypeptide
MAKGEGAESGSAAGLLPTSILQSTERPAQVKKEPKKKKQQLSVCNKLCYALGGAPYQVTGCALGFFLQIYLLDVAQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 1215 - protein, 415 aa, synthetic construct
  REGION 1..415: note = Synthetic polypeptide
MPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 1216 - protein, 543 aa, synthetic construct
  REGION 1..543: note = Synthetic polypeptide
MAKGEGAESGSAAGLLPTSILQSTERPAQVKKEPKKKKQQLSVCNKLCYALGGAPYQVTGCALGFFLQIYLLDVAQKDEEVVFCFSSFQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 1217 - protein, 502 aa, synthetic construct
  REGION 1..502: note = Synthetic polypeptide
MAKGEGAESGSAAGLLPTSILQSTERPAQVKKEPKKKKQQLSVCNKLCYALGGAPYQVTGCALGFFLQIYLLDVAQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 1218 - protein, 116 aa, synthetic construct
  REGION 1..116: note = Synthetic polypeptide
MSAPAQPPAEGTEGTAPGGGPPGPPPNMTSNRRLQQTQAQVEEVVDIIRVNVDKVLERDQKLSELDDRADALQAGASQFESSAAKLKRKYWWKNCKMMIMLGAICAIIVVVIVRRD

SEQ ID NO: 1219 - protein, 116 aa, synthetic construct; identical to SEQ ID NO: 1218
  REGION 1..116: note = Synthetic polypeptide

SEQ ID NO: 1220 - protein, 118 aa, synthetic construct
  REGION 1..118: note = Synthetic polypeptide
MSAPAQPPAEGTEGTAPGGGPPGPPPNMTSNRRLQQTQAQVEEVVDIIRVNVDKVLERDQKLSELDDRADALQAGASQFESSAAKLKRKYWWKNCKMMIMLGTICAIIVVVIVIYFFT

SEQ ID NO: 1221 - protein, 117 aa, synthetic construct
  REGION 1..117: note = Synthetic polypeptide
MSAPAQPPAEGTEGTAPGGGPPGPPPNMTSNRRLQQTQAQVEEVVDIIRVNVDKVLERDQKLSELDDRADALQAGASQFESSAAKLKRKYWWKNCKMMIMLGAICAIIVVVIVSKYR

SEQ ID NO: 1222 - protein, 118 aa, synthetic construct
  REGION 1..118: note = Synthetic polypeptide
MSAPAQPPAEGTEGTAPGGGPPGPPPNMTSNRRLQQTQAQVEEVVDIIRVNVDKVLERDQKLSELDDRADALQAGASQFESSAAKLKRKYWWKNCKMMIMLGAICAIIVVVIVIYFFT

SEQ ID NO: 1223 - protein, 117 aa, synthetic construct; identical to SEQ ID NO: 1221
  REGION 1..117: note = Synthetic polypeptide

SEQ ID NO: 1224 - protein, 116 aa, synthetic construct; identical to SEQ ID NO: 1218
  REGION 1..116: note = Synthetic polypeptide

SEQ ID NO: 1225 - protein, 116 aa, synthetic construct
  REGION 1..116: note = Synthetic polypeptide
MSAPAQPPAEGTEGTAPGGGPPGPPPNMTSNRRLQQTQAQVEEVVDIIRVNVDKVLERDQKLSELDDRADALQAGASQFESSAAKLKRKYWWKNCKMMIMLGAICAIIVVVIVRRG

SEQ ID NO: 1226 - skipped

SEQ ID NO: 1227 - protein, 118 aa, synthetic construct; identical to SEQ ID NO: 1222
  REGION 1..118: note = Synthetic polypeptide

SEQ ID NO: 1228 - protein, 338 aa, synthetic construct
  REGION 1..338: note = Synthetic polypeptide
MTETTVKTAAWFMANMQVSGGGPSISLVMKTPRVTKNEALWHPTLNLPLSPQGTVRTAVEFQVMTQTQSLSFLLGSSASLDCGFSMAPGLDLISVEWRLQHKGRGQLVYSWTAGQGQAVRKGATLEPAQLGMARDASLTLPGLTIQDEGTYICQITTSLYRAQQIIQLNIQASPKVRLSLANEALLPTLICDIAGYYPLDVVVTWTREELGGSPAQVSGASFSSLRQSVAGTYSISSSLTAEPGSAGATYTCQVTHISLEEPLGASTQVVPPERRTALGVIFASSLFLLALMFLGLQRRQAPTGLGLLQAERWETTSCADTQSSHLHEDRTARVSQPS

SEQ ID NO: 1229 - protein, 468 aa, synthetic construct
  REGION 1..468: note = Synthetic polypeptide
MGTQEGWCLLLCLALSGAAETKPHPAEGQWRAVDVVLDCFLAKDGAHRGALASSEDRARASLVLKQVPVLDDGSLEDFTDFQGGTLAQDDPPIIFEASVDLVQIPQAEALLHADCSGKEVTCEISRYFLQMTETTVKTAAWFMANMQVSGGGPSISLVMKTPRVTKNEALWHPTLNLPLSPQGTVRTAVEFQVMTQTQSLSFLLGSSASLDCGFSMAPGLDLISVEWRLQHKGRGQLVYSWTAGQGQAVRKGATLEPAQLGMARDASLTLPGLTIQDEGTYICQITTSLYRAQQIIQLNIQASPKVRLSLANEALLPTLICDIAGYYPLDVVVTWTREELGGSPAQVSGASFSSLRQSVAGTYSISSSLTAEPGSAGATYTCQVTHISLEEPLGASTQVVPPERRTALGVIFASSLFLLALMFLGLQRRQAPTGLGLLQAERWETTSCADTQSSHLHEDRTARVSQPS

SEQ ID NO: 1230 - skipped

SEQ ID NO: 1231 - protein, 118 aa, synthetic construct; identical to SEQ ID NO: 1222
  REGION 1..118: note = Synthetic polypeptide

SEQ ID NO: 1232 - protein, 117 aa, synthetic construct; identical to SEQ ID NO: 1221
  REGION 1..117: note = Synthetic polypeptide

SEQ ID NO: 1233 - protein, 118 aa, synthetic construct [sequence omitted]

SEQ ID NO: 1234-1236 - skipped (3 entries)

SEQ ID NO: 1237 - protein, 117 aa, synthetic construct
  REGION 1..117: note = Synthetic polypeptide
MSAPAQPPAEGTEGTAPGGGPPGPPPNMTSNRRLQQTQAQVEEVVDIIRVNVDKVLERDQKLSELDDRADALQAGASQFESSAAKLKRKYWWKNCKMMIMLGAICAIIVVVIVRRDD

SEQ ID NO: 1238 - protein, 116 aa, synthetic construct; identical to SEQ ID NO: 1218
  REGION 1..116: note = Synthetic polypeptide

SEQ ID NO: 1239 - protein, 116 aa, synthetic construct; identical to SEQ ID NO: 1218
  REGION 1..116: note = Synthetic polypeptide

SEQ ID NO: 1240 - protein, 116 aa, synthetic construct
  REGION 1..116: note = Synthetic polypeptide
MSATAATAPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFSS

SEQ ID NO: 1241 - protein, 116 aa, synthetic construct
  REGION 1..116: note = Synthetic polypeptide
MSATAATAPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFST

SEQ ID NO: 1242 - protein, 118 aa, synthetic construct
  REGION 1..118: note = Synthetic polypeptide
MDRSATAATAPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFST

SEQ ID NO: 1243 - protein, 116 aa, synthetic construct; identical to SEQ ID NO: 1241
  REGION 1..116: note = Synthetic polypeptide

SEQ ID NO: 1244 - protein, 116 aa, synthetic construct; identical to SEQ ID NO: 1241
  REGION 1..116: note = Synthetic polypeptide

SEQ ID NO: 1245 - protein, 116 aa, synthetic construct; identical to SEQ ID NO: 1241
  REGION 1..116: note = Synthetic polypeptide

SEQ ID NO: 1246 - protein, 116 aa, synthetic construct; identical to SEQ ID NO: 1240
  REGION 1..116: note = Synthetic polypeptide

SEQ ID NO: 1247 - protein, 116 aa, synthetic construct
  REGION 1..116: note = Synthetic polypeptide
MAGIELERCQQQANEVTEIMRNNFGKVLERGVKLAELQQRSDQLLDMSSTFNKTTQNLAQKKCWENIRYRICVGLVVVGVLLIILIVLLVVFLPQSSDSSSAPRTQDAGIASGPGN

SEQ ID NO: 1248 - skipped

SEQ ID NO: 1249 - protein, 116 aa, synthetic construct; identical to SEQ ID NO: 1241
  REGION 1..116: note = Synthetic polypeptide

SEQ ID NO: 1250 - skipped

SEQ ID NO: 1251 - protein, 100 aa, synthetic construct
  REGION 1..100: note = Synthetic polypeptide
MSTGPTAATGSNRRLQQTQNQVDEVVDIMRVNVDKVLERDQKLSELDDRADALQAGASQFETSAAKLKRKYWWKNCKMWAIGITVLVIFIIIIIVWVVSS

SEQ ID NO: 1252 - protein, 100 aa, synthetic construct; identical to SEQ ID NO: 1251
  REGION 1..100: note = Synthetic polypeptide

SEQ ID NO: 1253 - protein, 100 aa, synthetic construct
  REGION 1..100: note = Synthetic polypeptide
MSTGPTAATGSNRRLQQTQNQVDEVVDIMRVNVDKVLERDQKLSELDDRADALQAGASQFETSAAKLKRKYWWKNCEMWAIGITVLVIFIIIIIVWVVSS

SEQ ID NO: 1254 - protein, 100 aa, synthetic construct; identical to SEQ ID NO: 1251
  REGION 1..100: note = Synthetic polypeptide

SEQ ID NO: 1255 - protein, 100 aa, synthetic construct; identical to SEQ ID NO: 1251
  REGION 1..100: note = Synthetic polypeptide

SEQ ID NO: 1256 - protein, 122 aa, synthetic construct
  REGION 1..122: note = Synthetic polypeptide
MSTGPTAATGSNRRLQQTQNQVDEVVDIMRVNVDKVLERDQKLSELDDRADALQAGASQFETSAAKLKRKYWWKNCKMWAIGITVLVIFIIIIIGELPFSKLIGKVFSMCSQTIDFVYKNSD

SEQ ID NO: 1257 - protein, 100 aa, synthetic construct; identical to SEQ ID NO: 1251
  REGION 1..100: note = Synthetic polypeptide

SEQ ID NO: 1258 - skipped

SEQ ID NO: 1259 - protein, 116 aa, synthetic construct; identical to SEQ ID NO: 1247
  REGION 1..116: note = Synthetic polypeptide

SEQ ID NO: 1260 - protein, 100 aa, synthetic construct [sequence omitted]

SEQ ID NO: 1261 - protein, 116 aa, synthetic construct; identical to SEQ ID NO: 1247
  REGION 1..116: note = Synthetic polypeptide

SEQ ID NO: 1262 - protein, 116 aa, synthetic construct; identical to SEQ ID NO: 1247
  REGION 1..116: note = Synthetic polypeptide

SEQ ID NO: 1263 - protein, 260 aa, synthetic construct
  REGION 1..260: note = Synthetic polypeptide
MAILFAVVARGTTILAKHAWCGGNFLEVTEQILAKIPSENNKLTYSHGNYLFHYICQDRIVYLCITDDDFERSRAFNFLNEIKKRFQTTYGSRAQTALPYAMNSEFSSVLAAQLKHHSENKGLDKVMETQAQVDELKGIMVRNIVCHLQNYQQKSCSSHVYEEPQAHYYHHHRINCVHLYHCFTSLWWIYMAKLCEEIGKKKLPLTKDMREQGVKSNPCDSSLSHTDRWYLPVSSTLFSLFKILFHASRFIFVLSTSLFL

SEQ ID NO: 1264 - protein, 179 aa, synthetic construct
  REGION 1..179: note = Synthetic polypeptide
MAILFAVVARGTTILAKHAWCGGNFLEDFERSRAFNFLNEIKKRFQTTYGSRAQTALPYAMNSEFSSVLAAQLKHHSENKGLDKVMETQAQVDELKGIMVRNIDLVAQRGERLELLIDKTENLVDSSVTFKTTSRNLARAMCMKNLKLTIIIIIVSIVFIYIIVSPLCGGFTWPSCVKK

SEQ ID NO: 1265 - protein, 220 aa, synthetic construct
  REGION 1..220: note = Synthetic polypeptide
MAILFAVVARGTTILAKHAWCGGNFLEVTEQILAKIPSENNKLTYSHGNYLFHYICQDRIVYLCITDDDFERSRAFNFLNEIKKRFQTTYGSRAQTALPYAMNSEFSSVLAAQLKHHSENKGLDKVMETQAQVDELKGIMVRNIDLVAQRGERLELLIDKTENLVDSSVTFKTTSRNLARAMCMKNLKLTIIIIIVSIVFIYIIVSPLCGGFTWPSCVKK

SEQ ID NO: 1266 - protein, 220 aa, synthetic construct; identical to SEQ ID NO: 1265
  REGION 1..220: note = Synthetic polypeptide

SEQ ID NO: 1267 - protein, 220 aa, synthetic construct; identical to SEQ ID NO: 1265
  REGION 1..220: note = Synthetic polypeptide

SEQ ID NO: 1268 - protein, 100 aa, synthetic construct
  REGION 1..100: note = Synthetic polypeptide
MEEASEGGGNDRVRNLQSEVEGVKNIMTQNVERILARGENLEHLRNKTEDLEATSEHFKTTSQKVARKFWWKNVKMIVLICVIVFIIILFIVLFATGAFS

SEQ ID NO: 1269 - protein, 99 aa, synthetic construct
  REGION 1..99: note = Synthetic polypeptide
MEEASEGGGNDRVRNLQSEVEGVKNIMTQNVERILARGENLEHLRNKTEDLEATPGAEASCFREEVTPKLSKVWSLKFCVQRGGWLWLSESCVNSWLLV

SEQ ID NO: 1270 - protein, 100 aa, synthetic construct; identical to SEQ ID NO: 1268
  REGION 1..100: note = Synthetic polypeptide

SEQ ID NO: 1271 - protein, 566 aa, synthetic construct
  REGION 1..566: note = Synthetic polypeptide
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFINYEELREQLSSVSSFKRFEIFPKTSSWPNHDSNKGVTAACPHAGAKSFYKNLIWLVKKGNSYPKLSQSYINDKGKEVLVLWGIHHPSTTADQQSLYQNADAYVFVGTSRYSKKFKPEIAIRPKVRDQEGRMDYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVDKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 1272 - protein, 571 aa, synthetic construct
  REGION 1..571: note = Synthetic polypeptide
MDRAVNRVVLENEEREAKNTWRLVFRIAVLLLMVMTLAISAAALAYSMEASTPHDFAGISTVISKTEDKVTSLLSSSQDVIDRIYKQVALESPLALLNTESTIMNAITSLSYQINGAANNSGCGAPVHDPDYIGGIGKELIVDDISDVTSFYPSAYQEHLNFIPAPTTGFGCTRIPSFDMSTTHYCYTHNVILSGCRDHSHSHQYLALGVLRTSATGRVFFSTLHSINLDDAQNRKSCSVSATPLGCDMLCSKVTGTEEEDYKSAAPTSMVHGRLGFDGQYHEKDLDTTVLFKDWVANYPGVGGGSFIDDRVWFPVYGGLKPNSPSDTAQEGKYVIYKRHNNTCPDGQDYQIRMAKSSYKPGRFGGKRVQQAILSIKVSTSLGKDPVLTIPPNTITLMGAEGRILTVGTSHFLYQRGSSYFSPALLYPMTVNNKTATLHSPYTFNAFTRPGSVPCQASARCPNSCITGVYTDPYPLIFYRNHTLRGVFGTMLDAEQARLNPVSAVFDNISRSRVTRVSSSSTKAAYTTSTCFKVVKTNKAYCLSIAEISNTLFGEFRIVPLLVEILKDDRV

SEQ ID NO: 1273 - protein, 849 aa, synthetic construct
  REGION 1..849: note = Synthetic polypeptide
MRVMGIQKNYPLLWRWGMTIWGMAIFWIMIICNAEDLWVTVYYGVPVWKEAETTLFCASDAKAYDTEVHNVWATHACVPTDPNPQEIHLENVTENFNMWKNNMVEQMHEDVISLWDQSLKPCVKLTPLCVTLNCSNLNNITSNSSNSTSNNSTLSDIDMKNCSFIVTTELRDKKKREYALFYKQDVVQINENESNSSQYMLINCNTSTITQACPKVTFEPIPIHYCAPAGFAILKCNDREFNGTGPCENVSTVQCTHGIKPVVTTQLLLNGSTAEEEIIIRSEDIINNAKTIIVQLVKPVNITCVRPGNNTRKSVRIGPGQAFYTTGEVIGNIRKAYCIVNGTEWNDTFYRVAEKLAKYFNSKTIIFNQPSGGDVEITTHSFNCGGEFFYCNTSDLFNASSTWGNNITLPCRIKQIVNMWQKVGRAMYAPPIPGEIRCESNITGLLLTRDGGNNNNTNETFRPGGGDMRDNWRSELYKYKVVKIEPLGVAPTHAKRRVVQREKRAVGLGAVFLGFLGAAGSTMGAASITLTVQARQLLSGIVQQQSNLLKAIEAQQHLLRLTVWGIKQLQARVLALERYLKDQQLLGIWGCSGKLICHTTVPWNSSWSNKTFHDIWDNMTWLQWDKEISNYTSIIYGLIEESQNQQEKNEQDLLALDKWANLWNWFDITNWLWYIKIFIIIVGGLIGLRIIFAVLNIINRVRQGYSPLSFQTLTHHQREPDRPERIEEGGGEQDRDRSVRLVNGFLALAWDDLRSLCLFSYHQLRDFVLIVVRIVELLGHRGWEALKYLWNLLSYWGQELKNSAINLIDTTAIAVANWTDRVIEIGQRAGRAIRNIPTRIRQGLERALL

SEQ ID NO: 1274 - protein, 271 aa, synthetic construct
  REGION 1..271: note = Synthetic polypeptide
PVVSTQLLLNGSLAEEEVVIRSDNFTNNAKIIIVQLNESVNITCMRPNNNTRRDIHIGPGRAFYATDIIGDIRQAHCNLSEAKWNNTLKQIVTRLRDQFGNKTIVFNQSSGGDPEIVMHSFNCGGEFFFCNTNKLFNSIWHVNDTWNVTEKSNSTGGNETIILPCRIKQIINMWQEVGKAMYAPPIRGVIKCSSNITGLILTRDGGSGNGTTNNTETFRPGGGDMRDNWRSELYKYKVVQIEPLGIAPTRAKRRVVQREKRAVGIGALFLG

SEQ ID NO: 1275 - protein, 138 aa, synthetic construct
  REGION 1..138: note = Synthetic polypeptide
SGIVQQQSNLLLYIEAQQHLLKLTVWGIKQLQARVLAVERYLKDQQLLGIWGCSGKLICPTTVPWNSSWSNKSYSEIWDNMTWLQWDKEVNNYTQEIYRLIEQSQNQQEKNEQDLLALDKWANLWNWFDISSWLWYIK

SEQ ID NO: 1276 - protein, 3391 aa, synthetic construct
  REGION 1..3391: note = Synthetic polypeptide
MNDQRKKAKNTPFNMLKRERNRVSTVQQLTKRFSLGMLQGRGPLKLYMALVAFLRFLTIPPTAGILKRWGTIKKSKAINVLRGFRKEIGRMLNILNRRRRSAGMIIMLIPTVMAFHLTTRNGEPHMIVSRQEKGKSLLFKTEDGVNMCTLMAMDLGELCEDTITYKCPLLRQNEPEDIDCWCNSTSTWVTYGTCTTMGEHRRQKRSVALVPHVGMGLETRTETWMSSEGAWKHVQRIETWILRHPGFTMMAAILAYTIGTTHFQRALIFILLTAVTPSMTMRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSTTEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQADSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLIIGNMSFRDLGRVMVMVGATMTDDIGMGVTYLALLAAFKVRPTFAAGLLLRKLTSKALMMTTIGIVLSSQSTTPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGPLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLSKNGKKVIQLSRKTFDSEYAKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARTTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGILLFLMSGRGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPTTLTAALFLLVAHYAIIGPALQAKASREAQKRAAAGIMKNPTVDGITVIDLDPIPYDPKFEKQLGQVMLLVLCVTQVLMMRTTWALCEVLTLATGPISTLWEGNPGRFWNTTIAVSMANIFRGSYLAGAGLLFSIMKNTTNARRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVFRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNIMGKREKKLGEFGKAKGSRAIWYMWLGARFLEFEALGFLNEDHWFSRENSLSGVEGEGLHKLGYILRDVSKKEGGAMYADDTAGWDTRITLEDLKNEAMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRLPSALTALNDTGKIRKDIQQWEPSRGWNDWTQVPFCSHHFHELIMKDGRVLVVPCRNQDELIGRARISQGAGWSLRETACLGKSYDQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 1277 - protein, 495 aa, synthetic construct
  REGION 1..495: note = Synthetic polypeptide
MRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFLCSGKITGNLVQIENLEYTVVVTVHNGDTHAVGNDTSNHGVTATITPRSPSVEVKLPDYGELILDCEPRSGIDFNEMILMKMKKKTWLVHKQWFLDLPLPWTAGADTSELHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPFAENTNSVTNIELEPPFGDSYIVIGVGDSALTLHWFRKGSSIGKMFESTYRGAKRMAILGETAWDFGSVGGLFTSLGKAVHQVFGSVYTTMFGGVSWMVRILIGFLVLWIGTNSRNTSMAMTCIAVGGITLFLGFTVQA

SEQ ID NO: 1278 - protein, 501 aa, synthetic construct
  REGION 1..501: note = Synthetic polypeptide
FNCLGMSNRDFLEGVSGATWVDLVLEGDSCVTIMSKDKPTIDVKMMNMEAANLADVRSYCYLASVSDLSTRAACPTMGEAHNEKRADPAFVCKQGVVDRGWGNGCGLFGKGSIDTCAKFACTTKATGWIIQKENIKYEVAIFVHGPTTVESHGNYSTQIGATQAGRFSITPSAPSYTLKLGEYGEVTVDCEPRSGIDTSAYYVMSVGAKSFLVHREWFMDLNLPWSSAGSTTWRNRETLMEFEEPHATKQSVVALGSQEGALHQALAGAIPVEFSSNTVKLTSGHLKCRVKMEKLQLKGTTYGVCSKAFKFAGTPADTGHGTVVLELQYTGKDGPCKVPISSVASLNDLTPVGRLVTVNPFVSVATANSKVLIELEPPFGDSYIVVGRGEQQINHHWHKSGSSIGKAFTTTLRGAQRLAALGDTAWDFGSVGGVFTSVGKAIHQVFGGAFRSLFGGMSWITQGLLGALLLWMGINARDRSIAMTFLAVGGVLLFLSVNVHA

SEQ ID NO: 1279 - protein, 511 aa, synthetic construct
  REGION 1..511: note = Synthetic polypeptide
MKCLLYLAFLFIGVNCKFTIVFPHNQKGTWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNDICPTVHNSTTWHSDYKVKGLCDSNLISTDITFFSEDGELSSLGKGGTGFRSNHFAYETGDKACKMQYCRHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIVAPILSRMVGMISGTTTERELWDDWAPYEDVEIGPNGVLRTSSGYKFPLYMVGHGMLDSDLHLSSKAQVFEHPHIQDAASQLPDDETLFFGDTGLSKNPIELVEGWFSGWKSSIASFFFIIGLIIGLFLVLRVGIYLCIKLKHTKKRQIYTDIEMNRLGK

SEQ ID NO: 1280 - protein, 517 aa, synthetic construct
  REGION 1..517: note = Synthetic polypeptide
MLSYLILAIIVSPILGKIEIVFPQHTTGDWKRVPHEYNYCPTSADKNSHGTQTGIPVELTMPKGLTTHQVDGFMCHSALWMTTCDFRWYGPKYITHSIHNEEPTDYQCLEAIKAYNDGVSFNPGFPPQSCGYGTVTDAEAHIITVTPHSVKVDEYTGEWIDPHFIGGRCKGKICETVHNSTKWFTSSDGESVCSQLFTLVGGTFFSDSEEITSMGLPETGMRSNYFPYISTEGICKMPFCRKPGYKLKNDLWFQITDPDLDKTVRDLPHIKDCDLSSSIITPGEHATDISLISDVERILDYALCQNTWSKIEAGEPITPVDLSYLGPKNPGVGPVFTVINGSLHYFTSKYLRVELESPVIPRMEGRVAGTKIVRQLWDQWFPFGEAEIGPNGVLKTKQGYKFPLHIIGTGEVDSDIKMERIVKHWEHPHIEAAQTFLKKDDTEEVIYYGDTGVSKNPVELVEGWFSGWRSSIMGVVAVIIGFVILIFLIRLIGVLSSLFRQKRRPIYKSDVEMTHFR

SEQ ID NO: 1281 - protein, 171 aa, synthetic construct
  REGION 1..171: note = Synthetic polypeptide
EMADKALFAAARFPECPEGSSISAPSQTSVDVSLIQDIERILDYSLCQETWSKIRAGLPISPVDLSYLAPKNPGTGPVFTIINGTLKYFETRYIRVDIAAPILSRMVGMISGTTTERVLWDDWAPYEDVEIGPNGVLRTSSGYKFPLYMIGHGMLDSDLHLSSKAQVFEHP

SEQ ID NO: 1282 - protein, 171 aa, synthetic construct; identical to SEQ ID NO: 1281
  REGION 1..171: note = Synthetic polypeptide

SEQ ID NO: 1283 - protein, 171 aa, synthetic construct
  REGION 1..171: note = Synthetic polypeptide
EMADKALFAAARFPECPEGSSISAPSQTSVDVSLIQDIERILDYSLCQETWSKIRARLPISPVDLSYLAPKNPGTGPVFTIINGTLKYFETRYIRVDIAAPILSRMVGMISGTTTERVLWDDWAPYEDVEIGPNGVLRTSSGYKFPLYMIGHGMLDSDLHLSSKAQVFEHP

SEQ ID NO: 1284 - protein, 171 aa, synthetic construct
  REGION 1..171: note = Synthetic polypeptide
EMADKALFAAARFPECPEGSSISAPSQTSVDVSLIQDVERILDYSLCQETWSKIRAGLPISPVDLSYLAPKNPGTGPVFTIINGTLKYFETRYIRVDIAAPILSRMVGMISGTTTERVLWDDWAPYEDVEIGPNGVLRTSSGYKFPLYMIGHGMLDSDLHLSSKAQVFEHP

SEQ ID NO: 1285 - protein, 171 aa, synthetic construct; identical to SEQ ID NO: 1281
  REGION 1..171: note = Synthetic polypeptide

SEQ ID NO: 1286 - protein, 524 aa, synthetic construct
  REGION 1..524: note = Synthetic polypeptide
MVPQALLFVPLLVFPLCFGKFPIYTIPDKLGPWSPIDIHHLRCPNNLVVEDEGCTNLSGFSYMELKVGYILAIKMNGFTCTGVVTEAETYTNFVGYVTTTFKRKHFRPTPDACRAAYNWKMAGDPRYEESLHNPYPDYRWLRTVKTTKESLVIISPSVADLDPYDRSLHSRVFPSGKCSGVAVSSTYCSTNHDYTIWMPENPRLGMSCDIFTNSRGKRASKGSETCGFVDERGLYKSLKGACKLKLCGVLGLRLMDGTWVAMQTSNETKWCPPDQLVNLHDFRSDEIEHLVVEELVRKREECLDVLESIMTTKSVSFRRLSHLRKLVPGFGKAYTIFNKTLMEADAHYKSVRTWNEILPSKGCLRVGGRCHPHVNGVFFNGIILGPDGNVLIPEMQSSLLQQHMELLESSVIPLVHPLADPSTVFKDGDEAEDFVEVHLPDVHNQVSGVDLGLPNWGKYVLLSAGALTALMLIIFLMTCCRRVNRSEPTQHNLRGTGREVSVTPQSGKIISSWESHKSGGETRL

SEQ ID NO: 1287 - protein, 524 aa, synthetic construct
  REGION 1..524: note = Synthetic polypeptide
MILQALLFVPLLISSLCLGKFPIYTIPDKLGPWSPIDIHHLSCPNNLVVEDEGCTNLSGFSYMELKVGYISAIKVNGFTCTGVVTEAETYTNFVGYVTTTFKRKHFRPIPDACRAAYNWKMAGDPRYEESLQNPYPDYHWLRTVKTTKESLIIISPSVADLDPYDKSLHSRVFPGGKCLGITVSSTYCSTNHDYTIWMPEEPRLGTSCDIFTSSKGKKASKGGKTCGFVDERGLYKSLKGACKLKLCGVLGLRLMDGTWVSIQTSDDTKWCPSDQLVNLHDFHSDEIEHLVVEELVKKREECLDALESIMTTKSVSFRRLSYLRKLVPGFGKAYTIFNKTLMEADAHYKSVRTWNEIIPSKGCLKVRERCHPPVDGVFFNGIILGPDGNVLIPEMQSSLLQQHMELLESSVIPLMHPLADPSTVFKEGDEAEDFVEVHLPDVHKQISGVDLGLPSWGKYLLMIAGGLAALVLIICSMACCRRTKRTESRRRGSGESEKKVTATPQTRKVVSSWELYKSEGDARL

SEQ ID NO: 1288 - protein, 524 aa, synthetic construct
  REGION 1..524: note = Synthetic polypeptide
MIFQALLFVPLLISSLCLGKFPIYTIPDKLGPWSPIDIHHLSCPNNLVVEDEGCTNLSGFSYMELKVGYISAIKVNGFTCTGVVTEAETYTNFVGYVTTTFKRKHFRPIPDACRAAYNWKMAGDPRYEESLQNPYPDYHWLRTVKTTKESLIIISPSVADLDPYDKSLHSRVFPSGKCLGITVSSTYCSTNHDYTIWMPEEPRLGTSCDIFTSSKGKKASKGGKTCGFVDERGLYKSLKGACKLKLCGVLGLRLMDGTWVSIQTSDDTKWCPPDQLVNLHDFHSDEIEHLVVEELVKKREECLDALESIMTTKSVSFRRLSYLRKLVPGFGKAYTIFNKTLMEADAHYKSVRTWNEIIPSKGCLKVRERCHPPVDRVFFNGIILGPDGNVLIPEMQSSLLQQHMELLESSVIPLMHPLADPSTVFKEGDEAEDFVEVHLPDVHKQISGVDLGLPSWGKYLLMIAGGLATLVLIICSMACCRRTKRTESRRRGSRESEKKVTAAPQTRKVVSSWELYKSKGDARL

SEQ ID NO: 1289 - protein, 524 aa, synthetic construct
  REGION 1..524: note = Synthetic polypeptide
MILQALLFVPLLISSLCLGKFPIYTIPDKLGPWSPIDIHHLSCPNNLVVEDEGCTNLSGFSYMELKVGYISAIKVNGFTCTGVVTEAETYTNFVGYVTTTFKRKHFRPIPDACRAAYNWKMAGDPRYEESLQNPYPDYHWLRTVKTTKESLIIISPSVADLDPYDKSLHSRVFPGGKCLGITVSSTYCSTNHDYTIWMPEEPRLGTSCDIFTSSKGKKASKGGKTCGFVDERGLYKSLKGACKLKLCGVLGLRLMDGTWVSIQTSDDTKWCPPDQLVNLHDFHSDEIEHLVVEELVKKREECLDALESIMTTKSVSFRRLSYLRKLVPGFGKAYTIFNKTLMEADAHYKSVRTWNEIIPSKGCLKVRERCHPPVDGVFFNGIILVPDGNVLIPEMQSSLLQQHMELLESSVIPLMHPLADPSTVFKEGDEAEDFVEVHLPDVHKQISGVDLGLPSWGKYLLMIAGGLAALVLIICSMACCRRTKRTESRRRGSGESEKKVTATPQTRKVVSSWELYKSEGDARL

SEQ ID NO: 1290 - protein, 904 aa, synthetic construct
  REGION 1..904: note = Synthetic polypeptide
MRQGAPARGCRWFVVWALLGLTLGVLVASAAPSSPGTPGVAAATQAANGGPATPAPPALGAAPTGDPKPKKNKKPKNPTPPRPAGDNATVAAGHATLREHLRDIKAESTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMEHTEHKAKKKGTSALLSAKVTDMVMRKRRNTNYTQVPNKDGDADEDDL

SEQ ID NO: 1291 - protein, 904 aa, synthetic construct
  REGION 1..904: note = Synthetic polypeptide
MRQGAPARGRRWFVVWALLGLTLGVLVVSAAPSSPGTPGVAAATQAANGGPATPAPPAPGAPPTGDPKPKKNKKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMERTEHKAKKKGTSALLSAKVTDMVMRKRRNTNYTQVPNKDGDADEDDL

SEQ ID NO: 1292 - protein, 904 aa, synthetic construct
  REGION 1..904: note = Synthetic polypeptide
MRQGAPARGRRWFVVWALLGLTLGVLVASAAPSSPGTPGVAAATQAANGGPATPAPPAPGAPPTGDPKPKKNKKPKPPKPPRPAGDNATVAAGHATLREHLRDIKAENTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYNEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSVMERTEHKAKKKGTSALLSAKVTDMVMRKRRNTNYTQVPNKDGDADEDDL

SEQ ID NO: 1293 - protein, 676 aa, synthetic construct
  REGION 1..676: note = Synthetic polypeptide
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 1294 - protein, 676 aa, synthetic construct
  REGION 1..676: note = Synthetic polypeptide
MGGLSLLQLPRDKFRKSSFFVWVIILFQKAFSMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKIDNNTFVRLDRPHTPQFLFQLNDTIHLHQQLSNTTGRLIWTLDANINADIGEWAFWENKKNLSEQLRGEELSFEALSLNETEDDDAASSRITKGRISDRATRQYSDLVPKNPPGMVPLHIPEGETTLPSQNSTEGRRVSVNTQETITETAATIIGTNGNHMQISTIGIRPSSSQIPSSSPTTAPSPEAQTPTTHTSGPSVMATEEPTTPPGSSPGPTTEAPTLTTPENITTAVKTVLPQESTSNGLITSTVTGILGSLGLRKRSRRQTNTKATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 1295 - protein, 83 aa, synthetic construct
  REGION 1..83: note = Synthetic polypeptide
FRTGVPPKNVEYTEGEEAKTCYNISVTDPSGKSLLLDPPTNVRDYPKCKTIHHIQGQNPHAQGIALHLWGAFFLYDRIASTTM

SEQ ID NO: 1296 - protein, 81 aa, synthetic construct
  REGION 1..81: note = Synthetic polypeptide
RTGVPPKNVEYTEGEEAKTCYNISVTDPSGKSLLLDPPTNVRDYPKCKTIHHIQGQNPHAQGIALHLWGAFFLYDRIASTT

SEQ ID NO: 1297 - protein, 149 aa, synthetic construct
  REGION 1..149: note = Synthetic polypeptide
GQKVADSPLEASKRWAFRTGVPPKNVEYTEGEEAKTCYNISVTDPSGKSLLLDPPTNVRDYPKCKTIHHIQGQNPHAQGIALHLWGAFFLYDRIASTTMYRGKVFTEGNIAAMIVNKTVHKMIFSRQGQGYRHMNLTSTNKYWTSSNGT

SEQ ID NO: 1298 - protein, 681 aa, synthetic construct
  REGION 1..681: note = Synthetic polypeptide
MKTTCFLISLILIQGTKNLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSDWGVLTNLGILLLLSIAVLIALSCICRIFTKYIG

SEQ ID NO: 1299 - protein, 681 aa, synthetic construct
  REGION 1..681: note = Synthetic polypeptide
MRTTCFFISLILIQGIKTLPILEIASNDQPQNVDSVCSGTLQKTEDVHLMGFTLSGQKVADSPLEASKRWAFRTGVPPKNVEYTEGEEAKTCYNISVTDPSGKSLLLDPPTNVRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGTLQEYNSTKNQTCTPSKTPPPPPTAYPEIKPTSTSTDATRLNTTNPNSDDEDLTTSGSGSGEQEPDTTSDAVTKQGLSSTMPPTPSPQPGTPQQGGNNTNHSQDAATELDNTNTTAQPPTPSHNTTTISTNNTSKHNLSTLSELPQNTTNPNTQSMATENEKTSASPKTTLPPTESPTTEKSTNNTKSPTTMEPNTTNGHFTSPSSTPNSTTQHLIYFRRKRSILWREGDMFPFLDGLINAPIDFDPVPNTKTIFDESSSSGASAKEDQHASSNISLTLSYLPHTSENTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSRNISEQIDQIKKDEQKEGTGWGLGGKWWTSDWGVLTNLGILLLLSIAVLIALSCICRIFTKYIG

SEQ ID NO: 1300 - protein, 681 aa, synthetic construct
  REGION 1..681: note = Synthetic polypeptide
MKTIYFLISLILIQSIKTLPVLEIASNSQPQDVDSVCSGTLQKTEDVHLMGFTLSGQKVADSPLEASKRWAFRTGVPPKNVEYTEGEEAKTCYNISVTDPSGKSLLLDPPSNIRDYPKCKTVHHIQGQNPHAQGIALHLWGAFFLYDRVASTTMYRGKVFTEGNIAAMIVNKTVHRMIFSRQGQGYRHMNLTSTNKYWTSSNETQRNDTGCFGILQEYNSTNNQTCPPSLKPPSLPTVTPSIHSTNTQINTAKSGTMNPSSDDEDLMISGSGSGEQGPHTTLNVVTEQKQSSTILSTPSLHPSTSQHEQNSTNPSRHAVTEHNGTDPTTQPATLLNNTNTTPTYNTLKYNLSTPSPPTRNITNNDTQRELAESEQTNAQLNTTLDPTENPTTGQDTNSTTNIIMTTSDITSKHPTNSSPDSSPTTRPPIYFRKKRSIFWKEGDIFPFLDGLINTEIDFDPIPNTETIFDESPSFNTSTNEEQHTPPNISLTFSYFPDKNGDTAYSGENENDCDAELRIWSVQEDDLAAGLSWIPFFGPGIEGLYTAGLIKNQNNLVCRLRRLANQTAKSLELLLRVTTEERTFSLINRHAIDFLLTRWGGTCKVLGPDCCIGIEDLSKNISEQIDKIRKDEQKEETGWGLGGKWWTSDWGVLTNLGILLLLSIAVLIALSCICRIFTKYIG

SEQ ID NO: 1301 - protein, 221 aa, synthetic construct
  REGION 1..221: note = Synthetic polypeptide
MGTLVAKLLLPTLSSLAFLPTVSIAAKRRFHMEAMVYLFTLFFVALHHACNGPGLSVLCFMRHDILEYFSVYGTALSMWVSLMALADFDEPKRSTFVMFGVLTIAVRIYHDRWGYGVYSGPIGTAILIIAAKWLQKMKEKKGLYPDKSVYTQQIGPGLCFGALALMLRFFFEDWDYTYVHSFYHCALAMSFVLLLPKVNKKAGSPGTPAKLDCSTLCCACV

SEQ ID NO: 1302 - protein, 84 aa, synthetic construct
  REGION 1..84: note = Synthetic polypeptide
MPTPLLPLLLRLLLSCLLLPAARLARQYLLPLLRRLARRLGSQDMREALLGCLLFILSQRHSPDAGEASRVDRLERRERLGPQK

SEQ ID NO: 1303 - protein, 84 aa, synthetic construct; identical to SEQ ID NO: 1302
  REGION 1..84: note = Synthetic polypeptide

SEQ ID NO: 1304 - protein, 783 aa, synthetic construct
  REGION 1..783: note = Synthetic polypeptide
MSTFIRRYSRYLNEKAFSYRQMAFDFARVKKGADGVMRTMAPEKLLKSMPILQGQIDALLEFDVHPNELTNGVINAAFMLLFKDLIKLFACYNDGVINLLEKFFEMKKGQCKDALEIYKRFLTRMTRVSEFLKVAEQVGIDKGDIPDLTQAPSSLMETLEQHLNTLEGKKPGNNEGSGAPSPLSKSSPATTVTSPNSTPAKTIDTSPPVDLFATASAAVPVSTSKPSSDLLDLQPDFSSGGAAAAAAPAPPPPAGGATAWGDLLGEDSLAALSSVPSEAQISDPFAPEPTPPTTTAEIATASASASTTTTVTAVTAEVDLFGDAFAASPGEAPAASEGAAAPATPTPVAAALDACSGNDPFAPSEGSAEAAPELDLFAMKPPETSVPVVTPTASTAPPVPATAPSPAPAVAAAAAATTAATAAATTTTTTSAATATTAPPALDIFGDLFESTPEVAAAPKPDAAPSIDLFSTDAFSSPPQGASPVPESSLTADLLSVDAFAAPSPATTASPAKVDSSGVIDLFGDAFGSSASEPQPASQAASSSSASADLLAGFGGSFMAPSPSPVTPAQNNLLQPNFEAAFGTTPSTSSSSSFDPSGDLLMPTMAPAGQPAPVSMVPPSPAMAASKALGSDLDSSLASLVGNLGISGTTTKKGDLQWNAGEKKLTGGANWQPKVAPATWSAGVPPSAPLQGAVPPTSSVPPVAGAPSVGQPGAGFGMPPAGTGMPMMPQQPVMFAQPMMRPPFGAAAVPGTQLSPSPTPASQSPKKPPAKDPLADLNIKDFL

SEQ ID NO: 1305 - protein, 907 aa, synthetic construct
  REGION 1..907: note = Synthetic polypeptide
MSGQTLTDRIAAAQYSVTGSAVARAVCKATTHEVMGPKKKHLDYLIQATNETNVNIPQMADTLFERATNSSWVVVFKALVTTHHLMVHGNERFIQYLASRNTLFNLSNFLDKSGSHGYDMSTFIRRYSRYLNEKAFSYRQMAFDFARVKKGADGVMRTMAPEKLLKSMPILQGQIDALLEFDVHPNELTNGVINAAFMLLFKDLIKLFACYNDGVINLLEKFFEMKKGQCKDALEIYKRFLTRMTRVSEFLKVAEQVGIDKGDIPDLTQAPSSLMETLEQHLNTLEGKKPGNNEGSGAPSPLSKSSPATTVTSPNSTPAKTIDTSPPVDLFATASAAVPVSTSKPSSDLLDLQPDFSSGGAAAAAAPAPPPPAGGATAWGDLLGEDSLAALSSVPSEAQISDPFAPEPTPPTTTAEIATASASASTTTTVTAVTAEVDLFGDAFAASPGEAPAASEGAAAPATPTPVAAALDACSGNDPFAPSEGSAEAAPELDLFAMKPPETSVPVVTPTASTAPPVPATAPSPAPAVAAAAAATTAATAAATTTTTTSAATATTAPPALDIFGDLFESTPEVAAAPKPDAAPSIDLFSTDAFSSPPQGASPVPESSLTADLLSVDAFAAPSPATTASPAKVDSSGVIDLFGDAFGSSASEPQPASQAASSSSASADLLAGFGGSFMAPSPSPVTPAQNNLLQPNFEAAFGTTPSTSSSSSFDPSVFDGLGDLLMPTMAPAGQPAPVSMVPPSPAMAASKALGSDLDSSLASLVGNLGISGTTTKKGDLQWNAGEKKLTGGANWQPKVAPATWSAGVPPSAPLQGAVPPTSSVPPVAGAPSVGQPGAGFGMPPAGTGMPMMPQQPVMFAQPMMRPPFGAAAVPGTQLSPSPTPASQSPKKPPAKDPLADLNIKDFL

SEQ ID NO: 1306-1500 - skipped (195 entries)

SEQ ID NO: 1501 - protein, 233 aa, unidentified
  source 1..233: note = TNF polypeptide sequence
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 1502 - protein, 35 aa, unidentified
  source 1..35: note = TNF polypeptide sequence
MSTESMIRDVELAEEALPKKTGGPQGSRRCLFLSL

SEQ ID NO: 1503 - protein, 177 aa, unidentified
  source 1..177: note = TNF polypeptide sequence
GPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 1504 - protein, 205 aa, unidentified
  source 1..205: note = TNF polypeptide sequence
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 1505 - protein, 244 aa, unidentified
  source 1..244: note = TNF polypeptide sequence
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 1506 - protein, 18 aa, unidentified
  source 1..18: note = TNF polypeptide sequence
MGALGLEGRGGRLQGRGS

SEQ ID NO: 1507 - protein, 196 aa, unidentified
  source 1..196: note = TNF polypeptide sequence
QDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 1508 - protein, 240 aa, unidentified
  source 1..240: note = TNF polypeptide sequence
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 1509 - protein, 37 aa, unidentified
  source 1..37: note = TNF polypeptide sequence
MEESVVRPSVFVVDGQTDIPFTRLGRSHRRQSCSVAR

SEQ ID NO: 1510 - protein, 182 aa, unidentified
  source 1..182: note = TNF polypeptide sequence
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 1511 - protein, 167 aa, unidentified
  source 1..167: note = TNF polypeptide sequence
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 1512-1600 - skipped (89 entries)

SEQ ID NO: 1601 - protein, 349 aa, unidentified
  source 1..349: note = CTGF polypeptide sequence
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 1602 - protein, 348 aa, unidentified
  source 1..348: note = CTGF polypeptide sequence
MLASVAGPISLALVLLALCTRPAMGQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 1603-1700 - skipped (98 entries)

SEQ ID NO: 1701 - protein, 115 aa, synthetic construct
  REGION 1..115: note = Synthetic polypeptide
ESGGGSVQAGGSLRLSCAASGYTCSSYCMGWFRQAPGMGREGVARIGRSGVQYEDSVKGRFTISRDNAKNTLYLQMNSLPEDTAMYYCASRPFAWCFEWSFNDWGQGTQVTVSSA

SEQ ID NO: 1702 - protein, 6 aa, synthetic construct
  REGION 1..6: note = Synthetic 6xHis tag
HHHHHH

SEQ ID NO: 1703 - RNA, 120 nt, synthetic construct
  misc_feature 1..120: note = Synthetic polynucleotide
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa